SEQUENCE LISTING (7 sequences)

SEQ ID NO: 1 - RNA, 47 nt, synthetic construct
  source 1..47: mol_type = other RNA
gggaaagcagtgtagcggacggtgtgtcagttcaacccacgacagag

SEQ ID NO: 2 - RNA, 45 nt, synthetic construct
  source 1..45: mol_type = other RNA
gggaaagagtgtagcggacggtgtgtcagttcaacccacacagag

SEQ ID NO: 3 - DNA, 21 bp, synthetic construct
  source 1..21: mol_type = other DNA
ctctgtcgtgggttgaactga

SEQ ID NO: 4 - DNA, 19 bp, synthetic construct
  source 1..19: mol_type = other DNA
gggaaagcagtgtagcgga

SEQ ID NO: 5 - DNA, 47 bp, synthetic construct
  source 1..47: mol_type = other DNA
ctctgtcgtgggttgaactgacacaccgtccgctacactgctttccc

SEQ ID NO: 6 - DNA, 20 bp, synthetic construct
  source 1..20: mol_type = other DNA
gctctcatacgaactcgtcc

SEQ ID NO: 7 - DNA, 20 bp, synthetic construct
  source 1..20: mol_type = other DNA
gtctctgctcgactaaccac

(19) United States
(12) Patent Application Publication
(10) Pub. No.: US 2026/0201363 A1
(43) Pub. Date: Jul. 16, 2026

(54) COMPOSITIONS CONTAINING OLIGONUCLEOTIDES WITH THERANOSTIC APPLICATIONS

(71) Applicant: SIXFOLD BIOSCIENCE LTD., London (GB)

(72) Inventors: Salesia WERNER, London (GB); James Luke RUSHWORTH, London (GB); Ioanna MYLONAKI, London (GB); Auréle LACROIX, London (GB); Piotr KLIMKOWSKI, London (GB); George William FOOT, Dorchester (GB); Anna Perdrix ROSELL, London (GB)

(73) Assignee: SIXFOLD BIOSCIENCE LTD., London (GB)

(21) Appl. No.: 19/116,662

(22) PCT Filed: Sep. 29, 2023
(86) PCT No.: PCT/IB2023/000592

§ 371 Date: Mar. 28, 2025

(51) Int. Cl. C12N 15/10

(52) U.S. Cl. CPC C12N 15/1065

(57) ABSTRACT

The disclosure provides therapeutic compositions that include theranostic nucleic acid nanostructures for delivery of CA cargo and methods of using the same.

### FIELD OF THE DISCLOSURE

The disclosure relates generally to therapeutic compositions that include nucleic acid nanoparticles for delivery of cargo and methods of using the same.

### BACKGROUND

Nucleic Acids Therapies (NATs) are single or double stranded oligonucleotides that are exploited as modalities for a variety therapeutic and/or vaccine purposes. NATs include many different types of modalities, including, but not limited to, DNA based gene therapies, RNA interference (RNAi), microRNAs (miRNAs), antisense oligonucleotides (ASO), long non-coding RNA (lncRNA), messenger RNA (mRNA), aptamers, and self-amplifying RNA (saRNA). Compared to small molecule and antibody-based drugs, NATs hold tremendous potential in drugging the “undruggable”. NATs can act on virtually unrestricted choice of otherwise “undruggable” therapeutic targets, with high specificity and potency, while their ease of manufacturing facilitates rapid lead optimization. Therefore, NATs have the potential to effectively treat numerous indications. However, the major limiting factor for further success remains the lack of effective platforms for systemic delivery of NATs to specific diseased tissue. Current approaches, (GalNAc-conjugates, lipid nanoparticles and viral vectors), are sub-optimal given their limited cell targeting specificity beyond the liver or local administration, cargo loading capacity, high toxicity, and complex/expensive manufacturing that limit addressable disease indications (K. Paunovska, D. Loughrey, J. E. Dahlman, Drug delivery systems for RNA therapeutics, Nat. Rev. Genet. 0123456789 (2022). https://doi.org/10.1038/s41576-021-00439-4.).

Nucleic Acids (NA) can address the aforementioned limitations by providing an opportunity to assemble structures with precise control of their properties at the nanoscale. These properties can be fine-tuned and can provide an additional level of stability over their linear counterparts.

NA nanoparticles have been shown to be able to carry a wide range of cargo, including therapeutics, targeting molecules and imaging molecules. Despite these advances, the delivery of these carriers to specific tissues still remains a major challenge. Analysis of subsequent nanoparticle uptake to the cell cytosol is also a challenge; less than 3% of a nucleic-acid based construct reaches a target cell and escapes to the cytoplasm.

The compositions herein aim to address these challenges by disclosing modifications and structures to allow for efficient modulation of pharmacokinetic/pharmacodynamic (PK/PD) properties, combined with efficient imaging and barcoding techniques to ascertain whether the desired biological outcome, such as distribution to tissues, has been achieved.

### SUMMARY

The following compositions are designed with the aim of improving the biodistribution, stability and function of nucleic acid therapies (NATs) by linking to one or more oligonucleotides, followed by subsequent monitoring of these two parameters in an in vitro and in vivo setting. The present disclosure also provides methods that include modification and cleavage of oligonucleotides.

The compositions and methods of the disclosure provide a novel nucleic acid (NA) barcodes conjugated to, or a part of, gymnotic nucleic acids, such as GalNAc-conjugates or peptide-conjugates, where a NA barcode attached to the conjugate is not encapsulated, for example, in a lipid nanoparticle formulation. Generally, non-encapsulated nucleic acids rapidly degrade by the naturally occurring nucleases found in biological material, including when administered as a therapy in vivo. Although, NA barcodes are used in the development of lipid nanoparticulate delivery systems, where the encapsulation of minimally modified NA barcodes ensures the stability of NA strands against nuclease degradation, the disclosure is based on present Inventors' surprising discovery of methods for incorporating NA barcodes onto gymnotic nucleic acids and other “exposed” delivery systems, which are able to withstand nuclease degradation in biological environments. The present disclosure also finds use in methods whereby the identification of chemical entities in a biological context would be useful, including but not limited to CRISPR, proteins, peptides and NA material.

In certain aspects, the present disclosure provides a composition comprising: a nucleic acid analyte further comprising a nucleic acid barcode, wherein said nucleic acid barcode allows for the detection and unambiguous identification of the nucleic acid analyte in a biological sample, and wherein said nucleic acid barcode is not encapsulated in a lipid nanoparticle formulation. In certain aspects, said nucleic acid analyte and/or said nucleic acid barcode comprise DNA, RNA, XNA, or a combination thereof.

In certain aspects, the nucleic acid barcode is between 5-500 nucleotides in length, and preferably 5-30 nucleotides in length. In certain compositions, said nucleic acid barcode is between 6-12 nucleotides in length. In certain aspects, said nucleic acid barcode comprises a single-stranded portion, a double-stranded portion, a triple-stranded portion, a quadruple-stranded portion, a quintuple-stranded portion or combinations thereof.

The nucleic acid barcode may be single-stranded or is double-stranded.

In some embodiments, the nucleic acid barcode comprises one or more modified nucleotides, and wherein each of said one or more modified nucleotides is independently modified at the ribose group, the phosphate group, the nucleobase group, or a combination thereof. In certain embodiments, each nucleotide of said nucleic acid barcode is a modified nucleotide.

Preferred modifications at the ribose group include, and may be independently, for each occurrence, selected from 2′-O-methyl, 2′-fluoro, 2′-F-arabino, 2′-methoxyethyl, 2′-amino, 2′-deoxy, 2′-O-allyl, locked nucleic acid, unlocked nucleic acid, 2′,4′-constrained 2′-O-ethyl-bridged nucleic acid, arabinose, hexose, cyclohexenyl nucleic acid, hexitol nucleic acid, glycol nucleic acid, 4′-thioribonucleoside, and 4′-C-aminomethyl-2′-O-methyl. In certain aspects, the modification at the phosphate group is independently for each occurrence selected from phosphorothioate, phosphorodithioate, alkylated phosphorothioate, boranophosphate, methylphosphonate, phosphoramidate, or amide backbone. In certain embodiments, the modification at the nucleobase group is independently for each occurence selected from 5-methylcytosine, 5-hydroxymethylcytosine, 5-methyluracil, 5-ribosyluracil (pseudouracil), 7-methylguanine, inosine, xanthine, hypoxanthine, 3-methylcytidine, dihydrouridine, N6-methyldeoxyadenosine, N4-methylcytosine, 5-hydroxymethylcytosine, 5-formylcytosine, and 5-carboxylcytosine.

In preferred aspects, the nucleic acid barcode is a suboptimal substrate for nuclease-mediated degradation from one or more nucleases having enzyme commission numbers ranging from EC 3.1.11 to EC 3.1.31, such that the nucleic acid barcode is substantially protected from nuclease-mediated. In certain aspects, the nucleic acid barcode is a viable substrate of one or more nucleic acid binding enzymes having enzyme commission numbers other than EC 3.1.11 to EC 3.1.31. The one or more nucleic acid binding enzymes may be ligases that form phosphoric-ester bonds (E.C. 3.5.1). The one or more nucleic acid binding enzymes may be nucleotidyltransferases (E.C. 2.7.7). The nucleotidyltransferases may be selected from DNA-directed RNA polymerase (EC 2.7.7.6), DNA-directed DNA polymerase (EC 2.7.7.7), polynucleotide adenylyltransferase (EC 2.7.7.19), DNA nucleotidylexotransferase (terminal deoxyribonucleotidyl transferase) (EC 2.7.7.31), RNA-directed RNA polymerase (EC 2.7.7.48), RNA-directed DNA polymerase (reverse transcriptase) (EC 2.7.7.49), mRNA guanylyltransferase (mRNA capping enzyme) (EC 2.7.7.50), and combinations and recombinant and engineered versions thereof. In certain aspects, one or more nucleic acid binding enzymes are one or more polynucleotide 5′-hydroxyl-kinase (EC 2.7.1.78). In some embodiments, said nucleic acid barcode comprises alternating 2′-O-methyl and 2′-fluoro nucleotides.

In some embodiments, said nucleic acid barcode comprises at least 2, 3, 4, 5, 6, 7, 8, 9, or 10 contiguous 2′-O-methyl nucleotides.

In some embodiments, said nucleic acid barcode comprises at least 2, 3, 4, 5, 6, 7, 8, 9, or 10 consecutive 2′-methoxyethyl nucleotides. In some embodiments, said nucleic acid barcode comprises at least one locked nucleic acid nucleotide. In some embodiments, each internucleotide linkage of said nucleic acid barcode is a phosphodiester internucleotide linkage or a phosphorothioate internucleotide linkage. In some embodiments, at least one internucleotide linkage of said nucleic acid barcode is a phosphorothioate internucleotide linkage. In some embodiments, the nucleotides at positions 1-2, 1-3, 1-4, 1-5, 1-6, 1-7 or 1-8 from the 3′ end of said nucleic acid barcode are connected to adjacent nucleotides via phosphorothioate linkages. In some embodiments, the nucleotides at positions 1-2, 1-3, 1-4, 1-5, 1-6, 1-7 or 1-8 from the 5′ end of said nucleic acid barcode are connected to adjacent nucleotides via phosphorothioate linkages. In some embodiments, the terminal 2, 3, 4, 5, 6, 7, 8, 9 or 10 nucleotides at both the 5′ and 3′ terminus of said nucleic acid barcode, independently, are linked with phosphorothioate internucleotide linkages.

In preferred aspects, the nucleic acid analyte comprises a nucleic acid therapy (NAT). In preferred aspects, the nucleic acid analyte comprises a functional sequence which is recognized by a complementary hybridization or capture probe. In preferred aspects, the nucleic acid analyte is covalently conjugated to the nucleic acid barcode. In preferred aspects, the nucleic acid analyte is hybridized to the nucleic acid barcode. In preferred aspects, the nucleic acid barcode sequence is integrated within the sequence of the nucleic acid analyte.

In preferred aspects, the biological sample is a sample obtained from a person that received formulation comprising the nucleic acid analyte. In preferred aspects, the nucleic acid barcode of the nucleic acid analyte in the formulation is not encapsulated in a lipid nanoparticle formulation. In preferred aspects, the nucleic acid analyte in the formulation is not encapsulated in a lipid nanoparticle formulation.

In preferred aspects, the biological sample comprises cells. In preferred aspects, cells in the biological sample internalize the nucleic acid analyte.

The present disclosure further provides a composition comprising: (i) a nucleic acid nanostructure self-assembled from two or more oligonucleotide strands that are not nucleic acid therapies (NATs), said nanostructure not encapsulated in the lipid nanoparticle formulation; (ii) a cargo molecule that is linked to said nucleic acid nanostructure; and (iii) optionally a nucleic acid barcode that is part of or linked to said nucleic acid nanostructure.

In certain aspects, each of the two or more oligonucleotide strands of said nucleic acid nanostructure, independently, comprises DNA, RNA, XNA or a combination thereof.

In some embodiments, at least one of the two or more oligonucleotide strands of said nucleic acid nanostructure comprises one or more modified nucleotides, and wherein each of said one or more modified nucleotides is independently modified at the ribose group, the phosphate group, the nucleobase group, or a combination thereof. In some embodiments, each of the at least one modified nucleotides is independently selected from a 2′-O-methyl modified nucleotide, a 2′-fluoro modified nucleotide, a 2′-deoxy nucleotide, a 2′-hydroxyl nucleotide, a 2′-F-arabino nucleotide, a 2′-methoxyethyl modified nucleotide, a locked nucleotide, an unlocked nucleotide, a constrained ethyl nucleotide [2′,4′-constrained 2′-O-ethyl-bridged nucleic acid], a 2′-amino-modified nucleotide, a 2′-O-allyl modified nucleotide, a morpholino nucleotide, a phosphoramidate nucleotide, an arabinose modified nucleotide, a cyclohexenyl modified nucleotide, a hexose modified nucleotide, a hexitol modified nucleotide, an altritol modified nucleotide, a glycol modified nucleotide, a 4′ thiol modified nucleotide, a 4′-C-aminomethyl-2′-O-methyl modified nucleotide, a nucleotide comprising a phosphorothioate group, a nucleotide comprising a phosphorodithioate group, a nucleotide comprising an alkylated phosphorothioate group, a nucleotide comprising a methylphosphonate group, a nucleotide comprising a boranophosphate group, a nucleotide comprising a phosphoramidate group, a nucleotide comprising an amide group, a nucleotide comprising a 5′-phosphate, a 5-methylcytosine nucleotide, a 5-hydroxymethylcytosine nucleotide, a 5-methyluracil nucleotide, a 5-ribosyluracil (pseudouracil) nucleotide, a 7-methylguanine nucleotide, an inosine nucleotide, a xanthine nucleotide, a hypoxanthine nucleotide, a 3-methylcytidine nucleotide, a dihydrouridine nucleotide, a N6-methyldeoxyadenosine nucleotide, a N4-methylcytosine nucleotide, a 5-hydroxymethylcytosine nucleotide, a 5-formylcytosine nucleotdie, a 5-carboxylcytosine nucleotide, a 3-terminal deoxy-thymine (dT) nucleotide, an abasic nucleotide, and combinations thereof.

In certain aspects, each nucleotide of the nucleic acid nanostructure is modified; or each nucleotide of the nucleic acid nanostructure and/or each nucleotide of the barcode is modified. In certain compositions, the nucleic acid nanostructure comprises one or more 2′O-modified nucleotides, one or more phosphorothioate internucleotide linkages or a combination thereof. In certain aspects, the one or more 2′O modifications, the one or more phosphorothioate internucleotide linkages or the combination of both are used to modulate the physicochemical properties of the nucleic acid nanostructure.

In some embodiments, between 1 and 500 cargo molecules are linked to said nucleic acid nanostructure. In certain aspects, 1, 2, 3, 4, 5, 6, 7, 8, 9 or 10 cargo molecules are linked to said nucleic acid nanostructure. In certain aspects, each of the at least two or more oligonucleotide strands of the nucleic acid nanostructure is independently linked to one cargo molecule.In certain aspects, the cargo molecule is selected from the group comprising one or more NAT, nucleic acid barcode, peptide, protein, antibody, therapeutic small molecule, lipid, cholesterol, synthetic polymer, amino acid, amino acid analogue, PEG and hydrocarbon chain.

In preferred aspects, at least one of the two or more oligonucleotides of said nucleic acid nanostructure is linked to one or more NAT. The one or more NAT may be, for example, an ASO, an siRNA, a gRNA/CRISPR, an saRNA, or an mRNA. In certain aspects, the cargo molecule comprises a NAT and a nucleic acid barcode.

In certain aspects, at least one of the two or more oligonucleotide strands of the nucleic acid nanostructure is conjugated to the cargo molecule via a click reaction selected from the group comprising CuAAC, SPAAC, RuAAC, IEDDA, SuFEx, SPANC, hydrazone/oxime ether formation, thiol-ene radical reaction, thiol-yne radical reaction, thiol-Michael addition reaction, thiol-isocyanate reaction, thiol-epoxide click reaction, nucleophilic ring opening reaction (spring-loaded reactions), and traceless Staudinger ligation. In certain aspects, the nucleic acid nanostructure is conjugated to the cargo molecule via CuAAC. In certain aspects, the nucleic acid nanostructure is conjugated to the cargo molecule via IEDDA.

In certain aspects, the nucleic acid nanostructure comprises at least one structural motif selected from duplex, triplex, quadruplex, hairpin loop, internal loop/bulge/helix-loop-helix (such as kink-turn, S-turn T-loop), multi-branched loop/multi-junction, pseudoknot, kissing loop, A-minor, A-A platform, tetraloop, ribose zipper and combinations thereof. In certain aspects, at least one of the two or more oligonucleotide strands of said nucleic acid nanostructure has either partial or complete sequence complementarity to at least one other oligonucleotide of the nucleic acid nanostructure. In certain aspects, the nucleic acid nanostructure is self-assembled from two oligonucleotide strands and comprises a single duplex motif. In certain aspects, the nucleic acid nanostructure is self-assembled from three or more oligonucleotide strands and comprises a single duplex motif. In certain aspects, the nucleic acid nanostructure is self-assembled from two or more oligonucleotide strands and comprises at least one duplex motif and at least one hairpin loop motif. In certain aspects, the nucleic acid nanostructure is self-assembled from n (or more) oligonucleotide strands and comprises one n-way junction motif, where n can be any integer between 3 and 20. In certain aspects, the nucleic acid nanostructure is self-assembled from 10 or fewer oligonucleotide strands and comprises two or more n-way junction motifs, where n can be any integer between 3 and 20. In certain aspects, the nucleic acid nanostructure comprises one or more circularized oligonucleotide strands.

In certain aspects, the cargo molecule is the nucleic acid barcode. In certain aspects, the nucleic acid barcode is incorporated within at least one of the two or more oligonucleotide strands of the nucleic acid nanostructure. In certain aspects, the nucleic acid barcode is incorporated at the 5′ or the 3′ terminus within at least one of the two or more oligonucleotide strands of the nucleic acid nanostructure. In certain aspects, the nucleic acid barcode is incorporated in an internal loop or bulge motif of the nucleic acid nanostructure. In certain aspects, nucleic acid barcode and its reverse complement are incorporated in a duplex motif of the nucleic acid nanostructure.

In certain aspects, each of the two or more oligonucleotide strands of said nucleic acid nanostructure, independently, are between 5 to 500 nucleotides in length. In certain aspects, each of the two or more oligonucleotide strands of said nucleic acid nanostructure, independently, are between 5 to 80 nucleotides in length. In certain aspects, each of the two or more oligonucleotide strands of said nucleic acid nanostructure, independently, are no more than 35 nucleotides in length.

The present disclosure also provides a composition comprising an oligonucleotide of formula (I), or a salt thereof:

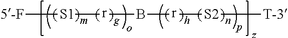

wherein: F is an optional 5′ terminal cargo that is either absent or present; T is an optional 3′ terminal cargo that is either absent or present; z is 1 or any positive integer greater than 1; o and p are each independently, and independently for each occurrence, 0 or any positive integer greater than 0; m, g, h, and n are each independently, and independently for each occurrence, 0 or 1; for any o>1, g≤m for each but the first occurrence; for any p>1, h≤n for each but the first occurrence; S1 and S2 are nucleotide sequences, each independently comprising at least 1 and not more than 100,000 nucleotides in length, where for any o>1 or p>1 or z>1 or combinations thereof: (i) each occurrence of S1, independently, represents a nucleic acid sequence identical to or different to the nucleic acid sequence of the first occurrence of S1; (ii) each occurrence of S2, independently, represents a nucleic acid sequence identical to or different to the nucleic acid sequence of the first occurrence of S2; r is a covalent or non-covalent linkage, where for any o>1 or p>1 or z>1 or combinations thereof each occurrence of r represents a linkage identical to or different to the linkage of the first occurrence of r; and B is a nucleic acid barcode comprising a nucleic acid sequence, where for z>1 each occurrence of B, independently, comprises a nucleic acid sequence identical to or different to the nucleic acid sequence of the first occurrence of B; and wherein said oligonucleotide of formula (I) is not encapsulated in a lipid nanoparticle formulation.

In some embodiments, the oligonucleotide of formula (I) comprises a NAT. In some embodiments, the oligonucleotide of formula (I) comprises one or more modified nucleotides, and wherein each of said one or more modified nucleotides is independently modified at the ribose group, the phosphate group, the nucleobase group, or a combination thereof.

In some embodiments, r is a bioconjugation linkage formed as product of a conjugation reaction, and is independently for each occurrence selected from an acid anhydride, an alkane, an alkene, an alkyne, an amide, an amine, a disulfide, a dithiourethane, an ether, ethylene glycol, an ester, a glucosyl, a hydrazone, an imine, an iminophosphorane, an isoxazoline, a ketone, an oxime, a phosphodiester, a phosphoramidate, a polyamide, a pyridazine, a pyrrolidine, a sulfonate ester, a sulfonamide, a thioether, a thiourethane, and a triazole linkage. In some embodiments, r comprises a cleavable linkage. In some embodiments, r is a hybridization linkage.

In some embodiments, S1 and/or S2 independently comprise a nucleic acid sequence selected from a NAT, a nucleic acid nanostructure, a random nucleotide sequence, a synthetic non-therapeutic nucleic acid, a probe hybridization sequence, a primer binding sequence, a cellular RNA sequence and combinations thereof. In some embodiments, the nucleic acid sequence of at least one of S1 and S2 independently comprises an mRNA, gRNA/CRISPR, saRNA, siRNA, ASO, miRNA, ribozyme, or aptamer sequence. In some embodiments, the nucleic acid sequence of at least one of S1 and S2 independently comprises an oligonucleotide sequence of a nucleic acid nanostructure self-assembled from two or more oligonucleotide strands that are not NATs.

In some embodiments: (i) the nucleic acid sequence of at least one of S1 and S2 independently comprises a unique molecular identifier sequence of random nucleotides; and (ii) each random nucleotide of the unique molecular identifier is optionally modified at the ribose group, the phosphate group, the nucleobase group or combinations thereof.

In some embodiments, the nucleic acid sequence of at least one of S1 and S2 independently comprises a sequence complementary to a hybridisation probe selected from a biotinylated capture probe, a molecular beacon probe, a ligation probe, a Scorpion probe, a TaqMan probe, an LNA probe, a cycling probe, and an RNAscope probe. In some embodiments, wherein the nucleic acid sequence of at least one of S1 and S2, independently, is designed to have no homology with endogenous nucleic acid sequences of the biological subject of interest. In some embodiments, the nucleic acid sequence at least one of S1 and S2, independently, comprises one or more modified nucleotides.

In some embodiments, the optional cargo molecules F and T are, independently, selected from the group comprising NAT, peptide, protein, antibody, small molecule, lipid, cholesterol, synthetic polymer. amino acid, amino acid analogue, fluorophore, multivalent moiety, PEG and hydrocarbon chain. In some embodiments, at least one of said cargo molecules F and T, independently, comprises one or more GalNAc derivatives attached through a monovalent, bivalent or trivalent linker. In some embodiments, at least one of said cargo molecules F and T, independently, comprises one or more lipid derivatives attached through a cleavable or non-cleavable linker. In some embodiments, at least one of said cargo molecules F and T, independently, comprises a cell-penetrating peptide through a cleavable or non-cleavable linker. In some embodiments, F comprises a nucleotide modified with a 5′ terminal modification selected from 5′ App, 5′ spacer, 5′ fluorophore, 5′ quencher, 5′ phosphate, 5′ biotin, 5′ digoxigenin and chemical analogs thereof.

In some embodiments, T comprises a nucleotide modified with a 3′ terminal modification selected from 3′ inverted dT, 3′ spacer, 3′ fluorophore, 3′ quencher, 3′ phosphate, 3′ biotin and chemical analogs thereof.

In some embodiments, z is 1. In some embodiments, o is 0 and p is 1. In some embodiments, h is 0 and n is 1. In some embodiments, both h and n are 1. In some embodiments, o is 1 and p is 0. In some embodiments, m is 1 and g is 0. In some embodiments, both m and g are 1. In some embodiments, both o and p are 1. In some embodiments, both m and n are 1, and both g and h are 0. In some embodiments, both m and n are 1, and g is 1, and h is 0. In some embodiments, both m and n are 1, and g is 0, and h is 1. In some embodiments, (i) the nucleic acid sequence of at least one of S1 and S2 comprises one or more modified nucleotides arranged in a modification pattern; (ii) B is of the same length as either S1 or S2; and (ii) B comprises the same modification pattern as either S1, S2 or both S1 and S2.

In some embodiments, the oligonucleotide of formula (I) comprises part of a library of barcoded nucleic acid analytes for use in a multiplex screening assay.

In some embodiments, each of the barcoded nucleic acid analytes of said library comprises a NAT for use in a multiplex assay that achieves screening for the delivery of NATs. In some embodiments, each of the barcoded nucleic acid analytes of said library comprises a nucleic acid barcode sequence B that differs in at least one nucleotide from all other sequences of B. In some embodiments, B comprises the sequence of a NAT selected from ASO, siRNA, saRNA and gRNA/CRISPR, such that a plurality of different NAT sequences can be screened in a multiplex assay.

In some embodiments, z is 1 and both o and p are 0.
In some embodiments, B comprises an identifier sequence that corresponds to a particular modification pattern of the nucleic acid sequence of S1, S2 or both S1 and S2 for allowing screening of a plurality of differently modified oligonucleotides in a multiplex assay. In some embodiments, B comprises an identifier sequence that corresponds to a particular cargo molecule F or T or both for allowing screening of a plurality of oligonucleotides linked to different cargo molecules in a multiplex assay.
The disclosure also provides, a composition comprising: an alkyl phosphonamidite of formula (II):

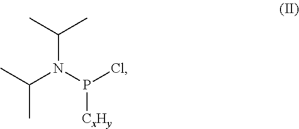

wherein CₓH_{Y} denotes an alkyl chain with the general formula CₙH₂ₙ₋₁, where x can be anywhere between 1 and 20 and x can be anywhere between 3 and 39. In some embodiments, the methyl phosphonamidite is incorporated into an oligonucleotide. In some embodiments, the oligonucleotide is incorporated into a nucleic acid nanostructure.

The present disclosure further provides acomposition comprising:
an alkyl phosphonamidite of formula (III):

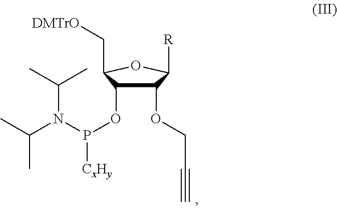

wherein R is a nucleotide base and CₓH_{Y} denotes an alkyl chain with the general formula CₙH₂ₙ₋₁, where x can be anywhere between 1 and 20 and x can be anywhere between 3 and 39. In some embodiments, the methyl phosphonamidite is incorporated into an oligonucleotide. In some embodiments, the oligonucleotide is incorporated into a nucleic acid nanostructure.

The present disclosure also provides a composition comprising: a phosphitylating reagent of formula (IV):

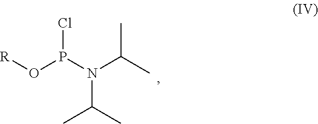

wherein R is a moiety that can modulate the physicochemical properties of an oligonucleotide. This includes moieties selected from the group consisting of O-alkylamino, O-alkylalkoxy, protected O-alkylamino, O-alkylaminoalkyl, O-alkyl imidazole, O-alkyl guanidine, polyamines of the formula (O-alkyl)ₘ, where m is about 1 to 20 and polyethers of the formula (O-alkyl)ₘ, where m is about 1 to 20. Preferred ethers are polyethylene glycols (PEGs).

In some embodiments, the phosphitylating reagent is used to convert an alcohol-bearing moiety to a phosphoramidite of formula (V):

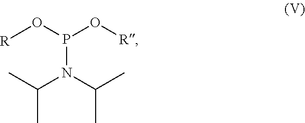

wherein R is a moiety that can modulate the physicochemical properties of an oligonucleotide. This includes moieties selected from the group consisting of O-alkylamino, O-alkylalkoxy, protected O-alkylamino, O-alkylaminoalkyl, O-alkyl imidazole, O-alkyl guanidine, polyamines of the formula (O-alkyl)ₘ, where m is about 1 to 20 and polyethers of the formula (O-alkyl)ₘ, where m is about 1 to 20. Preferred ethers are polyethylene glycols (PEGs). R″ is the alcohol-bearing molecule. R″ could be any molecule selected from the group consisting of, but not limited to, nucleotides, nucleosides, alkyl chains, PEGs, lipids, cholesterols, polymers, peptides, amino acids.

In some embodiments, wherein the phosphoramidite is incorporated into an oligonucleotide. In some embodiments, the oligonucleotide is incorporated into a nucleic acid nanostructure.

In some embodiments, there is a labile linker between the modification (R), and the P(III) center, as given by general formula (VI):

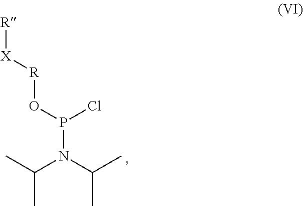

wherein R is a moiety that can modulate the physicochemical properties of an oligonucleotide. This includes moieties selected from the group consisting of O-alkylamino, O-alkylalkoxy, protected O-alkylamino, O-alkylaminoalkyl, O-alkyl imidazole, O-alkyl guanidine, polyamines of the formula (O-alkyl)ₘ, where m is about 1 to 20. X is a labile linker. Linkers include those, but are not limited to, moieties selected from the group consisting of thiol cleavable linkers such as dithiobismaleimidoethane, 1,4-bis[3-(2-pyridyldithio)propionamido]butane and 3-(2-pyridyldithio)propionyl hydrazide or base-cleavable linkers such as bis[2-(N-succinimidyl-oxycarbonyloxy)ethyl]sulfone or hydroxylamine-cleavable linkers such as (ethylene glycol bis(succinimidyl succinate)), oximes, hydrazones or cathepsin-responsive dipeptides. R″ is a moiety that can mask the properties of R. This includes moieties from the group consisting of, but is not limited to, polyethers of the formula (O-alkyl)ₘ, where m is about 1 to 20, carbohydrates and hydrophilic peptides.

The present disclosure also provides, a composition comprising: a nucleic acid nanostructure not formulated in a lipid nanoparticle comprising or linked to a nucleic acid barcode, and optionally, a cargo molecule that is covalently linked to the nucleic acid nanostructure.

In some embodiments, the cargo molecule is selected from the group consisting of a NAT, nucleic acid barcode, peptide, protein, antibody, therapeutic small molecule, lipid, cholesterol, synthetic polymer, amino acid, amino acid analogue, PEGS and hydrocarbon chain. In some embodiments, the cargo molecule is covalently linked to the nucleic acid nanostructure by a cleavable linker. In some embodiments, the cleavable linker is a dipeptide selected from the group consisting of Tyr-Arg, Phe-Arg, Val-Gln, Val-Cit, Tyr-Met, Leu-Gln, Val-Arg, Met-Thr, Phe-Gln, Thr-Thr, Val-Thr, Ala-Ala, Val-Met, Leu-Met, Ala-Asn, Val-Ala, Phe-Met. In some embodiments, the cleavable linker is cleaved enzymatically by an enzyme selected from the group consisting of cathepsin A, cathepsin B, cathepsin, C, cathepsin D, cathepsin E, cathepsin F, cathepsin G, cathepsin H, cathepsin K, cathepsin L1, cathepsin L2, cathepsin O, cathepsin S, cathepsin W and cathepsin Z.

In some embodiments, the dipeptide is further attached to a moiety that will allow for traceless release of the cargo. In some embodiments, the moiety that allows for traceless release of the cargo is para-aminocarbamate. In some embodiments, the moiety that allows for traceless release is selected from the group consisting of benzyl carbamate, hydroxylbenzylamine, hydrazone, disulfide and pyrophosphate diester. In some embodiments, the dipeptide unit has a phosphoramidite incorporated within the structure to allow for attachment to the oligonucleotide by solid-phase oligonucleotide synthesis. In some embodiments, the dipeptide unit has a reactive click handle incorporated within the structure to allow for attachment to the oligonucleotide via click chemistry. In some embodiments, the dipeptide unit has a further reactive click handle incorporated within the structure to allow for attachment to the cargo via click chemistry.

In some embodiments, the reactive is groups are orthogonal to each other and are reactive in a reaction selected from the group consisting of CuAAC, SPAAC, RuAAC, IEDDA, SuFEx, SPANC, hydrazone/oxime ether formation, thiol-ene radical reaction, thiol-yne radical reaction, thiol-Michael addition reaction, thiol-isocyanate reaction, thiol-epoxide click reaction, nucleophilic ring opening reaction (spring-loaded reactions), and traceless Staudinger ligation. In some embodiments, the dipeptide unit has both a phosphoramidite moiety and a click moiety. The click moiety is reactive in a reaction selected from the group consisting of CuAAC, SPAAC, RuAAC, IEDDA, SuFEx, SPANC, hydrazone/oxime ether formation, thiol-ene radical reaction, thiol-yne radical reaction, thiol-Michael addition reaction, thiol-isocyanate reaction, thiol-epoxide click reaction, nucleophilic ring opening reaction (spring-loaded reactions), and traceless Staudinger ligation.

The present disclosure also provides a composition comprising: a nucleic acid nanostructure, and a layer of hydrophilic molecules that are electrostatically bound to the nucleic acid phosphate backbone. In some embodiments, the hydrophilic layer consists of multiple molecules with the same identity. This number is given by n(P)-X, whereby n(P) is the number of phosphate moieties in the molecule and X can be any number between 1 and n(P). In some embodiments, the hydrophilic layer consists of multiple molecules with different identities. There could be 1, 2, 3, 4, 5, 6, 7, 8, 9, 10 or more different types of molecules in varied molar ratios.

In some embodiments, the nucleic acid nanostructure comprises DNA, RNA, or xeno-nucleic acid (XNA). In some embodiments, the electrostatic molecule contains a primary amine that can displace 4-(hexyloxy)anilinium on the nucleic acid phosphate backbone. In some embodiments, the electrostatic molecule is a PEG molecule with the general formula (VII):

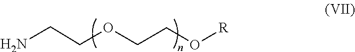

wherein R is H or Me.

In some embodiments, the electrostatic molecule is a PEG molecule with the general formula (VIII):

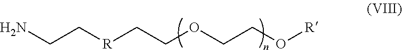

In some embodiments, R is a functional group that will allow for cleavage of the hydrocarbonchain in response to a range of stimuli, selected from the group consisting of disulfide, hydrazone, hydrazine, acetal, benzoic imine or thioketal. In some embodiments, R′ is a hydrogen or methyl group.

In some embodiments, the electrostatic molecule is a PEG molecule with the general formula (IX):

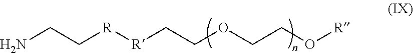

In some embodiments, R is defined as the “physicochemical modulator”, which is subsequently masked by the PEG moiety. This moiety can include, but is not limited to, spermine, ethylenediamine, methylethylenediamine, ethylethylenediamine, imidazole, spermine-imidazole-4-imine, N-ethyl-N′-(3-dimethylaminopropyl)-guanidinyl ethylene imine, dimethylaminoethyl acrylate, amino vinyl ether, 4-imidazoleacetic acid, diethylaminopropylamide, sulfonamides (e.g. sulfadimethoxine sulfamethoxazole, sulfadiazine, sulfamethazine), amino ketals, N-2-hydroxylpropyltimehyl ammonium chloride, imidazole-4-imines, methyl-imidazoles, 2-(aminomethyl)imidazole, 4-(aminomethyl)imidazole, 4(5)-(hydroxymethyl)imidazole, N-(2-aminoethyl)-3-((2-aminoethyl)(methyl)amino)propanamide, 2-(2-ethoxyethoxy)ethan-1-amine, bis(3-aminopropyl)amine, [N,N-dimethylamino)ethoxy]ethyl, N-(2-aminoethyl)-3-((2-aminoethyl)(ethyl)amino)propanamide, (N-(aminoethyl)carbamoyl)methyl, N-(2-((2-aminoethyl)amino)ethyl)acetamide 3,3′-((2-aminoethyl)azanediyl)bis(N-(2-aminoethyl)propanamide), guanidyl benzylamide, [3-(guanidinium)propyl], dimethylethanolamine, 1-(2,2-dimethyl-1,3-dioxolan-4-yl)-N,N-dimethylmethanamine, 2-(2,2-dimethyl-1,3-dioxolan-4-yl)-N,N-dimethylethan-1-amine, N-(2-((2-(2-aminoethoxy)propan-2-yl)oxy)ethyl)acetamide, aminobutyl, aminoethyl, 1-(2-aminoethyl)-3-(3-(dimethylamino)propyl)-2-ethylguanidine, 1-(3-amino-3-oxopropyl)-2,4,6-trimethylpyridin-1-ium, 1-(1,3-bis(carboxyoxy)propan-2-yl)-2,4,6-trimethylpyridin-1-ium, guanidinylethyl amine, ether hydroxyl triazole, or a β-aminoester or an alkyl chain between 1 and 30 carbons in length.

In some embodiments, R′ is defined as a functional group that will allow for cleavage of the hydrocarbon chain in response to a range of stimuli, selected from the group consisting of disulfide, hydrazone, hydrazine, acetal, benzoic imine or thioketal.

In some embodiments, the electrostatic molecule is a branched PEG molecule with the general formula:

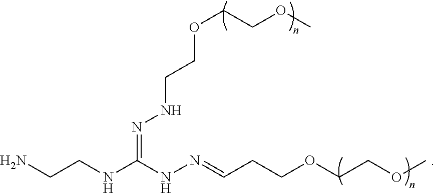

In some embodiments, the PEG chain length (n) could be anywhere between 1, 2, 3, 4, 5, 6, 7, 8, 9, 10, 15, 20, 30 units long or more.

In some embodiments, the electrostatic molecules are instead covalently bound to the nucleic acid nanostructure. In some embodiments, the electrostatic molecule is a sugar molecule that has been functionalized with a primary amine.

In some embodiments, the sugar molecule is selected from the group consisting of D-Ribulose 5-phosphate, D-Mannuronic acid, L-Fuconic acid, 1-Deoxy-D-xylulose-5-phosphate, D-Fructose-1,2-cyclic-6-bisphosphate, D-Erythritol 4-phosphate, D-galactose 6-phosphate, 2-Deoxy-D-ribonic acid, 6-Phosphogluconic acid, s-L-Fucose 1-phosphate bis(cyclohexylammonium), D-Fructose 1,6-bisphosphate, D-Mannose 6-phosphate, D-Xylonic acid, L-Glyceric acid, L-Threonic acid, 6-phospho-D-galactonate, N-Acetyl-D-glucosamine, D-Glyceric acid, D-Mannose, D-Ribose, Agaric acid, D-Mannitol, Dulcitol, L-Sorbose, D-Glucose diethylmercaptal, Dhurrin, Levoglucosenone, α-Homonojirimycin, Arbutin, Fusicoccin, L-(−)-Dithiothreitol, L-(+)-Ribose, D-(−)-Lactic acid, D-(−)-Tagatose, D-Mannoheptose, D-Psicose, D-(+)-Fucose, D-(+)-Sorbose, DL-Arabinose, L-(−)-Galactose, L-(+)-Erythrulose, L-(−)-Fucose, 3-Deoxyglucosone, D-(+)-Arabitol, D-Mannoheptulose, DL-Xylose, epi-Inositol, L-(−)-Glucose, L-(−)-Mannose, L-(+)-Gulose, Lactulose, Methyl α-D-galactopyranoside, 1,5-Anhydro-D-sorbitol, Isopropyl β-D-thioglucopyranoside, L(+)-Erythrose, Methyl-sP-D-galactopyranoside, Methyl-β-D-thiogalactoside, N-Acetylmuramic acid, β-D-Allose, D-(+)-Talose, D-Glucosaminic acid, Deoxyfuconojirimycin, 2-Deoxy-L-ribose, L-Galactono-1,4-lactone, L-Idaro-1,4-lactone, N-Acetyl-D-glucosamine, (−)-Sinigrin hydrate, 5-Thio-D-glucose, D-Threono-1,4-lactone, D-Xylono-1,4-lactone, D-(−)-Erythrose, L-Threono-1,4-lactone, Methyl α-D-mannopyranoside, D-Glucosone, N-Glycolylneuraminic acid, 2-Deoxy-D-ribose, 3-O-Methyl-D-glucopyranose, D-Glucosamine 3-sulfate, D-Altrose, D-(−)-Fructose, D-(+)-Xylose, Glycerol, D-Galactose, L-(−)-Sorbose, meso-Erythritol, Methyl α-D-glucopyranoside, Dextrose, L-Rhamnose, N-Acetyl-D-galactosamine, L-(+)-Arabinose.

In some embodiments, the electrostatic molecule is an oligosaccharide, consisting of 0, 1, 2, 3, 4, 5, 6, 7, 8, 9, 10, 15, 20 repeating units or more. In some embodiments, the monomeric sugar molecule is selected from the group consisting of D-Ribulose 5-phosphate, D-Mannuronic acid, L-Fuconic acid, 1-Deoxy-D-xylulose-5-phosphate, D-Fructose-1,2-cyclic-6-bisphosphate, D-Erythritol 4-phosphate, D-galactose 6-phosphate, 2-Deoxy-D-ribonic acid, 6-Phosphogluconic acid, β-L-Fucose 1-phosphate bis(cyclohexylammonium), D-Fructose 1,6-bisphosphate, D-Mannose 6-phosphate, D-Xylonic acid, L-Glyceric acid, L-Threonic acid, 6-phospho-D-galactonate, N-Acetyl-D-glucosamine, D-Glyceric acid, D-Mannose, D-Ribose, Agaric acid, D-Mannitol, Dulcitol, L-Sorbose, D-Glucose diethylmercaptal, Dhurrin, Levoglucosenone, α-Homonojirimycin, Arbutin, Fusicoccin, L-(−)-Dithiothreitol, L-(+)-Ribose, D-(−)-Lactic acid, D-(−)-Tagatose, D-Mannoheptose, D-Psicose, D-(+)-Fucose, D-(+)-Sorbose, DL-Arabinose, L-(−)-Galactose, L-(+)-Erythrulose, L-(−)-Fucose, 3-Deoxyglucosone, D-(+)-Arabitol, D-Mannoheptulose, DL-Xylose, epi-Inositol, L-(−)-Glucose, L-(−)-Mannose, L-(+)-Gulose, Lactulose, Methyl α-D-galactopyranoside, 1,5-Anhydro-D-sorbitol, Isopropyl β-D-thioglucopyranoside, L(+)-Erythrose, Methyl-sP-D-galactopyranoside, Methyl-β-D-thiogalactoside, N-Acetylmuramic acid, O-D-Allose, D-(+)-Talose, D-Glucosaminic acid, Deoxyfuconojirimycin, 2-Deoxy-L-ribose, L-Galactono-1,4-lactone, L-Idaro-1,4-lactone, N-Acetyl-D-glucosamine, (−)-Sinigrin hydrate, 5-Thio-D-glucose, D-Threono-1,4-lactone, D-Xylono-1,4-lactone, D-(−)-Erythrose, L-Threono-1,4-lactone, Methyl α-D-mannopyranoside, D-Glucosone, N-Glycolylneuraminic acid, 2-Deoxy-D-ribose, 3-O-Methyl-D-glucopyranose, D-Glucosamine 3-sulfate, D-Altrose, D-(−)-Fructose, D-(+)-Xylose, Glycerol, D-Galactose, L-(−)-Sorbose, meso-Erythritol, Methyl α-D-glucopyranoside, Dextrose, L-Rhamnose, N-Acetyl-D-galactosamine, L-(+)-Arabinose.

In some embodiments, the electrostatic molecule is a hydrophilic peptide. In some embodiments, the N-terminus is conjugated to a linker or PEG spacer with a primary amine terminus. In some embodiments, the electrostatic molecule is a hydrophilic polymer with the general formula:

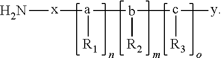

In certain aspects, x is a linker that may or may not be attached to a chain transfer agent, y is the polymer terminus, a, b and c are polymer blocks consisting of repeating units R₁, R₂ and R₃. These may be block copolymers or statistical or random copolymers, n, m and o are integers between 1 and 500. In certain aspects, R₁=R₂=R₃. In certain aspects, R₁≠R₂≠R₃. In certain aspects, R₁=R₂≠R₃. In certain aspects, R₁≠R₂=R₃.

The present disclosure also provides a composition comprising: a nucleic acid nanostructure, and a layer of molecules that can mediate endosomal escape, which are electrostatically bound to the nucleic acid phosphate backbone. In some embodiments, the electrostatic molecules are compounds, or derivatives, of the group selected from chloroquine, 1-[1-(6-Chloroquinolin-4-yl)piperidin-4-yl]piperidin-3-ol, 1-(7-chloroquinolin-4-yl)piperidin-4-ol, 2-[4-(7-Chloroquinolin-4-yl) morpholin-2-yl]ethanamine, [1-(7-Chloroquinolin-4-yl)piperidin-3-yl]methanol, 1R,2R)-2-N-(7-Chloroquinolin-4-yl)cyclohexane-1,2-diamine, (1S,2S)-2-N-(7-chloroquinolin-4-yl)cyclohexane-1,2-diamine, N′-(7-chloroquinolin-4-yl)-N-cyclohexylethane-1,2-diamine, N-(4-aminobutyl)-7-chloroquinolin-4-amine, (1R,2S)-2-N-(7-chloroquinolin-4-yl)cyclohexane-1,2-diamine, N′-(7-Chloroquinolin-4-yl)-N-ethylpropane-1,3-diamine, N-(7-Chloroquinolin-4-yl)-N′,N′-dimethylbutane-1,4-diamine, N4-(7-chloroquinolin-4-yl)-n1-methylpentane-1,4-diamine, Didesethyl Chloroquine, 3-N-(7-chloroquinolin-4-yl)-1-N,1-N-dimethylbutane-1,3-diamine, N′-(7-Chloroquinolin-4-yl)-N-(2-methylpropyl)propane-1,3-diamine, Desethyl Chloroquine, N′-(7-chloroquinolin-4-yl)-N-cyclohexylpropane-1,3-diamine, 1-(7-Chloroquinolin-4-yl)-N,N-dimethylpiperidin-3-amine, N-(7-chloroquinolin-4-yl)-N′,N′-diethylpropane-1,3-diamine, (3R)-1-(7-chloroquinolin-4-yl)azepan-3-amine, N-(7-chloroquinolin-4-yl)-N′-propan-2-ylpropane-1,3-diamine, 1-(7-chloroquinolin-4-yl)azepan-3-amine, N-(7-Chloroquinolin-4-yl)-N′,N′-di(propan-2-yl)ethane-1,2-diamine, N-(7-chloroquinolin-4-yl)-N′,N′-dimethylpropane-1,3-diamine, N′-(7-Chloroquinolin-4-yl)-N,N-diethylethane-1,2-diamine, 1-N-(7-Chloroquinolin-4-yl)-2-N,2-N-dimethylpropane-1,2-diamine, (1-Quinolin-4-ylpiperidin-4-yl) methanamine, N-(6-Chloroquinolin-4-yl)-N′,N′-diethylpropane-1,3-diamine, N-(7-Chloroquinolin-4-yl)-N′-methylpropane-1,3-diamine, (E)-N-(7-Chloroquinolin-4-yl)-N′,N′-diethylbut-2-ene-1,4-diamine, 7-Chloro-N-(5-pyrrolidin-1-ylpentan-2-yl)quinolin-4-amine, ethyl (8-((2-(dimethylamino)ethyl)amino)-2,3-diphenylpyrido[2,3-b]pyrazin-6-yl)carbamate or ethyl (8-((2-(dimethylamino)ethyl)amino)-2,3-diphenylpyrido[2,3-b]pyrazin-6-yl)carbamate.

The present disclosure also provides a composition comprising a nucleic acid nanostructure, and a layer of primary amine-containing small molecules that have a therapeutic effect.

In some embodiments, the small molecule is selected from the group consisting of aciclovir, adefovir dipivoxil, alfuzosin, amiloride, aminosalicylic acid, amisulpride, amlexanox, amprenavir, amrinone, anileridine, azacitidine, benzocaine, bleomycin, bromfenac, cefdinir, cefditoren, cefepime, cefixime, cefmenoxime, cefotaxime, ccfpodoxime, ceftazidime, ceftriaxone, chloriprocaine, cidofovir, cladribine, clenbuterol, clofarabine, cytarabine, dactinomycin, dapsone, darunavir, decitabine, doxazosin, emtricitabine, entecavir, famciclovir, flucytosine, fludarabine, fosamprenavir, ganciclovir, gemcitobine, imiquimod, lamivudine, lamotrigine, lenalidomide, mesalazine, methotrexate, metoclopramide, minoxidil, mitomycin, nelerabine, oxybuprocaine, pemetrexed, penciclovir, phenazopyridine, pramipexole, prazosin, procainamide, procaine, proflavine, proparacaine, pyrimethamine, riluzole, S-adenosylmethionine, sparfloxacin, sulfacetamide, sulfanilamide, tacrine, tamibarotene, tenofovir, terazosin, tetrahydrobiopterin, tetrahydrofolic acid, thiamine, thioguanine, triamterene, trimethoprim, tremexate, valaciclovir, valganciclovir, vidarabine, zalcitabine.

In some embodiments, the therapeutic molecules are formulated with any of the hydrophilic molecules provided herein. These could be formulated with 1, 2, 3, 4, 5, 6, 7, 8, 9, 10 or more different types of molecules in varied molar ratios.

In some embodiments, the hydrophilic layer is formulated with electrostatic binders from any of the hydrophilic molecules given in claims provided herein, plus a lipid selected from the group consisting of amine derivatives of cationic lipids including, but not limited to DOTMA, DOTAP, DOSPA or ePC; Amine derivatives of ionizable lipids including, but not limited to, DLin-MC3-DMA, ALC-0315, Lipid-H (SM-102), A2-Iso5-2DC18, BAME-016B, 9A1P9, C12-200, cKK-E12, OF-Deg-Lin, 306Oᵢ₁₀, TT3, or FTT5; Or amine derivatives of other types of lipids, including but not limited to, DSPC, PEG₂₀₀₀-DMG, ALC-0159, cholesterol, DC-cholesterol, 3-sitosterol and BHEM-cholesterol.

### BRIEF DESCRIPTION OF DRAWINGS

FIG. 1 is a schematic showing that Mergos (103 or 107; 104 or 108 or 110) can be linked directly and indirectly (105;106) to NATs (103 or 107; 104 or 108 or 110) and/or NA barcodes (103 or 107; 104 or 108 or 110). Mergo, NAT and NA barcodes can be linked for instance via complementary binding or click chemistry, or indirectly through their incorporation into a delivery material such as a lipid nanoparticle. The arrangement of these combinations depicted (101, 102) are not fixed and could be arranged in more than one order or combination, for example, in one embodiment 107 could be both a NA barcode and Mergo with 104 a NAT.

FIG. 2 is a schematic showing that NA barcodes (203, 208 or 210) can be directly or indirectly (205, 206) linked to cargo molecules (204) and NATs (203, 208 or 210), and a combination thereof (202).

FIG. 3 is a schematic showing that the NA barcode described herein, including modified single or duplexed NA barcodes (C) can be covalently or noncovalently linked (B) to a range of cargo molecules (A), beyond Mergos or NATs.

FIG. 4 shows exemplary designs of barcoded Mergos comprising various structural elements. Designs 401, 403 and 407 consist of two duplex motifs and incorporate a barcode in a single-stranded bulge region. Designs 402, 404 and 405 comprise a hairpin motif, and may incorporate the barcode within the hairpin, a 5′ or 3′ overhang region or a single-stranded bulge region. Design 407 comprises a circularized oligonucleotide strand which carries a barcode sequence. Conjugation chemistry, electrostatic interactions or hybridization regions can be used to attach additional cargoes.

FIG. 5 shows different configurations of Mergos, NATS and NA Barcodes (or ‘barcodes’) can be linked to form a constructs, with some constructs having just a Mergo and a NAT, whereas others may have a Mergo, NAT and NA Barcode: 501-502) NATs can be directly linked to a “ds” (double stranded) or “ss (single stranded) Mergo and/or Barcode, with optional overhangs; 503-506) NATs can be linked to a Mergo where the modified strands of the Mergo can be extended to allow for a greater number of modifications and/or small molecules to be incorporated into the construct and to alter the PK/PD through increased size and/or a NA Barcode, whereby the barcode or Mergo has an overhang, in a preferred embodiment the overhang is the NA Barcode; 507-508) a double stranded siRNA is linked via hybridization to a Mergo (ds or ss) and/or barcode (ds or ss), with an optional overhang (507); 509-510) single stranded Mergo, NA Barcode and NAT linked via hybridization, with the ability to extend the Mergo or NA Barcode (510); 511-512) ssNAT, e.g., ASO, linked to a Mergo (ds or ss) and/or NA Barcode (ds or ss), with optional overhangs; 513) dsNAT, e.g. siRNA, linked to a Mergo (ds or ss) and/or NA Barcode (ds or ss), with optional overhangs; 514) a NAT linked to another NAT, and a Mergo (ds or ss) and/or a Barcode (ds or ss); 515) NAT linked to a Barcode and/or Mergo with a bulge; 516) a NAT linked to a Mergo and a Barcode with a bulge in the Barcode; 517) a ssNAT hybridized to a Mergo or Barcode; 518) a NAT linked to a Mergo, with the ability to extend the overhang of the NAT or Mergo (519-520)—where additional nucleic acids incorporated onto the construct will allow for a greater number of modifications and/or small molecules to be incorporated into the construct and to alter the PK/PD through altered size; 521-523) NAT linked to a Mergo and/or Barcode, where the Mergo or Barcode form a bulge. Bulges in the Mergo can act as sites for additional modifications, and to make the barcode more easily readable, in some preferred embodiments the bulge could be of around 8 nucleotides. In a preferred embodiment of the designs above, the overhang ranges from 1-16 nucleotides, however is not limited to this length and may be longer. The overhangs can occur on both the 5′ and 3′. Legend: I) Single stranded NAT, Mergo or NA Barcode; II) Double stranded (or ‘duplexed’) NAT, Mergo or NA Barcode; III) NAT (including, but not limited to, siRNA), Mergo or Barcode, with a 3′ or 5′ overhang; IV) Duplexed NAT, Mergo or NA Barcode with bulge; V) Linker mechanism, which could include, but not limited to, chemical conjugation (e.g. IEDDA), hybridization/complementary binding of oligonucleotides, or during strand synthesis (e.g. solid phase oligonucleotide synthesis, which could include, but not be limited to, a phosphorothioate).

FIG. 6 shows the pipeline for Mergo screening. A. Different Mergo iterations are designed and produced. Each iteration is attached to a unique RNA barcode. B. The barcoded-Mergos are pooled and injected in a single injection in wild type animals. Seven days post-injection the organs of the animals are collected. C. Single cell RNA sequencing is performed to the organs of the animals to identify: 1. The cell types. 2. The barcodes identified in every cell type. 3. The knockdown detected per cell type. D. The correlation of the cell type, Mergo design and knockdown is investigated to identify the hits. E. On iteration 1, Mergo chemistries are selected quasi-randomly from the defined chemical space. On iteration 2, chemistries are selected to provide the biggest expected-improvement on siRNA knockdown utilising the knowledge gained from the hit Mergos.

FIG. 7 is a graph showing that Superscript III reverse transcriptase can efficiently use 2′OMe and 2′F modified oligonucleotide sequences as templates without reduction of enzymatic activity. Equal input amounts of unmodified and modified RNA strands were reverse transcribed and the so-generated cDNA then subjected to qPCR, resulting in equal quantification cycle values (Cq) corresponding well with the Cq value of the positive control.

FIG. 8 is a graph showing that a Mergo hybridized to a barcode-containing oligonucleotide can be detected in mouse liver lysates in a concentration-dependent manner by qPCR.

FIG. 9 is a denaturing PAGE gel (12%) demonstrating that adapter ligation to a modified barcode placed at the 5′ terminus of an oligonucleotide leads to a shift in electrophoretic mobility. Lane 1 shows Low Range ssRNA Ladder (N0364S, New England Biolabs), lanes 2-4 show strands C-1 to C-3 after 5′ adapter ligation, respectively, and lanes 5-7 show the same strands prior to 5′ adapter ligation.

FIG. 10 shows the design and assembly of two DNA nanostructures, each comprising a 3-way junction motif and a barcode of different lengths in the branched loop region. A and B are schematics showing the design of structures M-3 and M-4, respectively. C is a native PAGE gel (6%) showing the barcoded strands C-6 and C-7 (lanes 1 and 2), a combination of the two non-barcoded strands C-4 and C-5 (lane 3), a combination of strands C-5 and C-6 (lane 4), a combination of strands C-5 and C-7 (lane 5), the assembled construct M-3 (lane 6) and the assembled construct M-4 (lane 7).

FIG. 11 shows examples of barcode positioning within Mergos conjugated to nucleic acid therapeutics. Barcodes may be incorporated into single-stranded loop regions of different length (A-B), into single-stranded 5′ or 3′ overhangs (C and E) or into hairpin motifs (D). Alternatively, barcodes may be hybridized to a Mergo (F), included within a double-stranded region of the Mergo (G) or bioconjugated to a Mergo (H) via a cleavable or non-cleavable linker. The arrow labeled B indicates the barcode position.

FIG. 12 is a denaturing PAGE gel (12%) showing various TW construct designs which have barcodes incorporated into the structure.

FIG. 13 is a denaturing PAGE gel (12%) showing various TW construct designs which have barcodes incorporated into the structure.

FIG. 14 is a denaturing PAGE gel (12%) showing various duplex construct designs (and their component strands) which have barcodes incorporated into the structure.

FIG. 15 is a denaturing PAGE gel (12%) showing various duplex construct designs (and their component strands) which have barcodes incorporated into the structure.

FIG. 16 is a denaturing PAGE gel (12%) showing detection and amplification of unmodified nucleic acid, modified single-stranded and double-stranded nucleotides containing barcodes. Samples: Unmod: cDNA product from a native RNA Mod ss: cDNA product from modified single-stranded nucleotides containing barcode Mod ds: cDNA product from modified double-stranded nucleotides containing barcode.

FIG. 17 is a denaturing PAGE gel (12%) showing detection and amplification of modified barcodes that are incorporated within TW construct designs. 1: TW-11; 2: TW-38; 3: TW-41; 4: TW-49; 5: TW-50.

FIG. 18 is a native PAGE gel (15%) showing efficient reverse transcription and cDNA amplification of fully modified strands with internal barcodes flanked by primer binding sequences. Clean products can be observed.

FIG. 19 is a denaturing PAGE gel (12%) showing detection of modified barcoded strands by template-switching reverse transcription. 1 & 2: fully 2′OMe modified ssRNA with barcode GCGAGUAU; 3: Negative control (no template-switching oligo); 4: Water control; 5=Positive control.

FIG. 20 is a denaturing PAGE gel (15%) showing that modified RNA strands remain substrates for 5′ modifying enzymes such as TdT and ligases. 1: no TdT (30 min) 2: no TdT (o/n) 3: no T4 (30 min) 4: no T4 (o/n) 5: full reaction (30 min) 6: full reaction (o/n) 7: ladder.

FIG. 21 is a bar plot showing detection of modified single-stranded and double-stranded nucleotides containing barcodes in cultured cells at the single-cell level. NTC indicates the negative control (cells not incubated with barcodes).

FIG. 22 (A) Shows that certain barcode sequences were more efficiently detected than others, but there is no clear trend in terms of % GC/AT content of the sequences. (B) Shows the results of Mergo multiplexing and the impact of Mergo morphology on barcode detection at the scSEQ level. TW-41 is preferentially detected.

FIG. 23 Schematic depiction of different Mergo designs with their associated 8-nt barcode sequences encoding Mergo shape. Barcode positioning and reverse transcription primer binding sites are indicated with curly brackets.

FIG. 24 Feature plots showing detection of double-stranded nucleotides containing barcode and RNA therapeutics across three tissue types, liver (A), lung (B) and heart (C), in mice at the single-cell level. Each dot is a cell and color intensity represents the number of counts for the barcode.

FIG. 25 is a bar plot showing the length distribution of barcode reads as detected by single-cell sequencing of cells extracted 7 days after Mergo injection in mice. Shown are mean±standard deviation across three mouse tissues (heart, liver lung)..

FIG. 26 LCMS spectra confirming 5′ phosphorylation of modified and unmodified RNA strands after treatment with PNK.

FIG. 27 outlines a library preparation workflow for bulk RNA sequencing of short modified barcodes. Adapter ligation directly on the RNA strand allows sample multiplexing already at the cDNA level, reducing sample numbers for further processing.

FIG. 28 is a denaturing PAGE gel showing 10 different adapters were tested in vitro on modified ssRNA barcodes.

FIG. 29 shows schematic depictions of TW-17, TW-18 and MD-19 Mergo constructs that incorporate a barcode directly within the core.

FIG. 30 shows the Bioanalyzer traces of BulkSeq libraries detecting a lipid-conjugated oligonucleotide (lanes 12-18, 22) and multiplexed barcoded Mergos carrying an siRNA therapeutic as cargo (lanes 19, 21). Lanes to the left (12-17) show the final libraries after size selection, lanes to the right (18-22) show crude libraries prior to purification by size selection. Note that automatic detection of the upper and lower marker failed for most of the crude libraries. 12: Lipid-BC_Brain stem & Cerebellum; 13: Lipid-BC_Upper spinal cord; 14: Lipid-BC_Injection site; 15: Lipid-BC_Cortex; 16: Lipid-BC_Deep brain; 17: Lipid-BC_CSF; 18: CRUDE [Lipid-BC_Injection site]; 19: CRUDE [Multiplexed_Injection site]; 20: CRUDE [Vehicle_Deep brain]; 21: CRUDE [Multiplexed_Upper spinal cord]; 22: CRUDE [Lipid-BC_Cortex].

FIG. 31 shows the output of BulkSeq library preparation. A band can be detected at the right size and is not detectable in the vehicle controls.

FIG. 32 is a native PAGE gel (12%) showing the barcode-specific PCR products present in bulk RNA sequencing libraries. Lanes 1-3 show amplicons obtained from different barcodes, confirming the presence of three different barcodes. Lanes 4-5 show amplicons obtained from PCR amplification with universal primers binding to Illumina adapter sites.

FIG. 33 shows a Design of Experiments (DoE) optimization of oligonucleotide-lipid conjugations.

FIGS. 34A-34D show LC traces of an oligonucleotide conjugated to heptadecanoic acid.

FIGS. 35A-35D show LC traces of an oligonucleotide conjugated to DPSE-glutaric acid.

FIG. 36 is a schematic depicting: A) A barcode-lipid conjugate, consisting of a barcode region, a linker region (attached via click chemistry) and a lipid. The lipid could be, but is not limited to, the examples shown. B) A barcode conjugated to a highly modified oligonucleotide, whereby the oligonucleotide is modified via either the 2′O, the phosphate backbone or the nucleotide base.

FIGS. 37A-37B show the LCMS of a barcode-palmitic acid conjugate conjugate, with representative sequence [palmitic acid//5AmMC6/c*mA*amAumUcmCamUcmGu*mG*a-whereby 2′ F C/A/U is indicated with lowercase, 2′ O me A/U/G/C indicated with ‘m’, PTO indicated with *, ′5AmMC6′ represents a C6 amino modifier]

FIGS. 38A-38C show the LCMS of a barcode-hexadecanoic acid conjugate, with represenative sequence (C16//5AmMC6/mCmCmAmCmUmUmGmGmAmCmGmAmGmUmUmAmC, whereby 2′ O me A/U/G/C indicated with ‘m’ and ′5AmMC6′ represents a C6 amino modifier) with this methodology.

FIG. 39 shows Mergos based on natural RNA dimers. (A) is a Mergo based on the osk dimer and is formed via a GC-rich palindromic kissing loop. (B) is a Mergo based on the 3′ UTR domain III of bicoid (bed) mRNA, whereby the individual units are linked by the complementarity of the apical loop and an internal bulge.

FIG. 40 shows a IP-RP HPLC trace of an oligonucleotide-cholesterol conjugate.

FIG. 41 (A) denaturing PAGE showing an oligonucleotide (left) and oligonucleotide-peptide conjugate (right) and (B) an IP RP HPLC trace of an oligonucleotide-peptide conjugate.

FIG. 42 shows methyl phosphonamidites of adenosine and 2′O propargyl adenosine.

FIG. 43 outlines a general synthesis route to alkyl phosphonamidites via Grignard methodology.

FIG. 44 outlines the phosphorylation of 2′O propargyl nucleosides with alkyl phosphonamidite modifiers.

FIG. 45 outlines the synthesis of novel phosphitylating reagents. R may include, but is not limited to, These may include, but are not limited to, O-alkylamino, O-alkylalkoxy, protected O-alkylamino, O-alkylaminoalkyl, O-alkyl imidazole, O-alkyl guanidine, polyamines of the formula (O-alkyl)ₘ, where m is about 1 to 20 and polyethers of the formula (O-alkyl)ₘ, where m is about 1 to 20. Preferred ethers are polyethylene glycols (PEGs).

FIG. 46 outlines the synthesis of a hydrazone-based phosphitylating reagent that is compatible with an acid-free oligonucleotide synthesis process

FIG. 47 is a schematic showing Mergo attached to a cargo via a cathepsin-cleavable linker. (I) is the cargo, (II) is the cleavable linker.

FIG. 48 is a synthetic route towards a bi-functional cathepsin-cleavable linker that could be incorporated into an amine-modified macromolecule, including, but not limited to, oligonucleotides, peptides and proteins. The norbornene modifier can be used to couple Mergo to any given tetrazine-functionalized cargo.

FIG. 49 outlines the synthetic route towards a norbornene-terminated cathepsin cleavable phosphoramidite for incorporation into oligonucleotides via solid phase synthesis. The norbornene modifier can be used to couple Mergo to any given tetrazine-functionalized cargo.

FIG. 50 is a schematic outlining how electrostatic modifiers can be applied to Mergo. The oligonucleotide is first modified with any given modification via CuAAC. It then undergoes the electrostatic formulation with PEG to provide solubility. This can then be assembled into Mergo.

FIG. 51 shows examples of how electrostatic modifiers can be utilized with Mergo. A shows a double-stranded, modified Mergo conjugated to a NAT and functionalized with an electrostatic modifier layer for improved solubility (I) is the modification, (II) is the electrostatic modifier, (III) is the Mergo component, (IV) is the NAT. B shows a single-stranded, modified Mergo conjugated to a NAT and functionalized with an electrostatic modifier layer for improved solubility. (I) is the modification, (II) is the electrostatic modifier, (III) is the Mergo component, (IV) is the NAT.

FIG. 52 is a synthetic route outlining the synthesis of a guanidine-based electrostatic modifier. In some embodiments, R₂ is equal to R₃. In some embodiments R₂ is different from R₃.

FIG. 53 is a ¹H NMR of an oligonucleotide-ANI complex. Peaks integrated against 2× aromatic uracil protons (2 U in sequence). RNA has a charge state of 14 c=2 protons. 28/2=14 d=3 protons. 42/3=14. Suggests complete binding to backbone.

FIG. 54 is a ¹H NMR of an RNA-PEG complex. The integrals observed match up to the expected values for complete binding to the oligonucleotide backbone.

FIG. 55 is a 6% native PAGE gel showing electrostatic PEG assemblies. From left to right: 1) Unmodified Mergo with no electrostatic PEG; 2) Unmodified Mergo with electrostatic PEG; 3) Mergo modified with 19× chloroquine modifications with outer electrostatic PEG layer (assembled in PBS+2 mM MgCl₂); 4) Mergo modified with 19× chloroquine modifications with outer electrostatic PEG layer (assembled in pure H₂O); 5) Unmodified Mergo assembled in pure H₂O; 6-8) unmodified Mergo constructs assembled in PBS+2 mM MgCl₂.

### DETAILED DESCRIPTION

Nucleic acids (NA) are an excellent natural barcoding material due to the four alternating nucleotides that can be programmed into any given sequence. The ability of synthesizing NA strands with controlled patterns of alternating nucleotides has empowered researchers to design custom barcodes on demand; the recent development in sequencing technologies has also expedited the ability to read these barcodes and in the last decade, these technologies have become cheaper and hence accessible to the wider scientific community. This has ultimately allowed for the use of NA barcodes across the fields of genomics and bioinformatics.

DNA barcoding is known in art and has been used for high throughput in vivo screening for the development of drug delivery systems (U.S. Pat. No. 2,020,330,607; J. E. Dahlman, K. J. Kauffman, Y. Xing, T. E. Shaw, F. F. Mir, C. C. Dlott, R. Langer, D. G. Anderson, E. T. Wang, Barcoded nanoparticles for high throughput in vivo discovery of targeted therapeutics, Proc. Natl. Acad. Sci. 114 (2017) 2060-2065. https://doi.org/10.1073/pnas.1620874114). Prior to these developments, high throughput screenings that were performed in vitro were unreliable, as they showed little correlation with the in vivo output. Instead, hundreds of animals were used for high throughput in vivo screenings making this process costly and posing ethical questions. Dahlman et al. demonstrated the use of synthetic DNA barcodes consisting of different patterns of 53 nt. Each barcode was encapsulated in nanoparticles of different chemical compositions. The nanoparticles were then pooled and injected in a single group of animals. The tissues of the animals were then collected and their delivery was quantified by sequencing of the barcodes. Using this method, reliable in vivo data are generated by a single group of five animals used to screen >100 nanoparticles, reducing the number of animals used >100-fold (M. P. Lokugamage, C. D. Sago, J. E. Dahlman, Testing thousands of nanoparticles in vivo using DNA barcodes, Curr. Opin. Biomed. Eng. 7 (2018) 1-8. https://doi.org/10.1016/j.cobme.2018.08.001.). The NA barcoding method makes the use of non-human primates more accessible and more ethical, opening new horizons in drug development (M. Z. C. Hatit, M. P. Lokugamage, C. N. Dobrowolski, K. Paunovska, H. Ni, K. Zhao, D. Vanover, J. Beyersdorf, H. E. Peck, D. Loughrey, M. Sato, A. Cristian, P. J. Santangelo, J. E. Dahlman, Species-dependent in vivo mRNA delivery and cellular responses to nanoparticles, Nat. Nanotechnol. 17 (2022) 310-318. https://doi.org/10.1038/s41565-021-01030-y.).

Barcodes have not been used for screening delivery systems for the administration of conjugated and/or gymnotic nucleic acids, such as GalNAc-conjugates or peptide-conjugates, where a NA barcode attached to the conjugate would not be encapsulated and would hence rapidly degrade by the naturally occurring nucleases found in biological material. Indeed, NA barcodes have been widely used for the development of lipid nanoparticulate delivery systems, where the encapsulation of minimally modified NA barcodes ensures the stability of NA strands against nuclease degradation. Utilizing recent developments in nucleic acid modification, the present disclosure outlines ways to incorporate NA barcodes onto gymnotic nucleic acid and other “exposed” delivery systems or methods whereby the identification of chemical entities in a biological context would be useful, including but not limited to CRISPR, proteins, peptides and NA material.

The throughput of NA barcoding technology can be further improved by the reduction in the length of the barcodes. Currently, the field is relying on long strands of nucleic acids including primer binding sites and unique molecular identifiers. These long barcodes present an ease of library preparation for sequencing, as they already contain the two universal sites as well as the unique molecular identifiers. However, they also present a few disadvantages: i) they could affect the biodistribution of a conjugated nucleic acid therapeutic, or other conjugated cargos, and hence bias the result, and, ii) they require a costly and challenging synthesis process due to their length. A short barcode of a known nucleotide sequence but excluding the two universal sites and unique molecular identifier would address these challenges but would also require a longer library preparation to add the universal sites. Overall, both short and long barcodes present advantages and disadvantages and the researchers would need to decide themselves which format better suits their needs.

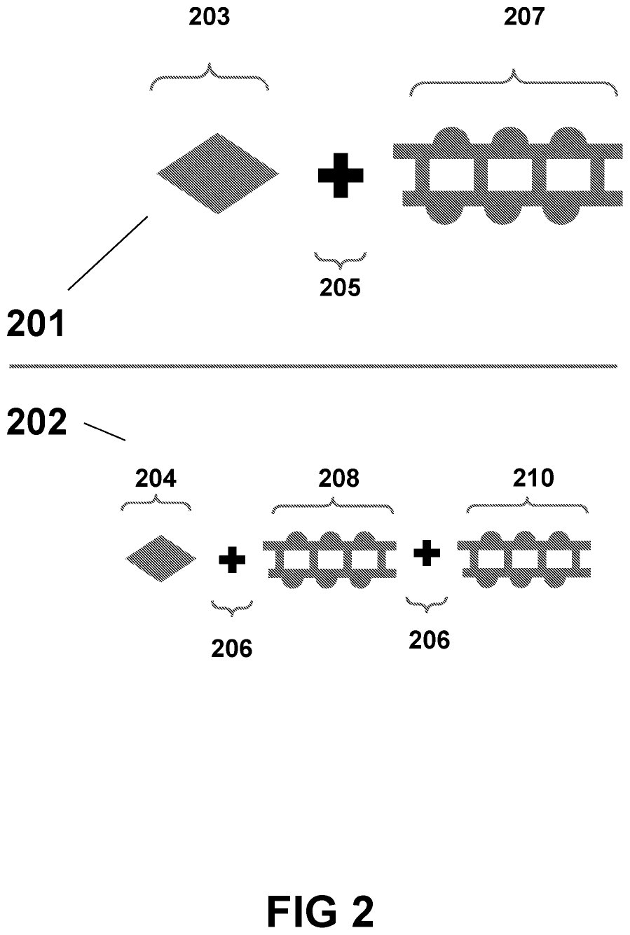

For reference throughout the term “Mergo” or “Mergos” is used to refer to a single or double stranded oligonucleotide with minimum length of 8 nucleotides, where the function is not explicitly of a NAT, but is instead intended to enhance the delivery and functionality of NATs. Mergos are designed to be directly or indirectly linked to one or more NATs (FIG. 2).

In some embodiments the component oligonucleotides in the Mergo contain chemically-modified nucleotides. Such nucleotides were described in US 2021/330,810, the contents of each of which are incorporated herein by reference in its entirety.

In some embodiments, the Mergos are modified with methyl phosphonamidites. Methyl phosphonamidites have been previously used to introduce one or more methyl phosphonate linkages in oligonucleotides. Methylphosphonate (MP) linkages are neutrally-charged. This allows the formation of backbones with reduced negative charge and increased hydrophobicity, with therefore potential to alter the physiochemical and biological properties of the oligonucleotides. Consequently, methylphosphonate linkages have been shown to be fully resistant to nuclease degradation (K. Paunovska, D. Loughrey, J. E. Dahlman, Drug delivery systems for RNA therapeutics, Nat. Rev. Genet. 0123456789 (2022). https://doi.org/10.1038/s41576-021-00439-4.); (M. A. Reynolds, R. I. Hogrefe, J. A. Jaeger, D. A. Schwartz, T. A. Riley, W. B. Marvin, W. J. Daily. M. M. Vaghefi, T. A. Beck, S. K. Knowles, R. E. Klem, L. J. Arnold, Synthesis and Thermodynamics of Oligonucleotides Containing Chirally Pure RP Methylphosphonate Linkages, Nucleic Acids Res. 24 (1996) 4584-4591. https://doi.org/10.1093/nar/24.22.4584.) or improve cellular uptake (compared to unmodified oligonucleotides) (Q. Zhao, S. Matson, C. J. Herrera, E. Fisher, H. yu, A. M. Krieg, Comparison of Cellular Binding and Uptake of Antisense Phosphodiester, Phosphorothioate, and Mixed Phosphorothioate and Methylphosphonate Oligonucleotides, Antisense Res. Dev. 3 (1993) 53-66. https://doi.org/10.1089/ard.1993.3.53.).

As presented herein, the present disclosure provides ways of synthesizing methyl phosphonamidites and incorporating them into oligonucleotides.

Accordingly, in the first embodiment, the disclosure features a synthetic route to methyl phosphonamidites, via modification of the 3′ hydroxyl on any given nucleoside.

In another embodiment, the disclosure outlines incorporation of the methyl phosphonamidite into an oligonucleotide.

In another aspect, the disclosure features an RNA molecule that is functionalized with one or more methyl phosphonates.

In another aspect, the disclosure features an R/DNA chimeric molecule that is functionalized with one or more methyl phosphonates.

In another aspect, the disclosure features a DNA molecule that is functionalized with one or more methyl phosphonates.

In a further embodiment, the disclosure features an oligonucleotide with unnatural RNA/DNA residues, whereby the backbone is modified as a methyl phosphonate and the 2′ position of one or more nucleotides is functionalized with either fluorine or a methoxy group.

In a further embodiment, the disclosure features an oligonucleotide with unnatural RNA/DNA residues, whereby the backbone is modified as a methyl phosphonate and the 2′ position of a component nucleotide is functionalized with a propargyl group.

In some embodiments the Mergo is a duplex oligonucleotide that is linked to a NAT.

In some embodiments, Mergo is designed in such a way that it incorporates a unique nucleic acid barcode within its sequence. The barcode serves as a label for the identity of every Mergo such that it can be used to distinguish between hundreds of different Mergo compositions in a pool of Mergo constructs, enabling detection and analysis of a plurality of Mergos in a single assay (multiplexing). The sequence of each barcode is designed to be sufficiently unique to allow its unambiguous differentiation from all other barcodes in the pool. For barcode detection, any sequencing or hybridization-based method known in the art can be employed, including but not limited to next-generation sequencing, microarray, in-situ hybridization, and branched DNA technologies.

In some embodiments, the barcode is used to encode the chemical composition of Mergos. This allows for the rapid identification and optimisation of chemistries that can enhance delivery of Mergo vehicles in high-throughput screenings performed in vivo or in vitro.

In some embodiments, the barcode serves as an identifier of the cargo molecules or NAT molecules that are directly or indirectly linked to each Mergo. Hereby, the barcode may function as a traceable and quantifiable reporter for the delivery of different cargo or NAT molecules, and allows comparison of delivery efficiencies. Similarly, in other embodiments, the barcode serves as a classifier for Mergo assembly protocols or cargo conjugation techniques. In further embodiments, the barcode is an indicator of the shape or size of Mergos. In yet other embodiments, the barcode can be used as a batch identifier.

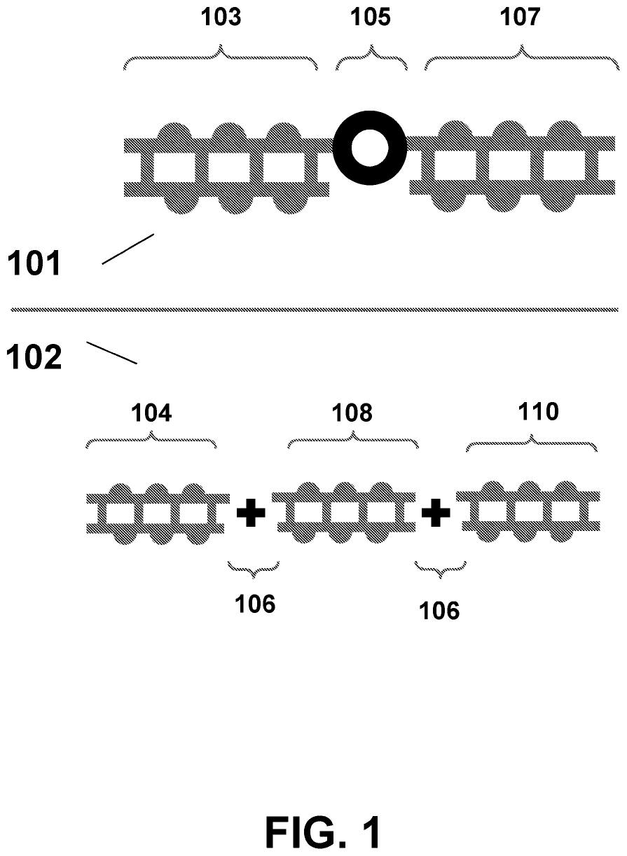

In some embodiments, the Mergo is directly or indirectly linked to one or more of following components (FIG. 1): (1) a NAT; (2) a chemical composition identifier (i.e. a unique barcode) that can be read via downstream assays; (3) one or more cargo molecules, including, but not limited to, molecules that promote a function and/or biological effect inside or outside a cell (e.g. IRES, ribosomal recruitment, cytokine stimulation), molecules that promote entry into a cell (e.g. peptides, endosomal escape compounds), molecules that bind to target cells (e.g. aptamers, antibodies, ligands), cytotoxic compounds (e.g. cytotoxic nucleosides), molecules that express a gene product inside a cell (e.g. mRNA), chemotherapeutic compounds (e.g. alkylating agents, antimetabolites, topoisomerase inhibitors), molecules that silence or alter a gene inside a cell (e.g. siRNA, miRNA, antisense therapy, lncRNA), CRISPR molecules (e.g. gRNA, Cas9 protein, Cas9 mRNA), small molecule therapies (e.g. protein-tyrosine kinase inhibitors, proteasome inhibitors), proteins, peptides, and diagnostic agents.

In further embodiments, the Mergo is conjugated to any given component via conjugation techniques previously disclosed in U.S. Ser. No. 17/241,920, the contents of which are incorporated herein by reference in its entirety. This includes, but is not limited to, copper (I)-catalyzed alkyne-azide cycloaddition (CuAAC), strain-promoted alkyne-azide cycloaddition (SPAAC), ruthenium-catalysed azide-alkyne cycloaddition (RuAAC), inverse electron demand Diels-Alder reaction (IEDDA), Sulfur Fluoride Exchange (SuFEx), strain-promoted alkyne-nitrone cycloaddition (SPANC), hydrazone/oxime ether formation, thiol-ene radical reaction, thiol-yne radical reaction, thiol-Michael addition reaction, thiol-isocyanate reaction, thiol-epoxide click reaction, nucleophilic ring opening reactions (spring-loaded reactions), traceless Staudinger ligation.

In some embodiments, the modified barcode is linked directly or indirectly to a chemical entity to determine its identity.

In another aspect, the modified barcode is linked directly or indirectly to a chemical entity that is designed to exert a biological outcome in a living system such as a mammal or plant, including but not limited to NATs, peptides, CRISPR, proteins, cytotoxic drugs, antibodies, carbohydrates, small molecules.

In another aspect, the modified barcode is linked directly or indirectly to a chemical entity that is used to deliver NATs to tissues in a living entity such as a mammal or plant, including, but not limited to, the following conjugates: antibodies, NAs. GalNac, lipids, proteins, folate, peptides, carbohydrates, small molecules.

In a preferred embodiment, the barcode may be 5-200 nucleotides in length, more preferably 8-20 nucleotides in length

Due to structural flexibility and the propensity of RNA to form tertiary structures, the design of RNA nanoparticles with well-defined shapes for in vivo delivery is not trivial. The most successful approaches to date have incorporated naturally occurring motifs as building blocks (E. Bindewald, C. Grunewald, B. Boyle, M. O'Connor, B. A. Shapiro, Computational strategies for the automated design of RNA nanoscale structures from building blocks using NanoTiler, J. Mol. Graph. Model. 27 (2008) 299-308. https://doi.org/10.1016/j.jmgm.2008.05.004.; Y. Shu, F. Haque, D. Shu, W. Li, Z. Zhu, M. Kotb, Y. Lyubchenko, P. Guo, Fabrication of 14 different RNA nanoparticles for specific tumor targeting without accumulation in normal organs. RNA. 19 (2013) 767-777. https://doi.org/10.1261/rna.037002.112.; Y. Shu, F. Pi, A. Sharma, M. Rajabi, F. Haque, D. Shu, M. Leggas, B. M. Evers, P. Guo, Stable RNA nanoparticles as potential new generation drugs for cancer therapy, Adv. Drug Deliv. Rev. 66 (2014) 74-89. https://doi.org/10.1016/j.addr.2013.11.006.). Based on this concept, the present disclosure outlines RNA delivery vehicles built around naturally occurring motifs.

In some embodiments, the Mergo forms as compacted dimeric structures with motifs found in the 3′ UTR osk (H. Jambor, C. Brunel, A. Ephrussi, Dimerization of oskar 3′ UTRs promotes hitchhiking for RNA localization in the Drosophila oocyte, Rna. 17 (2011) 2049-2057. https://doi.org/10.1261/ma.2686411.).

In some embodiments, the Mergo forms a compacted dimeric structure(s) with motifs found in the 3′ UTR domain III of bcd mRNA (C. Wagner, I. Palacios, L. Jaeger, D. St Johnston, B. Ehresmann, C. Ehresmann, C. Brunel, Dimerization of the 3′UTR of bicoid mRNA involves a two-step mechanism, J. Mol. Biol. 313 (2001) 511-524. https://doi.org/10.1006/jmbi.2001.5057.).

In some embodiments, the Mergo forms a dimeric structure(s) with motifs found in the hatchet ribozyme.

In some embodiments, the Mergo forms a dimeric structure(s) with motifs found in glycine riboswitches.

In some embodiments, the Mergo forms a dimeric structure(s) with motifs found in riboswitches, including, but not limited to, ZTP, THF, guandine II, glutamine II and glycine.

In further embodiments, the Mergo forms RNA nanoparticles consisting of naturally occurring three-way junctions (3WJ).

In further embodiments, the 3WJ motifs are assembled into tetrameric structures.

A key advantage of nucleic acid nanotechnology disclosed herein is the ability to precisely control cargo loading and the degree to which the constructs are modified. One challenge, however, and one that is commonly observed across the drug delivery field, is controlling when and where certain cargo are released. To address this, a wide variety of linker chemistries have been developed and are commonly used in the art (G. M. Church, M. Sismour, CHEMICALLY CLEAVABLE PHOSPHORAMIDITE LINKERS FOR SEQUENCING BY LIGATION, US 2010/0081140 A1, 2009.; E. Uhlmann, R. Subramanian, MULTIMERIC OLIGONUCLEOTIDE COMPOUNDS HAVING NON-NUCLEOTIDE BASED CLEAVABLE LINKERS, US 2015/0315585 A1, 2015.), the contents of which are incorporated herein by reference. Antibody-drug conjugates (ADCs) provide a prime example of how these chemistries can be incorporated into a delivery system (J. D. Bargh, A. Isidro-Llobet, J. S. Parker, D. R. Spring, Cleavable linkers in antibody-drug conjugates, Chem. Soc. Rev. 48 (2019) 4361-4374. https://doi.org/10.1039/c8cs00676h.).

ADC linkers were initially introduced to provide a non-internalizing mechanism of action, whereby linker cleavage and payload release occur in the extracellular tumor microenvironment. This means that ADC endocytosis is not required, and non-internalizing antigens may be selected as targets. In the case of oligonucleotides and oligonucleotide-based delivery systems, these molecules are highly charged and face several intracellular biological barriers, the most prominent of which is escaping the endosome (R. L. Juliano, Intracellular Trafficking and Endosomal Release of Oligonucleotides: What We Know and What We Don't, Nucleic Acid Ther. 28 (2018) 166-177. https://doi.org/10.1089/nat.2018.0727.).

Several ADC linker technologies have progressed to clinical trials or even the clinic. The three main types include acid cleavable, reducible disulfide and those that are cleavable by enzymes. Both acid-cleavable and reducible disulfide linkers have been demonstrated in oligonucleotide-based technologies, examples of which are incorporated herein by reference (F. Gauthier, J. R. Bertrand, J. J. Vasseur, C. Dupouy, F. Debart, Conjugation of Doxorubicin to siRNA Through Disulfide-based Self-immolative Linkers, Molecules. 25 (2020) 1-15. https://doi.org/10.3390/molecules25112714.; W. Tai, Current aspects of siRNA bioconjugate for in vitro and in vivo delivery, Molecules. 24 (2019). https://doi.org/10.3390/molecules24122211.; A. Biscans, J. Caiazzi, S. Davis, N. McHugh, J. Sousa, A. Khvorova, The chemical structure and phosphorothioate content of hydrophobically modified siRNAs impact extrahepatic distribution and efficacy, Nucleic Acids Res. 48 (2020) 7665-7680. https://doi.org/10.1093/nar/gkaa595.). Despite their widespread use in ADCs, there is a tendency away from the use of exogenous triggers in the field of cleavable linkers. Enzyme cleavable linkers provide a platform whereby the delivery system is completely stable until it reaches the desired enzyme, where it then releases its payload. This is in contrast to exogenous linkers, which can suffer from poor stability profiles in vivo (M. Dorywalska, P. Strop, J. A. Melton-Witt, A. Hasa-Moreno, S. E. Farias, M. Galindo Casas, K. Delaria, V. Lui, K. Poulsen, C. Loo, S. Krimm, G. Bolton, L. Moine, R. Dushin, T.-T. Tran, S.-H. Liu, M. Rickert, D. Foletti, D. L. Shelton, J. Pons, A. Rajpal, Effect of Attachment Site on Stability of Cleavable Antibody Drug Conjugates, Bioconjug. Chem. 26 (2015) 650-659. https://doi.org/10.1021/bc5005747.).

In one aspect provided herein, the present disclosure describes cleavable linkers, whereby the cleavage mechanism could occur intracellularly or extracellularly. In some embodiments, a cleavable linker described herein could be cleaved in response to a lowering in pH. In some embodiments, the linker could be cleaved in response to a reduced oxygen environment within the extracellular space. In some embodiments, the linker could be cleaved by an external small molecule, which may or may not be released as part of a delivery system. In some embodiments, the linker may be cleaved by extracellular enzymes, including, but not limited to, endonucleases, peptidases, proteases, matrix metalloproteinases and glycosidases. In some embodiments, the linker may be cleaved by intracellular enzymes, including, but not limited to, endonucleases, proteases and peptidases. In further embodiments, the linker may be cleaved by the antioxidant glutathione, either intracellularly or extracellularly.

In another aspect, the cleavable linkers described herein can be cleaved at least 1.25, 1.5, 1.75, 2, 3, 4, 5, 10, 25, 50, or 100 times faster in the presence of the stimulus that mediates cleavage, compared to conditions where this stimulus is not present.

Without limitations, the cleavable linkers that are described herein can be used for any molecule that requires cleavage at a specific site in the body. This may be for a direct therapeutic application, such as a prodrug approach, or may be used for other applications, including, but not limited to, cleavage of molecular components that aid in formulation or solubility.

In some embodiments, the linkers may be incorporated into oligonucleotides, where they are then used to conjugate a cargo that requires cleavage. The cargo could include, but is not limited to, an RNA molecule, DNA molecule, peptide, polypeptide, protein, cytotoxic drug or any combination thereof. The RNA molecule may be a NAT. The cargo molecule may be, or may encode, a CRISPR component. The cargo molecule may be, or may encode, a chimeric antigen receptor.

In another aspect, oligonucleotides that are conjugated to a cargo via a cleavable linker may be assembled into a nucleic acid nanoparticle.

In some embodiments, the composition may include multiple oligonucleotide molecules. Where the component oligonucleotides could be DNA, RNA or XNA. Where XNA is a xeno nucleic acid that has a different sugar backbone to DNA or RNA.

In some embodiments, the composition may contain 1, 2, 3, 4, 5, 6,7, 8, 9,10, 11,12, or more DNA, RNA or XNA oligonucleotide molecules.

The self-assembled construct may take the form of any number of morphologies including, but not limited to, a trimer, tetramer, pentamer or hexamer.

Polyethylene glycol (PEG) is a biocompatible hydrophilic polymer that has been used to improve solubility of formulations, and in particular nanoparticle formulation. PEGylation has also been used to stabilize formulation and reduce protein uptake, particularly in the context of lipid nanoparticles (B. Ensing, A. Tiwari, M. Tros, J. Hunger, S. R. Domingos, C. Pdrez, G. Smits, M. Bonn, D. Bonn, S. Woutersen, On the origin of the extremely different solubilities of polyethers in water, Nat. Commun. 10 (2019) 2893. https://doi.org/10.1038/s41467-019-10783-z.). However, PEG can also dramatically reduce the cellular uptake of nanoparticles (B. L. Mui, Y. K. Tam, M. Jayaraman, S. M. Ansell, X. Du, Y. Y. C. Tam, P. J. Lin, S. Chen, J. K. Narayanannair, K. G. Rajeev, M. Manoharan, A. Akinc, M. A. Maier, P. Cullis, T. D. Madden, M. J. Hope, Influence of Polyethylene Glycol Lipid Desorption Rates on Pharmacokinetics and Pharmacodynamics of siRNA Lipid Nanoparticles, Mol. Ther.—Nucleic Acids. 2 (2013) e139. https://doi.org/10.1038/mtna.2013.66.).

Systems where the PEG shell can dissociate from the formulation have been developed and used in approved lipid nanoparticle formulations (such as ONPATTRO® (patisiran)), whereby the dissociation happens within 30 minutes (S. Chen, Y. Y. C. Tam, P. J. C. Lin, M. M. H. Sung, Y. K. Tam, P. R. Cullis, Influence of particle size on the in vivo potency of lipid nanoparticle formulations of siRNA, J. Control. Release. 235 (2016) 236-244. https://doi.org/10.1016/j.jconrel.2016.05.059.). Transient modification of nanoparticles with PEG allows for effective formulation and improved physiochemical characteristics for administration. When the PEGs are removed upon administration, the underlying modifications are exposed and are able to exert their intended function (i.e., modulation of biodistribution).

Sheddable PEGs are widely used in the art in the context of polymers (A. M. Aldayel, Y. W. Naguib, H. L. O'Mary, X. Li, M. Niu, T. B. Ruwona, Z. Cui, Acid-Sensitive Sheddable PEGylated PLGA Nanoparticles Increase the Delivery of TNF-α siRNA in Chronic Inflammation Sites, Mol. Ther.—Nucleic Acids. 5 (2016) e340. https://doi.org/10.1038/mtna.2016.39.); (S.-D. Li, L. Huang, Stealth nanoparticles: High density but sheddable PEG is a key for tumor targeting, J. Control. Release. 145 (2010) 178-181. https://doi.org/10.1016/j.jconrel.2010.03.016.) and lipids (C. Zhao, H. Deng, J. Xu, S. Li, L. Zhong, L. Shao, Y. Wu, X. J. Liang, Sheddable PEG-lipid to balance the contradiction of PEGylation between long circulation and poor uptake, Nanoscale. 8 (2016) 10832-10842. https://doi.org/10.1039/c6nr02174c.); (M. Kanamala, B. D. Palmer, H. Ghandehari, W. R. Wilson, Z. Wu, PEG-Benzaldehyde-Hydrazone-Lipid Based PEG-Sheddable pH-Sensitive Liposomes: Abilities for Endosomal Escape and Long Circulation, Pharm. Res. 35 (2018) 154. https://doi.org/10.1007/s11095-018-2429-y.). However, limited work has been carried out in the context of RNA. Wang and co-workers attached an anionic polymer-PEG conjugate to a positively charged ssPEI₈₀₀/siRNA nanoparticle, whereby a lowered pH degraded amide bonds of the PEGylated polymer and generated positively charged groups that repelled the nanoparticle. (X.-Z. Yang, J.-Z. Du, S. Dou, C.-Q. Mao, H.-Y. Long, J. Wang, Sheddable Ternary Nanoparticles for Tumor Acidity-Targeted siRNA Delivery, ACS Nano. 6 (2012) 771-781. https://doi.org/10.1021/nn204240b.). Sheddable PEGs have also been described in the art on spherical nucleic acids (C. A. Mirkin, B. R. Meckes, W. Zhang, Spherical Nucleic Acids (SNAs) with Sheddable PEG Layers, WO 2019/070890 A1, 2018.).

In one aspect provided herein, the present disclosure describes nucleic acids that are conjugated with PEG via a cleavable linkage. The nucleic acid may be DNA, RNA or XNA. Where XNA is a xeno nucleic acid that has a different sugar backbone to DNA or RNA.

In some embodiments, the linker between the PEG and the nucleotide may be a cleavable linker that can be cleaved in response to external stimuli. These may or may not be the same linkers that can be used for attachment of alternative cargo to oligonucleotides. The cleavage mechanism could occur intracellularly or extracellularly. In some embodiments, a cleavable linker described herein could be cleaved in response to a lowering in pH. In some embodiments, the linker could be cleaved in response to a reduced oxygen environment within the extracellular space. In some embodiments, the linker could be cleaved by an external small molecule, which may or may not be released as part of a delivery system. In some embodiments, the linker may be cleaved by extracellular enzymes, including, but not limited to, endonucleases, peptidases, proteases, matrix metalloproteinases and glycosidases. In some embodiments, the linker may be cleaved by intracellular enzymes, including, but not limited to, endonucleases, proteases and peptidases. In further embodiments, the linker may be cleaved by the antioxidant glutathione, either intracellularly or extracellularly. In some embodiments, the enzyme is present in the tumor microenvironment.

In some embodiments, the PEG molecule may be conjugated on the sugar or the base.

In further embodiments, the nucleic acids may form part of a larger oligonucleotide chain.

Accordingly, the PEG molecule may also be covalently conjugated to the phosphate backbone via a cleavable linkage. In further embodiments, the PEG molecule may be electrostatically bound to the phosphate backbone.

In some aspects the disclosure provides a nucleic acid nanoparticle that is formulated from the component oligonucleotides that contain the sheddable PEG.

In some embodiments, the nucleic acid nanoparticle is highly modified with unnatural nucleotides that modulate biological properties, for example and without limitation, biodistribution, cell uptake, endosomal escape and gene silencing. In some embodiments, these modifications are hidden by the sheddable PEG layer.

Accordingly, the nucleic acid nanoparticle may be conjugated to a cargo, which may be conjugated to one or more sheddable PEG moieties.

In further embodiments, the electrostatic molecule might be any other molecule with a primary amine. These may be electrostatically bound to the phosphate backbone to aid in formulation. Alternatively, these may be electrostatically bound to the phosphate backbone to exert another effect, such as a therapeutic effect.

Accordingly, modifications may also include, but are not limited to, carbohydrates, peptides, proteins, polymers and small molecules.

Such methodology allows for high loading of any given electrostatic molecule. This methodology could be particularly useful in formulation of a delivery vehicle with a carrier drug molecule.

Accordingly, in other aspects, electrostatic modifiers may be used where solubility is particularly challenging. This could include oligonucleotides whereby the component nucleotides are heavily modified, either on the ribose or the base, or a combination thereof.

In further aspects, electrostatic modifiers may be used to solubilise hydrophobic oligonucleotide conjugates, including, but not limited to, oligonucleotide-peptide conjugates, oligonucleotide-lipid conjugates, oligonucleotide-polymer conjugates and oligonucleotide-small molecule conjugates. Such conjugates may be conjugated at the 3′ or 5′ position, or a combination thereof.

### Definitions

The following definitions will be useful in understanding the present disclosure. Certain terms employed in the specification, examples and appended claims are collected here. These definitions should be read in light of the remainder of the disclosure and understood by a person of skill in the art. Unless defined otherwise, all technical and scientific terms used herein have the same meaning as those commonly used in the art. Standard techniques may be used for chemical synthesis, chemical analysis and biological assays.

In certain embodiments, compositions of the disclosure include nanoparticles. As used herein, “nanoparticle” refers to particles having dimensions that are measured on the nanometer scale. For example, a nanoparticle may have a diameter, length, width, or depth of from 1 to 1000 nm. The nanoparticles disclosed in this disclosure incorporate various chemistries that were previously disclosed (COMPOSITIONS CONTAINING NUCLEIC ACID NANOPARTICLES WITH MODULAR FUNCTIONALITY, WO 2021/220.053, 2021.). This is hereby incorporated herein by reference in its entirety and key components that are relevant to the present disclosure are also included.

As used herein, “nanostructure”, “nanoscaffold” or “nanoconstruct” refers generally to a nanoparticle to which other molecules can be attached. These may also be referred to, interchangeably, as Mergos. The term “Mergo” or “Mergos” refers to a single or double stranded oligonucleotide with minimum length of 8 nucleotides, where the function is not explicitly of a NAT, but is instead intended to enhance the delivery and functionality of NATs. Mergos are designed to be directly or indirectly linked to one or more therapeutic cargo molecules. Cargo molecules could be NATs. Alternatively, cargo molecules could be, but are not limited to non-NAT molecules such as, peptides, proteins, protein domains, antibodies, antibody fragments, antibody mimetics, lectins, vitamins, lipids, carbohydrates, benzamides and therapeutic small molecules, or combinations thereof.

Additionally, the minimum requirements for the definition of a nucleic acid nanostructure, as defined herein, pertains to duplexes of oligonucleotides with a minimum length of 10 bp. When considering B-DNA, described by James Watson and Francis Crick, which is believed to predominate in cells, extends to about 34 Å (3.4 nm) per 10 bp of sequence; A-DNA extends about 23 Å (2.3 nm) per 10 bp of sequence, and Z-DNA extends about 38 Å (3.8 nm) per 10 bp of sequence. As such, duplexes of DNA, RNA and XNA, with a length of 10 bp can be considered as nanostructures.

As used herein, The terms “polynucleotide”, “nucleic acid”, or “oligonucleotide” refer to a polymer of nucleotides. The terms “polynucleotide”, “nucleic acid”, and “oligonucleotide”, may be used interchangeably. Typically, a polynucleotide comprises at least two nucleotides. DNAs and RNAs are polynucleotides.

The terms “polypeptide”, “peptide”, and “protein”, may be used interchangeably to refer a string of at least three amino acids linked together by peptide bonds.

The terms “polysaccharide”, “carbohydrate”, or “oligosaccharide” may be used interchangeably to refer to a polymer of sugars. Typically, a polysaccharide comprises at least two sugars.

As used herein, the term “small molecule”, refers to an organic or inorganic compound, either synthesized in the laboratory or found in nature, which has a molecular weight of less than about 2000 g/mol, or less than about 1000 g/mol, and even less than about 500 g/mol.

As used herein, the term “moiety” refers to a portion of a molecule with a specific function. This can include functional groups that are able to undergo specific reactions. These may be used to attach molecules to nucleic acids and oligonucleotides.

As used herein, the term “Nucleic Acids Therapies (NATs)” refer to single or double stranded oligonucleotides that are exploited as modalities for a variety therapeutic and/or vaccine purposes. NATs include many different types of modalities, including, but not limited to, DNA-based gene therapies, RNA interference (RNAi), microRNAs (miRNAs), antisense oligonucleotides (ASO), long non-coding RNA (lncRNA), messenger RNA (mRNA), aptamers, and self amplifying RNA (saRNA).

As used herein, the term “theranostic” refers to a molecule that has both a therapeutic component and a diagnostic function. The therapeutic component encompasses NATs, but could also encompass other therapeutic molecules, including, but not limited to, small molecules, peptides and oligosaccharides. The diagnostic component encompasses a moiety that can be used to detect the theranostic molecule in a particular tissue. This could include, but is not limited to, a fluorophore, a radiolabel or an oligonucleotide barcode.

As used herein, the term “nucleic acid barcode” or “NA barcode” refers to a single or double stranded oligonucleotide sequence that is designed to unambiguously identify a particular chemical entity, or its chemical composition, usually to discriminate between multiple different entities in a multiplex assay. When linked directly or indirectly to a chemical entity, the barcode can be used to quantify the amount of that particular entity present in a sample, even within complex biological materials such as tissues, blood and urine. Chemical entities may include, but are not limited to, oligonucleotides, NATs, Mergos, lipid nanoparticles, polymers or antibodies. The nucleic acid barcode may be covalently or noncovalently linked or otherwise associated, encapsulated, embedded or incorporated with or within the chemical entity. The barcode sequences are then determined for example, using techniques include, but are not limited to, next-generation sequencing, microarray, quantitative PCR, in-situ hybridisation, and branched DNA assays. Suitable sequencing techniques include, but are not limited to, bridge amplification sequencing/Solexa (Illumina), ion semiconductor sequencing, GenapSys sequencing, combinatorial probe-anchor synthesis (cPAS), sequencing by ligation (SOLiD sequencing), single molecule real-time sequencing (SMRT), Heliscope single molecule sequencing, nanopore sequencing, pyrosequencing, and Sanger sequencing.

As used herein, “nucleic acid analyte”, refers to a nucleic acid molecule that is the target of an analysis method aimed at detecting, identifying and/or quantifying the analyte in a biological sample. The term includes but is not limited to nucleic acid molecules that are man-made, for example by chemical or enzymatic synthesis, rather than of natural origin. Analytical methods used to detect, identify and/or quantify the nucleic acid analyte include but are not limited to quantitative real-time polymerase chain reaction, digital PCR, ligation-dependent probe amplification, RCA-assisted single-molecule flow cytometry, microarray, DNA or RNA sequencing, single cell sequencing, and RNA in situ hybridization.

As used herein, the term “multiplex” or “multiplexing” refers to the parallel analysis of pooled oligonucleotide samples.

As used herein, the term “biological matrix” or “biological matrices” refers to a material (tissue) that is collected by a living organism and may or may not connect eukaryotic cells.

As used herein, the term “unique molecule identifier” or “UMI” refers to a random sequence of nucleotides whereby the nucleobase at each randomized position is stochastically generated from a mixture of more than one type of nucleobases (e.g. A, C, G, T, U) during oligonucleotide synthesis.

### Nanoparticles, Including Nucleic Acid Nanoparticles

Various nucleic acid nanostructures having ordered two-dimensional or three-dimensional structures are known, including, for example and without limitation, three-way junctions (3WJ) nanoarrays, nanocages, nanocubes, nanoprisms, nanorings, nanoscaffolds, and nanotubes. Nanorings may be symmetrical structures that include 3, 4, 5, 6, 7, 8, or more oligonucleotides arrayed around an axis. Thus, nanorings may be trimers, tetramers, pentamers, hexamers, heptamers, oxamers, or higher-numbered polymers. Nanorings may be circular, triangular, square, pentagonal, hexagonal, heptagonal, octagonal, or otherwise polygonal in shape. Other types of nucleic acid nanoparticles, such as sheets, cages, dendrimers and clusters, are also possible and within the scope of the disclosure.

Nucleic acid nanoparticles may contain naturally occurring nucleotides, or they may contain chemically-modified nucleotides. Chemically modified nucleotides are known in the art and described in, for example, WO 2018/118,587, the contents of which are incorporated herein by reference. For example, and without limitation, nucleic acid nanoparticles, therapeutics and aptamers may contain one or more of a 2′ fluoro, 2′ O-methyl, 2-thiouridine, 2′-O-methoxyethyl, 2′-amine, 5-methoxyuridine, pseudouridine, 5-methylcytidine, N1-methyl-pseudouridine, locked nucleic acid (LNA), morpholino, and phosphorothioate modification. Other modified nucleotides include 5caC, 5fC, 5hoC, 5hmC, 5meC/5fu, 5meC/5moU, 5meC/thG, 5moC, 5meC/5camU, 5meC, ψ, 5meC/ψ, 5moC/5moU, 5moC/5meU, 5hmC/5meU, me1ψ, 5meC/me1ψ, 5moU, 5camU, m6A, 5hmC/ψ, 5moC/ψ, me6DAP, me4C, 5fu, 5-methoxyuridine, 2-aminoadenine, 2-thiocytosine, 2-thiothymine, 2-thiouracil, 3-methyladenine, 4-thiouracil, 5,6-dehydrouracil, 5-allylcytosine, 5-allyluracil, 5-aminoallylcytosine, 5-aminoallyluracil, 5-bromouracil, 5-ethynylcytosine, 5-ethynyluracil, 5-fluorouracil, 5-hydroxymethylcytosine, 5-hydroxymethyluracil, 5-iodouracil, 5-methylcytosine, 5-methyluracil, 5-propynylcytosine, 5-propynylcytosine, 5-propynyluracil, 5-propynyluracil, 6-O-methylguanine, 6-thioguanine, 7-deaza-8-azaguanine, 7-deazaadenine, 7-deazaguanine, 7-deazaguanine, 8-oxoadenine, 8-oxoguanine, 5-methylcytidine, pseudouridine, inosine, 2′-O-methyladenosine, 2′-O-methylcytidine, 2′-O-methylguanosine, 2′-O-methyluridine, 2′-O-methyl-pseudouridine, 2′-O-methyl 3′-phosphorothioate adenosine, 2′-O-methyl 3′-phosphorothioate cytidine, 2′-O-methyl 3′-phosphorothioate guanosine, 2′-O-methyl 3′-phosphorothioate uridine, a conformationally-restricted nucleotide, and 2′-O-methyl 3′-phosphorothioate pseudouridine.

The nucleic acids of the nanoparticles may contain sugar modifications. For example and without limitation, the nucleic acids of the nanoparticles may contain one or more of 2′MOE, 2′OMe, 2′F, 2-′O-acetalesters, GMEBuOM, AMPrOM, AMEBuOM, PivOM, 2′ amino locked nucleic acids (LNA) modified with amines or peptides mentioned above, 2′-O-[N,N-dimethylamino)ethoxy]ethyl, 2′-N-[N,N-dimethylamino)ethoxy]ethyl, 2′-N-imidazolacetyamide, 2′-O-[3-(guanidinium)propyl], 2′-N-[3-(guanidinium)propyl], 2′-O-[3-(guanidinium)ethyl], 2′-N-[3-(guanidinium)ethyl], 2′-O-(N-(aminoethyl)carbamoyl)methyl, 2′-N-(N-(aminoethyl)carbamoyl)methyl, 2′-O-[N-(2-((2-aminoethyl)amino)ethyl)]acetamide, 2′-N-[N-(2-((2-aminoethyl)amino)ethyl)]acetamide, 2′-N-2,2,3,3,4,4,5,5,6,6,7,7,8,8,8-pentadecafluorooctanamide, 2′-N-imidazolacetamide, 2′-O-imidazole methyl, 2′-N-guanidylbenzylamide, and 4′-C-guanidinincarbohydrazidomethyl, 2′-O-imidazolemethyl, 2′-N-imidazolemethylamine ethyl.

In embodiments, the disclosure provides compositions comprising a cargo molecule and a nucleic acid nanoparticle attached to the cargo molecule, wherein the nucleic acid nanoparticle is functionalized to promote a biological activity of the cargo molecule in a subject.

In embodiments, the nucleic acid nanoparticle is functionalized to promote internalisation into a cell.

In embodiments, the cargo molecule is functionalized to promote internalisation into a cell.

In embodiments, the cargo molecule is an anchored cholesterol molecule that promotes permeation through the lipid bilayer of the cell.

In embodiments, the functionalization promotes internalisation into the cell via clathrin-mediated endocytosis, non-clathrin/non-caveolae endocytosis, caveolae-mediated endocytosis, passive diffusion, simple diffusion, facilitated diffusion, transcytosis, macropinocytosis, phagocytosis, receptor mediated endocytosis, receptor diffusion, vesicle-mediated transport, active transport.

In embodiments, the nucleic acid nanoparticle may enter the cell, or be processed, via the endosome, lysosome, pinosome, or phagosome.

In embodiments, the nucleic acid nanoparticle may enter the cell across a biological membrane.

In embodiments, the functionalization may take effect in the cell cytoplasm. nucleus, mitochondria or other cellular compartment.

In embodiments, the cargo molecule is selected from the group consisting of mRNA, gRNA/CRISPR, siRNA, ASO, miRNA, InRNA, shRNA, saRNA, AD-gRNA, ribozymes, aptamers, peptides, proteins, antibodies, carbohydrates, and therapeutic small molecules.

In embodiments, the oligonucleotides are assembled into a nucleic acid nanoparticle.

In embodiments, the disclosure provides compositions comprising a first cargo molecule and a second cargo molecule linked to the first cargo molecule.

In embodiments, the first cargo molecule has a biological function.

In embodiments, the first cargo molecule is selected from the group consisting of mRNA, gRNA/CRISPR, siRNA, ASO, miRNA, InRNA, shRNA, saRNA, AD-gRNA, ribozymes, aptamers, peptides, proteins, antibodies, carbohydrates and therapeutic small molecules.

In embodiments, the first cargo molecule is a cell-or tissue-targeting ligand comprising an aptamer, lectin, glycoprotein, lipid, antibody, nanobody, or DARPIN.

In embodiments, at least one of the first and second cargo molecules comprises GalNAc or a GalNAc derivative that is linked via a monovalent, bivalent, or trivalent branched linker.

In embodiments, at least one of the first and second cargo molecules comprises cholesterol or a derivative thereof.

In embodiments, at least one of the first and second cargo molecules comprises a phospholipid.

In embodiments, at least one of the first and second cargo molecules comprises a cationic lipid, optionally comprising a quaternary ammonium ion.

In embodiments, at least one of the first and second cargo molecules comprises an anionic lipid, optionally comprising a phosphate group.

In embodiments, at least one of the first and second cargo molecules comprises an ionizable lipid.

In embodiments, at least one of the first and second cargo molecules comprises a branched lipid.

In embodiments, the first cargo molecule is linked to the second cargo molecule by a cleavable linker.

In embodiments, the first cargo molecule and the second cargo molecule are siRNAs.

In embodiments, the first cargo molecule and the second cargo molecule are linked via an oligonucleotide spacer from the group consisting of (dT)n, (dA)n, d(C)n, d(G)n, (rU)n, (rA)n, (rC)n, (rG)n, and combinations thereof, wherein n is 1-16.

In embodiments, at least one of the first and second cargo molecules is linked to a third cargo molecule.

In embodiments, at least one of the first and second cargo molecules is linked to the third cargo molecule by a thiol-cleavable linker comprising dithiobismaleimidoethane and 1,4-bis[3-(2-pyridyldithio)propionamido]butane.

In embodiments, at least one of the first and second cargo molecules is linked to the third cargo molecule by a hydroxylamine-cleavable linker comprising ethylene glycol bis(succinimidyl succinate.

In embodiments, at least one of the first and second cargo molecules is linked to the third cargo molecule by a base-cleavable linker comprising bis[2-(N-succinimidyl-oxycarbonyloxy)ethyl]sulfone.

In embodiments, at least one of the first and second cargo molecules is linked to the third cargo molecule by a Meldrum's acid derivative comprising 5-(bis(methylthio)methylene)-2,2-dimethyl-1,3-dioxane-4,6-dione.

In embodiments, at least one of the first and second cargo molecules is linked to the third cargo molecule via a covalent bond.

In embodiments, at least one of the first and second cargo molecules is linked to the third cargo molecule by a dicer substrate.

In embodiments, at least one of the first and second cargo molecules is linked to the third cargo molecule with a linker selected from the group consisting of 1,8-bismaleimido-diethyleneglycol, 1,11-bismaleimido-triethyleneglycol, 1,4-bismaleimidobutane, bismaleimidohexane, bismaleimidoethane, tris(2-maleimidoethyl)amine), N-α-maleimidoacet-oxysuccinimide ester, N-β-maleimidopropyl-oxysuccinimide ester, N-ε-maleimidocaproic acid, N-γ-maleimidobutyryl-oxysuccinimide ester, succinimidyl 4-(N-maleimidomethyl)cyclohexane-1-carboxy-(6-amidocaproate), succinimidyl 6-(3(2-pyridyldithio)propionamido)hcxanoate, m-maleimidobenzoyl-N-hydroxysuccinimide ester, succinimidyl iodoacetate, succinimidyl (4-iodoacetyl)aminobenzoate, PEGylated, long-chain SMCC crosslinkers, succinimidyl 4-(p-maleimidophenyl)butyrate and sulfo-NHS equivalents), p-maleimidophenyl isocyanate.

In embodiments, at least one of the first and second cargo molecules is linked to a nanoparticle.

In embodiments, the disclosure provides compositions comprising at least two cargo molecules and a nucleic acid nanoparticle attached to each of the at least two cargo molecules.

In embodiments, each of the at least two cargo molecules has a biological function.

In embodiments, each of at least two cargo molecules is selected from the group consisting of mRNA, gRNA/CRISPR, siRNA, ASO, miRNA, lnRNA, shRNA, ribozymes, aptamers, peptides, proteins, antibodies and therapeutic small molecules.

In embodiments, more than one of the at least two cargo molecules are conjugated to the same nucleic acid nanoparticle.

In embodiments, the more than one of the at least two cargo molecules are different.

In embodiments, the more than one cargo molecules are the same.

In embodiments, the different cargo molecules are conjugated to the nanoparticle in unequal amounts.

In embodiments, the at least two cargo molecules are conjugated via a stable covalent bond.

In embodiments, the at least two molecules are conjugated via a stable covalent bond to a stimuli-responsive linker.

In embodiments, the disclosure provides compositions comprising an oligonucleotide covalently linked to one or more cargo molecules.

In embodiments, the oligonucleotides are functionalized with reactive sites that allow for conjugation and conjugated to a nucleic acid nanoparticle.

In embodiments, the nucleic acid nanoparticle is a tertiary structure of three or more junctions, said junctions are formed by at least two oligonucleotide strands of 3 to 200 nucleotides in length that partially interact with one another through hydrogen bonding or base-stacking interactions.

In embodiments, each nucleotide optionally comprises a modification including, but not limited to, 2′-O-methyl, 2′-fluoro, 2′-F-arabinonucleic acid, 2′-O-methoxyethyl, locked nucleic acid, unlocked nucleic acid, 4′-thioribonucleoside, 4′-C-aminomethyl-2′-O-methyl, cyclohexenyl nucleic acid, hexitol nucleic acid, glycol nucleic acid, phosphorothioate, boranophosphate, 5′-C-methyl, 5′(E)-vinylphosphonate, and 2′ thiouridine.

In embodiments, the nucleic acid nanoparticle is attached to a cargo molecule, wherein the cargo molecule promotes a biological activity of the cargo molecule in a subject.

In embodiments, the nucleic acid nanoparticle performs at least one biological activity selected from the group consisting of (i) binding to a serum protein in blood, or to a receptor in a cell or at the cell surface, (ii) promoting endosomal escape of the cargo molecule in a receptor-independent manner, (iii) targeting a tissue in an animal or subject, (iv) modulating biodistribution, (v) inducing or preventing an immunological response, (vi) enhancing cellular uptake, (vii) modulating gene expression, (viii) inducing cytotoxicity, and (ix) having a therapeutic effect, or combinations thereof.

In embodiments, the one or more cargo molecules are comprised of at least one of mRNA, gRNA/CRISPR, siRNA, shRNA, ASO, saRNA, miRNA, lnRNA, ribozyme, aptamer, peptide, protein, protein domain, antibody, antibody fragment, antibody mimetic, lectin, vitamin, lipid, carbohydrate, benzamides and therapeutic small molecules, or combinations thereof.

In embodiments, the functionalization promotes internalisation into the cell, wherein the internalisation mechanism comprises at least one of clathrin-mediated endocytosis, non-clathrin/non-caveolae endocytosis, caveolae-mediated endocytosis, passive diffusion, simple diffusion, facilitated diffusion, transcytosis, macropinocytosis, phagocytosis, receptor mediated endocytosis, receptor diffusion, vesicle-mediated transport, and active transport.

In embodiments, the attachment of the nucleic acid nanoparticle to at least one cargo molecule is obtainable by a method comprising at least one reaction that comprises at least one of the following features: (i) the reaction occurs in one pot, (ii) the reaction is not disturbed by water, (iii) the reaction generates minimal byproducts, and (iv) the reaction comprises a high thermodynamic driving force that affords a single reaction product.

In embodiments, the attachment reaction comprises: (i) attaching a first cargo molecule via a first reaction comprising at least one of the features described above and (ii) attaching a second cargo molecule via a second reaction comprising at least one of the features of described above, wherein the first reaction and the second reaction are orthogonal.

In embodiments, the oligonucleotide 5′, 3′ or internal position (at any given position on a nucleotide) is modified with a moiety that will allow for the formation of covalent bonds via reactions selected from the group consisting of CuAAC, SPAAC, RuAAC, IEDDA, SuFEx, SPANC, hydrazone/oxime ether formation, thiol-ene radical reaction, thiol-yne radical reaction, thiol-Michael addition reaction, thiol-isocyanate reaction, thiol-epoxide click reaction, nucleophilic ring opening reactions (spring-loaded reactions), traceless Staudinger ligation. These linkages may be formed by carrying out coupling reactions with any oligonucleotide or cargo molecule modified with a chemical moiety from the group consisting of, but not limited to, ADIBO-PEG4, N-[(1R,8S,9s)-bicyclo[6.1.0]non-4-yn-9-ylmethyloxycarbonyl]-1,8-diamino-3,6-dioxaoctane, (1R,8S,9s)-bicyclo[6.1.0]non-4-yn-9-ylmethanol, bromoacetamido-dPEG®4-amido-DBCO, bromoacetamido-dPEG®12-amido-DBCO, bromoacetamido-dPEG®24-amido-DBCO, dibenzocyclooctyne-acid, dibenzocyclooctyne-N-hydroxysuccinimidyl ester, dibenzocyclooctyne-PEG4-acid, dibenzocyclooctyne-PEG4-alcohol, dibenzocyclooctyne-PEG4-N-hydroxysuccinimidyl ester, (4-(1,2,4,5-tetrazin-3-yl)phenyl)methanamine hydrochloride, (E)-cyclooct-4-enol, (E)-cyclooct-4-enyl 2,5-dioxo-1-pyrrolidinyl carbonate, 2,5-Dioxo-1-pyrrolidinyl 5-[4-(1,2,4,5-tetrazin-3-yl)benzylamino]-5-oxopentanoate, 5-[4-(1,2,4,5-tetrazin-3-yl)benzylamino]-5-oxopentanoic acid, 5-norbornene-2-acetic acid succinimidyl ester, 5-norbornene-2-endo-acetic acid, methyltetrazine-NHS ester, methyltetrazine-PEG4-NHS ester, TCO PEG4 succinimidyl ester, TCO-amine, tetrazine-PEG5-NHS ester, alkyne-PEG5-acid, (R)-3-amino-5-hexynoic acid hydrochloride, (S)-3-amino-5-hexynoic acid hydrochloride, (S)-3-(boc-amino)-5-hexynoic acid, N-boc-4-pentyne-1-amine, boc-propargyl-Gly-OH, 3-ethynylaniline, 4-ethynylaniline, propargylamine hydrochloride, propargyl chloroformate, propargyl-N-hydroxysuccinimidyl ester, propargyl-PEG2-acid. 3-(4-azidophenyl)propionic acid, 3-azido-1-propanamine, 3-azido-1-propanol, 4-carboxybenzenesulfonazide, 0-(2-aminoethyl)-O′-(2-azidoethyl)heptaethylene glycol, 0-(2-aminoethyl)-O′-(2-azidoethyl)nonaethylene glycol, 0-(2-aminoethyl)-O′-(2-azidoethyl)pentaethylene glycol, azido-dPEG®4(n)acid (where n could be 4, 8, 12, 24), azido-dPEG®(n)-amine (where n could be 7, 11, 23, 35), azido-dPEG@4(n) NHS ester (where n could be 4, 8, 12, 24), azido-dPEG®(n)-TFP ester (where n could be 4, 8, 12, 24, 36), 2-[2-(2-azidoethoxy)ethoxy]ethanol, O-(2-azidoethyl)-O-[2-(diglycolyl-amino)ethyl]heptaethylene glycol, 0-(2-azidoethyl)heptaethylene glycol, 0-(2-azidoethyl)-O′-methyl-triethylene glycol, 0-(2-azidoethyl)-O′-methyl-undecaethylene glycol, 17-azido-3,6,9,12,15-pentaoxaheptadecan-1-amine, 14-azido-3,6,9,12-tetraoxatetradecanoic acid, 11-azido-3,6,9-trioxaundecan-1-amine, bromoacetamido-dPEG®(n)azide (where n could be 3, 11, 23) and combinations thereof.

In embodiments, the nucleic acid nanoparticle is attached to a first cargo molecule, a second cargo molecule linked to the first cargo molecule, and optionally, further cargo molecules linked to the second or first cargo molecule.

In embodiments, the first cargo molecule is selected from the group consisting of at least one of mRNA, gRNA/CRISPR, siRNA, shRNA, ASO, saRNA, miRNA, lnRNA, ribozyme, aptamer, peptide, protein, protein domain, antibody, antibody fragment, antibody mimetic, lectin, vitamin, lipid, carbohydrate, benzamides and therapeutic small molecules, or combinations thereof.

In embodiments, the first cargo molecule is linked to the second cargo molecule by a cleavable linker.

In embodiments, at least one of the first and second cargo molecules is linked to a third cargo molecule by either a thiol-cleavable linker comprising dithiobismaleimidoethane and 1,4-bis[3-(2-pyridyldithio)propionamido]butane, a hydroxylamine-cleavable linker comprising ethylene glycol bis(succinimidyl) succinate, a base-cleavable linker comprising bis[2-(N-succinimidyl-oxycarbonyloxy)ethyl]sulfone or a Meldrum's acid derivative comprising 5-(bis(methylthio)methylene)-2,2-dimethyl-1,3-dioxane-4,6-dione.

In embodiments, at least one of the first and second cargo molecules is linked to a third cargo molecule by a dicer substrate or extended nucleic acid spacer region that is amenable to cleavage, including, but not limited to, the sequences oligo(T), oligo(A), oligo(G), oligo(C), and combinations thereof.

In embodiments, at least one of the first and second cargo molecules is linked to a third cargo molecule with a linker selected from the group consisting of 1,8-bismaleimido-diethyleneglycol, 1,11-bismaleimido-triethyleneglycol, 1,4-bismaleimidobutane, bismaleimidohexane, bismaleimidoethane, tris(2-maleimidoethyl)amine), N-α-maleimidoacet-oxysuccinimide ester, N-β-maleimidopropyl-oxysuccinimide ester, N-ε-maleimidocaproic acid, N-γ-maleimidobutyryl-oxysuccinimide ester, succinimidyl 4-(N-maleimidomethyl)cyclohexane-1-carboxy-(6-amidocaproate), succinimidyl 6-(3(2-pyridyldithio)propionamido)hexanoate, m-maleimidobenzoyl-N-hydroxysuccinimide ester, succinimidyl iodoacetate, succinimidyl (4-iodoacetyl)aminobenzoate, PEGylated, long-chain SMCC crosslinkers, succinimidyl 4-(p-maleimidophenyl)butyrate and sulfo-NHS equivalents), and p-maleimidophenyl isocyanate.

In embodiments, the second cargo molecule is linked to any given number of cargo molecules in a polymeric fashion.

In embodiments, the first cargo molecule is linked to the nucleic acid nanoparticle via reactions selected from the group consisting of CuAAC, SPAAC, RuAAC, IEDDA, SuFEx, SPANC, hydrazone/oxime ether formation, thiol-ene radical reaction, thiol-yne radical reaction, thiol-Michael addition reaction, thiol-isocyanate reaction, thiol-epoxide click reaction, nucleophilic ring opening reactions (spring-loaded reactions), traceless Staudinger ligation. These linkages may be formed by carrying out coupling reactions with any oligonucleotide or cargo molecule modified with a chemical moiety from the group consisting of, but not limited to, ADIBO-PEG4, N-[(1R,8S,9s)-bicyclo[6.1.0]non-4-yn-9-ylmethyloxycarbonyl]-1,8-diamino-3,6-dioxaoctane, (1R,8S,9s)-bicyclo[6.1.0]non-4-yn-9-ylmethanol, bromoacetamido-dPEG®4-amido-DBCO, bromoacetamido-dPEG®12-amido-DBCO, bromoacetamido-dPEG®24-amido-DBCO, dibenzocyclooctyne-acid, dibenzocyclooctyne-N-hydroxysuccinimidyl ester, dibenzocyclooctyne-PEG4-acid, dibenzocyclooctyne-PEG4-alcohol, dibenzocyclooctyne-PEG4-N-hydroxysuccinimidyl ester, (4-(1,2,4,5-tetrazin-3-yl)phenyl)methanamine hydrochloride, (E)-cyclooct-4-enol, (E)-cyclooct-4-enyl 2,5-dioxo-1-pyrrolidinyl carbonate, 2,5-Dioxo-1-pyrrolidinyl 5-[4-(1,2,4,5-tetrazin-3-yl)benzylamino]-5-oxopentanoate, 5-[4-(1,2,4,5-tetrazin-3-yl)benzylamino]-5-oxopentanoic acid, 5-norbornene-2-acetic acid succinimidyl ester, 5-norbornene-2-endo-acetic acid, methyltetrazine-NHS ester, methyltetrazine-PEG4-NHS ester, TCO PEG4 succinimidyl ester, TCO-amine, tetrazine-PEG5-NHS ester, alkyne-PEG5-acid, (R)-3-amino-5-hexynoic acid hydrochloride, (S)-3-amino-5-hexynoic acid hydrochloride, (S)-3-(boc-amino)-5-hexynoic acid, N-boc-4-pentyne-1-amine, boc-propargyl-Gly-OH, 3-ethynylaniline, 4-ethynylaniline, propargylamine hydrochloride, propargyl chloroformate, propargyl-N-hydroxysuccinimidyl ester, propargyl-PEG2-acid, 3-(4-azidophenyl)propionic acid, 3-azido-1-propanamine, 3-azido-1-propanol, 4-carboxybenzenesulfonazide, 0-(2-aminoethyl)-O′-(2-azidoethyl)heptaethylene glycol, 0-(2-aminoethyl)-O′-(2-azidoethyl)nonaethylene glycol, 0-(2-aminoethyl)-O′-(2-azidoethyl)pentaethylene glycol, azido-dPEG®4(n)acid (where n could be 4, 8, 12, 24), azido-dPEG®(n)-amine (where n could be 7, 11, 23, 35), azido-dPEG®4(n) NHS ester (where n could be 4, 8, 12, 24), azido-dPEG®(n)-TFP ester (where n could be 4, 8, 12, 24, 36), 2-[2-(2-azidoethoxy)ethoxy]ethanol, O-(2-azidoethyl)-O-[2-(diglycolyl-amino)ethyl]heptaethylene glycol, 0-(2-azidoethyl)heptaethylene glycol, 0-(2-azidoethyl)-O′-methyl-triethylene glycol, 0-(2-azidoethyl)-O′-methyl-undecaethylene glycol, 17-azido-3,6,9,12,15-pentaoxaheptadecan-1-amine, 14-azido-3,6,9,12-tetraoxatetradecanoic acid, 11-azido-3,6,9-trioxaundecan-1-amine, bromoacetamido-dPEG®(n)azide (where n could be 3, 11, 23) and combinations thereof.

In embodiments, each of at least two cargo molecules has a biological function.

In embodiments, the disclosure provides compositions comprising an oligonucleotide covalently linked to one or more cargo molecules.

In embodiments, the composition comprises oligonucleotides that are functionalized with reactive sites that allow for conjugation and assembled into a nucleic acid nanoparticle.

In embodiments, the nucleic acid nanoparticle is attached to a cargo molecule, wherein the nucleic acid nanoparticle is functionalized to promote a biological activity of the cargo molecule in a subject.

In embodiments, the nucleic acid nanoparticle is trimeric, tetrameric, pentameric or hexameric.

In embodiments, the disclosure provides methods comprising attaching a nucleic acid nanoparticle to at least one cargo molecule via at least one reaction that comprises at least one of the following features: the reaction occurs in one pot, the reaction is not disturbed by water, the reaction generates minimal byproducts, and the reaction comprises a high thermodynamic driving force that affords a single reaction product.

In embodiments, the method comprises attaching a first cargo molecule via a first reaction comprising at least one of the features and attaching a second cargo molecule via a second reaction comprising at least one of the features, wherein the first reaction and the second reaction are orthogonal.

In embodiments, the first reaction comprises modification on a strand of at least one oligonucleotide in the nucleic acid nanoparticle.

In embodiments, the nucleic acid nanoparticle is attached to the first cargo molecule via copper (I) azide-alkyne cycloaddition.

In embodiments, the nucleic acid nanoparticle is attached to first the cargo molecule via strain-promoted azide-alkyne cycloaddition.

In embodiments, the nucleic acid nanoparticle is attached to the first cargo molecule via an inverse electron demand Diels Alder reaction.

In embodiments, the nucleic acid nanoparticle is attached to the first cargo molecule via a disulphide linkage.

In embodiments, the nucleic acid nanoparticle is attached to the first cargo molecule via sulfur (VI) fluoride exchange.

In embodiments, the nucleic acid nanoparticle is attached to the first cargo molecule via hydrazone formation.

In embodiments, the nucleic acid nanoparticle is attached to the first cargo molecule via a thiol-ene radical addition.

In embodiments, the nucleic acid nanoparticle is attached to the first cargo molecule via a thiol-yne reaction.

In embodiments, the nucleic acid nanoparticle is attached to the first cargo molecule via thiol-Michael addition.

In embodiments, the nucleic acid nanoparticle is attached to the first cargo molecule via thiol-isocyanate chemistry.

In embodiments, the nucleic acid nanoparticle is attached to the first cargo molecule via thiol-epoxide chemistry.

In embodiments, the nucleic acid nanoparticle is attached to the first cargo molecule via a nucleophilic ring opening reaction.

In embodiments, the nucleic acid nanoparticle is attached to the first cargo molecule via a traceless Staudinger ligation.

In embodiments, the oligonucleotide 5′, 3′ or internal position (at any given position on a nucleotide) is modified with a moiety that will allow for the formation of covalent bonds outlined via these methods. These linkages may be formed by carrying out coupling reactions with any oligonucleotide or cargo molecule modified with a chemical moiety from the group consisting of, but not limited to, ADIBO-PEG4, N-[(1R,8S,9s)-bicyclo[6.1.0]non-4-yn-9-ylmethyloxycarbonyl]-1,8-diamino-3,6-dioxaoctane, (1R,8S,9s)-bicyclo[6.1.0]non-4-yn-9-ylmethanol, bromoacetamido-dPEG®₄-amido-DBCO, bromoacetamido-dPEG ©₁₂-amido-DBCO, bromoacetamido-dPEG®₂₄-amido-DBCO, dibenzocyclooctyne-acid, dibenzocyclooctyne-N-hydroxysuccinimidyl ester, dibenzocyclooctyne-PEG4-acid, dibenzocyclooctyne-PEG4-alcohol, dibenzocyclooctyne-PEG4-N-hydroxysuccinimidyl ester, (4-(1,2,4,5-tetrazin-3-yl)phenyl)methanamine hydrochloride, (E)-cyclooct-4-enol, (E)-cyclooct-4-enyl 2,5-dioxo-1-pyrrolidinyl carbonate, 2,5-Dioxo-1-pyrrolidinyl 5-[4-(1,2,4,5-tetrazin-3-yl)benzylamino]-5-oxopentanoate, 5-[4-(1,2,4,5-tetrazin-3-yl)benzylamino]-5-oxopentanoic acid, 5-norbornene-2-acetic acid succinimidyl ester, 5-norbornene-2-endo-acetic acid, methyltetrazine-NHS ester, methyltetrazine-PEG4-NHS ester, TCO PEG4 succinimidyl ester, TCO-amine, tetrazine-PEG5-NHS ester, alkyne-PEG5-acid, (R)-3-amino-5-hexynoic acid hydrochloride, (S)-3-amino-5-hexynoic acid hydrochloride, (S)-3-(boc-amino)-5-hexynoic acid, N-boc-4-pentyne-1-amine, boc-propargyl-Gly-OH, 3-ethynylaniline, 4-ethynylaniline, propargylamine hydrochloride, propargyl chloroformate, propargyl-N-hydroxysuccinimidyl ester, propargyl-PEG2-acid, 3-(4-azidophenyl)propionic acid, 3-azido-1-propanamine, 3-azido-1-propanol, 4-carboxybenzenesulfonazide, 0-(2-aminoethyl)-O′-(2-azidoethyl)heptaethylene glycol, 0-(2-aminoethyl)-O′-(2-azidoethyl)nonaethylene glycol, 0-(2-aminoethyl)-O′-(2-azidoethyl)pentaethylene glycol, azido-dPEG®4(n)acid (where n could be 4, 8, 12, 24), azido-dPEG®(n)-amine (where n could be 7, 11, 23, 35), azido-dPEG®4(n) NHS ester (where n could be 4, 8, 12, 24), azido-dPEG®(n)-TFP ester (where n could be 4, 8, 12, 24, 36), 2-[2-(2-azidoethoxy)ethoxy]ethanol, 0-(2-azidoethyl)-O-[2-(diglycolyl-amino)ethyl]heptaethylene glycol, 0-(2-azidoethyl)heptaethylene glycol, 0-(2-azidoethyl)-O′-methyl-triethylene glycol, 0-(2-azidoethyl)-O′-methyl-undecaethylene glycol, 17-azido-3,6,9,12,15-pentaoxaheptadecan-1-amine, 14-azido-3,6,9,12-tetraoxatetradecanoic acid, 11-azido-3,6,9-trioxaundecan-1-amine, bromoacetamido-dPEG®(n)azide (where n could be 3, 11, 23) and combinations thereof.

In embodiments, the nucleic acid nanoparticle is attached to the first cargo via a linker responsive to a stimulus.

In embodiments, the stimulus is selected from the group consisting of pH, light, temperature, reduction potential or oxygen concentration.

In embodiments, the nucleic acid nanoparticle is covalently stabilized.

Rationally Designed RNA and Nucleic Acid-Based Delivery Vehicles with Theranostic Capabilities

The current disclosure discloses several nucleic acid delivery vehicles that incorporate components of natural and unnatural origin. Delivery vehicles that incorporate naturally occurring components are known in the art (P. Guo, pRNA MULTIVALENT JUNCTION DOMAIN FOR USE IN STABLE MULTIVALENT RNA NANOPARTICLES, WO 2012/170,372, 2012.; A. Hill, S. Schroeder, NOVEL pRNA THREE-WAY JUNCTIONS, WO 2017/147,557, 2017; P. Guo, MULTIVALENT RNA NANOPARTICLES FOR DELIVERY OF ACTIVE AGENTS TO A CELL, WO 2007/016,507, 2007.; P. Guo, pRNA CHIMERA, WO 2005/003,293, 2005; B. A. Shapiro, Y. G. Yingling, E. Bindewald, W. Kasprzak, L. Jaeger, I. Severcan, C. Geary, K. Afonin, RNA NANOPARTICLES AND NANOTUBES, U.S. Pat. No. 9,732,337, 2017; P. Guo, D. Shu, Y. Shi, H. Li, F. Haque, MIRNA FOR TREATMENT OF BREAST CANCER, U.S. Pat. No. 11,085,044, 2021.) and are incorporated herein by reference.

In some embodiments, the present disclosure outlines compositions based on modified natural RNA dimers. These dimers include, but are not limited to, the 3′UTR region of oskar mRNA (osk) (H. Jambor, C. Brunel, A. Ephrussi, Dimerization of oskar 3′ UTRs promotes hitchhiking for RNA localization in the Drosophila oocyte, Rna. 17 (2011) 2049-2057. https://doi.org/10.1261/ma.2686411.), the 3′ UTR domain III of bicoid (bcd) mRNA (D. Ferrandon, I. Koch, E. Westhof, C. Nusslein-Volhard, RNA-RNA interaction is required for the formation of specific bicoid mRNA 3′ UTR-STAUFEN ribonucleoprotein particles, EMBO J. 16 (1997) 1751-1758. https://doi.org/10.1093/emboj/16.7.1751.), the hatchet ribozyme (L. Zheng, C. Falschlunger, K. Huang, E. Mairhofer, S. Yuan, J. Wang, D. J. Patel, R. Micura, A. Ren, Hatchet ribozyme structure and implications for cleavage mechanism, Proc. Natl. Acad. Sci. U.S.A 166 (2019) 10783-10791. https://doi.org/10.1073/pnas.1902413116.) and various riboswitches.

The osk dimer is formed from a conserved six-nucleotide, GC-rich palindromic loop sequence in the region of 714-827 of the 3′UTR and homodimerization was shown in vitro. Such GC-rich dimers can be taken and used to form artificial constructs with kissing interactions.

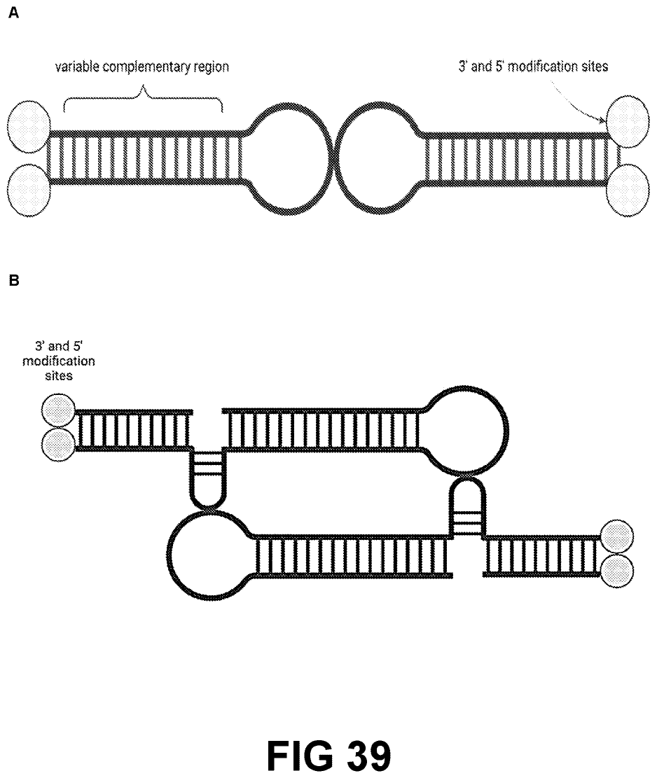

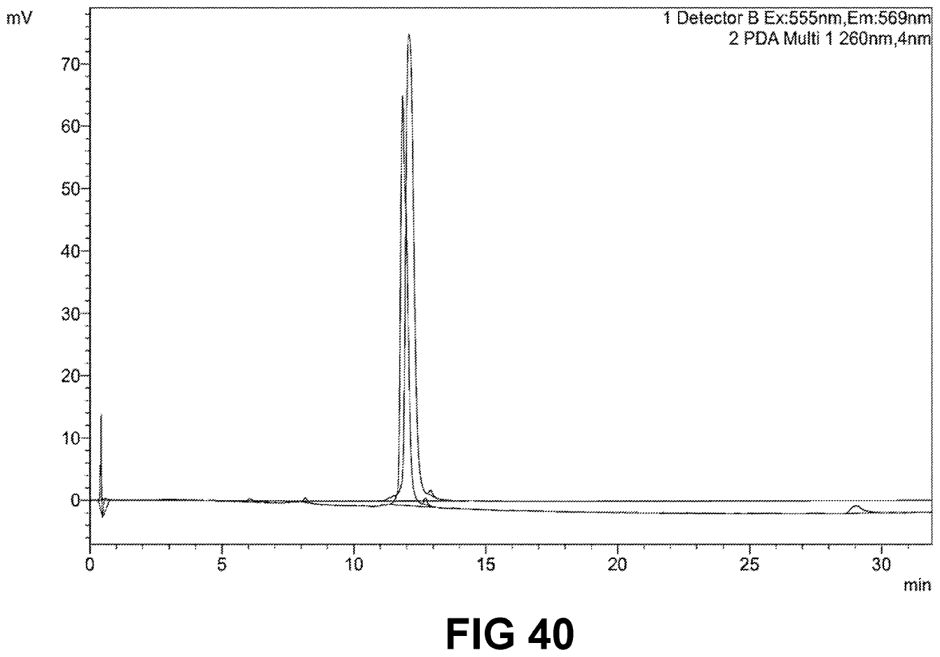

Examples of osk dimers in the present disclosure may contain the following sequence, or closely matching variations thereof (FIG. 39):

[complimentary sequence 1]-CACAAAATCAATACCGCGGTTGATTTTATAAAT-
[complimentary sequence 2]

Whereby the nucleic acid could be DNA, RNA, XNA, or combinations thereof. For RNA-containing sequences, the thymidine residues are switched for uridine residues. This sequence may form part of a larger sequence and will form a stem-loop within the dimerizing fragment (P. J. Webster, J. Suen, P. M. Macdonald, Drosophila virilis oskar transgenes direct body patterning but not pole cell formation or maintenance of mRNA localization in D. melanogaster. Development. 120 (1994) 2027-2037. https://doi.org/10.1242/dev.120.7.2027.).

Complementary sequence 1 has complementarity with complementary sequence 2; this sequence extends the stem-loop where appropriate.

In some embodiments, the osk dimers are modified at either the 5′ or 3′ end, or a combination of both. Modifications include, but are not limited to, ligation handles, linkers, cargo molecules, PK/PD modifications and targeting molecules. Cargo molecules include, but are not limited to, mRNA, gRNA/CRISPR, siRNA, shRNA, ASO, saRNA, miRNA, InRNA, ribozyme, aptamer, peptide, protein, protein domain, antibody, antibody fragment, antibody mimetic, lectin, vitamin, lipid, carbohydrate, benzamides and therapeutic small molecules, or combinations thereof.

In embodiments, the present disclosure may contain dimers formed from shortened sequences from the 3′ UTR domain III of bicoid (bcd) mRNA (D. Ferrandon, I. Koch, E. Westhof, C. Nüsslein-Volhard, RNA-RNA interaction is required for the formation of specific bicoid mRNA 3′ UTR-STAUFEN ribonucleoprotein particles, EMBO J. 16 (1997) 1751-1758. https://doi.org/10.1093/emboj/16.7.1751). In an in vitro setting, this dimer is formed via complementarity of the apical loop and an internal bulge. This can lead to both open and closed dimers which can further oligomerize. The naturally occurring dimer consists of two strands with the following sequence:

AUACGCUAUUCGCCUUAGAUGUAUCUAUCUUGGGUGGCUGCUCCACUAAA
GCCCGGGAAUAUGCAACCAGUUACAUUUGAGGCCAUUUGGGCUUAAGCGU
A

This 101 nt sequence is not viable for chemical synthesis, therefore a shortened form is disclosed as:

AUACGCUAUUCGCCUUCUCCACUA-[link]-
AAGCCCGGGAAUGAGGCCAUUUGGGCUUAAGCGUA

Whereby the nucleic acid could be DNA, RNA, XNA, or combinations thereof. The -[link]-could be any given natural oligonucleotide linkage, such as a phosphodiester, or could be a chemically modified linkage formed via a click ligation.

In some embodiments, the bicoid dimers are modified at either the 5′ or 3′ end, or a combination of both. Modifications include, but are not limited to, ligation handles, linkers, cargo molecules, PK/PD modifications and targeting molecules. Cargo molecules include, but are not limited to, mRNA, gRNA/CRISPR, siRNA, shRNA, ASO, saRNA, miRNA, lnRNA, ribozyme, aptamer, peptide, protein, protein domain, antibody, antibody fragment, antibody mimetic, lectin, vitamin, lipid, carbohydrate, benzamides and therapeutic small molecules, or combinations thereof.

Compositions in the present disclosure may incorporate a chemical identifier that allows for the generation of a detectable signal in a biological sample of interest. The biological sample can be a cellular organelle, a single cell, a plurality of cells, an organ, a tissue, a tissue extract, a biofluid (including, but not limited to, blood, urine, and saliva) or an entire organism.

In some embodiments, the chemical identifier is a nucleic acid barcode, whereby the nucleic acid could be DNA, RNA, XNA or a combination of one or more types.

In particularly preferred embodiments, the nucleic acid barcode is designed such that no two barcoded entities share the same barcode sequence. Therefore, any two entities out of a given set will be distinguishable by their unique barcodes and their barcodes will differ in at least one nucleotide position. In a preferred embodiment of the disclosure, each barcode will differ in at least 3 nucleotide positions from all other barcodes in the set.

In representative embodiments, the barcoded strand is of formula (I)

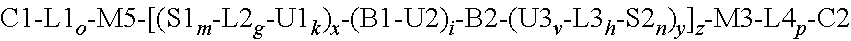

wherein
C1 is an optional cargo molecule,
L1 is an optional linker covalently bound to C1 and one of either
   M5 (if M5 is present),
   S1 when x and m are each independently greater than 0 and M5 is absent, or
   B1 when x is 0 and i is greater than 0 and M5 is absent, or
   B2 when x and i are each independently 0 and M5 is absent,
o is 0 or 1, wherein o is always 0 in the absence of C1,
M5 is an optional 5′ modification,
S1 is an optional nucleic acid sequence upstream of the barcode,
m is 0 or 1, wherein m is less than or equal to x,
L2 is an optional linker covalently bound to S1 and one of either
   U1 when k equals 1, or
   B1 when k equals 0 and i is greater than 0, or
   B2 when k and i each independently equal 0,
g is 0 or 1, wherein g is less than or equal to m,
U1 is either an optional unique molecular identifier sequence or an optional part of a multipartite UMI,
k is either 0 or 1, wherein k is less than or equal to m,
x is 0, 1, 2, 3, 4, 5, 6, 7, 8, 9 or 10, wherein x is greater than or equal to m,
B1 is an optional nucleic acid barcode sequence in instances where more than 1 barcode is included,
U2 is either an optional unique molecular identifier sequence or an optional part of a multipartite UMI,
i is 0, 1, 2, 3, 4, 5, 6, 7, 8, 9 or 10,
B2 is a nucleic acid barcode sequence,
U3 is either an optional unique molecular identifier sequence or an optional part of a multipartite UMI,
v is either 0 or 1, wherein v is less than or equal to n,
L3 is an optional linker covalently bound to S2 and one of either
   U3, when v equals 1, or
   B2, when v equals 0,
h is either 0 or 1, wherein h is less than or equal to n,
S2 is an optional nucleic acid sequence downstream of the barcode,
n is either 0 or 1, wherein n is less than or equal to y,
y is 0, 1, 2, 3, 4, 5, 6, 7, 8, 9 or 10, wherein y is greater than or equal to n,
z is 1, 2, 3, 4, 5, 6, 7, 8, 9 or 10,
M3 is an optional 3′ modification,
L4 is an optional linker covalently bound to C2 and one of either
   M3 (if M3 is present),
   S2 when y and n are each independently greater than 0 and M3 is absent, or
   B2 when y is 0 and M3 is absent,
p is 0 or 1, wherein p is always 0 in the absence of C2,
C2 is an optional cargo molecule.

In a preferred embodiment of the disclosure, the barcoded strand is of formula (II):

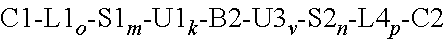

wherein
C1 is an optional cargo molecule,
L1 is an optional linker covalently bound to C1 and one of either
   S1 when m equals 1, or
   B2 when m equals 0,
o is 0 or 1, wherein o is always 0 in the absence of C1,
S1 is an optional nucleic acid sequence upstream of the barcode,
m is 0 or 1, wherein m is greater than or equal to k,
U1 is either an optional unique molecular identifier sequence or an optional part of a bipartite UMI,
k is either 0 or 1, wherein k is less than or equal to m,
B2 is a nucleic acid barcode sequence,
U3 is either an optional unique molecular identifier sequence or an optional part of a multipartite UMI,
v is either 0 or 1, wherein v is less than or equal to n,
S2 is an optional nucleic acid sequence downstream of the barcode,
n is either 0 or 1, wherein n is greater than or equal to v,
L4 is an optional linker covalently bound to C2 and one of either
   S2 when n equals 1, or
   B2 when n equals 0,
p is 0 or 1, wherein p is always 0 in the absence of C2,
C2 is an optional cargo molecule.

In some embodiments, the barcode sequence may be 2-500 or more nucleotides in length, and generally can be of any length manufacturable by oligonucleotide synthesis. In a preferred embodiment, the barcode may be 5-200 nucleotides in length, more preferably 8-20 nucleotides in length, including any length within these ranges, such as 3, 4, 5, 6, 7, 8, 9, 10, 11, 12, 13, 14, 15, 16, 17, 18, 19, 20, 25, 30, 40, 50, 60, 70, 80, 90, 100, 125, 150, 175, 200, 225, 250, 300, 400 or 500 nucleotides in length.

In some embodiments, each nucleotide in the barcode sequence optionally contains one or more modified nucleotides comprising a modification in a ribose group, a modification in a phosphate group, a modification in a nucleobase group, or a combination thereof. Chemical modifications to the ribose group may include, but are not limited to, 2′-O-methyl, 2′-fluoro, 2′-F-arabino, 2′-O-methoxyethyl, 2′-amino, 2′-deoxy, 2′-O-allyl, locked nucleic acid, unlocked nucleic acid, 2′,4′-constrained 2′-O-ethyl-bridged nucleic acid, arabinose, hexose, cyclohexenyl nucleic acid, hexitol nucleic acid, glycol nucleic acid, 4′-thioribonucleoside, and 4′-C-aminomethyl-2′-O-methyl. Chemical modifications to the phosphate group may include, but are not limited to, phosphorothioate, phosphorodithioate, alkylated phosphorothioate, boranophosphate, methylphosphonate, phosphoramidate, and amide linkages. Additional chemical modifications to the nucleobase, the ribose, and the phosphate linkage are well known to those of skill in the art and include modifications described in, for example, WO 2019/195,519 and WO 2022/011,214, the contents of which are incorporated herein by reference.

In certain embodiments, the chemically modified barcodes may comprise at least about 1-10%, 10-15%, 15-20%, 20-25%, 25-30%, 30-35%, 35-40%, 40-45%, 45-50%, 50-55%, 55-60%, 60-65%, 65-70%, 70-75%, 75-80%, 80-85%, 85-90%, 90-95%, 95-100%, or 100% modified nucleotides, including any percentage within these ranges.

In certain embodiments, the percentage of nucleotides of the barcode comprising a modified ribose group within the barcode sequence may be up to 1-10%, 10-15%, 15-20%, 20-25%, 25-30%, 30-35%, 35-40%, 40-45%, 45-50%, 50-55%, 55-60%, 60-65%, 65-70%, 70-75%, 75-80%, 80-85%, 85-90%, 90-95%, 95-100%, or 100% of all ribose groups, including any percentage within these ranges.

In certain embodiments, the percentage of nucleotides of the barcode comprising a modified phosphate group or internucleoside linkage within the barcode sequence may be up to 1-10%, 10-15%, 15-20%, 20-25%, 25-30%, 30-35%, 35-40%, 40-45%, 45-50%, 50-55%, 55-60%, 60-65%, 65-70%, 70-75%, 75-80%, 80-85%, 85-90%, 90-95%, 95-100%, or 100% of all internucleoside linkages, including any percentage within these ranges.

Chemically modified barcodes of the disclosure that comprise greater than 50% modified nucleotides have several advantages over the unmodified or minimally modified barcodes widely used in the art. Unmodified nucleic acid barcodes can lead to immune stimulation and off-target effects (C. D. Sago, S. Kalathoor, J. P. Fitzgerald, G. N. Lando, N. Djeddar, A. V. Bryksin, J. E. Dahlman. Barcoding chemical modifications into nucleic acids improves drug stability in vivo, J. Mater. Chem. B. 6 (2018) 7197-7203. https://doi.org/10.1039/C8TBO1642A.). Chemically modified nucleic acid barcodes, however, confer enhanced stability in biological matrices and reduced immune stimulation compared with unmodified or minimally modified barcodes and are therefore particularly useful in applications where the delivery vehicle or NAT is not encapsulated or otherwise protected from nucleolytic degradation (also referred to as naked delivery or gymnotic delivery). Further to chemical modification, off-target effects can also be reduced by using a shorter barcode. Barcodes below the standard size for NATs (<19 nt) reduce the potential for off-target binding.

Optionally, in some embodiments, the nucleic acid barcode includes one or more unique molecular identifiers (UMIs). UMIs encompass a heterogeneous population of degenerate nucleotide sequences in which, preferably, each molecule comprises a unique UMI sequence that is distinct from the UMI sequences of all other molecules within the population. When subjected to amplification, for example during PCR or RCA reactions, sequences that share the same UMI can be identified as copies of one common input molecule. By removing all but one of the UMI duplicates, an accurate count of the number of input molecules present pre-amplification is obtained. Therefore, the use of UMIs in deep-sequencing experiments, or other experiments that involve PCR amplification, can increase the accuracy of nucleic acid quantification.

The skilled artisan will be aware that the sequence diversity of a given UMI population is pre-determined by the length of the UMI. For example, UMIs with a length of 4 nucleotides can only provide up to 256 unique molecules, whereas UMIs of 10 nucleotides in length can give rise to a population of over one million unique sequences (V. Svensson, K. N. Natarajan, L.-H. Ly, R. J. Miragaia, C. Labalette, I. C. Macaulay, A. Cvejic, S. A. Teichmann, Power analysis of single-cell RNA-sequencing experiments., Nat. Methods. 14 (2017) 381-387. https://doi.org/10.1038/nmeth.4220). Typically, UMIs are designed to be between 3-100 nucleotides in length, preferably between 8-20 nucleotides in length, including any length within these ranges. Methods to design UMIs of appropriate length for deep-sequencing applications, as well as methods for UMI deduplication, are known in the art (K. Clement, R. Farouni, D. E. Bauer, L. Pinello, AmpUMI: design and analysis of unique molecular identifiers for deep amplicon sequencing, Bioinformatics. 34 (2018) i202-i210. https://doi.org/10.1093/bioinformatics/bty264.; T. Smith, A. Heger, I. Sudbery, UMI-tools: modeling sequencing errors in Unique Molecular Identifiers to improve quantification accuracy, Genome Res. 27 (2017) 491-499. http://genome.cshlp.org/content/27/3/491.abstract.) and are incorporated herein by reference in their entirety.

In some embodiments, the UMI comprises DNA, RNA, XNA or combinations thereof.

In certain embodiments, the UMI may comprise at least about 1-10%, 10-15%, 15-20%, 20-25%, 25-30%, 30-35%, 35-40%, 40-45%, 45-50%, 50-55%, 55-60%, 60-65%, 65-70%, 70-75%, 75-80%, 80-85%, 85-90%, 90-95%, 95-100%, or 100% chemically modified nucleotides, including any percentage within these ranges.

In certain embodiments, the percentage of nucleotides comprising a modified ribose group within the UMI sequence may be up to 1-10%, 10-15%, 15-20%, 20-25%, 25-30%, 30-35%, 35-40%, 40-45%, 45-50%, 50-55%, 55-60%, 60-65%, 65-70%, 70-75%, 75-80%, 80-85%, 85-90%, 90-95%, 95-100%, or 100% of all nucleotides, including any percentage within these ranges.

In certain embodiments, the percentage of internucleoside linkages comprising a modified internucleoside linkage within the UMI sequence may be up to 1-10%, 10-15%, 15-20%, 20-25%, 25-30%, 30-35%, 35-40%, 40-45%, 45-50%, 50-55%, 55-60%, 60-65%, 65-70%, 70-75%, 75-80%, 80-85%, 85-90%, 90-95%, 95-100%, or 100% of all internucleoside linkages, including any percentage within these ranges.

Chemically modified UMIs of the disclosure that comprise greater than 50% modified nucleotides confer higher stability and reduced immunotoxicity compared with the unmodified or minimally modified UMIs widely used in the art.

In some embodiments, the UMI included within the barcode comprises a continuous stretch of consecutive random nucleotides. UMIs with consecutive random nucleotides are known in the art and described in. for example, J. E. Dahlman, K. J. Kauffman, Y. Xing, T. E. Shaw, F. F. Mir, C. C. Dlott, R. Langer, D. G. Anderson, E. T. Wang, Barcoded nanoparticles for high throughput in vivo discovery of targeted therapeutics, Proc. Natl. Acad. Sci. 114 (2017) 2060-2065. https://doi.org/10.1073/pnas.1620874114., the contents of which are incorporated herein by reference.

In other embodiments, the UMI is split into two segments that flank the barcode sequence. Such bipartite UMI design is known in the art and described in, for example, K. Paunovska, C. D. Sago, C. M. Monaco, W. H. Hudson, M. G. Castro, T. G. Rudoltz, S. Kalathoor, D. A. Vanover, P. J. Santangelo, R. Ahmed, A. V Bryksin, J. E. Dahlman, A Direct Comparison of in Vitro and in Vivo Nucleic Acid Delivery Mediated by Hundreds of Nanoparticles Reveals a Weak Correlation, Nano Lett. 18 (2018) 2148-2157. https://doi.org/10.1021/acs.nanolett.8b00432., the contents of which are incorporated herein by reference.

In certain embodiments, the UMI is split into at least 3, 4, 5, 6, 7, 8, 9, 10 or more segments. Such multipartite UMI designs are known in the art and described in, for example, Y. Fu, P.-H. Wu, T. Beane, P. D. Zamore, Z. Weng, Elimination of PCR duplicates in RNA-seq and small RNA-seq using unique molecular identifiers, BMC Genomics. 19 (2018) 531. https://doi.org/10.1186/s12864-018-4933-1., the contents of which are incorporated herein by reference.

Separating the UMI into two or more segments serves to prevent undesired secondary structure formation and minimizes the risk of hybridization-driven interactions with cellular DNA or RNA present in a biological sample, which ultimately improves the quality of sequencing data.

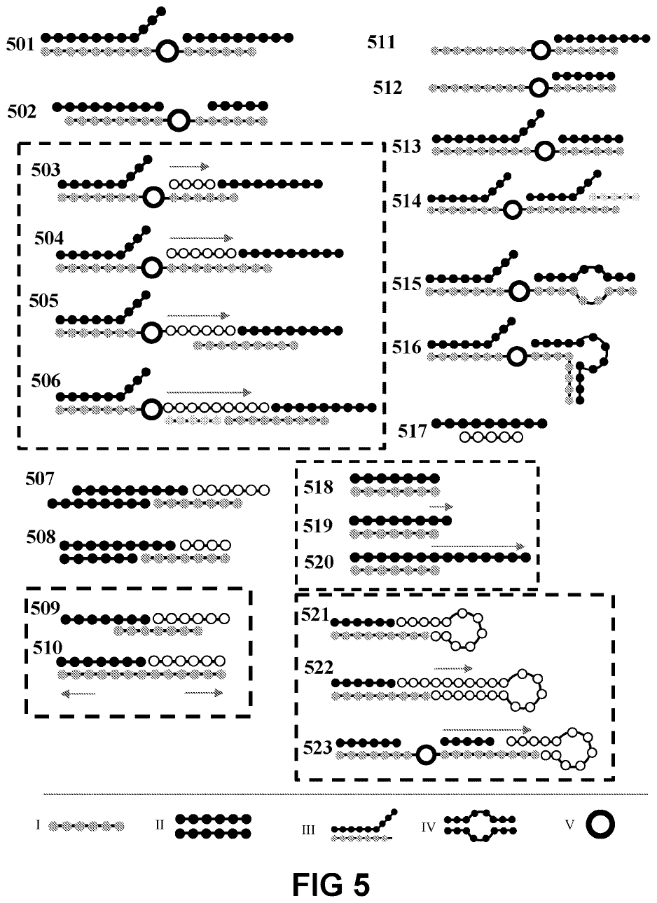

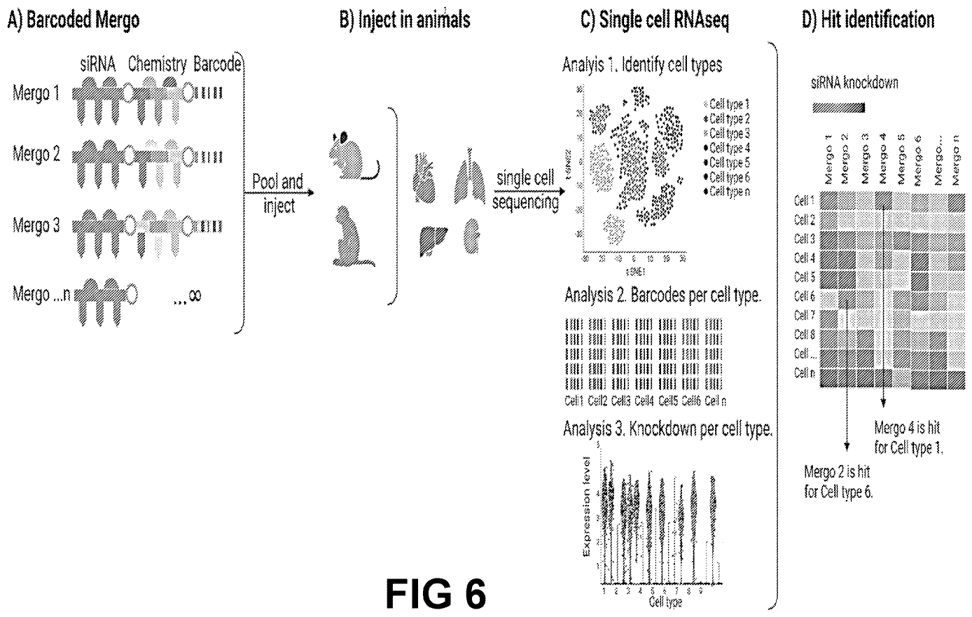

In some embodiments, the barcode is covalently or noncovalently linked to a nucleic acid molecule with known sequence, whereby said nucleic acid molecule may comprise a single-stranded, double-stranded or triple-stranded DNA, RNA or XRNA. In one embodiment, said nucleic acid molecule constitutes a NAT (for example, but not limited to, ASO, siRNA, saRNA, miRNA, sgRNA, mRNA). In a preferred embodiment, said nucleic acid molecule is part of a nucleic acid delivery vehicle (for example, but not limited to, a Mergo) or nucleic acid nanoparticle. In an alternative embodiment, said nucleic acid molecule comprises a synthetic sequence that shares no substantial identity or complementarity with any endogenous DNA or RNA in the biological species under study. In another embodiment, said nucleic acid molecule comprises a poly(A) or poly(dT) sequence to enable simultaneous reverse transcription and amplification of barcode and total mRNA sequences. In still another embodiment, said nucleic acid molecule comprises a sequence identical or partially identical to a target DNA or RNA molecule of interest to enable co-detection of barcode and target sequences. In yet another embodiment, said nucleic acid molecule comprises an Illumina-compatible adapter sequence or primer binding site (PCR handle), or any other sequence, not necessarily Illumina-compatible, that allows direct sample processing with a minimal number of steps (for example, but not limited to, TruSeq™ adapter sequences, TruSeq™ RNA PCR primer sequences, NEBNext® adapter sequences, Nextera transposase adapter sequences, Nextera PCR primer sequences, AmpliSeq adapter sequences, or TruSight adapter sequences, 10× Genomics Capture sequence 1, or 10× Genomics Capture sequence 2). In another preferred embodiment, said nucleic acid molecule comprises a sequence complementary to a universal hybridization arm to allow for hybridization-based attachment of the barcoded strand (FIG. 5) to another nucleic acid strand.

As used herein, the terms “barcoded nucleic acid”, “barcoded strand” and “barcoded sequence” are used interchangeably to refer to said nucleic acid molecule that is covalently or noncovalently linked to a nucleic acid barcode.

In some embodiments, the barcode is incorporated within the barcoded nucleic acid sequence. For example, the barcode can be included as a 5′ or 3′ extension of the barcoded strand, or both. Alternatively, the barcode may be included in an internal loop, hairpin or bulge region of the barcoded strand, or combinations thereof. In another instance, the barcode may be included in an internal double-helical motif of the barcoded strand; said design however may require the synthesis of a strand complementary to both the barcode and the barcoded strand for each barcode in addition to synthesis of the barcoded strand containing the barcode, doubling synthesis needs.

In instances where the barcode is included at an internal position within the barcoded strand, the barcode sequence is flanked by adjacent upstream and downstream sequences. Said flanking sequences, if at least 5 or more nucleotides in length, may serve as universal primer binding sites in PCR amplification reactions. Methods of designing specific primer sequences are well known to those of skill in the art. Alternatively, said flanking sequences may enable hybridization of splint oligonucleotides, splint adapters or padlock probes for splint ligation reactions. In certain embodiments, either one or both of the flanking sequences may be complementary to one or more capture probes for hybridization capture, primer extension capture or fishing approaches.

In instances where the barcode is included at the 5′ or 3′ terminus of the barcoded strand, the barcoded terminus may be extended with a universal sequence by methods known in the art. Such methods may include, but are not limited to, 5′ or 3′ adapter ligation, self-circularization, reverse transcription with tailed degenerate primers, template switching reverse transcription, PCR with tailed degenerate primers, and ligation-dependent probe amplification.

In preferred embodiments, incorporation of the barcode occurs during oligonucleotide synthesis (for example, but not limited to, solid-phase oligonucleotide synthesis, enzymatic oligonucleotide synthesis). In other embodiments, incorporation of the barcode into the barcoded strand is performed post-synthetically by, for example, but not limited to, ligation, conjugation, reverse transcription, template-switching reverse transcription or primer extension PCR. Suitable conjugation techniques include those previously disclosed in U.S. Ser. No. 17/241,920, the contents of which are incorporated herein by reference in its entirety. This includes, but is not limited to, copper (I)-catalyzed alkyne-azide cycloaddition (CuAAC), strain-promoted alkyne-azide cycloaddition (SPAAC), ruthenium-catalysed azide-alkyne cycloaddition (RuAAC), inverse electron demand Diels-Alder reaction (IEDDA), Sulfur Fluoride Exchange (SuFEx), strain-promoted alkyne-nitrone cycloaddition (SPANC), hydrazone/oxime ether formation. thiol-ene radical reaction, thiol-yne radical reaction, thiol-Michael addition reaction, thiol-isocyanate reaction, thiol-epoxide click reaction, nucleophilic ring opening reactions (spring-loaded reactions), traceless Staudinger ligation.

In certain embodiments, the barcoded strand is noncovalently associated with at least one other molecule. For example, the barcoded strand may hybridize to a complementary nucleic acid via Watson-Crick base-pairing. In other instances, the barcoded strand may electrostatically interact with a cationic or anionic target molecule. Examples of cationic target molecules include, but are not limited to deoxyribonucleic guanidine oligomers, deoxynucleic methylthioureas, 2-aminopyridine modified PNAs, and cationic peptides comprising at least one or more Arg or Lys amino acid.

In another embodiment, the barcoded strand may be encapsulated within a lipid nanoparticle.

In some embodiments, the incorporation of a nucleic acid barcode unique to each chemical entity, or its composition, allows for the identification of the identity or source of pooled samples in multiplexed assays. The number of chemical entities processed in parallel determines the minimum number of barcodes to be included in a given pool of samples. For example, if in a given use case the pool of samples to be processed simultaneously comprises 2, 5, 12, 48, 96, or 384 samples, a set of at least 2 or more, 5 or more, 12 or more, 48 or more, 96 or more, or 384 or more distinct barcodes will be required for unambiguous sample identification.

It will be readily apparent to those skilled in the art that the length of the barcode may limit the number of samples that can be processed in parallel. For a given barcode length L, the maximum possible number of unique barcode sequences is 4^{L}. If for instance, a barcode length of 3 nucleotides is selected, the number of barcodes that can be multiplexed is no more than 64. In practice, the maximum number of unique barcode sequences appropriate for a desired application may be lower due to experimental constraints. Exemplary constraints for the rational design of a set of barcode sequences suitable for deep sequencing applications include, but are not limited to, (i) tolerance to sequencing errors. (ii) exclusion of homopolymer stretches such as GGG or AAAA or repetitive nucleotide units such as GAGAGAGA, (iii) avoidance of certain nucleotide sequences known to cause sequencing read errors or result in sequencing bias, (iv) balanced GC content, (v) avoidance of sequences that can form stable secondary structures or G quadruplexes, (vi) avoidance of self- or cross-complementary sequences, and (vii) exclusion of sequences that are substantially identical or complementary to cellular DNA or RNA. Methods for designing sets of barcode sequences are described, for example, in U.S. Pat. No. 6,235,475, P. Somervuo, P. Koskinen, P. Mei, L. Holm, P. Auvinen, L. Paulin, BARCOSEL: a tool for selecting an optimal barcode set for high-throughput sequencing., BMC Bioinformatics. 19 (2018) 257. https://doi.org/10.1186/s12859-018-2262-7.; C. Tr6beau, J. de Monvel, F. Wong Jun Tai, C. Petit, R. Etournay, DNABarcodeCompatibility: an R-package for optimizing DNA-barcode combinations in multiplex sequencing experiments, Bioinformatics. 35 (2018) 2690-2691. https://doi.org/10.1093/bioinformatics/bty1030.; I. S. Yang, S. W. Bae, B. Park, S. Kim, Development of a program for in silico optimized selection of oligonucleotide-based molecular barcodes., PLoS One. 16 (2021) e0246354. https://doi.org/10.1371/journal.pone.0246354., the contents of each of which are incorporated herein by reference in their entirety.

In some embodiments, the nucleic acid barcode is used as an identifier of the chemical composition of a chemical entity. It may correlate with one or multiple chemical characteristics of the chemical entity including, but not limited to, a particular type of modification, a particular pattern (or arrangement) of multiple modifications, a particular number of modifications present, a particular physicochemical property or a particular set of physicochemical properties, a particular conjugation chemistry used for cargo attachment, a particular type of chemical linker introduced during intermolecular conjugation, a particular coating molecule or mix of coating molecules, a particular method used for vehicle assembly, or combinations thereof. It may further correlate with non-chemical properties such as shape (for example, but not limited to, helix, circle, triangle, square, pentagon, hexagon, heptagon, octagon, nonagon, decagon, cube, cylinder, sphere, pyramid, prism, tetrahedron, octahedron), dimension (for example, but not limited to, diameter, length, width, height, depth, radius, circumference, area, volume, or combinations thereof), nucleic acid structure (for example, but not limited to, duplex, triplex, quadruplex, hairpin, loop, bulge, pseudoknot, 2-way junction, 3-way junction, 4-way junction, 5-way junction, 6-way junction, or combinations thereof), particular cargo load (for example, but not limited to, oligonucleotides, including NATs, peptides, proteins, including antibodies, lipids, carbohydrates, polymers, fluorophores, and combinations thereof) or number of covalently or non-covalently attached cargoes, and the number of molecular constituents of the chemical entity (for example, but not limited to, one, two, three, four, five, six, seven, eight, nine, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 constituents or sums or multiples thereof), or combinations thereof. Alternatively or in combination with the above, a barcode may uniquely identify a particular production batch of delivery vehicles or a particular pre-treatment method (for example, but not limited to, incubation with a particular protein or mix of proteins, incubation in a particular buffer, medium or biological matrix, a particular storage condition, uptake by a particular cell line or cell type, a particular time point of a stability experiment, or combinations thereof). In other instances, the nucleic acid barcode may serve as an identifier of a particular location, such as the location of a well in a multiwell plate.

Optionally, qualitative or quantitative methods are applied to detect the one or more nucleic acid barcodes which may or may not include one or more UMIs. Exemplary detection techniques include, but are not limited to, next-generation sequencing, microarray, quantitative PCR, in-situ hybridisation, and branched DNA assays. Suitable sequencing techniques include, but are not limited to, bridge amplification sequencing/Solexa (Illumina), ion semiconductor sequencing, GenapSys sequencing, combinatorial probe-anchor synthesis (cPAS), sequencing by ligation (SOLiD sequencing), single molecule real-time sequencing (SMRT), Heliscope single molecule sequencing, nanopore sequencing, pyrosequencing, and Sanger sequencing.

In some embodiments, the nucleic acid barcode does not include a UMI; instead, a UMI is optionally introduced prior to the first of one or more nucleic acid amplification steps. For example, a UMI may be included on a 5′ adapter, a 3′ adapter, or both, and introduced into the barcoded nucleic acid strand by adapter ligation techniques. The design of UMI-containing adapters is known in the art and described in, for example, Y. Fu, P.-H. Wu, T. Beane, P. D. Zamore, Z. Weng, Elimination of PCR duplicates in RNA-seq and small RNA-seq using unique molecular identifiers, BMC Genomics. 19 (2018) 531. https://doi.org/10.1186/s12864-018-4933-1., the contents of which are incorporated herein by reference.

In another example, the primer used during reverse transcription may be designed to contain a UMI sequence, such that a UMI is incorporated into the cDNA copy of the barcoded molecule during cDNA synthesis. The use of UMI-containing reverse transcription primers is known in the art and described in, for example, T. Hashimshony, N. Senderovich, G. Avital, A. Klochendler, Y. de Leeuw, L. Anavy, D. Gennert, S. Li, K. J. Livak, O. Rozenblatt-Rosen, Y. Dor, A. Regev, I. Yanai, CEL-Seq2: sensitive highly-multiplexed single-cell RNA-Seq, Genome Biol. 17 (2016) 77. https://doi.org/10.1186/s13059-016-0938-8., the contents of which are incorporated herein by reference.

Alternatively, a UMI may be included within a template switching oligonucleotide (TSO) and introduced into the cDNA copy of the barcoded nucleic acid strand during template switching reverse transcription. The use of UMI-containing TSOs is known in the art and described in, for example, M.-J. Arguel, K. LeBrigand, A. Paquet, S. Ruiz Garcia, L.-E. Zaragosi, P. Barbry, R. Waldmann, A cost effective 5′ selective single cell transcriptome profiling approach with improved UMI design, Nucleic Acids Res. 45 (2017) e48-e48. https://doi.org/10.1093/nar/gkw1242, the contents of which are incorporated herein by reference.

Another option is to use at least one or more hybridization probes containing one or more UMI sequences, such that the probes can serve, similarly to cDNA, as templates in subsequent amplification reactions. The use of UMI-containing hybridization probes is known in the art and described in, for example, J. J. Credle, M. L. Robinson, J. Gunn, D. Monaco, B. Sic, A. Tchir, J. Hardick, X. Zheng, K. Shaw-Saliba, R. E. Rothman, S. H. Eshleman, A. Pekosz, K. Hansen, H. Mostafa, M. Steinegger, H. B. Larman, Highly multiplexed oligonucleotide probe-ligation testing enables efficient extraction-free SARS-CoV-2 detection and viral genotyping, Mod. Pathol. 34 (2021) 1093-1103. https://doi.org/10.1038/s41379-020-00730-5., the contents of which are incorporated herein by reference.

In some embodiments, the barcoded strand includes one barcode. In other embodiments, the barcoded strand includes more than one barcode, for example 2, 3, 4, 5, 6, 7, 8, 9 or more barcodes (n). Each of the second, third, fourth, fifth, sixth, seventh, eighth, nineth or nth barcode may comprise a sequence identical or different to the first barcode. Alternatively, each of the barcode sequences may be identical or different to at least one or more other barcode sequences in the barcoded molecule.

In instances where the barcoded entity comprises more than one molecule, preferably more than one nucleic acid strand, at least one or more of the constituting molecules may be covalently or noncovalently associated with the one or more barcodes. In some embodiments, each of the constituting molecules of the barcoded entity may carry one or more barcodes. In a preferred embodiment, exactly one of the constituting molecules is barcoded.

It will be readily apparent to those skilled in the art that other suitable modifications and adaptations of the methods described herein may be made using suitable equivalents without departing from the scope of the embodiments disclosed herein.

In some embodiments, the detection and quantification of the nucleic acid barcodes described herein requires amplification of the one or more barcodes, including the one or more UMIs if present. For example, sequencing library preparation for deep sequencing applications on Illumina instruments generally involves PCR amplification. Those skilled in the art will appreciate that any amplification method known in the art, including isothermal amplification methods, may be applied without departing from the scope of the present disclosure.

In some embodiments, the innate activity of certain DNA-dependent DNA polymerases can be exploited for simultaneous, 1-step reverse transcription and amplification of barcoded nucleic acid strands comprising RNA, XNA or combinations thereof. DNA polymerases with said activity include, but are not limited to, BcaBEST Polymerase, Bst 3.0 DNA Polymerase, Volcano2G DNA polymerase, Volcano3G DNA polymerase, OmniAmp DNA polymerase, phi29 DNA polymerase, Phusion DNA polymerase, Q5 DNA polymerase, and Deep Vent DNA Polymerase. Furthermore, polymerases specifically engineered to read and amplify XNA are known in the art and are described in, for example, G. Houlihan, S. Arangundy-Franklin, B. T. Porebski, N. Subramanian, A. I. Taylor, P. Holliger, Discovery and evolution of RNA and XNA reverse transcriptase function and fidelity, Nat. Chem. 12 (2020) 683-690. https://doi.org/10.1038/s41557-020-0502-8.; A. S. Thompson, S. E. Barrett, A. G. Weiden, A. Venkatesh, M. K. C. Seto, S. Z. P. Gottlieb, A. M. Leconte, Accurate and Efficient One-Pot Reverse Transcription and Amplification of 2′-Fluoro-Modified Nucleic Acids by Commercial DNA Polymerases, Biochemistry. 59 (2020) 2833-2841. https://doi.org/10.1021/acs.biochem.0c00494.; L. Sun, X. Ma, B. Zhang, Y. Qin, J. Ma, Y. Du, T. Chen, From Polymerase Engineering to Semi-Synthetic Life: Artificial Expansion of the Central Dogma, RSC Chem. Biol. (2022). https://doi.org/10.1039/D2CB00116K.; T. Chen, F. E. Romesberg, Enzymatic Synthesis, Amplification, and Application of DNA with a Functionalized Backbone, Angew. Chemie Int. Ed. 56 (2017) 14046-14051. https://doi.org/https://doi.org/10.1002/anie.201707367.; A. Thompson, S. Barrett, A. Weiden, A. Leconte, Reverse transcription and amplification of 2′F DNA by commercially available DNA polymerases, FASEB J. 34 (2020) 1. https://doi.org/https://doi.org/10.1096/fasebj.2020.34.sl.00614., which are incorporated herein by reference. Examples of these so-called engineered XNA polymerases include, but are not limited to, SFM4-3 polymerase, SFM4-6 polymerase, KOD DLK polymerase, RT-C8exo+ polymerase, RT-H4exo+ polymerase and SFP1 polymerase.

In some embodiments, amplification and quantitative detection of nucleic acid barcodes can be performed simultaneously. Exemplary amplification methods that allow for direct quantification during amplification include, but are not limited to, quantitative real-time polymerase chain reaction (qPCR and qRT-PCR), digital PCR, digital droplet PCR (ddPCR), ligation-dependent probe amplification (MLPA), digital MLPA (dMLPA), real-time multiplex loop-mediated isothermal amplification (RT-LAMP), RCA-assisted CRISPR/Cas9 cleavage (RACE), RCA-FRET DNA assay, RCA-assisted single-molecule flow cytometry and the like.

Other amplification techniques include, but are not limited to, rolling circle amplification (RCA), recombinase polymerase amplification (RPA), loop-mediated isothermal amplification (LAMP), exponential amplification reaction (EXPAR), duplex-specific nuclease signal amplification (DSNSA), nicking enzyme amplification reaction (NEAR), nucleic acid sequence-based amplification (NASBA), strand displacement amplification (SDA), multiple displacement amplification (MDA), helicase-dependent amplification (HDA), hybridization chain reaction (HCR), catalyzed hairpin amplification (CHA), and single chimeric primer isothermal amplification (SPIA).

### Barcoded Mergo

The modifications of the barcode described in some embodiments allow for the use of it in biological matrices, as the modifications infer stability and immune stimulus reduction. Prior art leveraging barcoding technology uses modified NA for conferring stability in cell lysates, for example US Patent Application Publication US2017/0145476, which is herein incorporated by reference.

Alternatively, prior art indicates that the non-modified or minimally modified NA barcode is encapsulated in drug delivery systems to be administered in vivo—for example Lipid Nanoparticles described in J. E. Dahlman, K. J. Kauffman, Y. Xing, T. E. Shaw, F. F. Mir, C. C. Dlott, R. Langer, D. G. Anderson, E. T. Wang, Barcoded nanoparticles for high throughput in vivo discovery of targeted therapeutics, Proc. Natl. Acad. Sci. 114 (2017) 2060-2065. https://doi.org/10.1073/pnas.1620874114.

In some embodiments the modified barcodes presented in this disclosure may interact with biological matrices including, but not limited, to single celled organisms, multicellular organisms, cell suspensions, cell lysates, extracellular matrices, blood serum, whole blood, urine, feces, lacrimal fluid, sputum, semen, cerebrospinal fluid, saliva, culture swabs, skin samples and skin samples.

In some embodiments, one or more cells can be collected by any part of the subject. For example, any part of the subject could include, but is not limited to, organs and tissues such as liver, lung, heart, kidney, pancreas, spleen, skin, adipose tissue, eye, brain, spinal cord, muscle, testes, ovaries, uterus, mouth, esophagus, stomach, small intestine, large intestine, and gallbladder. Optionally the cells can be tumors, including malignant or not.

In other embodiments barcodes administered in multicellular organisms are used to assess the biodistribution also known as pharmacokinetic properties of the barcoded Mergo. Examples of subjects include but are not limited to humans, non-human primates, mice, rats, dogs, pigs, sheep

r example, the content of Mergos tissue or organ that, when administered to the subject, preferentially are attached to a unique barcode.

In other embodiments, the sequencing of the tissues to detect the barcode can be performed as a single cell sequencing. By identifying the barcode content in every cell type, the skilled craftsman can reveal, for example, the content of Mergo per cell type. Cell types can include but are not limited to hepatocytes, macrophages, hepatic stellate cells, kupffer cells, pneumocytes, endothelial cells, epithelial cells, cardiomyocytes, fibroblasts, and cancer cells.

In other embodiments, the single cell sequencing of the tissue can be designed to detect the efficacy of the cargo therapeutic carried by the barcoded Mergo, in parallel to the detection of the barcode and the cell type. The skilled craftsman can assess the efficacy of a nucleic acid therapeutic by RNA sequencing. The efficacy can be related, but is not limited, to gene activation or by gene silencing by knockdown or knockout.

### Barcoded Cargo Conjugates

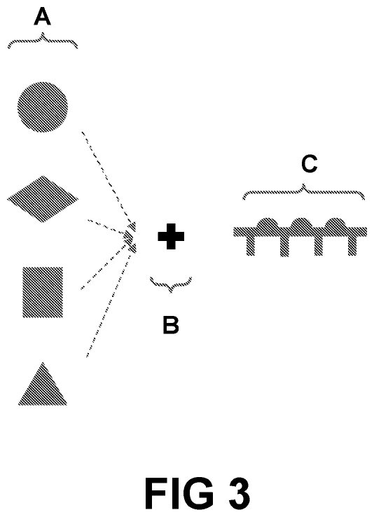

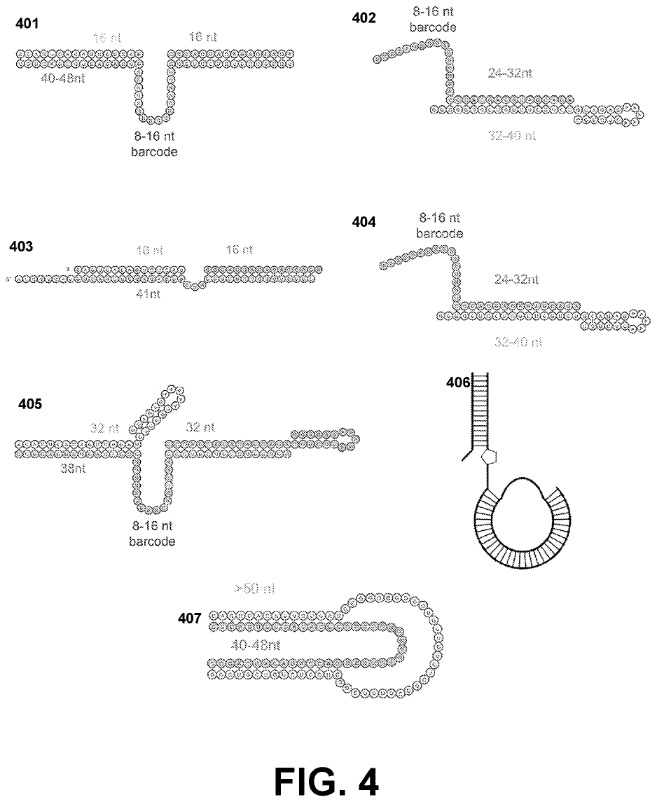

The NA Barcode described above, including but not limited to its embodiments as a ‘short’ or ‘modified’ NA barcode, can be further linked covalently or noncovalently to cargo molecules that are not explicitly a Mergo or NAT (FIG. 2; FIG. 3)

In some embodiments, the NA Barcode is incorporated into a cargo molecule where the multiplexed identification of chemical entities in a complex biological context would be useful.

This could include, but is not limited to, CRISPR, proteins, peptides, small molecules, NA material, antibody fragments and conjugated NAT delivery systems such as antibodies (ADCs), GalNac and lipid conjugates.

In further embodiments, the NA Barcode is incorporated into a cargo molecule that also contains a NAT, thereby supporting the multiplexed identification of chemical entities as well as the evaluation of their mechanism of action and biological activity in complex biological matrices. In this embodiment, analysis methods known in the art can be used orthogonally with barcode sequencing. Such methods may include, but are not limited to, receptor binding, target engagement, subcellular identification, pharmacokinetic and pharmacodynamic readouts, affinity, reactivity, interaction-dependent PCR, CITEseq, and Immuno-SABER.

In some embodiments the NA barcode linked to a cargo molecule is designed to be the same length of the finally conjugated therapeutically relevant NAT and contains similar chemical modification patterns, as to exert a similar influence on biological processes, PK/PD profiles and influence on distribution. Thus, in the final administered formulation someone skilled in the art could replace the NA barcode with a therapeutically-relevant molecule. For example, ASOs are 15-21 bp long so the NA barcode would be 15-21 bp long. In other embodiments the NA barcode could be hybridized to a complementary sequence to mimic the same PK/PD and biodistribution of a double-stranded therapeutic molecule such as siRNA or miRNA.

In some embodiments, the NA Barcode is conjugated to a polypeptide cargo molecule which may include, but not be limited to, peptides with cell-penetrating, endosomal escape, cell or tissue targeting capabilities or proteins with targeting functions (antibodies), enzyme or protein replacement capabilities.

In some embodiments, the peptide to oligonucleotide conjugate may be synthesized through solid-phase synthesis. For example, according to a method outlined in K. Klabenkova, A. Fokina, D. Stetsenko, Chemistry of peptide-oligonucleotide conjugates: A review, Molecules. 26 (2021) 1-36. https://doi.org/10.3390/molecules26175420., a bifunctional handle containing a hydroxyl group can be reversibly attached to a CPG solid support. The handle is selected to simultaneously ensure oligonucleotide chain extension while selectively cleaving the conjugate from the support at the end of the synthesis. At first, the barcoding sequence is synthesized, followed by the introduction of a second linker carrying a temporarily protected amine group which supports the synthesis of the peptide cargo. Trifunctional branched linkers containing both hydroxyl and amine can also be employed, and are generally protected by the orthogonal DMTr and Fmoc groups.

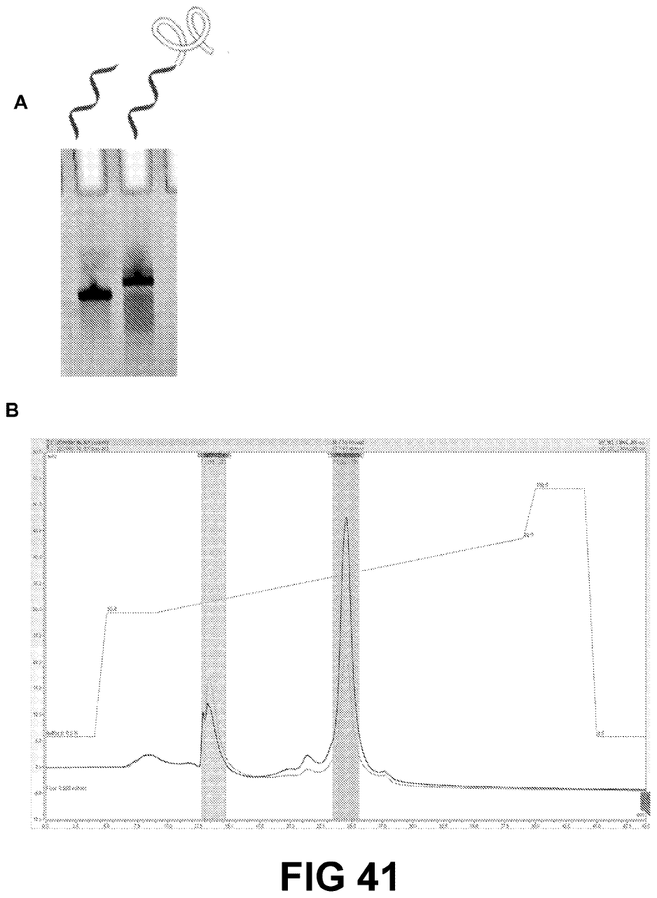

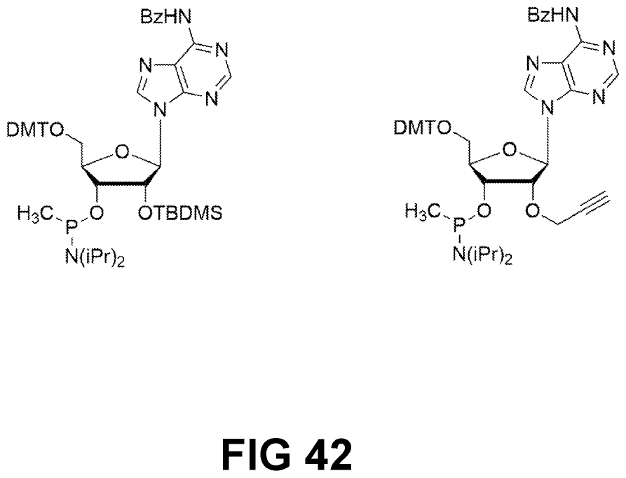

In other embodiments, post-synthetic oligonucleotide to peptide conjugations may be employed. These are known in the art and are described in, for example, US 2022/0072143, U.S. Pat. Nos. 7,964,578, 7,833,992, US 2005/0106598, U.S. Pat. No. 7,772,387, the contents of which are incorporated herein by reference. The synthesis of an example 5′ thiol functionalized barcode to a maleimide-functionalized peptide is given in FIG. 41.

Oligonucleotide-to-protein-conjugations are known in the art and are described in, for example U.S. Pat. Nos. 8,846,875, 5,635,602, WO 2017/190020A1, the contents of which are incorporated herein by reference. Such conjugates are also commercially available and can be purchased from, for example, abcam (cat no. ab218260).

In further embodiments, the NA barcode is linked to a peptide that is used to deliver NATs.

In further embodiments, the NA barcode is linked to a peptide and a NAT (FIG. 2).

In some embodiments, the NA barcode is conjugated to a lipidic cargo molecule, which may include, but not be limited to, lipids with the ability to modulate biodistribution, cell transfection, endosomal escape, cell metabolism, biosensing or interact with the endocrine system.

In preferred embodiments, incorporation of the lipid to the barcode occurs during oligonucleotide synthesis (for example, but not limited to, solid-phase oligonucleotide synthesis, enzymatic oligonucleotide synthesis). In other embodiments, conjugation of the lipid into the barcoded strand is performed post-synthetically. Suitable conjugation techniques include those previously disclosed in U.S. Ser. No. 17/241,920, the contents of which are incorporated herein by reference in its entirety. This includes, but is not limited to, copper (I)-catalyzed alkyne-azide cycloaddition (CuAAC), strain-promoted alkyne-azide cycloaddition (SPAAC), ruthenium-catalysed azide-alkyne cycloaddition (RuAAC), inverse electron demand Diels-Alder reaction (IEDDA), Sulfur Fluoride Exchange (SuFEx), strain-promoted alkyne-nitrone cycloaddition (SPANC), hydrazone/oxime ether formation, thiol-ene radical reaction, thiol-yne radical reaction, thiol-Michael addition reaction, thiol-isocyanate reaction, thiol-epoxide click reaction, nucleophilic ring opening reactions (spring-loaded reactions), traceless Staudinger ligation. Additional known conjugations in the art and are described in, for example (M. E. Ostergaard, M. Jackson, A. Low, A. E Chappell, R. G Lee, R. Q. Peralta, J. Yu, G. A. Kinberger, A. Dan, R. Carty, M. Tanowitz, P. Anderson, T. W. Kim, L. Fradkin, A. E. Mullick, S. Murray, F. Rigo, T. P. Prakash, C. F. Bennett, E. E. Swayze, H. J. Gaus, P. P. Seth, Conjugation of hydrophobic moieties enhances potency of antisense oligonucleotides in the muscle of rodents and non-human primates, Nucleic Acids Res. 47 (2019) 6045-6058. https://doi.org/10.1093/nar/gkz360.; [1]T. Kubo, Y. Nishimura, Y. Sato, K. Yanagihara, T. Seyama, Sixteen Different Types of Lipid-Conjugated siRNAs Containing Saturated and Unsaturated Fatty Acids and Exhibiting Enhanced RNAi Potency, ACS Chem. Biol. 16 (2021) 150-164. https://doi.org/10.1021/acschembio.0c00847.).

In certain embodiments, there can be a linker between the lipid and the oligo, which can facilitate its release from bound plasma or intracellular proteins and support its activity. These linkers may include, amongst others, unmodified phosphodiester (PO)-trinucleotide linkers, hexylamino or hexanediol spacers and combinations thereafter.

In some embodiments the NA barcode is linked to a Cholesterol, C16, C14, C12 molecule. Lipid moieties can be directly attached to a NA via a peptide bond to a CPG containing a C7 linker as described in A. Biscans, A. Coles, R. Haraszti, Di. Echeverria, M. Hassler, M. Osborn, A. Khvorova, Diverse lipid conjugates for functional extra-hepatic siRNA delivery in vivo, Nucleic Acids Res. 47 (2019) 1082-1096. https://doi.org/10.1093/nar/gky1239.

In further embodiments the NA barcode is linked to a lipidic molecule, such as Cholesterol, that is designed to deliver NATs.

In further embodiments, the NA barcode is linked to a lipidic molecule and a NAT (FIG. 2).

In some embodiments, the NA barcode is linked to a small molecule. DNA-encoded libraries are known in the art and are described in, for example, US 2021/0002630, U.S. Pat. No. 11,168,321, US 2020/0123534, US 2012/0071329, U.S. Pat. No. 10,240,147, WO 2022/084,486, C. J. Gerry, M. J. Wawer, P. A. Clemons, S. L. Schreiber, DNA Barcoding a Complete Matrix of Stereoisomeric Small Molecules, J. Am. Chem. Soc. 141 (2019) 10225-10235. https://doi.org/10.1021/jacs.9b01203.; R. E. Kleiner, C. E. Dumelin, D. R. Liu, Small-molecule discovery from DNA-encoded chemical libraries, Chem. Soc. Rev. 40 (2011) 5707-5717. https://doi.org/10.1039/c1cs15076f, the contents of which are incorporated herein by reference.

The NA barcoded small molecule libraries can be generated by DNA-templated organic synthesis whereby through Watson-Crick base pairing, an oligonucleotide directs bond-forming reactions by bringing DNA-linked reagents into proximity. Alternatively, a “split-and-pool” synthesis approach can be used, whereby library diversity is generated over repeated cycles of division, synthesis, and pooling enzyme-catalyzed polymerization or ligation.

In further embodiments, the NA barcode is linked to a small molecule and a NAT (FIG. 2).

In some embodiments, the barcoded strand might be incorporated onto a multi-valent molecule, wherein the two nucleic acids are connected to one another by one or more moieties independently selected from a linker, a spacer and a branching point. These have been previously shown to favorably alter the PK/PD properties of NATs. Suitable multivalent units and synthesis routes include those previously disclosed in PCT/US22/24991 and known in the art, and described in, for example, US 2022/0042015, J. F. Alterman, B. M. D. C. Godinho, M. R. Hassler, C. M. Ferguson, D. Echeverria, E. Sapp, R. A. Haraszti, A. H. Coles, F. Conroy, R. Miller, L. Roux, P. Yan, E. G. Knox, A. A. Turanov, R. M. King, G. Gernoux, C. Mueller, H. L. Gray-Edwards, R. P. Moser, N.C. Bishop, S. M. Jaber, M. J. Gounis, M. Sena-Esteves, A. A. Pai, M. DiFiglia, N. Aronin, A. Khvorova, A divalent siRNA chemical scaffold for potent and sustained modulation of gene expression throughout the central nervous system, Nat. Biotechnol. 37 (2019) 884-894. https://doi.org/10.1038/s41587-019-0205-0.; C. M. Ferguson, S. Hildebrand, B. M. D. C. Godinho, J. Buchwald, D. Echeverria, A. Coles, A. Grigorenko, L. Vanjielli, J. Sousa, N. McHugh, M. Hassler, F. Santarelli, M. T. Heneka, E. Rogaev, A. Khvorova, Silencing of ApoE with Divalent siRNAs Drives Activation of Immune Clearance Pathways and Improves Amyloid Pathology in Mouse Models of Alzheimer's Disease, BioRxiv. (2022) 2022.06.28.498012. https://doi.org/10.1101/2022.06.28.498012., the contents of which are incorporated herein by reference in its entirety.

The stoichiometry of NAT versus barcode can vary substantially within different embodiments. In some, all valency points will be occupied by barcoding strands.

In other embodiments, each valency point will be occupied by a NAT and a barcoding strand.

In further embodiments, a combination of NAT and barcode will be produced, (for example, but not limited 1:1, 1:2, 1:3, 1:4, 1:5, 1:6, 1:7, 1:8, 2:1, 2:2, 2:3, 2:4, 2:5, 2:6, 2:7, 2:8 or sums or multiples thereof). Suitable orthogonal conjugation techniques include, but are not limited, to those previously disclosed in U.S. Ser. No. 17/241,920.

RNA Nanoparticles Consisting of Oligonucleotides with a Modified Phosphonamidite Backbone

The current disclosure contains compositions that incorporate alkyl phosphonamidites.

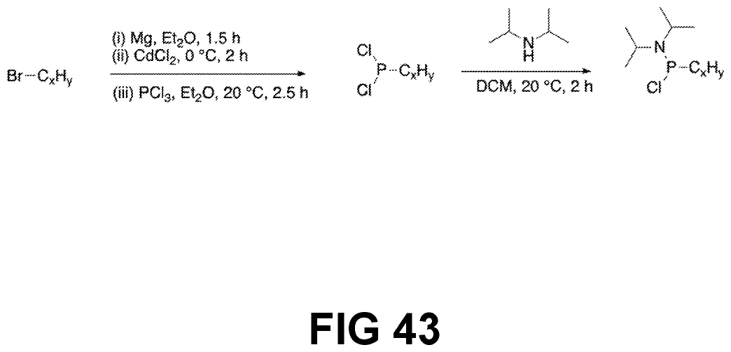

Alkyl phosphonamidites are defined as compounds containing the general formula (III):

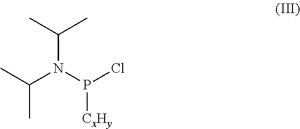

Where R′ can be any given nucleoside or sugar modification outlined above (vide supra) and CₓH_{Y} denotes an alkyl chain with the general formula CₙH₂ₙ₋₁, where x can be anywhere between 1 and 20 and x can be anywhere between 3 and 39. Alkyl phosphonamidites are known in the art and are described in, for example, WO 2006/088,490, the contents of which are incorporated herein by reference. The synthesis of methylphosphonamidites is also known in the art; the synthesis route outlined in the present disclosure is based on a Grignard approach outlined in, for example, US 2005/335,760 (FIG. 43).

Despite prior art showing incorporation of alkyl phosphonates into oligonucleotides, there is limited art showing their use in nanoparticles. Phosphonates have been shown in metal-binding nanoparticles (U.S. Pat. No. 8,603,532) and in polyvalent RNA-nanoparticle compositions (WO 2010/060,110), however, the compositions described in these disclosures are not representative of the compositions described in the present disclosure. Indeed, the polyvalent RNA-nanoparticle compositions outlined in WO 2010/060,110 are based on RNA gold nanoparticles. Accordingly, this disclosure outlines the synthesis of methyl phosphonate-linked oligonucleotides via literature known methyl phosphonamidites and their incorporation into a nucleic acid nanoparticle in the absence of any metal. The commercial methyl phosphonamidites used in this disclosure include, but are not limited to, dA-Me Phosphonamidite (cat no. 10-1100-XX, Glen Research); Ac-dC-Me Phosphonamidite (cat no. 10-1115-XX, Glen Research); dG-Me Phosphonamidite (cat no. 10-1120-XX, Glen Research); dT-Me Phosphonamidite (cat no. 10-1130-XX, Glen Research); (N-Bz)-5′-O-DMT-2′-O-TBDMS-adenosine-3′-O-(P-methyl)phosphonamidite (US2006/287260); (N-Ac)-5′-O-DMT-2′-O-TBDMS-cytidine-3′-O-(P-methyl)phosphonamidite (US2006/287260); 5-Me-5′-O-DMT-2′-O-TBDMS-uridine-3′-O-(P-methyl)phosphonamidite (US2006/287260); (N-iBU)-5′-O-DMT-2′-O-TBDMS-guanosine-3′-O-(P-methyl)phosphonamidite (US2006/287260).

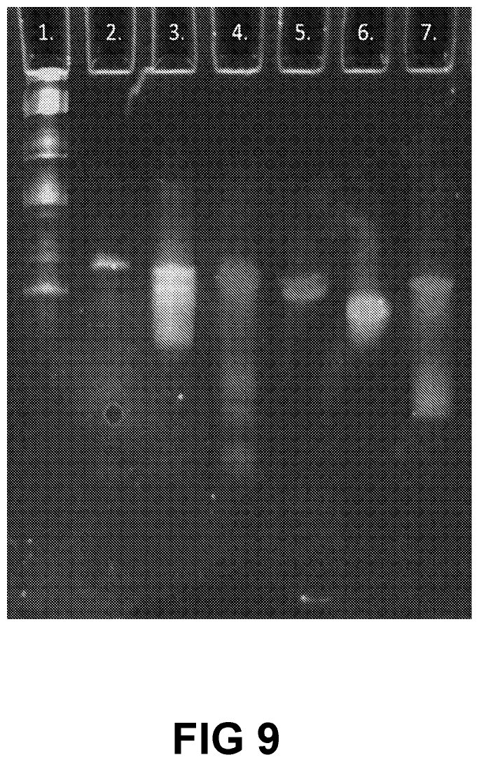

In addition to commercial methyl phosphonamidites, the current disclosure outlines the synthesis of alkyl phosphonamidites functionalized with a propargyl moiety at the 2′ position of the sugar. These compounds have the general formula (IV):

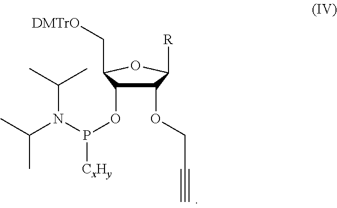

Where R can be any given base as previously outlined in WO 2018/118587 and CₓH_{Y} denotes an alkyl chain with the general formula CₙH₂ₙ₋₁, where x can be anywhere between 1 and 20 and x can be anywhere between 3 and 39. The propargyl group may be used for further functionalization with copper azide-alkyne cycloaddition (CuAAC) chemistry after incorporation into an oligonucleotide. The general synthesis of this compound is outlined in FIG. 9. The 2′O propargyl nucleoside undergoes a standard phosphitylation procedure with compound of general formula (III).

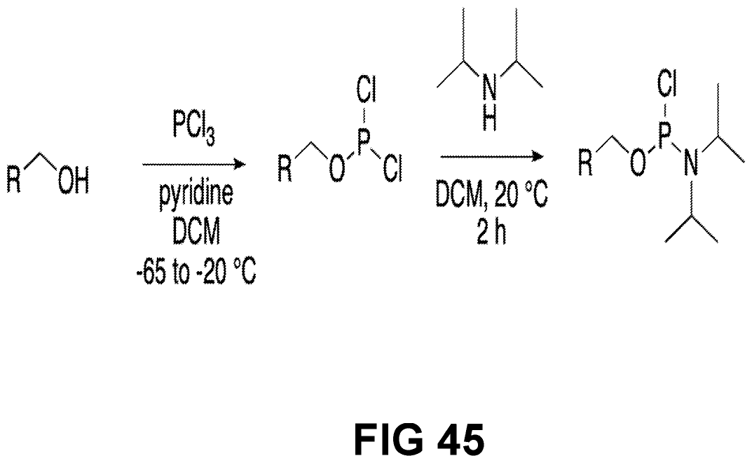

Beyond general functionalization as an alkyl phosphonate, the phosphorous centre may be functionalized with any given modification, as shown in general formula (V):

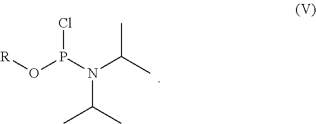

Where R may be any given modification that could modulate the physicochemical properties of the oligonucleotide backbone. These may include, but are not limited to, O-alkylamino, O-alkylalkoxy, protected O-alkylamino, O-alkylaminoalkyl, O-alkyl imidazole, O-alkyl guanidine, polyamines of the formula (O-alkyl)ₘ, where m is about 1 to 20 and polyethers of the formula (O-alkyl)ₘ, where m is about 1 to 20. Preferred ethers are polyethylene glycols (PEGs). The general synthesis of this compound is outlined in FIG. 45 and proceeds via functionalization of the alcohol of interest with phosphorous trichloride, followed by treatment with diisopropylamine.

In certain embodiments, there may be a labile linker between the modification (R) and the phosphorus centre. This linker is cleaved in response to external stimuli, which could include, but is not limited to, change in pH, change in redox potential, enzymatic activity and the presence of exogenous small molecules. Once cleaved, the effect of the modification is reduced. For example, a nucleic acid nanoparticle with a high loading of PEG phosphonate would have an impaired biodistribution. It would be advantageous, therefore, to remove this in circulation so that a more hydrophobic backbone is exposed. This would aid in cell uptake.

Linker molecules may include, but are not limited to thiol cleavable linkers such as dithiobismaleimidoethane, 1,4-bis[3-(2-pyridyldithio)propionamido]butane and 3-(2-pyridyldithio)propionyl hydrazide or base-cleavable linkers such as bis[2-(N-succinimidyl-oxycarbonyloxy)ethyl]sulfone or hydroxylamine-cleavable linkers such as (ethylene glycol bis(succinimidyl succinate)). Direct covalent attachment may be achieved by, for example, thiol arylation using palladium complexes (E. V. Vinogradova et al., Organometallic palladium reagents for cysteine bioconjugation, Nature. 526 (2015) 687-691. https://doi.org/10.1038/nature15739.), oxime ligation (J. Y. Axup et al. Synthesis of site-specific antibody-drug conjugates using unnatural amino acids, Proc. Natl. Acad. Sci. U.S.A 109 (2012) 16101-16106. https://doi.org/10.1073/pnas.1211023109.), hydrazone formation (D. K. Kölmel et al., Oximes and Hydrazones in Bioconjugation: Mechanism and Catalysis, Chem. Rev. 117 (2017) 10358-10376. https://doi.org/10.1021/acs.chemrev.7b00090.) or via a cathepsin B-responsive linker (F. Bryden et al., Impact of cathepsin B-sensitive triggers and hydrophilic linkers on: In vitro efficacy of novel site-specific antibody-drug conjugates, Org. Biomol. Chem. 16 (2018) 1882-1889. https://doi.org/10.1039/c7ob02780j.). Additional linkers may include any that are highlighted by Su et al. (Z. Su, D. Xiao, F. Xie, L. Liu, Y. Wang, S. Fan, X. Zhou, S. Li, Antibody-drug conjugates: Recent advances in linker chemistry, Acta Pharm. Sin. B. 11 (2021) 3889-3907. https://doi.org/10.1016/j.apsb.2021.03.042.); and Bargh et al. (J. D. Bargh, A. Isidro-Llobet, J. S. Parker, D. R. Spring, Cleavable linkers in antibody-drug conjugates, Chem. Soc. Rev. 48 (2019) 4361-4374. https://doi.org/10.1039/c8cs00676h.). An example of such a design, based on a hydrazone-linked PEG, is given below as formula (VI).

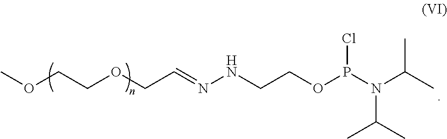

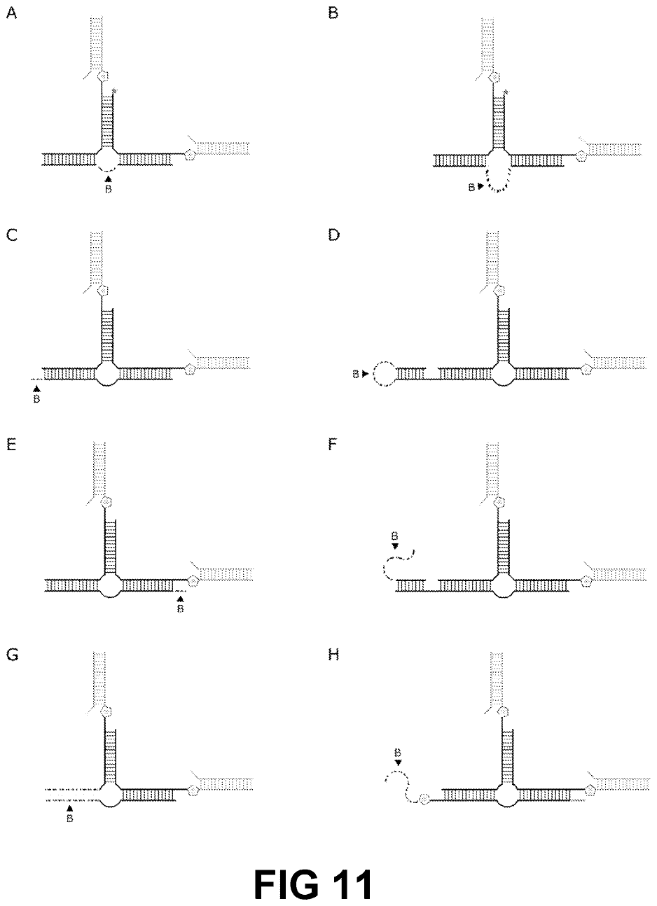

This modified phosphitylating reagent may be used to phosphitylate nucleosides that have protecting groups that are orthogonal to the acid-based strategy used in standard solid-phase RNA synthesis. The synthesis if outlined in FIG. 11. Thermally-labile protecting groups, such as those described by Chmielewski et al. (M. K. Chmielewski, V. Marchin, J. Cieslak, A. Grajkowski, V. Livengood, U. MUnch, A. Wilk, S. L. Beaucage, Thermolytic Carbonates for Potential 5′-Hydroxyl Protection of Deoxyribonucleosides, J. Org. Chem. 68 (2003) 10003-10012. https://doi.org/10.1021/jo035089g.) may be used as an alternative. An example of which is shown in formula (VII) below. The hydrazone moiety would be cleaved in response to an acidic environment, which in turn would expose the underlying amine moiety which would assist in the process of endosomal escape.

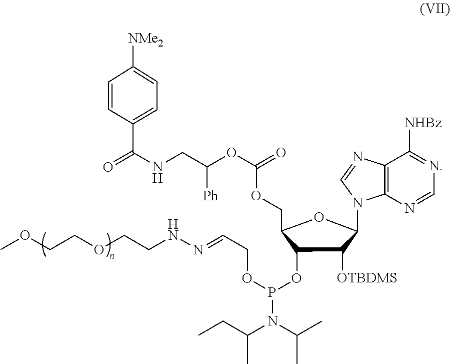

RNA Nanoparticles with Cleavable Linkers

Antisense oligonucleotides (ASOs) and short interfering RNAs (siRNAs) have recently emerged as a viable treatment option for a wide range of diseases (B. Hu, L. Zhong, Y. Weng, L. Peng, Y. Huang, Y. Zhao, X. J. Liang, Therapeutic siRNA: state of the art, Signal Transduct. Target. Ther. 5 (2020). https://doi.org/10.1038/s41392-020-0207-x.). These molecules act via RNA interference mechanisms and are able to recognize a homologous mRNA sequence in a cell and induce its degradation. Despite their promise in vitro, there are still major translatability issues to an in vivo setting. These issues are a result of several physicochemical and biological factors, the biggest of which is the problem of cell uptake and endosomal escape. This effect is amplified when the therapeutic is attached to a nucleic acid nanoparticle.

The multimeric nucleic acid nanoparticles provided herein contain cleavable linkers to help with the problem of uptake and endosomal escape.

As used herein, the term “cleavage” refers to breaking of one or more chemical bonds in a large molecule to produce two or more smaller molecules. One or more of these smaller molecules may be referred to as the cargo, which will go on to elicit a biological effect. In some embodiments, as used herein, the term “cleavable” refers to rapidly degradable linkers whereby the covalent bonds are easily broken to form two or more smaller molecules from a larger molecule (e.g., linkers that are rapidly cleaved by cathepsin B enzymes). The term “non-cleavable” refers to more stable linkages (i.e., nuclease resistant linkages).

In one example, the nucleic acid nanoparticle with a cleavable linker has the general formula (VIII):

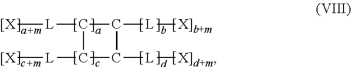

wherein C is a core component oligonucleotide that makes the nucleic acid nanoparticle (upon annealing). In some embodiments, a is 0, 1, 2, 3, 4, 5, 6, 7, 8, 9, 10, 15, 20 or more. In some embodiments, b is 0, 1, 2, 3, 4, 5, 6, 7, 8, 9, 10, 15, 20 or more. In some embodiments, c is 0, 1, 2, 3, 4, 5, 6, 7, 8, 9, 10, 15, 20 or more. In some embodiments, d is 0, 1, 2, 3, 4, 5, 6, 7, 8, 9, 10, 15, 20 or more. L is a cleavable linker and X is a cargo (i.e., a modality that has a function when cleaved). The number of cargo molecules is given by [a+m], [b+m], [c+m], [d+m], wherein the number of cargo molecules is equivalent to the number of oligonucleotide strands, plus [m], where m could be 0, 1, 2, 3, 4, 5, 6, 7, 8, 9, 10, 15, 20 or more, depending on branching and additional points of attachment.

Cargo molecules include, but are not limited to molecules that promote a function and/or biological effect inside or outside a cell (e.g. IRES, ribosomal recruitment, cytokine stimulation), molecules that promote entry into a cell (e.g. peptides, endosomal escape compounds), molecules that bind to target cells (e.g. aptamers, antibodies, ligands), cytotoxic compounds (e.g. cytotoxic nucleosides), molecules that express a gene product inside a cell (e.g. mRNA), chemotherapeutic compounds (e.g. alkylating agents, antimetabolites, topoisomerase inhibitors), molecules that silence or alter a gene inside a cell (e.g. siRNA, miRNA, antisense therapy, lncRNA), CRISPR molecules (e.g. gRNA, Cas9 protein, Cas9 mRNA), small molecule therapies (e.g. protein-tyrosine kinase inhibitors, proteasome inhibitors), proteins, peptides, and diagnostic agents.

In some embodiments, the cleavable linker is a modality that can be cleaved enzymatically by endopeptidases. In some embodiments the linkage is cleaved by the group of proteases known as cathepsins. This group of proteases includes cathepsin A, cathepsin B, cathepsin, C, cathepsin D, cathepsin E, cathepsin F, cathepsin G, cathepsin H, cathepsin K, cathepsin L1, cathepsin L2, cathepsin O, cathepsin S, cathepsin W and cathepsin Z.

Accordingly, in some embodiments, the cleavable linker is a dipeptide that can be cleaved by cathepsin B. Such linkers are widely known in the art, and are described in, for example, U.S. Pat. No. 10,844,375; WO 2015/136,545; U.S. Pat. No. 9,732,341; WO 2015/138,615, the contents of each of which are incorporated herein by reference. Examples of dipeptides include, but are not limited to, Tyr-Arg, Phe-Arg, Val-Gln, Val-Cit, Tyr-Met, Leu-Gln, Val-Arg, Met-Thr, Phe-Gln, Thr-Thr, Val-Thr, Ala-Ala, Val-Met, Leu-Met, Ala-Asn, Val-Ala, Phe-Met. These amino acid pairs may be cleaved either extracellularly (K. Porter, Y. Lin, P. B. Liton, Cathepsin B Is Up-Regulated and Mediates Extracellular Matrix Degradation in Trabecular Meshwork Cells Following Phagocytic Challenge, PLoS One. 8 (2013) e68668. https://doi.org/10.1371/journal.pone.0068668.) or intracellularly (M. C. Yoon, A. Solania, Z. Jiang, M. P. Christy, S. Podvin, C. Mosier, C. B. Lietz, G. Ito, W. H. Gerwick, D. W. Wolan, G. Hook, A. J. O'Donoghue, V. Hook, Selective Neutral pH Inhibitor of Cathepsin B Designed Based on Cleavage Preferences at Cytosolic and Lysosomal pH Conditions, ACS Chem. Biol. 16 (2021) 1628-1643. https://doi.org/10.1021/acschembio.1c00138.). In certain embodiments, the cathepsin-cleavable linker is cleaved in tumour cells.

In certain embodiments, the dipeptide is conjugated to a unit that will allow for traceless release of the cargo. For example, this could be apara-aminocarbamate (PABC) linkage. In further embodiments, the PABC linkage is conjugated to a handle that will allow for click chemistry to the cargo. The term “click chemistry” is used to describe any facile reaction that occurs in high yields, under mild conditions and with the formation of limited to no by-products. This compound can be synthesized as a bifunctional linker that can be used to functionalize the 5′ end of amine-modified oligonucleotides. In some embodiments this linker can also be used for peptide, protein and antibody functionalization. This compound is shown in general formula (IX) below.

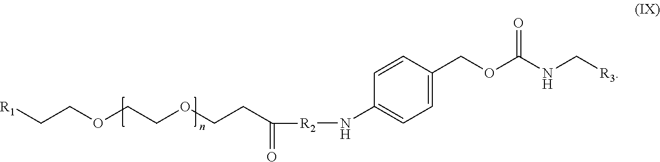

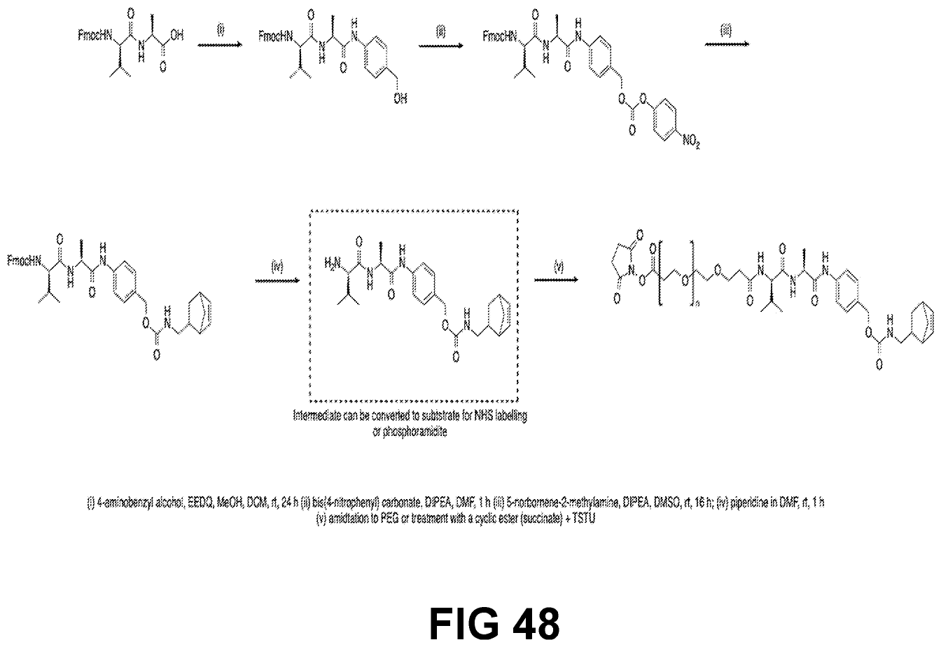

R₁ is reactive in a reaction selected from the group consisting of NHS-based amidation, CuAAC, SPAAC, RuAAC, IEDDA, SuFEx, SPANC, hydrazone/oxime ether formation, thiol-ene radical reaction, thiol-yne radical reaction, thiol-Michael addition reaction, thiol-isocyanate reaction, thiol-epoxide click reaction, nucleophilic ring opening reaction (spring-loaded reactions), and traceless Staudinger ligation and is orthogonal to R₃. R₁ should be reacted with a central core strand in the nucleic acid nanoparticle. R₂ is a dipeptide selected from the group consisting of, but not limited to, Tyr-Arg, Phe-Arg, Val-Gln, Val-Cit, Tyr-Met, Leu-Gln, Val-Arg, Met-Thr, Phe-Gln, Thr-Thr, Val-Thr, Ala-Ala, Val-Met, Leu-Met, Ala-Asn, Val-Ala, Phe-Met. R₃ is reactive in a reaction selected from the group consisting of CuAAC, SPAAC, RuAAC, IEDDA, SuFEx, SPANC, hydrazone/oxime ether formation, thiol-ene radical reaction, thiol-yne radical reaction, thiol-Michael addition reaction, thiol-isocyanate reaction, thiol-epoxide click reaction, nucleophilic ring opening reaction (spring-loaded reactions), and traceless Staudinger ligation and is orthogonal to R₁. R₃ should be reacted with the cargo molecule to be attached to the nucleic acid nanoparticle. The PEG could consist of 0, 1, 2, 3, 4, 5, 6, 7, 8, 9, 10, 15, 20 or more units (n). The synthesis of an example linker with a norbornene modifier for IEDDA conjugation is given in FIG. 48.

In further embodiments, alternative traceless linkers may be used. These include, but are not limited to benzyl carbamates (DEC) (M. He, J. Li, H. Han, C. A. Borges, G. Neiman, J. J. Rsise, P. Hadaczek, R. Mendonsa, V. R. Holm, R. C. Wilson, K. Bankiewicz, Y. Zhang, C. M. Sadlowski, K. Healy, L. W. Riley, N. Murthy, A traceless linker for aliphatic amines that rapidly and quantitatively fragments after reduction, Chem. Sci. 11 (2020) 8973-8980. https://doi.org/10.1039/DOSC00929F.), hydroxylbenzylamines (D. A. Rose, J. W. Treacy, Z. J. Yang, J. H. Ko, K. N. Houk, H. D. Maynard, Self-Immolative Hydroxybenzylamine Linkers for Traceless Protein Modification, J. Am. Chem. Soc. 144 (2022) 6050-6058. https://doi.org/10.1021/jacs.2c01136.), hydrazones, disulfides and pyrophosphate diesters.

In some embodiments, this linker is conjugated to the 5′ end of amine-modified oligonucleotides via R₁. In further embodiments, the functionalized oligonucleotide can be further reactive with the orthogonal terminus (R₃) to a cargo molecule. In further embodiments, the functionalized and conjugated oligonucleotide can be assembled into a nucleic acid nanoparticle of general formula (VIII), whereby L is the linker described in general formula (IX) and X is the cargo molecule that is conjugated via click chemistry.

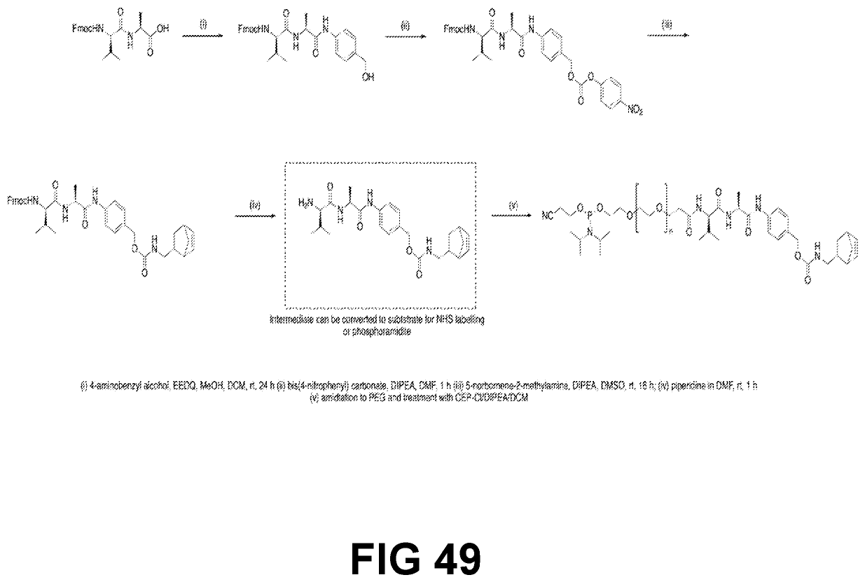

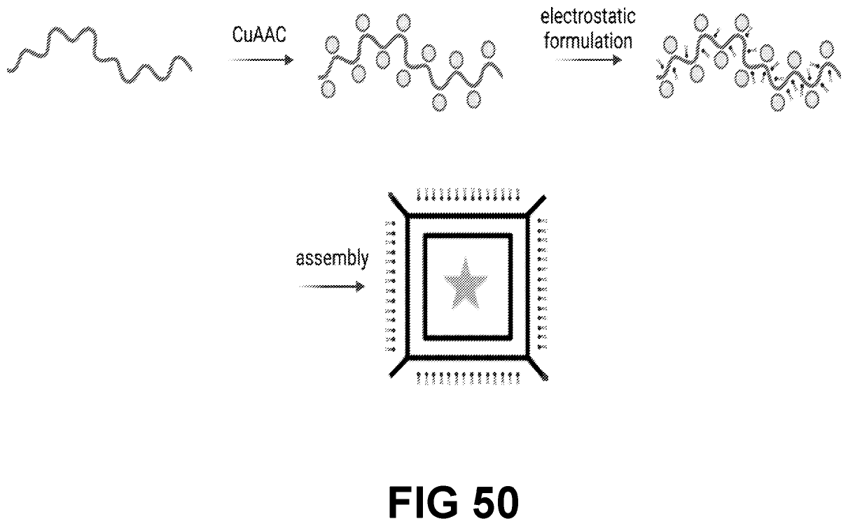

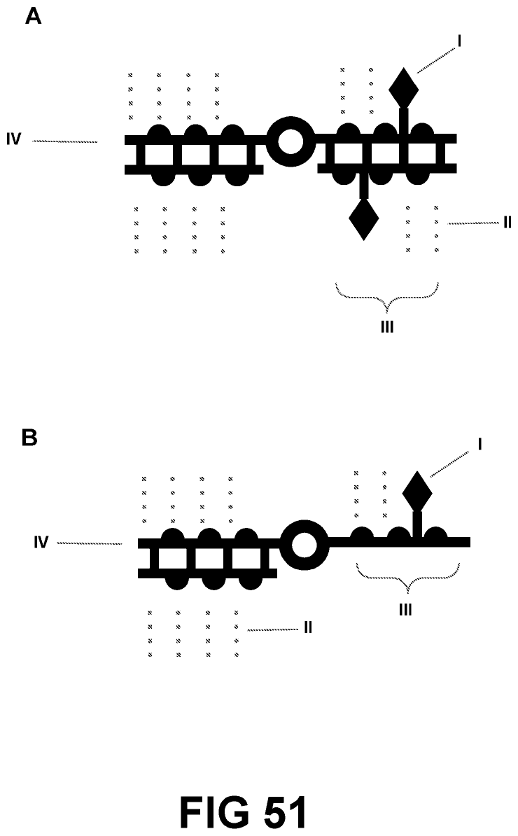

In some embodiments, the cathepsin-cleavable linker is incorporated into an oligonucleotide via phosphoramidite chemistry. Such phosphoramidites have recently been described by Jin et al. (C. Jin, A. H. EI-Sagheer, S. Li, K. A. Vallis, W. Tan, T. Brown, Engineering Enzyme-Cleavable Oligonucleotides by Automated Solid-Phase Incorporation of Cathepsin B Sensitive Dipeptide Linkers, Angew. Chemic—Int. Ed. 61 (2022) 1-7. https://doi.org/10.1002/anie.202114016.). As an extension of this concept, in some embodiments, the current disclosure includes a series of novel phosphoramidites that have the general formula (X):

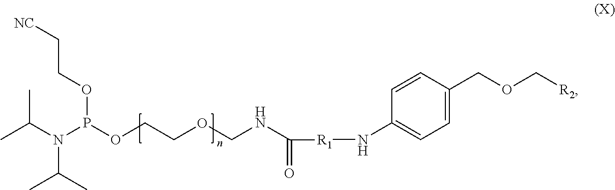

wherein R₁ is a dipeptide selected from the group consisting of, but not limited to, Tyr-Arg, Phe-Arg, Val-Gln, Val-Cit, Tyr-Met, Leu-Gln, Val-Arg, Met-Thr, Phe-Gln, Thr-Thr, Val-Thr, Ala-Ala, Val-Met, Leu-Met, Ala-Asn, Val-Ala, Phe-Met. R₂ is reactive in a reaction selected from the group consisting of CuAAC, SPAAC, RuAAC, IEDDA, SuFEx, SPANC, hydrazone/oxime ether formation, thiol-ene radical reaction, thiol-yne radical reaction, thiol-Michael addition reaction, thiol-isocyanate reaction, thiol-epoxide click reaction, nucleophilic ring opening reaction (spring-loaded reactions), and traceless Staudinger ligation. The PEG could consist of 0, 1, 2, 3, 4, 5, 6, 7, 8, 9, 10. 15, 20 or more units (n). The PABC group used in the bifunctional linker is replaced with a p-aminophenethyl alcohol (PAP) group due to reported concerns with instability during oligonucleotide synthesis. The synthesis of an example phosphoramidite with a norbornene modifier for IEDDA conjugation is given in FIG. 49.

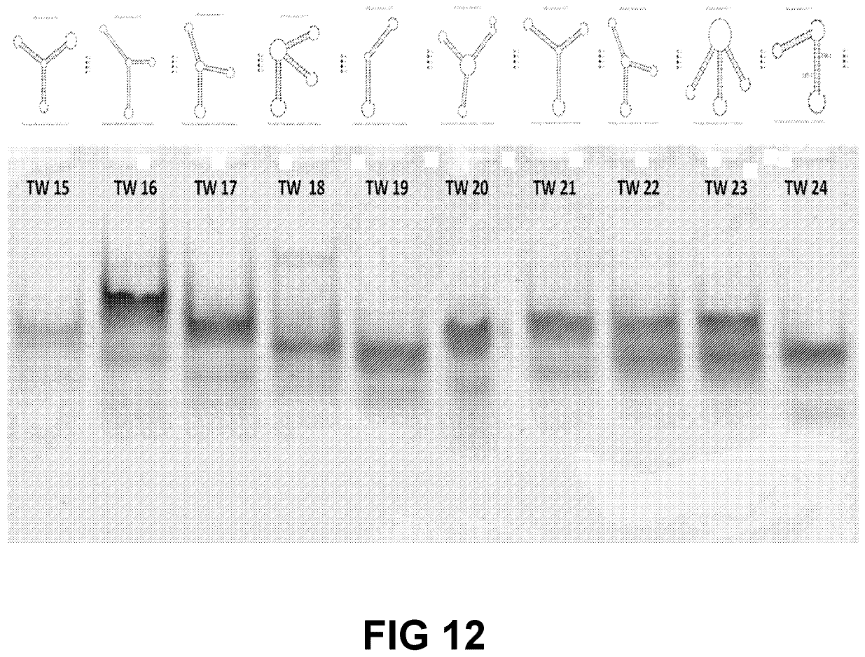

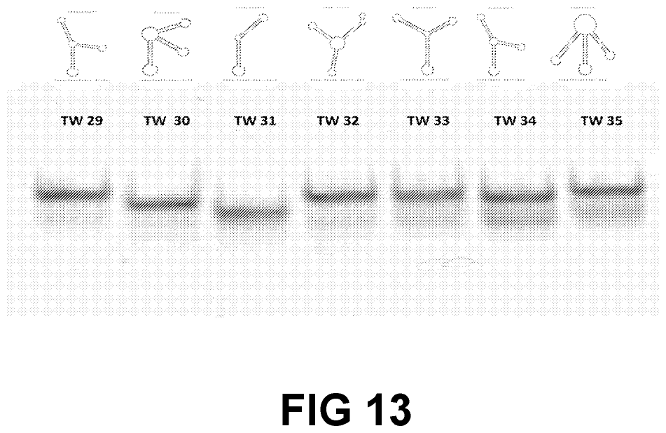

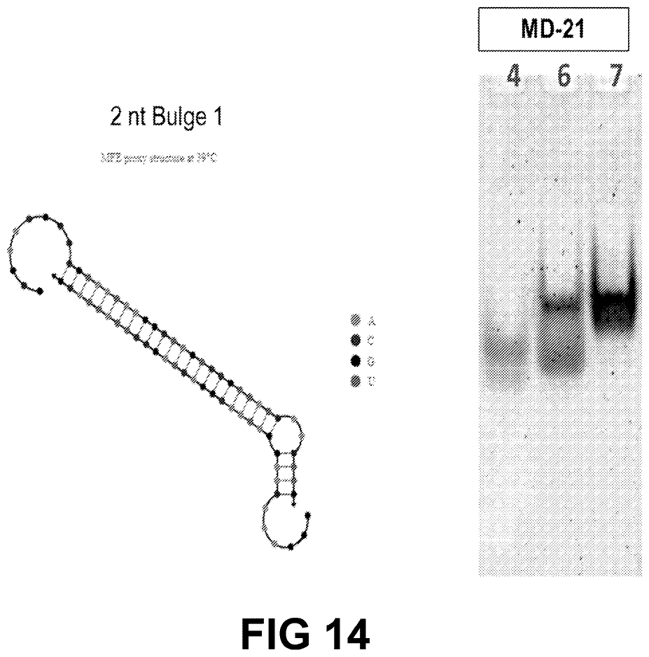

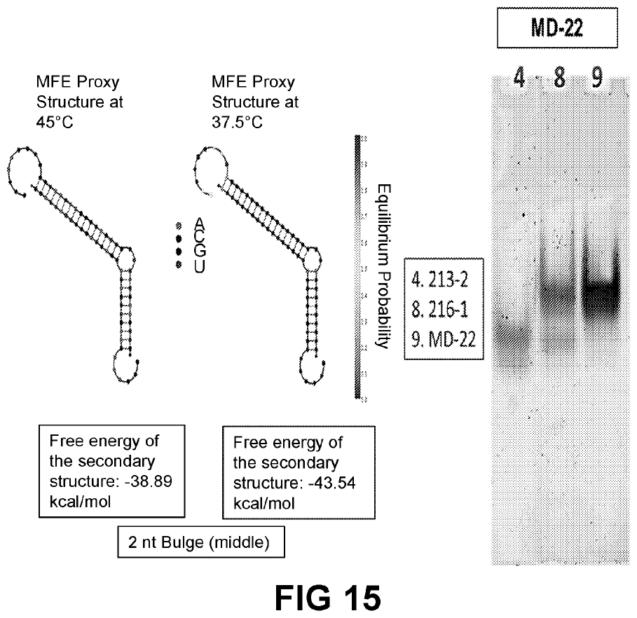

In further aspects, the oligonucleotides that are functionalized with the dipeptide phosphoramidite can be conjugated to a cargo at the R₂ position. In further embodiments, the functionalized and conjugated oligonucleotide can be assembled into a nucleic acid nanoparticle of general formula (VIII), whereby L is the linker described in general formula (IX) and X is the cargo molecule that is conjugated via click chemistry. In some embodiments, the nucleic acid nanoparticle is a Mergo. The cleavage of the linker and release of cargo is shown in FIG. 12.

RNA Nanoparticles with Sheddable Modifications and Therapeutics

In some embodiments, the current disclosure describes non-covalent attachment of PEG to an oligonucleotide and subsequent nucleic acid nanoparticle. Electrostatic attachment of PEG to an oligonucleotide has been shown by Chakraborty et al. (G. Chakraborty, K. Balinin, G. Portale, M. Loznik, E. Polushkin, T. Weil, A. Herrmann, Electrostatically PEGylated DNA enables salt-free hybridization in water, Chem. Sci. 10 (2019) 10097-10105. https://doi.org/10.1039/c9sc02598g.). To obtain this electrostatic interaction, anion exchange is carried out on ssDNA by precipitating the molecule out of aqueous medium by electrostatic complexation with 4-(hexyloxy)anilinium (ANI). The DNA-ANI complex is then freeze-dried and resuspended in methanol. An excess of PEG with a terminal primary amine is then dissolved in methanol and added to the freeze-dried material to afford a DNA-PEG complex. The DNA-PEG complex can then be hybridized in salt-free conditions. Furthermore, the physicochemical characteristics imparted by the PEG aid in overcoming solubility challenges observed with highly modified oligonucleotides. The major challenge that remains, however, is dePEGylation upon either hybridization to form a nucleic acid nanoparticle, or when administered in an in vivo setting.

Accordingly, in some embodiments, the current disclosure provides solutions to the “dePEGylation” problem. PEGs used as electrostatic binders have general formula (XI):

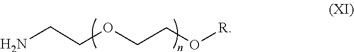

Where R is either H or a methyl group. The PEG length (n) could be anywhere between 1 to 30 units long. Such PEG molecules are commercially available or are known in the art.

In further embodiments, the PEG molecule used in this disclosure can have the general formula (XII):

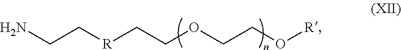

where R is a functional group that will allow for cleavage of the hydrocarbon chain in response to a range of stimuli. This may include, but is not limited to, a redox-responsive disulfide (J. Winkler, Oligonucleotide conjugates for therapeutic applications, Ther. Deliv. 4 (2013) 791-809. https://doi.org/10.4155/tde.13.47.), pH responsive hydrazone (N. Olivier, C. Olivier, C. Gouyette, T. Huynh-Dinh, H. Gras-Masse, O. Melnyk, Synthesis of oligonucleotide-peptide conjugates using hydrazone chemical ligation, Tetrahedron Lett. 43 (2002) 997-999. https://doi.org/10.1016/S0040-4039(01)02315-2.), hydrazine (S. Raddatz, J. Mueller-Iheler, J. Kluge, L. WaB, G. Burdinski, J. R. Havens, T. J. Onofrey, D. Wang, M. Schweitzer, Hydrazide oligonucleotides: New chemical modification for chip array attachment and conjugation, Nucleic Acids Res. 30 (2002) 4793-4802. https://doi.org/10.1093/nar/gkf594.), acetal (S. Matysiak, R. Frank, W. Pfleiderer, Acetal oligonucleotide conjugates in antisense strategy, Nucleosides and Nucleotides. 16 (1997) 855-861. https://doi.org/10.1080/07328319708002963.). or benzoic imine (Y. Wang, Q. Luo, R. Sun, G. Zha, X. Li, Z. Shen, W. Zhu, Acid-triggered drug release from micelles based on amphiphilic oligo(ethylene glycol)-doxorubicin alternative copolymers, J. Mater. Chem. B. 2 (2014) 7612-7619. https://doi.org/10.1039/c4tb01231c.), or ROS-responsive thioketal (Y. Zhang, J. Zhou, S. Ma, Y. He, J. Yang, Z. Gu, Reactive Oxygen Species (ROS)-Degradable Polymeric Nanoplatform for Hypoxia-Targeted Gene Delivery: Unpacking DNA and Reducing Toxicity, Biomacromolecules. 20 (2019) 1899-1913. doi.org/10.1021/acs.biomac.9b00054.). Additional linkers may include any that are highlighted by Su et al. (Z. Su, D. Xiao, F. Xie, L. Liu, Y. Wang, S. Fan, X. Zhou, S. Li, Antibody-drug conjugates: Recent advances in linker chemistry, Acta Pharm. Sin. B. 11 (2021) 3889-3907. https://doi.org/10.1016/j.apsb.2021.03.042.); and Bargh et al. (J. D. Bargh, A. Isidro-Llobet, J. S. Parker, D. R. Spring, Cleavable linkers in antibody-drug conjugates, Chem. Soc. Rev. 48 (2019) 4361-4374. https://doi.org/10.1039/c8cs00676h.). R′ is either H or a methyl group. The terminal primary amine can undergo anion exchange and form an electrostatic interaction with the phosphate backbone. The PEG length (n) could be anywhere between 1 to 30 units long.

In embodiments, the electrostatic component may be any molecule that aids in the formulation of oligonucleotides that are heavily modified with hydrophobic modifications. These include, but are not limited to, PEGs, carbohydrates, peptides, proteins, polymers and small molecules.

In further embodiments, the electrostatic PEGs in the present disclosure may have formula (XIII):

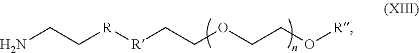

where R is a masked modification that is able to modulate the physicochemical properties of an oligonucleotide or nucleic acid nanoparticle. This includes, but is not limited to, spermine, ethylenediamine, methylethylenediamine, ethylethylenediamine, imidazole, spermine-imidazole-4-imine, N-ethyl-N′-(3-dimethylaminopropyl)-guanidinyl ethylene imine, dimethylaminoethyl acrylate, amino vinyl ether, 4-imidazoleacetic acid, diethylaminopropylamide, sulfonamides (e.g. sulfadimethoxine sulfamethoxazole, sulfadiazine, sulfamethazine), amino ketals, N-2-hydroxylpropyltimehyl ammonium chloride, imidazole-4-imines, methyl-imidazoles, 2-(aminomethyl)imidazole, 4-(aminomethyl)imidazole, 4(5)-(hydroxymethyl)imidazole, N-(2-aminoethyl)-3-((2-aminoethyl)(methyl)amino)propanamide, 2-(2-ethoxyethoxy)ethan-1-amine, bis(3-aminopropyl)amine, [N,N-dimethylamino)ethoxy]ethyl, N-(2-aminoethyl)-3-((2-aminoethyl)(ethyl)amino)propanamide, (N-(aminoethyl)carbamoyl)methyl, N-(2-((2-aminoethyl)amino)ethyl)acetamide 3,3′-((2-aminoethyl)azanediyl)bis(N-(2-aminoethyl)propanamide), guanidyl benzylamide, [3-(guanidinium)propyl], dimethylethanolamine, 1-(2,2-dimethyl-1,3-dioxolan-4-yl)-N,N-dimethylmethanamine, 2-(2,2-dimethyl-1,3-dioxolan-4-yl)-N,N-dimethylethan-1-amine, N-(2-((2-(2-aminoethoxy)propan-2-yl)oxy)ethyl)acetamide, aminobutyl, aminoethyl, 1-(2-aminoethyl)-3-(3-(dimethylamino)propyl)-2-ethylguanidine, 1-(3-amino-3-oxopropyl)-2,4,6-trimethylpyridin-1-ium, 1-(1,3-bis(carboxyoxy)propan-2-yl)-2,4,6-trimethylpyridin-1-ium, guanidinylethyl amine, ether hydroxyl triazole, or a β-aminoester. Where R′ is a functional group that will allow for cleavage of the hydrocarbon chain in response to a range of stimuli. This may include, but is not limited to, a redox-responsive disulfide (J. Winkler, Oligonucleotide conjugates for therapeutic applications, Ther. Deliv. 4 (2013) 791-809. https://doi.org/10.4155/tde.13.47.), pH responsive hydrazone (N. Ollivier, C. Olivier, C. Gouyette, T. Huynh-Dinh, H. Gras-Masse, O. Melnyk, Synthesis of oligonucleotide-peptide conjugates using hydrazone chemical ligation, Tetrahedron Lett. 43 (2002) 997-999. https://doi.org/10.1016/S0040-4039(01)02315-2.), hydrazine (S. Raddatz, J. Mueller-Ibeler, J. Kluge, L. WaB, G. Burdinski, J. R. Havens, T. J. Onofrey, D. Wang, M. Schweitzer, Hydrazide oligonucleotides: New chemical modification for chip array attachment and conjugation, Nucleic Acids Res. 30 (2002) 4793-4802. https://doi.org/10.1093/nar/gkf594.), acetal (S. Matysiak, R. Frank, W. Pfleiderer, Acetal oligonucleotide conjugates in antisense strategy, Nucleosides and Nucleotides. 16 (1997) 855-861. https://doi.org/10.1080/07328319708002963.), or benzoic imine (Y. Wang, Q. Luo, R. Sun, G. Zha, X. Li, Z. Shen, W. Zhu, Acid-triggered drug release from micelles based on amphiphilic oligo(ethylene glycol)-doxorubicin alternative copolymers, J. Mater. Chem. B. 2 (2014) 7612-7619. https://doi.org/10.1039/c4tb01231c.), or ROS-responsive thioketal (Y. Zhang, J. Zhou, S. Ma, Y. He, J. Yang, Z. Gu, Reactive Oxygen Species (ROS)-Degradable Polymeric Nanoplatform for Hypoxia-Targeted Gene Delivery: Unpacking DNA and Reducing Toxicity, Biomacromolecules. 20 (2019) 1899-1913. doi.org/10.1021/acs.biomac.9b00054.). Additional linkers may include any that are highlighted by Su et al. (Z. Su, D. Xiao, F. Xie, L. Liu, Y. Wang, S. Fan, X. Zhou, S. Li, Antibody-drug conjugates: Recent advances in linker chemistry, Acta Pharm. Sin. B. 11 (2021) 3889-3907. https://doi.org/10.1016/j.apsb.2021.03.042.); and Bargh et al. (J. D. Bargh, A. Isidro-Llobet, J. S. Parker, D. R. Spring, Cleavable linkers in antibody-drug conjugates, Chem. Soc. Rev. 48 (2019) 4361-4374. https://doi.org/10.1039/c8cs00676h.). R″ is either H or a methyl group. The terminal primary amine can undergo anion exchange and form an electrostatic interaction with the phosphate backbone. The PEG chain length (n) could be anywhere between 1, 2, 3, 4, 5, 6, 7, 8, 9, 10, 15, 20, 30 units long or more.

An example of a guanidine-based compound with general formula (XIV) is given below.

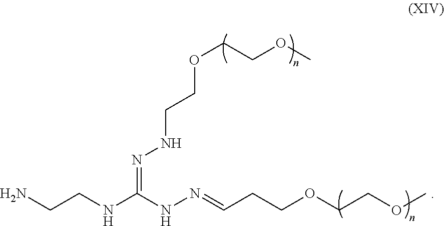

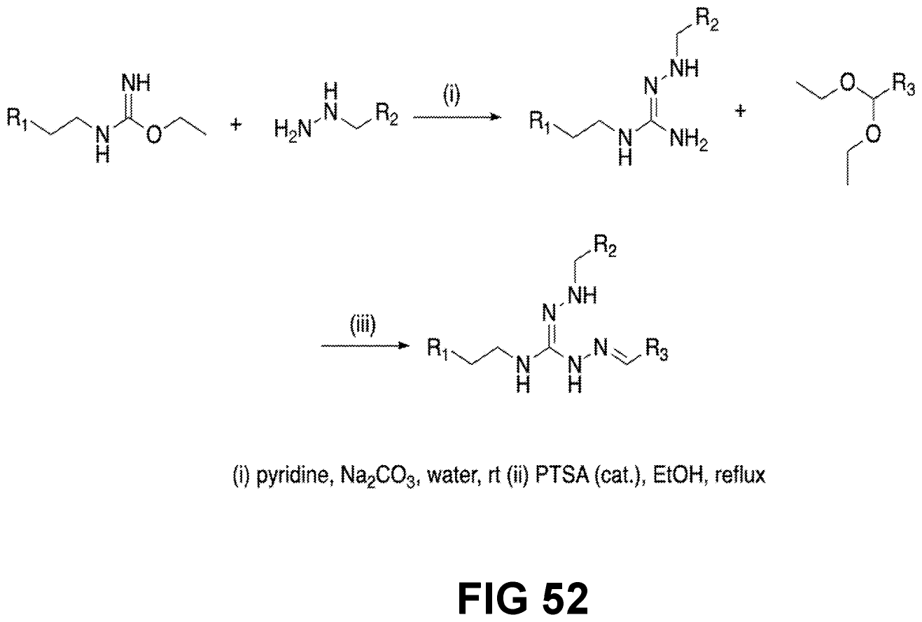

The synthetic route towards this compound is shown in FIG. 52. The R₁-ethyl carbamimidate can be treated with the hydrazine form of R₂ in the presence of pyridine, Na₂CO₃ and water, according to a method outlined in US 2010/120,741. The subsequent intermediate is then refluxed the ethyl acetal protected R₃ in the presence of catalytic toluene-4-sulfonic acid to afford the protected bis-hydrazone ([A. Pogorzelska, J. Slawinski, B. Zolnowska, K. Szafranski, A. Kawiak, J. Chojnacki, S. Ulenberg, J. Zielinska, T. Bgczek, Novel 2-(2-alkylthiobenzenesulfonyl)-3-(phenylprop-2-ynylideneamino)guanidine derivatives as potent anticancer agents—Synthesis, molecular structure, QSAR studies and metabolic stability, Eur. J. Med. Chem. 138 (2017) 357-370. https://doi.org/10.1016/j.ejmech.2017.06.059.). In some embodiments, R₂ is equal to R₃. In some embodiments R₂ is different from R₃.

Upon exposure to an acidic environment, the hydrazones are cleaved and the guanidine units are exposed.

In some aspects, the PEGs described in this disclosure can be electrostatically linked to oligonucleotides. In further aspects, the PEG-functionalized oligonucleotides can be assembled into Mergos.

Upon exposure to a salt-containing solution, the electrostatic molecules may be substituted for metal cations. The average concentration of sodium in blood is 136 to 145 mM and it has been shown that a sodium concentration of 100 mM can lead to substitution of the hydrostatic PEGs. (G. Chakraborty, K. Balinin, G. Portale, M. Loznik, E. Polushkin, T. Weil, A. Herrmann, Electrostatically PEGylated DNA enables salt-free hybridization in water, Chem. Sci. 10 (2019) 10097-10105. https://doi.org/10.1039/C9SC02598G.). In this respect, delivery of PK/PD modulating modifications may be more of a co-treatment strategy, whereby the small molecule modifications are detached from the delivery system prior to delivery. This could be particularly advantageous for compounds that mediate endosomal escape. As such, compounds that may also be electrostatically bound include, but are not limited to, chloroquine, 1-[1-(6-Chloroquinolin-4-yl)piperidin-4-yl]piperidin-3-ol, 1-(7-chloroquinolin-4-yl)piperidin-4-ol, 2-[4-(7-Chloroquinolin-4-yl) morpholin-2-yl]ethanamine, [1-(7-Chloroquinolin-4-yl)piperidin-3-yl]methanol, 1R,2R)-2-N-(7-Chloroquinolin-4-yl)cyclohexane-1,2-diamine, (1S,2S)-2-N-(7-chloroquinolin-4-yl)cyclohexane-1,2-diamine, N′-(7-chloroquinolin-4-yl)-N-cyclohexylethane-1,2-diamine, N-(4-aminobutyl)-7-chloroquinolin-4-amine, (1R,2S)-2-N-(7-chloroquinolin-4-yl)cyclohexane-1,2-diamine, N′-(7-Chloroquinolin-4-yl)-N-ethylpropane-1,3-diamine, N-(7-Chloroquinolin-4-yl)-N′,N′-dimethylbutane-1,4-diamine, N4-(7-chloroquinolin-4-yl)-n1-methylpentane-1,4-diamine, Didesethyl Chloroquine, 3-N-(7-chloroquinolin-4-yl)-1-N,1-N-dimethylbutane-1,3-diamine, N′-(7-Chloroquinolin-4-yl)-N-(2-methylpropyl)propane-1,3-diamine, Desethyl Chloroquine, N′-(7-chloroquinolin-4-yl)-N-cyclohexylpropane-1,3-diamine, 1-(7-Chloroquinolin-4-yl)-N,N-dimethylpiperidin-3-amine, N-(7-chloroquinolin-4-yl)-N′,N′-diethylpropane-1,3-diamine, (3R)-1-(7-chloroquinolin-4-yl)azepan-3-amine, N-(7-chloroquinolin-4-yl)-N′-propan-2-ylpropane-1,3-diamine, 1-(7-chloroquinolin-4-yl)azepan-3-amine, N-(7-Chloroquinolin-4-yl)-N′,N′-di(propan-2-yl)ethane-1,2-diamine, N-(7-chloroquinolin-4-yl)-N′,N′-dimethylpropane-1,3-diamine, N′-(7-Chloroquinolin-4-yl)-N,N-diethylethane-1,2-diamine, 1-N-(7-Chloroquinolin-4-yl)-2-N,2-N-dimethylpropane-1,2-diamine, (1-Quinolin-4-ylpiperidin-4-yl) methanamine, N-(6-Chloroquinolin-4-yl)-N′,N′-diethylpropane-1,3-diamine, N-(7-Chloroquinolin-4-yl)-N′-methylpropane-1,3-diamine, (E)-N-(7-Chloroquinolin-4-yl)-N′,N′-diethylbut-2-ene-1,4-diamine, 7-Chloro-N-(5-pyrrolidin-1-ylpentan-2-yl)quinolin-4-amine, ethyl (8-((2-(dimethylamino)ethyl)amino)-2,3-diphenylpyrido[2,3-b]pyrazin-6-yl)carbamate or ethyl (8-((2-(dimethylamino)ethyl)amino)-2,3-diphenylpyrido[2,3-b]pyrazin-6-yl)carbamate.

In further embodiments, the electrostatic carbohydrates in the present disclosure are amino sugars, whereby the 2′-carbon hydroxyl substituent is replaced by an —NH₂ amine group. These include, but are not limited to, N-acetylglucosamine, galactosamine, glucosamine, sialic acid, L-duanosamine and polymers thereof. Carbohydrates could be monosaccharides, disaccharides, olugosaccharides or polysaccharides.

In embodiments, the electrostatic carbohydrate could be chitosan.

In embodiments, the electrostatic carbohydrates could be any given amine-containing oligosaccharides. Examples of amine-containing oligosaccharides include amine-functionalised heparin oligosaccharides (S. Maza, G. MacChione, R. Ojeda, J. L6pez-Prados, J. Angulo, J. L. De Paz, P. M. Nieto, Synthesis of amine-functionalized heparin oligosaccharides for the investigation of carbohydrate-protein interactions in microtiter plates, Org. Biomol. Chem. 10 (2012) 2146-2163. https://doi.org/10.1039/c2ob06607f.), Streptococcus mutans hexasaccharides (R. Castelli, H. S. Overkleeft, G. A. Van Der Marel, J. D. C. Codde, 2,2-dimethyl-4-(4-methoxy-phenoxy) butanoate and 2,2-dimethyl-4-azido butanoate: Two new pivaloate-ester-like protecting groups, Org. Lett. 15 (2013) 2270-2273. https://doi.org/10.1021/o14008475.) and those that are known in the art, including aminoglycosides with free amines used for medical devices (P. J. Hanson, J. Courage, AMINE-FUNCTIONALIZED POLYMERIC COMPOSITIONS FOR MEDICAL DEVICES, U.S. Pat. No. 10,688,217, 2017.) and carbohydrate conjugates as delivery agents for oligonucleotides (M. Manoharan, K. G. Rajeev, J. Nair, M. Maier, CARBOHYDRATE CONJUGATES AS DELIVERY AGENTS FOR OLIGONUCLEOTIDES, US 2018/032,6070, 2018.).

Such electrostatic carbohydrate conjugates may have general formula (XV):

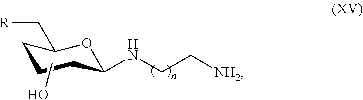

wherein n is 0, 1, 2, 3, 4, 5, 6, 7, 8, 9, 10, 15, 20 or more. R may be H, OH, COOH, CONH₂, CONHMe, CONMe₂. Such carbohydrates are commercially available or known in the art.

In further embodiments, electrostatic carbohydrate conjugates may have general formula (XVI):

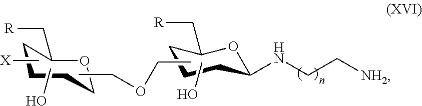

wherein n is 0, 1, 2, 3, 4, 5, 6, 7, 8, 9, 10, 15, 20 or more. R may be H, OH, COOH, CONH₂, CONHMe, CONMe₂. X is further repeating units of the sugar monomers. The number of repeats could be 0, 1, 2, 3, 4, 5, 6, 7, 8, 9, 10 or more monomers. Sugars may be linked at any given position on the ring. Ring sizes can vary between five and six carbons, depending on the sugar monomer. Sugar monomers can include, but are not limited to, D-Ribulose 5-phosphate, D-Mannuronic acid, L-Fuconic acid, 1-Deoxy-D-xylulose-5-phosphate, D-Fructose-1,2-cyclic-6-bisphosphate, D-Erythritol 4-phosphate, D-galactose 6-phosphate, 2-Deoxy-D-ribonic acid, 6-Phosphogluconic acid, β-L-Fucose 1-phosphate bis(cyclohexylammonium), D-Fructose 1,6-bisphosphate, D-Mannose 6-phosphate, D-Xylonic acid, L-Glyceric acid, L-Threonic acid, 6-phospho-D-galactonate, N-Acetyl-D-glucosamine, D-Glyceric acid, D-Mannose, D-Ribose, Agaric acid, D-Mannitol, Dulcitol, L-Sorbose, D-Glucose diethylmercaptal, Dhurrin, Levoglucosenone, α-Homonojirimycin, Arbutin, Fusicoccin, L-(−)-Dithiothreitol, L-(+)-Ribose, D-(−)-Lactic acid, D-(−)-Tagatose, D-Mannoheptose, D-Psicose, D-(+)-Fucose, D-(+)-Sorbose, DL-Arabinose, L-(−)-Galactose, L-(+)-Erythrulose, L-(−)-Fucose, 3-Deoxyglucosone, D-(+)-Arabitol, D-Mannoheptulose, DL-Xylose, epi-Inositol, L-(−)-Glucose, L-(−)-Mannose, L-(+)-Gulose, Lactulose, Methyl α-D-galactopyranoside, 1,5-Anhydro-D-sorbitol, Isopropyl β-D-thioglucopyranoside, L(+)-Erythrose, Methyl-β-D-galactopyranoside, Methyl-β-D-thiogalactoside, N-Acetylmuramic acid, β-D-Allose, D-(+)-Talose, D-Glucosaminic acid, Deoxyfuconojirimycin, 2-Deoxy-L-ribose, L-Galactono-1,4-lactone, L-Idaro-1,4-lactone, N-Acetyl-D-glucosamine, (−)-Sinigrin hydrate, 5-Thio-D-glucose, D-Threono-1,4-lactone, D-Xylono-1,4-lactone, D-(−)-Erythrose, L-Threono-1,4-lactone, Methyl α-D-mannopyranoside, D-Glucosone, N-Glycolylneuraminic acid, 2-Deoxy-D-ribose, 3-O-Methyl-D-glucopyranose, D-Glucosamine 3-sulfate, D-Altrose, D-(−)-Fructose, D-(+)-Xylose, Glycerol, D-Galactose, L-(−)-Sorbose, meso-Erythritol, Methyl α-D-glucopyranoside, Dextrose, L-Rhamnose, N-Acetyl-D-galactosamine, L-(+)-Arabinose.

Additionally, carbohydrate electrostatic conjugates may be conjugated to PEG molecules. These compounds have the general formula (XVII):

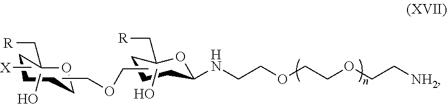

wherein n is 0, 1, 2, 3, 4, 5, 6, 7, 8, 9, 10, 15, 20 or more. R may be H, OH, COOH, CONH₂, CONHMe, CONMe₂. X is further repeating units of the sugar monomers. The number of repeats could be 0, 1, 2, 3, 4, 5, 6, 7, 8, 9, 10 monomers. Sugars may be linked at any given position on the ring. Ring sizes can vary between five and six carbons, depending on the sugar monomer. Sugar monomers can be any from those given above.

In embodiments, the electrostatic hydrophilic molecule might be a hydrophilic peptide. There are many hydrophilic peptides known in the art. Examples of these are incorporated herein by reference. These include small linear peptide antigens (M. Manoharan, K. G. Rajeev, J. Nair, M. Maier, CARBOHYDRATE CONJUGATES AS DELIVERY AGENTS FOR OLIGONUCLEOTIDES, US 2018/032,6070, 2018.) and biologically active peptides (J. A. Fuselier, D. H. Coy, Conjugates of therapeutic or cytotoxic agents and biologically active peptides, WO2003074551A2, 2003.).

Peptides will consist of the 20 common amino acids. Hydrophobic residues are defined as Ala, Ile, Leu, Met, Phe, Trp, Val. Uncharged hyophilic residues are defined as Asn, Cys, Gly, Gln, Pro, Ser, The, Tyr. Acidic hydrophilic residues are defined as Asp, Glu. Basic hydrophilic residues are defined as His, Lys, Arg.

Generally, hydrophilic peptides will adhere to general solubility rules. Peptides may be shorter than 5 residues. Longer peptides will contain >25% charged residues and <25% hydrophobic residues.

The N-terminus of the electrostatic peptides will be free for binding to the phosphate backbone.

The N-terminus of the peptide may be further conjugated to a linker or PEG spacer. This spacer will have a primary amine at the terminus.

In embodiments, the hydrophilic electrostatic molecule may be a hydrophilic polymer. There are numerous examples of hydrophilic polymers in the art; these are often used as an alternative to PEG and can form conjugates for higher order structures such as micelles (H. Cabral, K. Miyata, K. Osada, K. Kataoka, Block Copolymer Micelles in Nanomedicine Applications, Chem. Rev. 118 (2018) 6844-6892. https://doi.org/10.1021/acs.chemrev.8b00199.) and polymersomes (J. S. Lee, J. Feijen, Polymersomes for drug delivery: Design, formation and characterization, J. Control. Release. 161 (2012) 473-483. https://doi.org/10.1016/j.jconrel.2011.10.005.) or hydrogels (E. Larra5eta, S. Stewart, M. Ervine, R. Al-Kasasbeh, R. F. Donnelly, Hydrogels for hydrophobic drug delivery. Classification, synthesis and applications, J. Funct. Biomater. 9 (2018). https://doi.org/10.3390/jfb9010013.).

Further examples of the use of hydrophilic polymers for drug delivery in the art are given in U.S. Pat. No. 7,160,557-B2, U.S. Pat. No. 8,586,098-B2, U.S. Pat. No. 9,265,836-B2, U.S. Ser. No. 10/213,528-B2, U.S. Pat. No. 8,674,032-B2.

Polymers may be formed by chain-growth or step-growth polymerization.

Chain-growth polymerization could include radical polymerization, ionic polymerization, coordination polymerization, living polymerization, ring-opening polymerization and reversible-deactivation polymerization.

Step-growth polymers could include polyethers.

All polymers will be fully N-protected, where appropriate, besides one primary amine. This may or may not be at the polymer terminus.

Polymers suitable as electrostatic binders have general formula (XVIII):

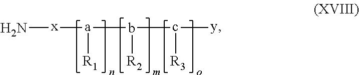

whereby x is a linker that may or may not be attached to a chain transfer agent, y is the polymer terminus, a, b and c are polymer blocks consisting of repeating units R₁, R₂ and R₃. In some embodiments, R₁=R₂=R₃. In some embodiments, R₁≠R₂≠R₃. In further embodiments, R₁=R₂≠R₃. In further embodiments, R₁≠R₂=R₃. These may be block copolymers or statistical or random copolymers, n, m and o are integers between 1 and 500.

An example of an amine-terminated polymer is given in the art (S. Aroua, E. G. V. Tiu, M. Ayer, T. Ishikawa, Y. Yamakoshi, RAFT synthesis of poly(vinylpyrrolidone) amine and preparation of a water-soluble C60-PVP conjugate, Polym. Chem. 6 (2015) 2616-2619. https://doi.org/10.1039/c4py01333f.). This polymer is synthesized via modification of a RAFT agent with a terminal amine. Such RAFT agents could be used to generate polymers with hydrophilic monomers. Examples of monomers that could be used with such a RAFT agent include, but are not limited to, (HEMA-10) poly ethoxy (10) ethyl methacrylate, hydroxypolyethoxy (10) allyl ether, N, N-dimethylacrylamide, ethylene glycol dimethacrylate, methacrylic acid, beta-carboxyethyl acrylate, 2-cyanoethyl acrylate, 2-hydroxyethyl methacrylate, triethylene glycol methyl ether methacrylate, poly(propylene glycol) methacrylate, alginate methacrylate, chitosan glycidyl methacrylate, diethylene glycol butyl ether methacrylate, di(ethylene glycol) methyl ether methacrylate, 3-sulfopropyl methacrylate, glycosyloxyethyl methacrylate solution, hyaluronic acid methacrylate, poly(2-dimethylamino)ethyl methacrylate) methyl chloride quaternary salt, [2-(methacryloyloxy)ethyl]trimethylammonium chloride.

Amine-terminated polymers are also shown in the art with many different variations. For example, in U.S. Pat. Nos. 4,133,947A, 4,157,429A, 8,562,966B2, 7,432,440B2.

In further embodiments, the compounds described may be covalently conjugated to an oligonucleotide via the terminal primary amine.

In some embodiments, the electrostatic compounds may be formulated into a hydrophilic layer on the Mergo, whereby the hydrophilic layer consists of multiple molecules with the same identity. This number is given by n(P)-X, whereby n(P) is the number of phosphate moieties in the molecule and X can be any number between 1 and n(P).

In further embodiments, hydrophilic layer can consist of multiple molecules with different identities. There could be 1, 2, 3, 4, 5, 6, 7, 8, 9, 10 or more different types of molecules in varied molar ratios.

In addition to formulation, the electrostatic compounds may have a therapeutic effect. This electrostatic binding mechanism allows for high loading of any given small molecule with a primary amine. Examples of known drugs with primary amines that can be used with the present disclosure include, without limitation, aciclovir, adefovir dipivoxil, alfuzosin, amiloride, aminosalicylic acid, amisulpride, amlexanox, amprenavir, amrinone, anileridine, azacitidine, benzocaine, bleomycin, bromfenac, cefdinir, cefditoren, cefepime, cefixime, cefmenoxime, cefotaxime, cefpodoxime, ceftazidime, ceftriaxone, chloriprocaine, cidofovir, cladribine, clenbuterol, clofarabine, cytarabine, dactinomycin, dapsone, darunavir, decitabine, doxazosin, emtricitabine, entecavir, famciclovir, flucytosine, fludarabine, fosamprenavir, ganciclovir, gemcitobine, imiquimod, lamivudine, lamotrigine, lenalidomide, mesalazine, methotrexate, metoclopramide, minoxidil, mitomycin, nelerabine, oxybuprocaine, pemetrexed, penciclovir, phenazopyridine, pramipexole, prazosin, procainamide, procaine, proflavine, proparacaine, pyrimethamine, riluzole, S-adenosylmethionine, sparfloxacin, sulfacetamide, sulfanilamide, tacrine, tamibarotene, tenofovir, terazosin, tetrahydrobiopterin, tetrahydrofolic acid, thiamine, thioguanine, triamterene, trimethoprim, tremexate, valaciclovir, valganciclovir, vidarabine, zalcitabine.

Electrostatic small molecule coatings may be used in combination with oligonucleotide therapeutics.

Small molecules with indirect therapeutic effects include those that can assist in delivery of the oligonucleotide therapeutics into the cell. Such molecules include amine and hydrazine-derivatives of chloroquine such as hydroxychloroquine-hydrazine, hydroxychloroquine-amine (US200760499), 2-((4-((7-chloroquinolin-4-yl)amino)pentyl)(ethyl)amino)ethyl hydrogen ((((R)-1-(6-amino-9-purin-9-yl)propan-2-yl)oxy)methyl)phosphonate (WO2021/202669), 9-N-[5-(Diethylamino)pentan-2-yl]-2,4-difluoro-7-methoxyacridine-3,9-diamine (WO2022/93871.).

In further embodiments, the electrostatic compounds may be formulated into a hydrophilic layer where the layer consists of molecules with a different identity. Such molecules could be any aforementioned hydrophilic electrostatic binder, endosomal escape-mediating binder or therapeutic binder. Further molecules that could be formulated include amine derivatives of cationic lipids including, but not limited to DOTMA, DOTAP, DOSPA or ePC; Amine derivatives of ionizable lipids including, but not limited to, DLin-MC3-DMA, ALC-0315, Lipid-H (SM-102), A2-Iso5-2DC18, BAME-016B, 9A1P9, C12-200, cKK-E12, OF-Deg-Lin, 306Oᵢ₁₀, TT3, or FTT5; Or amine derivatives of other types of lipids, including but not limited to, DSPC, PEG₂₀₀₀-DMG, ALC-0159, cholesterol, DC-cholesterol, β-sitosterol and BHEM-cholesterol. Such lipids are widely used in the art for LNP formulation (X. Hou, T. Zaks, R. Langer, Y. Dong, Lipid nanoparticles for mRNA delivery, Nat. Rev. Mater. 6 (2021) 1078-1094. https://doi.org/10.1038/s41578-021-00358-0.).

In embodiments, the electrostatic layer may coat a single stranded Mergo that is heavily modified, which is further conjugated to one or more NATs.

In further embodiments, the electrostatic layer may coat a double stranded Mergo that is heavily modified, which is further conjugated to one or more NATs.

In other aspects, oligonucleotides that are coated with an electrostatic layer may be assembled into Mergos in the absence of metal salts.

Such modifications allow for the presence of a multitude of modifications in a compact space; electrostatic modifiers can be used with Mergos where every possible position is modified.

### EXAMPLES

### Example 1—Oligonucleotide Synthesis

Oligonucleotides were synthesized on 1-10 μmol scale using a K&A synthesizer (H-16). All protocols were modified depending on the sequence requirements. Phosphoramidites and CPGs with standard protecting groups were purchased from ChemGenes and Glen Research. Adenosine phosphoramidites containing amino acids, amino acid analogues, PEGs and hydrocarbon chains were synthesized in-house. The detritylation step was carried out with 3% TCA in DCM, followed by coupling with 0.1 M phosphoramidite solutions and 0.25 M BMT in MeCN. Capping was performed using THF/lutidine/acetic anhydride (80/10/10) as capping A and 16% N-methylimidazole in THF as capping B, respectively. The oxidation step was accomplished with 0.02 M iodine solution in THF/Pyr/water (90.6/0.4/9).

All synthesized oligonucleotides were cleaved and deprotected using aq. methylamine/ammonium hydroxide solution (1:1) for 3 h at RT for a solid support with a first base attached or for 1 h at 65° C. for a universal CPG. The removal of tert-butyl silyl protecting groups was performed by incubating an intermediate product in DMSO Et₃N·3HF for 3 h at 65° C. Crude oligonucleotides were subsequently precipitated from ethanolic solution containing sodium acetate. After 2 h at −70° C. the precipitate was harvested by 25 min centrifugation at 4° C. (14,000 rpm). The supernatant was separated, and the remaining pellet was washed repeatedly with 70% EtOH. After a final wash, the crude sample was dried under vacuum in a speedvac and redissolved in water for purification.

Crude RNA strands were purified either by IEX-HPLC or by IP-RP HPLC.

IEX was carried out with a preparative DNAPac PA200 (ThermoFisher), 22×250 mm column, or PL-SAX (Agilent) 22×150 mm 1000 Å column at 75° C. with a flow rate of 15 mL/min and UV detection at 260 nm. Elution was performed with a linear gradient selected based on crude impurity profile, determined by analytical testing using either a DNAPac PA200RS UPLC column or PL-SAX analytical column. Buffer A: 25 mM Tris·HCl, pH 8.0, 20% acetonitrile, 10 mM sodium perchlorate; buffer B: 25 mM Tris-HCl, pH 8.0, 20% acetonitrile, 600 mM sodium perchlorate, OR, Buffer A: 25 mM Tris-HCl, pH 8.0, 20% acetonitrile, 25 mM sodium chloride; buffer B: 25 mM Tris-HCl, pH 8.0, 20% acetonitrile, 1.5 M sodium chloride.

RP-HPLC was carried out with a BEH C18 300 Å (Waters) 19×150 mm at 60° C., with a flow rate of 25 mL/min and UV detection at 260 nm. Buffer A: TEAA (0.1 M, pH=7); buffer B: MeCN, OR, Buffer A: HAA (0.1 M, pH 7); buffer B: MeCN.

Fractions containing RNA were assessed for purity by analytical PAGE, IEX and RP-HPLC, then pooled and subject to final QC on PAGE, IEX and RP-HPLC, acetonitrile removed in vacuo. The purified oligos were then desalted with Gel-Pak desalting columns (Glen). The solution was lyophilized, and the RNA dissolved in nuclease-free water for concentration determination by UV absorbance and quality assessment via denaturing PAGE.

### Adaptation to Synthesis Procedure for Modified Strands

5′ amino—10% DEA solution in MeCN was applied onto the oligonucleotide while still on CPG. After 5 min treatment the column was rinsed with MeCN and processed further.

5′ Cy3—MMTr group at 5′-end of Cy3 containing sequences was removed during RPC MMT-ON purification.

5′ cholesterol modification—10% DEA solution in MeCN was applied onto the oligonucleotide while still on CPG. After 5 min treatment the column was rinsed with MeCN, and the protecting group (DMT) was removed while still on solid support prior to cleavage and deprotection steps.

### Example 2—Conjugation of Oligonucleotides

To install the appropriate reactive groups to enable conjugation chemistry, 5′ amino modified RNA strands were treated with heterobifunctional NHS-linkers containing the same.

### General Coupling Procedure

The amino-modified oligonucleotide was prepared as a stock solution or dry aliquot. The heterobifunctional NHS-ester (NHS-SM) was dissolved at a concentration of 100 mM in anhydrous DMSO.

Amino-modified oligonucleotide was diluted to a final concentration of 100-200 μM, followed by the addition of DMSO (50% total volume), bicarbonate buffer (0.5 M, pH=8.4, 20% total volume) and NHS-SM (5-20 eq). The reaction mixture was agitated at 30° C. for 1-3 h and was then purified by RP-HPLC. With higher volumes, EtOH precipitation and resuspension in H₂O is recommended.

### Modified Coupling Procedure for Tetrazine NHS

The amino-modified oligonucleotide was prepared as a stock solution or dry aliquot. The heterobifunctional NHS-ester (NHS-SM) was dissolved at a concentration of 100 mM in anhydrous DMF.

Amino-modified oligonucleotide was diluted to a final concentration of 200-500 PM, followed by the addition of DMF (35% total volume), sodium chloride/bicarbonate buffer (100 mM NaCl, 0.05 M, pH=8.4, 30% total volume) and NHS-Tetrazine (5-20 eq). The reaction mixture was agitated at 30° C. for 1 h and was then purified by EtOH precipitation and resuspension in H₂O.

### Example of IEDDA Click Procedure

Norbornene modified core strand (5 nmol, 1.0 eq, 1400 M final concentration) was mixed with siRNA functionalized via tetrazine-NHS (15 nmol, 1.6 eq) in PBS buffer. The reaction mixture was agitated at RT for 12 h, followed by purification with IEX chromatography, using DNAPac PA100 22×250 mm column at 75° C., at a flow rate of 25 mL/min. 40% to 60% B in 30 min (A: 0.1 M NaCl pH 7, B: 1.0 M NaCl), fractions containing product were concentrated and desalted, resulting in 44% isolated yield.

### Example 3—Construct and Barcode Sequences Used in this Disclosure

| Construct | Construct | | |
|---|---|---|---|
| name | type | Component sequences | Notes |
| TW-1 | Three-way | 1) dGdGdGdAdAdAdCdG | |
| | junction | dAdCdTdAdAdCdCdA | |
| | | dCdTdCdCdAdCdTdAd | |
| | | CdAdCdTdGdC | |
| | | 2) dGdGdGdAdAdAdGdC | |
| | | dAdGdTdGdTdAdGdT | |
| | | dGdGdTdGdGdAdCdG | |
| | | dAdGdTdTdCdG | |
| | | 3) dGdGdGdAdAdAdCdG | |
| | | dAdAdCdTdCdGdTdC | |
| | | dCdNdWdNdHdTdGdA | |
| | | dTdAdTdTdGdNdWdN | |
| | | dHdGdTdGdGdTdTdA | |
| | | dGdTdCdG | |
| TW-2 | Three-way | 1) dGdGdGdAdAdAdCdG | |
| | junction | dAdCdTdAdAdCdCdA | |
| | | dCdTdCdCdAdCdTdAd | |
| | | CdAdCdTdGdC | |
| | | 2) dGdGdGdAdAdAdGdC | |
| | | dAdGdTdGdTdAdGdT | |
| | | dGdGdTdGdGdAdCdG | |
| | | dAdGdTdTdCdG | |
| | | 3) dGdGdGdAdAdAdCdG | |
| | | dAdAdCdTdCdGdTdC | |
| | | dCdTdGdAdTdAdTdTd | |
| | | GdGdTdGdGdTdTdAd | |
| | | GdTdCdG | |
| TW-4 | Three-way | 1) dGdGdGdAdAdAdCdT | |
| | junction | dGdCdTdCdAdAdCdT | |
| | | dAdAdCdCdAdCdTdC | |
| | | dCdAdCdTdAdCdAdC | |
| | | dTdCdAdTdCdTdC | |
| | | 2) dGdGdGdAdAdAdGdA | |
| | | dGdAdTdGdAdGdTdG | |
| | | dTdAdGdTdGdGdTdG | |
| | | dGdAdCdGdAdGdTdT | |
| | | dCdGdTdAdTdGdAdG | |
| | | 3) dGdAdTdGdCdTdCdTd | |
| | | CdAdTdAdCdGdAdAd | |
| | | CdTdCdGdTdCdCdTd | |
| | | GdAdTdAdTdTdGdGd | |
| | | TdGdGdTdTdAdGdTd | |
| | | TdGdAdGdCdAdG | |
| TW-9 | Three-way | 1) 5′ | Lower case indicates 2′Fluoro |
| | junction | /Norbornene/rG*rG*rG | u/c, * phosphorothioate, r |
| | | *rA*rA*rA*c*rG*rA*c | indicates RNA A/G |
| | | *u*rA*A*c*c*rA*c*u | (unmodified RNA) |
| | | *c*c*rA*c*u*rA*c*rA | 2′ F C/A/U indicated with |
| | | *c*u*rG*c | lowercase, 2′ O me A/U/G/C |
| | | 2) 5′ Tetrazine-PEG5- | indicated with ′m′, PTO |
| | | CONH- | indicated with * |
| | | c*mA*amAumUcmCa | Strand (1) is conjugated to (2) |
| | | mUcmGu*mG*a | via IEDDA (3) is conjugated to |
| | | 3) 5′ | another equivalent of (1) via |
| | | /Norbornene/rG*rG*rG | IEDDA |
| | | *rA*rA*rA*rG*c*rA*r | Barcode incorporated within |
| | | G*u*rG*u*rA*rG*u*r | strand 4 |
| | | G*rG*u*rG*rG*A*c*r | 2 x PPIB siRNA used as a |
| | | G*rA*G*u*u*c*rG [3′ | therapeutic |
| | | Cy3] | |
| | | 4) c*mG*a*mAcmUcmGu | |
| | | mCcmUcmCumGcmGc | |
| | | mGumGgmUumAg*m | |
| | | U*c*mG | |
| | | 5) mU*c*mAcmGamUgm | |
| | | GamAumU*u*mG*c* | |
| | | mU*g*mU*u | |
| TW-10 | Three-way | 1) 5′ | m indicates 2′OMe G/A/U/C |
| | junction | /Norbornene/mGmGmG | 2′ F C/A/U indicated with |
| | | mAmAmAmCmGmAm | lowercase, 2′ O me A/U/G/C |
| | | CmUmAmAmCmCmA | indicated with ′m′, PTO |
| | | mCmUmCmCmAmCm | indicated with * |
| | | UmAmCmAmCmUmG | Barcode incorporated within |
| | | mC | strand 4 |
| | | 2) 5′ Tetrazine-PEG5- | 2 x PPIB siRNA used as a |
| | | CONH- | therapeutic |
| | | c*mA*amAumUcmCa | |
| | | mUcmGu*mG*a | |
| | | 3) 5′ | |
| | | /Norbornene/mGmGmG | |
| | | mAmAmAmGmCmAm | |
| | | GmUmGmUmAmGmU | |
| | | mGmGmUmGmGmAm | |
| | | CmGmAmGmUmUmC | |
| | | mG [3′ Cy3] | |
| | | 4) c*mG*a*mAcmUcmGu | |
| | | mCcmCumGamGcmCa | |
| | | mGumGgmUumAg*m | |
| | | U*c*mG | |
| | | 5) mU*c*mAcmGamUgm | |
| | | GamAumU*u*mG*c* | |
| | | mU*g*mU*u | |
| TW-11 | Three-way | 1) 5′ | Lower case indicates 2′Fluoro |
| | junction | /Norbornene/mGmGmG | u/c, * phosphorothioate, r |
| | | mAmAmAmCmGmAm | indicates RNA A/G |
| | | CmUmAmAmCmCmA | (unmodified RNA) |
| | | mCmUmCmCmAmCm | 2′ F C/A/U indicated with |
| | | UmAmCmAmCmUmG | lowercase, 2′ O me A/U/G/C |
| | | mC | indicated with ′m′, PTO |
| | | 2) 5′ Tetrazine-PEG5- | indicated with * |
| | | CONH- | Strand (1) is conjugated to (2) |
| | | c*mA*amAumUcmCa | via IEDDA (3) is conjugated to |
| | | mUcmGu*mG*a | another equivalent of (1) via |
| | | 3) 5′ | IEDDA |
| | | /Norbornene/mGmGmG | Barcode incorporated within |
| | | mAmAmAmGmCmAm | strand 4 |
| | | GmUmGmUmAmGmU | 2 x PPIB siRNA used as a |
| | | mGmGmUmGmGmAm | therapeutic |
| | | CmGmAmGmUmUmC | |
| | | mG [3′ Cy3] | |
| | | 4) g*mC*g*mAamCgmCc | |
| | | cmGamAcmUcmGumC | |
| | | cmAamGumGgmUum | |
| | | Ag*mU*c*mG | |
| | | 5) mU*c*mAcmGamUgm | |
| | | GamAumU*u*mG*c* | |
| | | mU*g*mU*u | |
| TW-12 | Three-way | 1) 5′ | Lower case indicates 2′Fluoro |
| | junction | /Norbornene/rG*rG*rG | u/c, * phosphorothioate, r |
| | | *rA*rA*rA*c*G*rA*c | indicates RNA A/G |
| | | *u*rA*rA*c*c*rA*c*u | (unmodified RNA) |
| | | *c*c*rA*c*u*rA*c*rA | 2′ F C/A/U indicated with |
| | | *c*u*rG*c | lowercase, 2′ O me A/U/G/C |
| | | 2) 5′ Tetrazine-PEG5- | indicated with ′m′, PTO |
| | | CONH- | indicated with * |
| | | c*mA*amAumUcmCa | Strand (1) is conjugated to (2) |
| | | mUcmGu*mG*a | via IEDDA (3) is conjugated to |
| | | 3) 5′ | another equivalent of (1) via |
| | | /Norbornene/rG*G*rG | IEDDA |
| | | *A*rA*rA*rG*c*rA*r | Barcode incorporated within |
| | | G*u*rG*u*rA*rG*u*r | strand 4 |
| | | G*rG*u*rG*rG*rA*c*r | 2 x PPIB siRNA used as a |
| | | G*rA*rG*u*u*c*rG [3′ | therapeutic |
| | | Cy3] | |
| | | 4) g*mC*g*mAamCgmCc | |
| | | cmGamAcmUcmGumC | |
| | | cmAamGumGgmUum | |
| | | Ag*mU*c*mG | |
| | | 5) mU*c*mAcmGamUgm | |
| | | GamAumU*u*mG*c* | |
| | | mU*g*mU*u | |
| TW-13 | Three-way | 1) 5′ | Lower case indicates 2′Fluoro |
| | junction | /Norbornene/mGmGmG | u/c, * phosphorothioate, r |
| | | mAmAmAmCmGmAm | indicates RNA A/G |
| | | CmUmAmAmCmCmA | (unmodified RNA) |
| | | mCmUmCmCmAmCm | 2′ F C/A/U indicated with |
| | | UmAmCmAmCmUmG | lowercase, 2′ O me A/U/G/C |
| | | mC | indicated with ′m′, PTO |
| | | 2) 5′ Tetrazine-PEG5- | indicated with * |
| | | CONH- | Strand (1) is conjugated to (2) |
| | | c*mA*amAumUcmCa | via IEDDA (3) is conjugated to |
| | | mUcmGu*mG*a | another equivalent of (1) via |
| | | 3) 5′ | IEDDA |
| | | /Norbornene/mGmGmG | Barcode incorporated within |
| | | mAmAmAmGmCmAm | strand 4 |
| | | GmUmGmUmAmGmU | 2 x PPIB siRNA used as a |
| | | mGmGmUmGmGmAm | therapeutic |
| | | CmGmAmGmUmUmC | |
| | | mG [3′ Cy3] | |
| | | 4) g*mC*g*mAamCgmCc | |
| | | cmGamAcmUcmGumC | |
| | | cmAamGumGgmUum | |
| | | Ag*mU*c*mG | |
| | | 5) 5′Cy5- | |
| | | mU*c*mAcmGamUgm | |
| | | GamAumU*u*mG*c* | |
| | | mU*g*mU*u | |
| TW-14 | Three-way | 1) 5′ | Lower case indicates 2′Fluoro |
| | junction | /Norbornene/rG*rG*rG | u/c, * phosphorothioate, r |
| | | *rA*rA*rA*c*rG*rA*c | indicates RNA A/G |
| | | *u*rA*rA*c*c*rA*c*u | (unmodified RNA) |
| | | *c*c*rA*c*u*rA*c*rA | 2′ F C/A/U indicated with |
| | | *c*u*rG*c | lowercase, 2′ O me A/U/G/C |
| | | 2) 5′ Tetrazine-PEG5- | indicated with ′m′, PTO |
| | | CONH- | indicated with * |
| | | c*mA*amAumUcmCa | Strand (1) is conjugated to (2) |
| | | mUcmGu*mG*a | via IEDDA (3) is conjugated to |
| | | 3) 5′ | another equivalent of (1) via |
| | | /Norbornene/rG*G*rG | IEDDA |
| | | *rA*rA*rA*rG*c*rA*r | Barcode incorporated within |
| | | G*u*rG*u*rA*rG*u*r | strand 4 |
| | | G*rG*u*G*rG*rA*c*r | 2 x PPIB siRNA used as a |
| | | G*rA*rG*u*u*c*rG [3′ | therapeutic |
| | | Cy3] | |
| | | 4) g*mC*g*mAamCgmCc | |
| | | cmGamAcmUcmGumC | |
| | | cmAamGumGgmUum | |
| | | Ag*mU*c*mG | |
| | | 5) 5′Cy5- | |
| | | mU*c*mAcmGamUgm | |
| | | GamAumU*u*mG*c* | |
| | | mU*g*mU*u | |
| TW-17 | Three-way | 1) rGrCrGrArArCrGrCrCr | r indicates 2′ OH G/A/U/C |
| | junction | CrGrUrArArUrArArGr | |
| | | GrCrUrArGrUrCrCrGr | |
| | | UrUrArUrCrArArCrUr | |
| | | UrG | |
| | | 2) rGrGrGrArArArCrArAr | |
| | | GrUrUrGrArUrArArCr | |
| | | GrUrCrCrArCrUrArCr | |
| | | ArCrUrGrC | |
| | | 3) rGrGrGrArArArGrCrAr | |
| | | GrUrGrUrArGrUrGrGr | |
| | | UrUrArGrCrCrUrUrAr | |
| | | UrUrArCrG | |
| TW-18 | Three-way | 1) rGrCrGrArArCrGrCrCr | r indicates 2′ OH G/A/U/C |
| | | CrGrUrArArUrArArGr | |
| | junction | GrCrUrArGrUrCrCrGr | |
| | | UrUrArUrCrArArCrUr | |
| | | UrG | |
| | | 2) rGrGrGrArArArCrArAr | |
| | | GrUrUrGrArUrArArCr | |
| | | GrUrCrCrArCrUrArCr | |
| | | ArCrUrGrC | |
| | | 3) rGrGrGrArArArGrCrAr | |
| | | GrUrGrUrArGrUrGrGr | |
| | | UrUrArGrCrCrUrUrAr | |
| | | UrUrArCrG | |
| TW-25 | Three-way | 1) rGrCrGrArArCrGrCrCr | r indicates 2′ OH G/A/U/C |
| | junction | GrUrArArCrUrCrGrUr | m indicates 2′OMe G/A/U/C |
| | | CrCrArArGrUrGrGrUr | |
| | | UrArGrUrCrGrArAUrC | |
| | | rArC | |
| | | 2) mGmGmGmAmAmA | |
| | | mGmUmG mAmUmU | |
| | | mAmUmUmAmUmAm | |
| | | AmCmCmAmCmUmC | |
| | | mCmAmCmUmAmCm | |
| | | AmCmUmGmC | |
| | | 3) mGmGmGmAmAmAm | |
| | | GmCmAmGmUmGmU | |
| | | mAmGmUmGmGmUm | |
| | | GmGmAmCmGmAmG | |
| | | mUmUmAmC | |
| TW-26 | Three-way | 1) g*mC*g*mAamC | Lower case indicates 2′Fluoro |
| | junction | gmCc mGumA amCu | u/c/a/g, m indicates 2′OMe |
| | | mCgmU cmCa mAgmU | G/A/U/C, * phosphorothioate |
| | | gmGu mUamG umCg | |
| | | mA*a*mU *c | |
| | | 2) mGmGmGmAmAmAm | |
| | | GmAmUmUmCmGmA | |
| | | mCmUmAmAmCmCm | |
| | | UmGmUmGmCmAmC | |
| | | mUmAmCmAmCmUm | |
| | | GmC | |
| | | 3) mGmGmGmAmAmAm | |
| | | GmCmAmGmUmGmU | |
| | | mAmGmUmGmGmUm | |
| | | GmGmAmCmGmAmG | |
| | | mUmUmAmC | |
| TW-27 | Three-way | 1) g*mC*g*mAamCgmCc | Lower case indicates 2′Fluoro |
| | junction | mGumAamCumCgmUc | u/c/a/g, m indicates 2′OMe |
| | | mCamAgmUgmGumUa | G/A/U/C, * phosphorothioate |
| | | mGumCgmAamUc*mG | |
| | | *a*mG | |
| | | 2) mGmGmGmAmAmAm | |
| | | CmUmCmGmAmUmU | |
| | | mCmGmAmCmGmUm | |
| | | UmAmCmGmUmGmC | |
| | | mUmGmA | |
| | | 3) mGmGmGmAmAmA | |
| | | mUmCmA mGmCmA | |
| | | mCmGmU mAmA mC | |
| | | mGmGmAmCmGmAm | |
| | | GmUmUmAmC | |
| TW-28 | Three-way | 1) g*mC*g*mAamCgmCc | Lower case indicates 2′Fluoro |
| | junction | mGumAamCumCgmUc | u/c/a/g, m indicates 2′OMe |
| | | mCamAgmUgmGumUa | G/A/U/C, * phosphorothioate |
| | | mGumCg*mA*a*mU | |
| | | 2) mCmGmUmCmGmAm | |
| | | CmUmAmAmCmCmA | |
| | | mC | |
| | | 3) mGmGmGmAmAmA | |
| | | mGmGmAmCmGmAm | |
| | | GmUmUmAmC | |
| TW-38 | Three-way | 1) g*mA*c*mUamUcmUc | Lower case indicates 2′Fluoro |
| | junction | mGumAamCumCgmUc | u/c/a/g, m indicates 2′OMe |
| | | mCamAgmUgmGumUa | G/A/U/C, * phosphorothioate |
| | | mGumCgmAamU*c*m | |
| | | A*c[3AmMO] | |
| | | 2) mGmGmGmAmAmA | |
| | | mGmAmUmUmCmGm | |
| | | AmCmUmAmAmCmC | |
| | | mUmGmUmGmCmAm | |
| | | CmUmAmCmAmCmU | |
| | | mGmC | |
| | | 3) mGmGmGmAmAmAm | |
| | | GmCmAmGmUmGmU | |
| | | mAmGmUmGmGmUm | |
| | | GmGmAmCmGmAmG | |
| | | mUmUmAmC | |
| TW-41 | Three-way | 1) g*mU*a*mUamUcmUa | Lower case indicates 2′Fluoro |
| | junction | mGumAamCumCgmUc | u/c/a/g, m indicates 2′OMe |
| | | mCamAgmUgmGumUa | G/A/U/C, * phosphorothioate |
| | | mGumCgmAamU*c*m | |
| | | A*c[3AmMO] | |
| | | 2) mCmGmUmCmGmAm | |
| | | CmUmAmAmCmCmA | |
| | | mC | |
| | | 3) mGmGmGmAmAmA | |
| | | mGmGmAmCmGmAm | |
| | | GmUmUmAmC | |
| TW-49 | Three-way | 1) g*mC*g*mAamCgmCc | Lower case indicates 2′Fluoro |
| | junction | mGumAamCumCgmUc | u/c/a/g, m indicates 2′OMe |
| | | mCamAgmUgmGumUa | G/A/U/C, * phosphorothioate |
| | | mGumCgmAamU*c*m | |
| | | A*c | |
| | | 2) mGmGmGmAmAmAm | |
| | | GmAmUmUmCmGmA | |
| | | mCmUmAmAmCmCm | |
| | | UmGmUmGmCmAmC | |
| | | mUmAmCmAmCmUm | |
| | | GmC | |
| | | 3) mGmGmGmAmAmAm | |
| | | GmCmAmGmUmGmU | |
| | | mAmGmUmGmGmUm | |
| | | GmGmAmCmGmAmG | |
| | | mUmUmAmC | |
| TW-50 | Three-way | 1) g*mA*a*mUgmCumAc | Lower case indicates 2′Fluoro |
| | junction | mGumAamCumCgmUc | u/c/a/g, m indicates 2′OMc |
| | | mCamAgmUgmGumUa | G/A/U/C, * phosphorothioate |
| | | mGumCgmAamU*c*m | |
| | | A*c [3AmMO] | |
| | | 2) mGmGmGmAmAmAm | |
| | | CmUmCmGmAmUmU | |
| | | mCmGmAmCmGmUm | |
| | | UmAmCmGmUmGmC | |
| | | mUmGmA | |
| | | 3) mGmGmGmAmAmAm | |
| | | UmCmAmGmCmAmC | |
| | | mGmUmAmAmCmGm | |
| | | GmAmCmGmAmGmU | |
| | | mUmAmC | |
| MD-1 | Duplex | 1) 5′ | Lower case indicates 2′Fluoro |
| | | /Norbornene/G*G*G*A | u/c, * phosphorothioate, upper |
| | | *A*A*G*c*A*G*u*G* | case indicates RNA A/G |
| | | u*A*G*c*G*G*A*c*G | (unmodified) |
| | | *G*u*G*u*G [3′ Cy3] | Barcode embedded within |
| | | 2) g*mU*c*mUamAcmUg | strand (2) |
| | | mCamCamCcmGumCc | |
| | | mGcmUamCamC*u*m | |
| | | G*c | |
| MD-2 | Duplex | 1) 5′ | m indicates 2′OMe G/A/U/C |
| | | /Norbornene/mGmGmG | lower case indicates 2′Fluoro |
| | | mAmAmAmGmCmAm | u/c/a/g, m indicates 2′OMe |
| | | GmUmGmUmAmGmC | G/A/U/C, * phosphorothioate |
| | | mGmGmAmCmGmGm | Barcode embedded within |
| | | UmGmUmG [3′ Cy3] | strand (2) |
| | | 2) g*mU*c*mUamAcmUg | |
| | | mCamCamCcmGumCc | |
| | | mGcmUamCamC*u*m | |
| | | G*c | |
| MD-3 | Duplex | 1) 5′ | Lower case indicates 2′Fluoro |
| | | /Norbornene/G*G*G*A | u/c, * phosphorothioate, upper |
| | | *A*A*G*c*A*G*u*G* | case indicates RNA A/G |
| | | u*A*G*c*G*G*A*c*G | (unmodified) |
| | | *G*u*G*u*G [3′ Cy3] | Barcode embedded within |
| | | 2) 5′ Tetrazine-PEG5- | strand (3) |
| | | CONH- | Includes PPIB siRNA as a |
| | | c*mA*amAumUcmCa | therapeutic |
| | | mUcmGu*mG*a | |
| | | 3) g*mU*c*mUamAcmUg | |
| | | mCamCamCcmGumCc | |
| | | mGcmUamCamC*u*m | |
| | | G*c | |
| | | 4) mU*c*mAcmGamUgm | |
| | | GamAumU*u*mG*c* | |
| | | mU*g*mU*u | |
| MD-4 | Duplex | 1) 5′ | m indicates 2′OMe G/A/U/C |
| | | /Norbornene/mGmGmG | lower case indicates 2′Fluoro |
| | | mAmAmAmGmCmAm | u/c/a/g, m indicates 2′OMe |
| | | GmUmGmUmAmGmC | G/A/U/C, * phosphorothioate |
| | | mGmGmAmCmGmGm | Barcode embedded within |
| | | UmGmUmG [3′ Cy3] | strand (3) |
| | | 2) 5′ Tetrazine-PEG5- | Includes PPIB siRNA as a |
| | | CONH- | therapeutic |
| | | c*mA*amAumUcmCa | |
| | | mUcmGu*mG*a | |
| | | 3) g*mU*c*mUamAcmUg | |
| | | mCamCamCcmGumCc | |
| | | mGcmUamCamC*u*m | |
| | | G*c | |
| | | 4) mU*c*mAcmGamUgm | |
| | | GamAumU*u*mG*c* | |
| | | mU*g*mU*u | |
| MD-11 | Duplex | 1) 5′ | m indicates 2′OMe G/A/U/C |
| | | /Norbornene/G*G*G*A | lower case indicates 2′Fluoro |
| | | *A*A*G*c*A*G*u*G* | u/c/a/g, m indicates 2′OMe |
| | | u*A*G*c*G*G*A*c*G | G/A/U/C, * phosphorothioate |
| | | *G*u*G*u*G [3′ Cy3] | Barcode embedded within |
| | | 2) 5′ Tetrazine-PEG5- | strand (3) |
| | | CONH- | Includes PPIB siRNA as a |
| | | c*mA*amAumUcmCa | therapeutic |
| | | mUcmGu*mG*a | |
| | | 3) g*mU*c*mUamAcmUg | |
| | | mCamCamCcmGumCc | |
| | | mGcmUamCamC*u*m | |
| | | G*c | |
| | | 4) 5′Cy5- | |
| | | mU*c*mAcmGamUgm | |
| | | GamAumU*u*mG*c* | |
| | | mU*g*mU*u | |
| MD-12 | Duplex | 1) 5′ | m indicates 2′OMe G/A/U/C |
| | | /Norbornene/mGmGmG | lower case indicates 2′Fluoro |
| | | mAmAmAmGmCmAm | u/c/a/g, m indicates 2′OMe |
| | | GmUmGmUmAmGmC | G/A/U/C, * phosphorothioate |
| | | mGmGmAmCmGmGm | Barcode embedded within |
| | | UmGmUmG [3′ Cy3] | strand (3) |
| | | 2) 5′ Tetrazine-PEG5- | Includes PPIB siRNA as a |
| | | CONH- | therapeutic |
| | | c*mA*amAumUcmCa | |
| | | mUcmGu*mG*a | |
| | | 3) g*mU*c*mUamAcmUg | |
| | | mCamCamCcmGumCc | |
| | | mGcmUamCamC*u*m | |
| | | G*c | |
| | | 4) 5′Cy5- | |
| | | mU*c*mAcmGamUgm | |
| | | GamAumU*u*mG*c* | |
| | | mU*g*mU*u | |
| MD-16 | Duplex | 1) 5′ | lower case indicates 2′Fluoro |
| | | /Norbornene/G*G*G*A | u/c, * phosphorothioate, upper |
| | | *A*A*G*c*A*G*u*G* | case indicates RNA A/G |
| | | u*A*G*c*G*G*A*c*G | (unmodified) |
| | | *G*u*G*u*G [3′ Cy3] | Barcode embedded within |
| | | 2) 5′ Tetrazine-PEG5- | strand (3) |
| | | CONH- | Includes PPIB siRNA as a |
| | | c*mA*amAumUcmCa | therapeutic |
| | | mUcmGu*mG*a | |
| | | 3) g*mU*c*mUamAcmUg | |
| | | mCamCamCcmGumCc | |
| | | mGcmUamCamC*u*m | |
| | | G*c | |
| | | 4) /5BiotinTEG/T*C* | |
| | | ACG ATG GAA TTT | |
| | | GCT G*T*T | |
| MD-17 | Duplex | 1) 5′ | lower case indicates 2′Fluoro |
| | | /Norbornene/mGmGmG | u/c, * phosphorothioate, upper |
| | | mAmAmAmGmCmAm | case indicates RNA A/G |
| | | GmUmGmUmAmGmC | (unmodified) |
| | | mGmGmAmCmGmGm | Barcode embedded within |
| | | UmGmUmG [3′ Cy3] | strand (3) |
| | | 2) 5′ Tetrazine-PEG5- | Includes PPIB siRNA as a |
| | | CONH- | therapeutic |
| | | c*mA*amAumUcmCa | |
| | | mUcmGu*mG*a | |
| | | 3) g*mU*c*mUamAcmUg | |
| | | mCamCamCcmGumCc | |
| | | mGcmUamCamC*u*m | |
| | | G*c | |
| | | 4) /5BiotinTEG/T*C* | |
| | | ACG ATG GAA TTT | |
| | | GCT G*T*T | |
| MD-19 | Duplex | 1) rGrGrGrArArA | |
| | | rCrArArGrUrUrGrArUr | |
| | | ArArCrGrGrArCrUrAr | |
| | | GrCrCrUrUrArUrUrAr | |
| | | CrG | |
| | | 2) rGrCrGrArArCrGrCrC | |
| | | rCrGrU | |
| | | rArArUrArArGrGrCrUr | |
| | | ArGrUrCrCrGrUrUrAr | |
| | | UrCrArArCrUrUrG | |
| MD-21 | Duplex | 1) rGrGrGrArArA | |
| | | rCrArArGrGrArUrArAr | |
| | | CrGrGrArCrUrArGrCr | |
| | | CrUrUrArUrUrArCrG | |
| | | 2) rGrCrGrArArCrGrCrC | |
| | | rCrGrU | |
| | | rArArUrArArGrGrCrUr | |
| | | ArGrUrCrCrGrUrUrAr | |
| | | UrCrArArCrUrUrG | |
| MD-22 | Duplex | 1) rGrGrGrArArA | |
| | | rCrArArGrUrUrGrArUr | |
| | | ArGrGrArCrUrArGrCr | |
| | | CrUrUrArUrUrArCrG | |
| | | 2) rGrCrGrArArCrGrCrC | |
| | | rCrGrU | |
| | | rArArUrArArGrGrCrUr | |
| | | ArGrUrCrCrGrUrUrAr | |
| | | UrCrArArCrUrUrG | |
| MD-23 | Duplex | 1) mGmGmGmAmAmAm | Lower case indicates 2′Fluoro |
| | | GmUmGmUmAmAmU | u/c/a/g, m indicates 2′OMe |
| | | mUmGmAmUmCmAm | G/A/U/C, * phosphorothioate |
| | | AmUmCmUmGmUmU | |
| | | mUmGmGmAmUmAm | |
| | | AmAmGmUmUmGmU | |
| | | 2) g*mA*u*mAamUamUa | |
| | | mGumAamCumCgmUc | |
| | | mCamAgmUgmGumUa | |
| | | mGumCgmAamU*c*m | |
| | | A*c[3AmMO] | |

Oligonucleotides may be modified with a propargyl unit via the 2′O of the ribose sugar at any given position.

### Example 4—Barcode Methodology

### General Sequences for Barcode Detection and Amplification

Primer, probe and target sequences used in the examples below are summarized in Table 1.

TABLE 1
List of primer, probe and target sequences. Modifications are indicated in IDT
notation: r indicates 2′ OH nucleotides (RNA), m indicates 2′OMe, /i2F/ indicates
an internal 2′F modification.

| SEQ ID | | | |
|---|---|---|---|
| NO | EXAMPLE | DESCRIPTION | SEQUENCE (5′→3′) |
| 1 | 3 | Unmodified RNA | rGrGrG rArArA rGrCrA rGrUrG rUrArG |
| | | template | rCrGrG rArCrG rGrUrG rUrGrU rCrArG |
| | | | rUrUrC rArArC rCrCrA rCrGrA rCrArG rArG |
| 2 | 3 | Modified RNA | mGmGmG mAmAmA mG/i2FC/mA |
| | | template | mG/i2FU/mG/i2FU/mAmG/i2FC/mGmG |
| | | | mA/i2FC/mG mG/i2FU/mG/i2FU/mG/i2FU/ |
| | | | /i2FC/mAmG/i2FU//i2FU//i2FC/ |
| | | | mAmA/i2FC//i2FC//i2FC/mA/i2FC/mGmA |
| | | | /i2FC/mAmG mAmG |
| 3 | 3 | RT primer & | CTCTGTCGTGGGTTGAACTGA |
| | | qPCR primer | |
| | | (rev) | |
| 4 | 3 | qPCR primer | GGGAAAGCAGTGTAGCGGA |
| | | (fwd) | |
| 5 | 3 | Positive control | CTCTGTCGTGGGTTGAACTGACACACCG |
| | | | TCCGCTACACTGCTTTCCC |
| 6 | 4 | RT primer & | GCTCTCATACGAACTCGTCC |
| | | qPCR primer | |
| | | (rev) | |
| 7 | 4 | qPCR primer | GTCTCTGCTCGACTAACCAC |
| | | (fwd) | |

### Modified RNA Barcode can be Read by Reverse Transcriptase

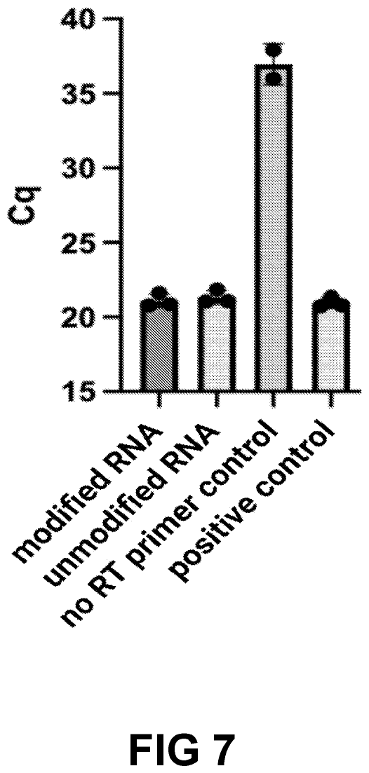

To compare the reverse transcription efficiency of modified versus unmodified RNA templates, a reaction mix comprising 1.25 pM of RNA template (SEQ ID NO 1 or SEQ ID NO 2), 0.1 μM of template-specific primer (SEQ ID NO 3), 0.5 mM dNTP mix, 1× First-Strand Buffer, 5 μM DTT and 200 units of Superscript III reverse transcriptase (18080093, Invitrogen, Thermo Fisher Scientific) was subjected to reverse transcription by incubation at 55° C. for 45 minutes followed by heat inactivation at 70° C. for 15 minutes. The so-generated cDNA was quantified by real-time PCR on a Quantstudio 5 thermal cycler (Applied Biosystems, Thermo Fisher Scientific) using PowerUp SYBR Green Master Mix (A25742, Applied Biosystems, Thermo Fisher Scientific). SEQ ID NO 5 was used as a positive control. As a negative control, a RT reaction lacking the template-specific primer was used. The results shown in FIG. 7 validate the concept that chemically modified barcode sequences can be viable templates of Superscript III reverse transcriptase and do not inhibit enzymatic activity.

### Example 5—Modified RNA Barcode can be Detected by PCR

Mouse liver stored in RNAlater (Sigma, R0901) was pierced using 2 mm punches and lysed using a TissueLyser II (Qiagen) according to manufacturer instructions in 300 μL of mirVana lysis buffer. The lysates were then spiked with 5-fold serially diluted RNA nanostructures (M-1 or M-2). Total RNA was extracted using a mirVana kit (AM1560, Invitrogen, Thermo Fisher Scientific), reverse transcribed with target-specific SEQ ID 6 reverse transcription primer using Superscript III reverse transcriptase (18080093, Invitrogen, Thermo Fisher Scientific) and the cDNA was subjected to real-time PCR on a Quantstudio 5 thermal cycler (Applied Biosystems, Thermo Fisher Scientific) using PowerUp SYBR Green Master Mix (A25742, Applied Biosystems, Thermo Fisher Scientific).

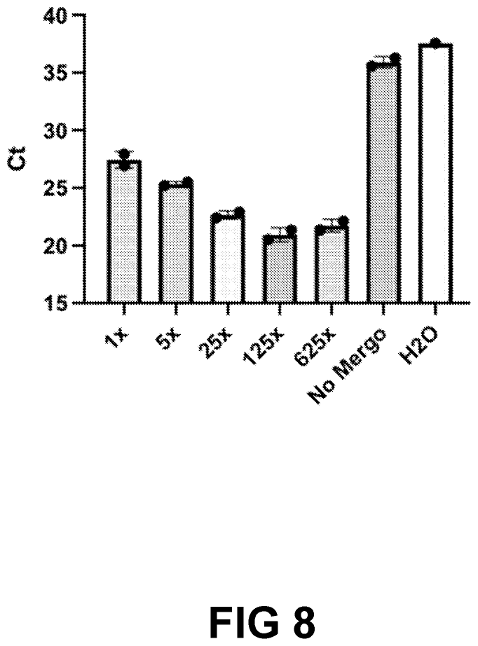

As shown in FIG. 8, the nucleic acid barcodes were detected in a concentration-dependent manner over three logs. The results indicate that a modified barcode strand that is flanked by primer binding sequences and hybridized to a nucleic acid nanostructure can be used to quantify tissue delivery of RNA-based delivery systems. Moreover, the data suggest that reverse transcription with Superscript III reverse transcriptase is a viable step during library preparation for RNA sequencing.

### Example 6—Detection of Short Single-Stranded and Double-Stranded Barcodes by Template-Switching Reverse Transcription

To compare the NGS library preparation efficiency of fully modified single-stranded and double-stranded RNA containing a short (8-nt) 5′ barcode, a reaction mix comprising 0.1 μM of RNA template, 1 pM of template-specific primer and 1 mM dNTP mix was heated to 53° C. for 5 minutes. Unmodified single-stranded RNA was used as a control template. Template-switching reverse transcription was performed using Template Switching RT Enzyme Mix (M0466S, New England Biolabs) according to manufacturer's instructions for cDNA synthesis and amplification. The generated cDNA was further amplified by PCR for 18 cycles using NEBNext Q5 Hot Start High-Fidelity 2X Master Mix (M0494, New England Biolabs) and the PCR products were loaded on a denaturing PAGE gel (12%) and visualized using Gelred nucleic acid gel stain.

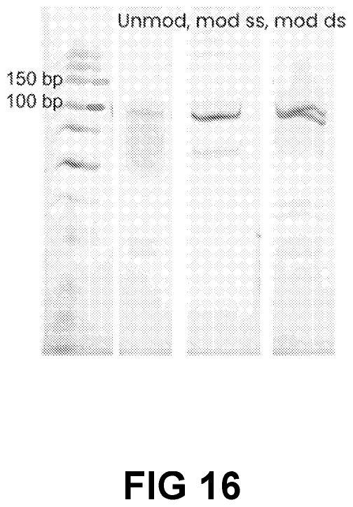

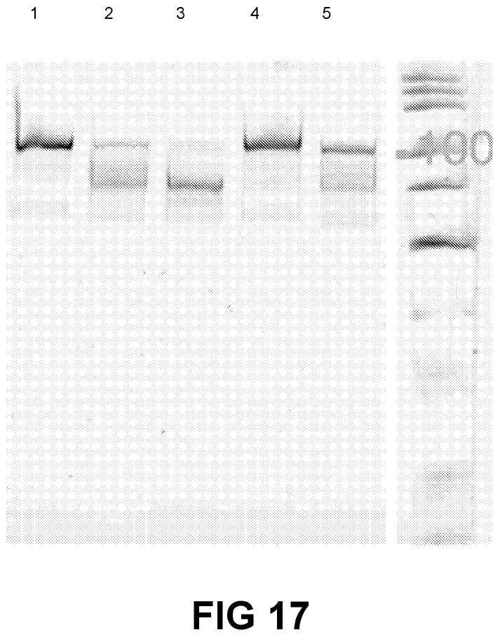

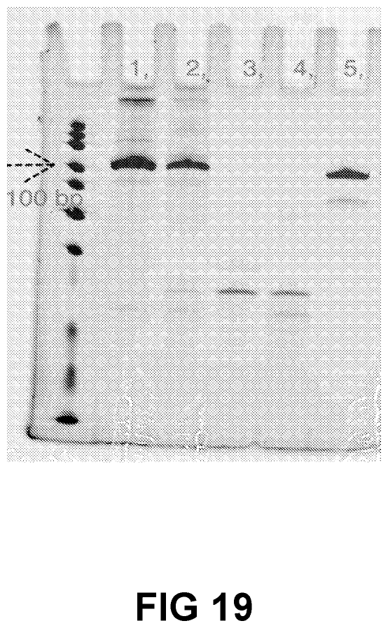

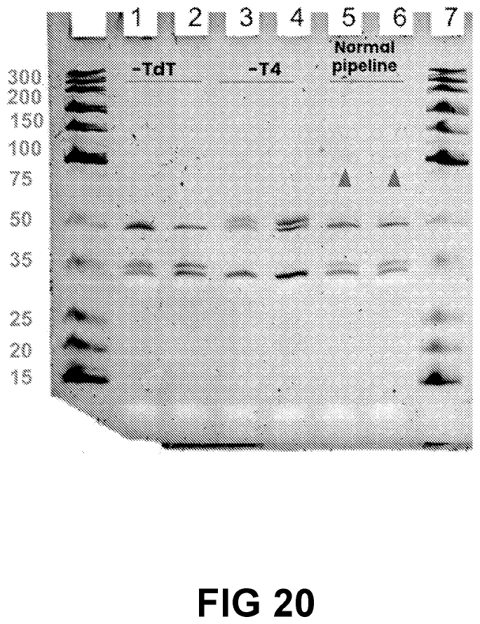

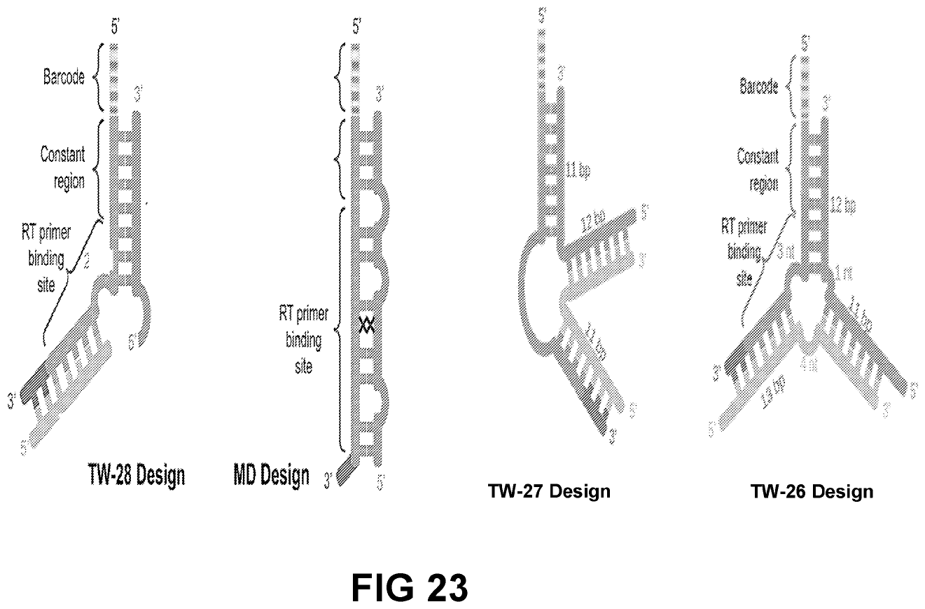

The gel in FIG. 19 confirms the feasibility of detecting fully 2′OMe modified single-stranded RNA barcodes using a library preparation pipeline based on template-switching reverse transcription. As shown in FIG. 16, both fully modified single-stranded and double-stranded barcodes resulted in the expected product band with roughly equal band intensity, indicating similar levels of library preparation efficiency. Moreover, the 2′OMe RNA modifications used protected the barcodes from nuclease-mediated degradation whilst template degradation was prominent in the unmodified RNA control. The results in FIG. 17 suggest that the incorporation of 8-nt barcodes in different 3-way junction constructs, whose designs are exemplified in FIG. 23, does not hamper library preparation. Whilst the choice of the barcode sequence may affect detection efficiency, the effect of construct morphology appeared to be negligible.

### Example 7—Detection of Barcodes by Single-Cell Sequencing

### Detection of Barcodes In Vitro in Single Cells

A549 cells were transfected with Mergo structures containing unique barcodes (designs outlined in FIG. 23) and single-stranded modified RNA oligos, either in singleplex or multiplex (pool of 5 Mergos or 6 ssRNAs) conditions. Each barcode was unique to the Mergo design/shape used. After 24 hours, the cells were trypsinized and processed for single-cell RNA sequencing using the 10× Genomics Chromium platform. Libraries were either treated with CRISPRclean (Jumpcode Genomics) to remove uninformative reads or left untreated (standard protocol), and sequencing was performed at 5K read pairs/cell for targeted Mergo libraries. Sequencing reads were demultiplexed based on UMI and barcode identity using a custom analysis pipeline.

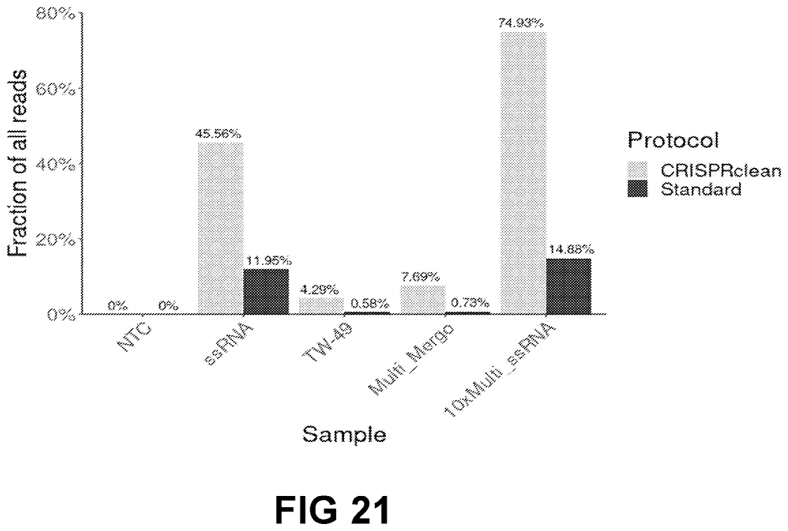

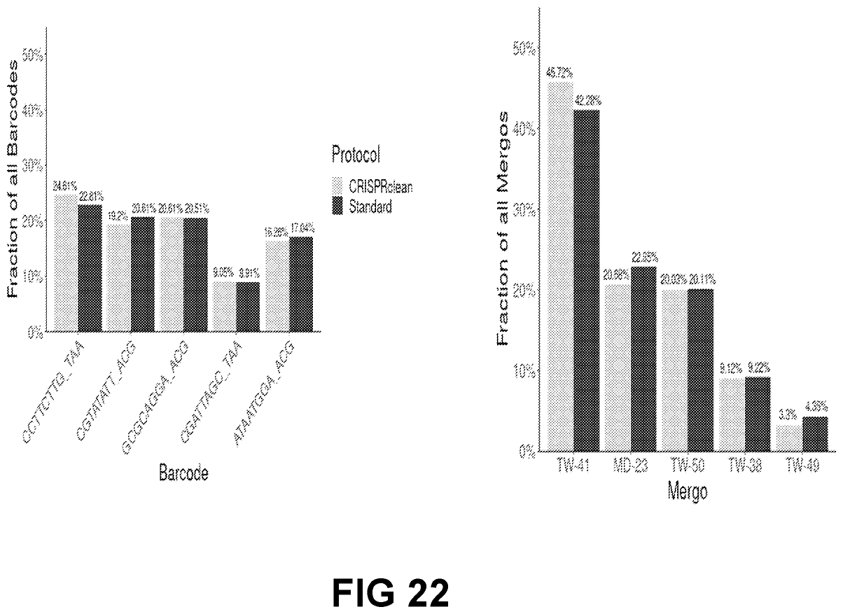

FIG. 21 shows the fraction of reads mapping to Mergo sequences for each sample. No barcode reads were detectable in untransfected control cells (NTC), and the fraction of mapping reads was consistently higher after CRISPRclean treatment. FIG. 22 summarizes the relative abundance of different barcodes within multiplexed samples. Each of six single-stranded barcodes co-transfected at equal concentration was detectable by single-cell sequencing (FIG. 22A), however, certain barcode sequences were more efficiently detected than others. Results from multiplexing five different Mergo constructs (FIG. 22B) imply that Mergo shape can influence barcode detection at the scSEQ level.

### Detection of Barcodes In Vivo in Mouse Tissues at the Single-Cell Level

To obtain tissue-specific data, fresh mouse tissues were derived from mice injected with barcoded Mergo structures carrying siRNA therapeutics (multiplexed pool of two Mergos containing different modifications, with the barcoded strands being either fully modified with phosphorothioate or 2′OMe modifications, respectively). The barcodes were 8 nucleotides in length and positioned at the 5′ terminus of a Mergo core strand. Mice were sacrificed 7 days post-injection and single-cell suspensions were prepared from three organs (liver, lung and heart). Sequencing was performed at 40K read pairs/cell for GEX libraries and 5K read pairs/cell for targeted Mergo libraries. Data were processed using Cellranger and Seurat pipelines. Cell types were annotated using scMCA.

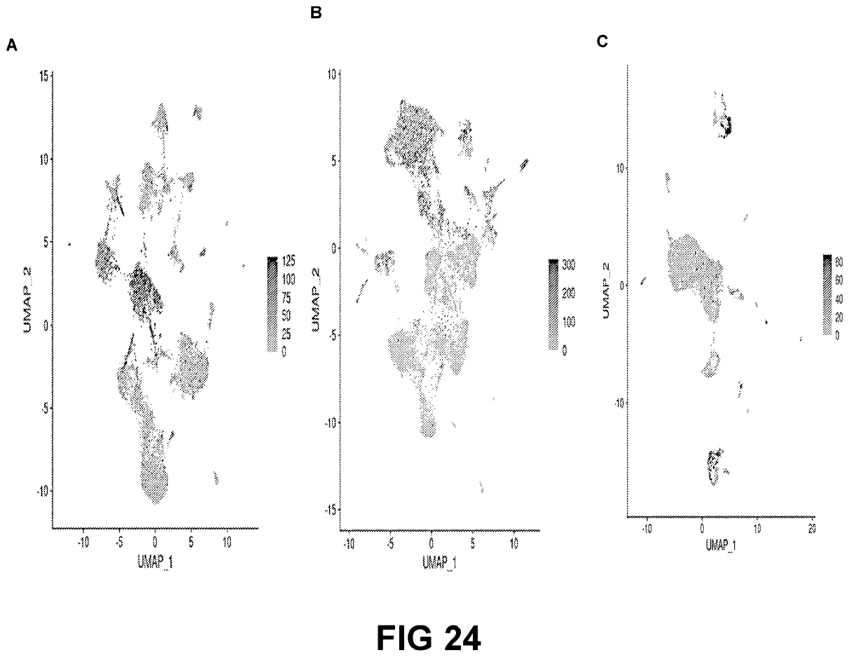

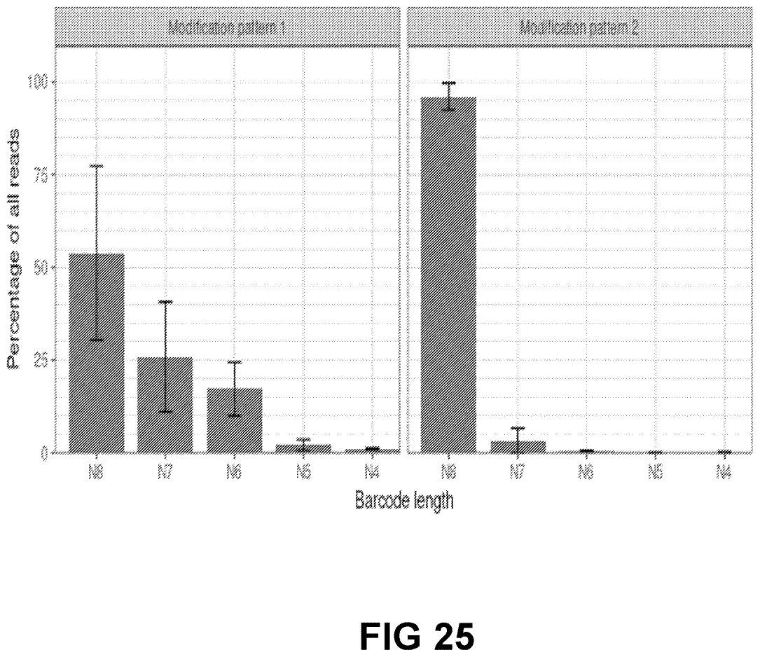

As shown in FIG. 24, some cell type clusters showed higher Mergo uptake than others, consistent with the expected biodistribution profile of the constructs used. FIG. 25 outlines the length distribution of the detected barcodes as observed by single-cell sequencing. Barcodes incorporated into an RNA strand modified with phosphorothioates (modification pattern 1) remained somewhat susceptible to nuclease-mediated degradation; approximately 45% of barcodes lacked one or more 5′ nucleotides. In contrast, barcodes incorporated into RNA strands fully modified with 2′OMe were protected from nucleolytic degradation, with less than 5% of detected barcode reads lacking one or more nucleotides.

### Example 8—Adapter Ligation to Modified RNA Barcode

This experiment was designed to validate if 5′ adapter ligation is a feasible RNAseq library preparation strategy for the detection of modified nucleic acid barcodes incorporated at the 5′ terminus of an oligonucleotide of interest. In a first step three 5′ barcoded RNA oligonucleotide strands, two of which were chemically modified, were 5′ phosphorylated using T4 polynucleotide kinase (M0201, New England Biolabs) according to the manufacturer's protocol. After RNA clean-up using Zymo RNA Clean & Concentrator-5 columns, 5′ adapters (NEBNext 5′ SR Adaptor for Illumina) were ligated using the NEBNext Multiplex Small RNA Library Prep Set for Illumina (E7300S, New England Biolabs) kit following the manufacturer's protocol. Ligated products were loaded on a denaturing PAGE gel (12%) and visualized using Gelred nucleic acid gel stain.

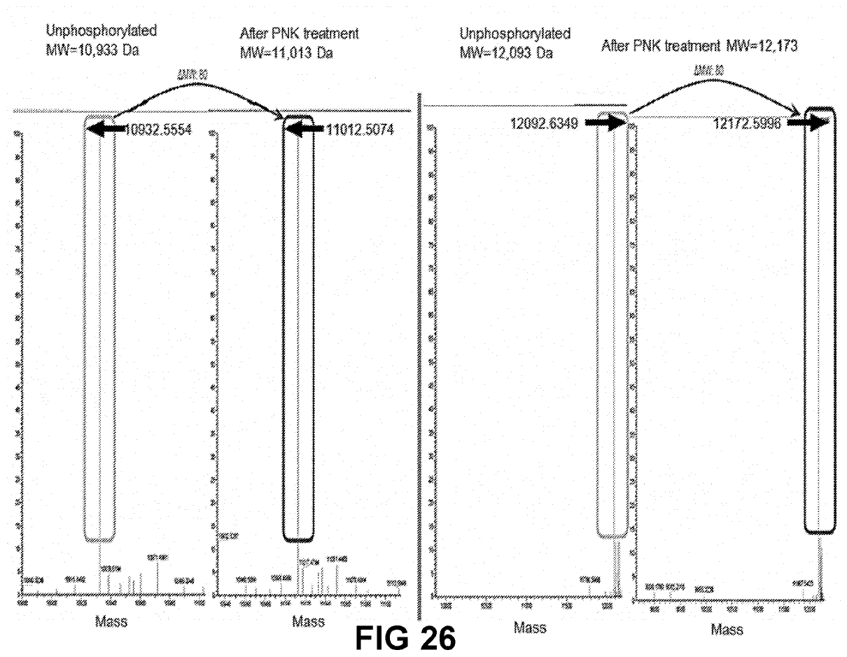

As shown in FIG. 9, adapter ligated samples (lanes 2-4) have a reduced electrophoretic mobility compared to the corresponding unligated samples (lanes 5-7), indicating successful incorporation of the adapter sequence. Representative LC-MS traces confirming 5′ phosphorylation of modified oligos are shown in FIG. 26.

### Example 9—Detection of Internal Barcodes

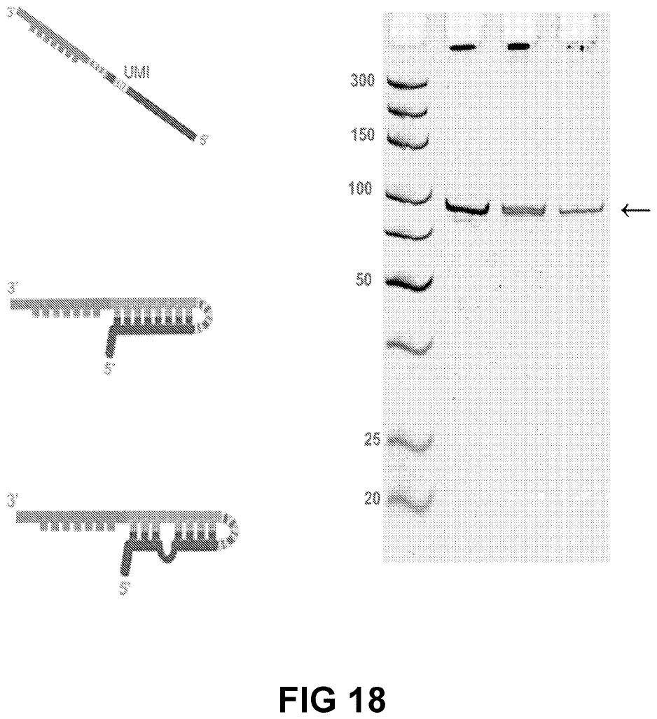

FIG. 18 shows three different designs of barcoded RNA strands with internal short barcodes. These designs also include a 10-nt unique molecular identifier of random nucleotides, flanking the barcode. To validate the feasibility of detecting these designs by NGS, the strands were captured onto magnetic Dynabeads via target-specific capture oligonucleotide. The capture oligonucleotide simultaneously served as primer for reverse transcription using Superscript IV reverse transcriptase, which was run according to manufacturer instructions. Following PCR amplification, the obtained products were loaded on a native PAGE gel (15%) and visualized using Gelred nucleic acid gel stain. All three designs resulted in the expected product band (FIG. 18).

### Example 10—Detection of Barcodes and Lipid-Barcode-Conjugates by Bulk-Tissue RNA-Sequencing

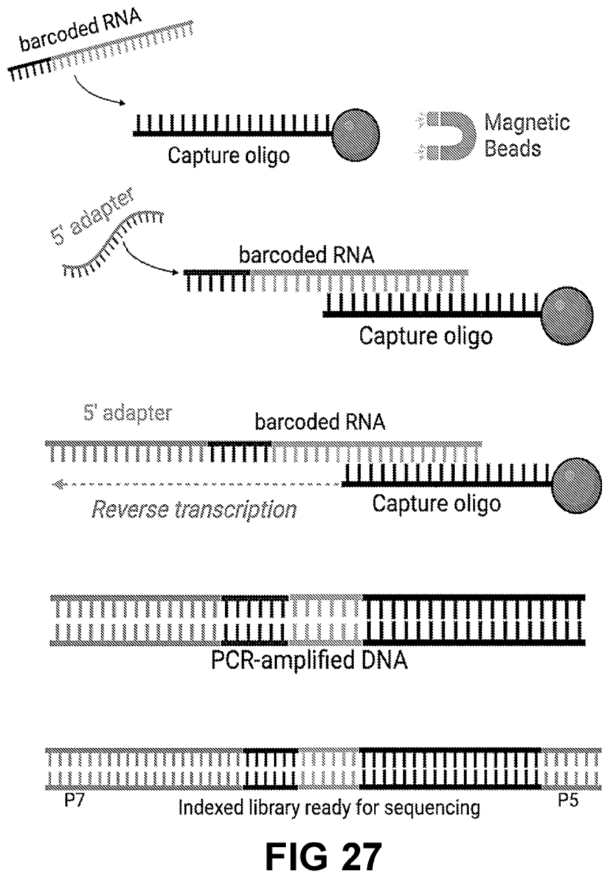

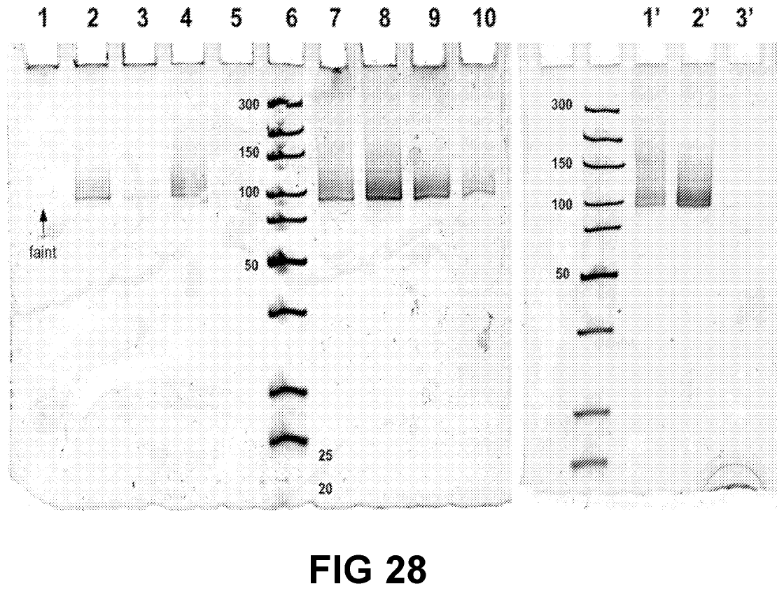

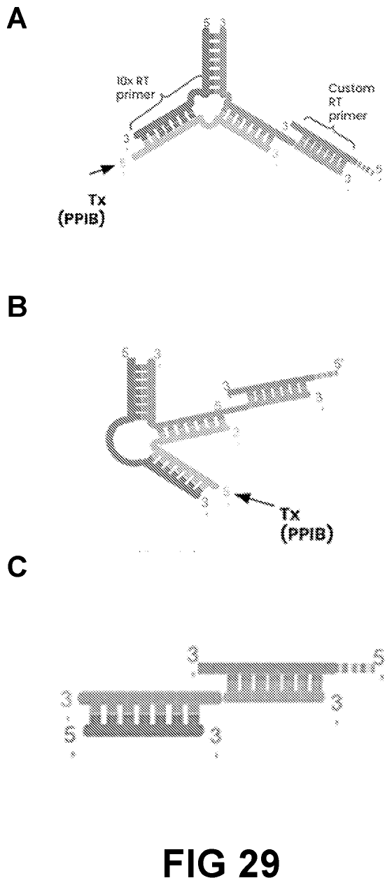

To demonstrate barcode detection at the bulk tissue level, 8-10 week old female Sprague Dawley rats were injected intrathecally either with a double-stranded lipid-barcode-conjugate, or with 9 multiplexed Mergos of 3 different shapes as outlined in FIG. 29. Each design was independently modified with 3 different modification patterns. The unique barcodes on each construct were 8 nucleotides in length and positioned at the 5′ terminus of a fully modified RNA strand hybridized to a complementary strand that was bioconjugated to the Mergo core. Tissues were collected from different brain regions 7 days post-injection, lysed and Mergo strands were captured onto magnetic Dynabeads via target-specific capture oligonucleotide. The full library preparation workflow is outlined in FIG. 27. For 5′ adapter ligation, a set of 10 different adapters was used, each carrying a different 6-nt sample barcode and a 13-nt unique molecular identifier to allow for sample multiplexing and deduplication of PCR duplicates, respectively. As shown in FIG. 28, the different adaptors were initially tested in vitro on single-stranded fully modified barcoded RNA strands to confirm their suitability for use in the library processing pipeline.

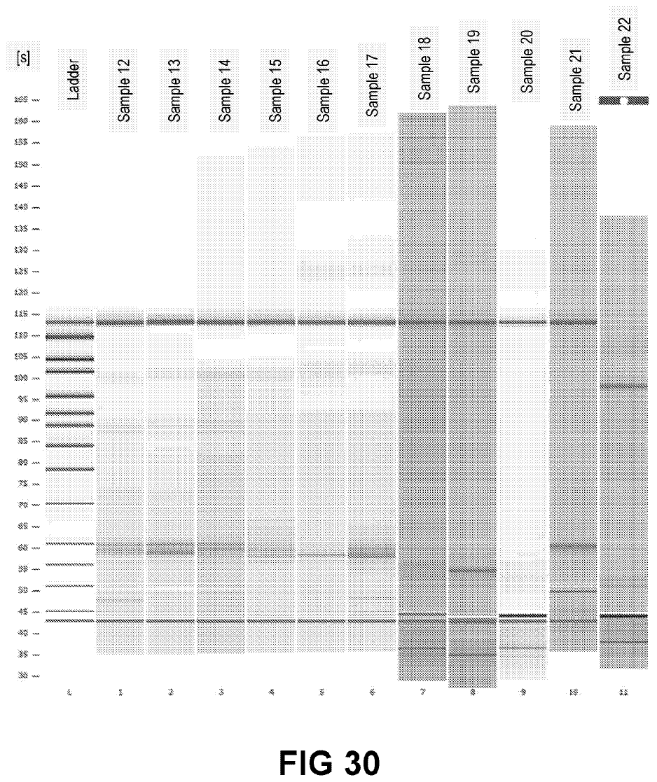

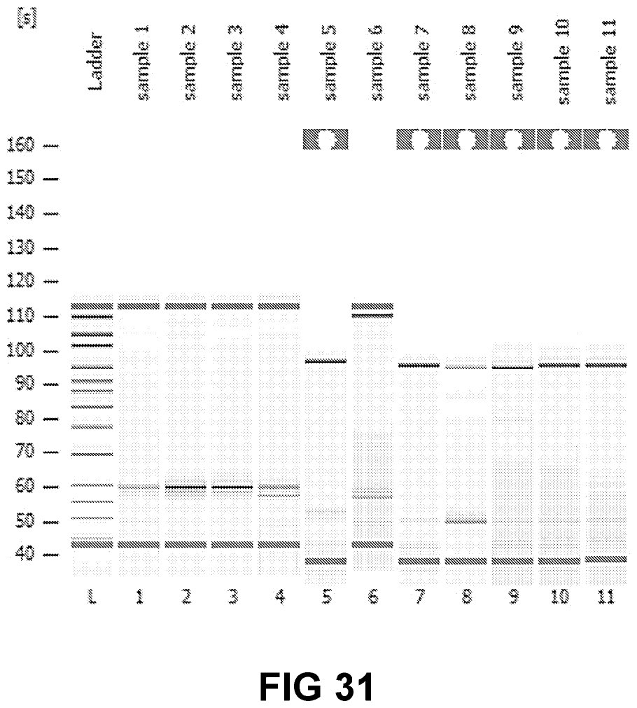

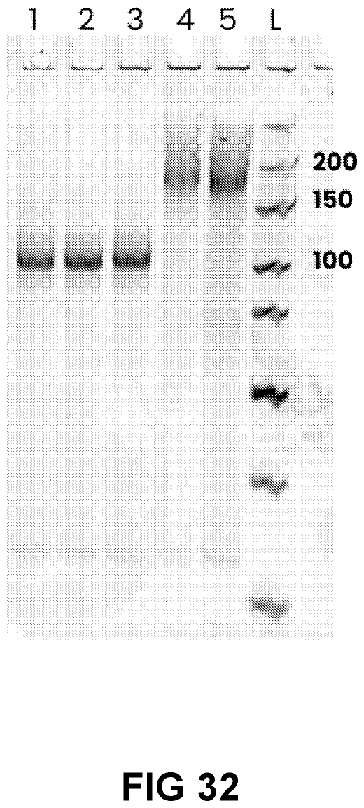

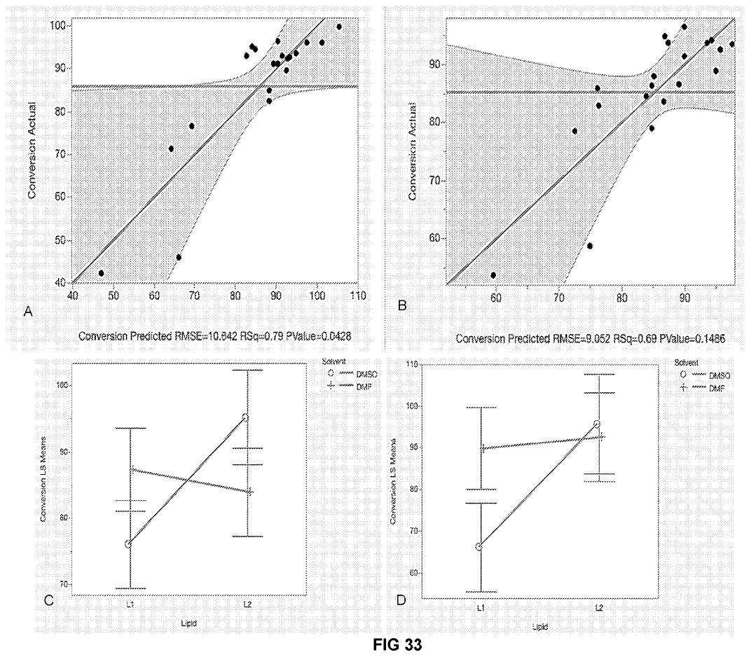

After adapter ligation and sample pooling, libraries were reverse transcribed on solid-phase using Superscript IV reverse transcriptase, PCR amplified using Q5 Hot Start High-Fidelity DNA polymerase (New England Biolabs), indexed with unique dual Illumina indices, purified by size selection and QC-ed using the Agilent Bioanlyzer 2100 expert High Sensitivity DNA Assay. Analytical data of representative rat samples are shown in FIG. 30 and FIG. 31. The presence of individual barcodes was confirmed by qPCR using barcode-specific reverse primers. Obtained qPCR products were assessed by 12% native PAGE and representative results are shown in FIG. 32.

### Example 11—Barcode Conjugation to Peptides

Nucleic acid barcodes can be conjugated to peptides utilizing methodology outlined in US 2021/0330810, the contents of which are incorporated herein by reference. This includes conjugation of 5′ thiol functionalized oligonucleotides to maleimide-functionalized peptides. In an exemplary procedure, a thiol-terminated lyophilized oligonucleotide (234 nmol) was dissolved in water (1 mL) and split into two separate vials. To each vial was added Et₃N (10 μL) and 50 μL of a 1 M solution of DTT. The resultant solution was then agitated at rt for 2.5 h. Excess DTT was then removed with a GelPak desalting column (Glen Research) and the RNA concentration was measured. 96 nmol of fully desalted RNA was recovered. The two desalted oligos were pooled together to give a total volume in H₂O of 3.6 mL. To this, TEAA (100 mM, pH=7, 0.8 mL) was added. The peptide (10 equiv. relative to RNA) was dissolved, in a separate vial, in 20% formamide in MeCN to give a concentration of 50 mM. This was added to the freshly desalted oligo solution, which led to noticeable precipitation. DMSO (4.4 mL) was added to give a 1:1 (v/v) mixture of TEAA/DMSO to solubilise the peptide and assist in denaturation of the oligo. The reaction mixture was agitated at rt for 16 h. This can be purified directly by RP HPLC as the difference in RNA retention time vs. RNA-peptide conjugate is approximately 20 min.

Purification of the RNA-peptide conjugates is carried out by IEX preparative HPLC using a PL-SAX (Agilent) 22×150 mm 1000 Å column at 75° C. with a flow rate of 15 mL/min and UV detection at 260 nm. Elution was performed with a linear gradient selected based on impurity profile, determined by analytical testing using either a DNAPac PA200RS UPLC column or PL-SAX analytical column. Buffer A: 25 mM Tris-HCl, pH 8.0, 20% acetonitrile, 10 mM sodium perchlorate; buffer B: 25 mM Tris-HCl, pH 8.0, 20% acetonitrile, 600 mM sodium perchlorate.

This methodology can be further extended to protein and antibody conjugation.

### Example 12—Design of Experiments (DoE) Optimization of Lipid-Oligonucleotide Conjugations

Heptadecanoic acid (L1) and lithocholic acid (L2) were conjugated to barcode-length oligonucleotides via amide coupling chemistry, using HATU as the amide coupling reagent. The DoE experiment consisted of several variables, including a solvent screen, varied HATU:lipid ratios, varied HATU:oligo ratios, and variations in the percentage of organic solvent (sumarized in Table 2). The lipids were dissolved in the relevant solvent and aliquoted onto 96 well plate. An Opentrons liquid handler was then used to dispense the coupling reagent, base (DIPEA) and oligonucleotides from pre-prepared stock solutions. The plate was then agitated at 900 rpm at rt and an aliquot was taken at 2 h and precipitated in acetone. The rest of the reaction mixtures were agitated for 16 h at rt. Precipitation of the main set of reaction mixtures were carried out by adding NaOAc and acetone and agitating at 3700 rpm for 30 min, followed by removal of the acetone and a repeated washing step. The well was then airdried before resuspension in H₂O.

TABLE 2
variables used in the DoE experiment to optimize
the coupling of nucleic acid barcodes to lipids.

| Lipid | Solvent | Eq. lipid | Eq. CR:lipid | Conversion |
|---|---|---|---|---|
| L2 | DMSO | 1 | 20 | 94.75 |
| L1 | DMSO | 1 | 20 | 53.67 |
| L1 | DMF | 2 | 40 | 86.29 |
| L2 | DMSO | 2 | 20 | 92.38 |
| L2 | DMF | 2 | 20 | 84.44 |
| L2 | DMF | 1.5 | 30 | 87.81 |
| L1 | DMSO | 1.5 | 30 | 58.63 |
| L2 | DMF | 2 | 40 | 78.43 |
| L1 | DMSO | 2 | 40 | 93.73 |
| L1 | DMF | 1 | 20 | 96.31 |
| L2 | DMF | 1 | 40 | 86.5 |
| L1 | DMSO | 1 | 40 | 82.81 |
| L1 | DMF | 2 | 20 | 83.47 |
| L2 | DMF | 1 | 20 | 88.73 |
| L2 | DMSO | 1 | 40 | 94.1 |
| L1 | DMF | 2 | 40 | 78.94 |
| L1 | DMSO | 2 | 20 | 85.72 |
| L1 | DMF | 1 | 40 | 93.53 |
| L2 | DMSO | 2 | 40 | 93.49 |
| L1 | DMF | 1 | 20 | 91.43 |

The key variables were found to be the lipid identity relevant to the solvent (i.e. more aromatic=DMSO preferable, more aliphatic=DMF preferable), solvent identity vs. equiv. lipid, lipid identity in general (i.e. sterically bulky vs. non bulky, solubility considerations). Following optimisation, barcodes were conjugated to the lipids shown in table 3.

TABLE 3
Lipids conjugated to oligonucleotide barcodes

| Lipid | CAS |
|---|---|
| cis-4,7,10,13,16,19-Docosahexaenoic acid | 6217-54-5 |
| Docosanoic acid | 112-85-6 |
| cis-5,8,11,14,17-Eicosapentaenoic acid | 10417-94-4 |
| Cholesteryl hemisuccinate | 1510-21-0 |
| Lithocholic acid | 434-13-9 |
| Retinoic acid | 302-79-4 |
| Heptadecanoic acid | 506-12-7 |
| 4-Phenylbutyric acid | 1821-12-1 |
| 3-(2-Furyl)acrylic acid | 539-47-9 |
| 2-Hexyldecanoic acid | 25354-97-6 |
| Linoleic acid | 60-33-3 |

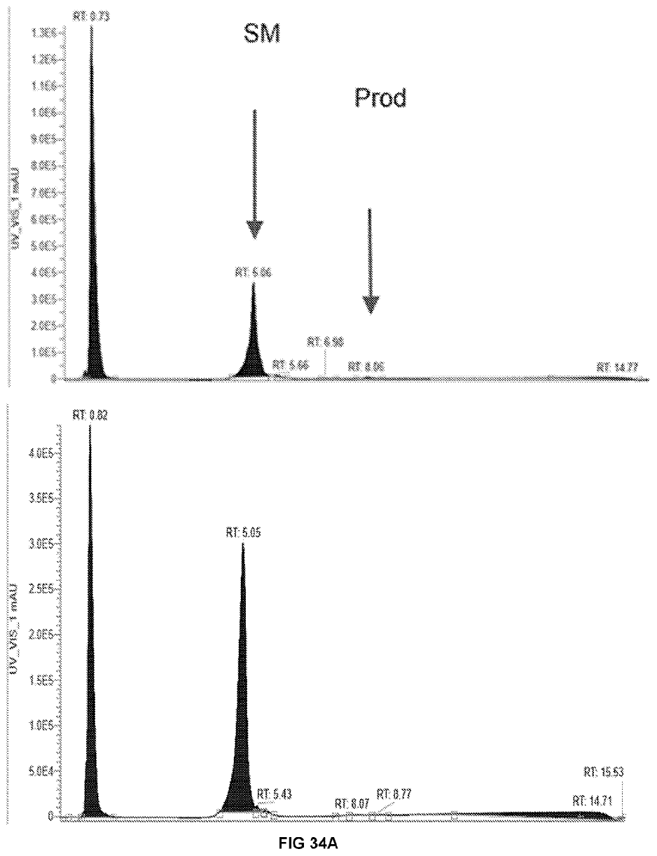

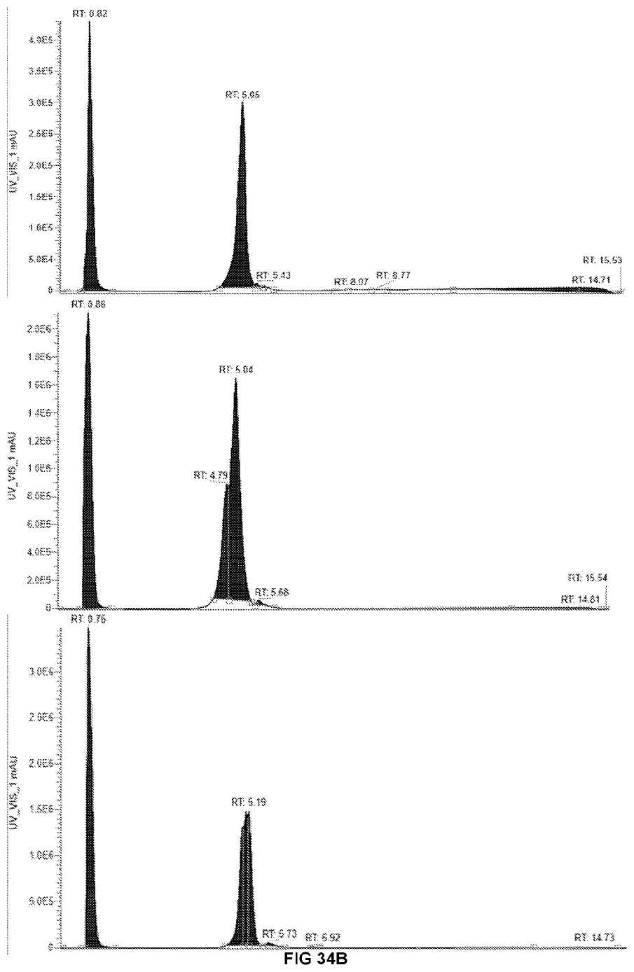

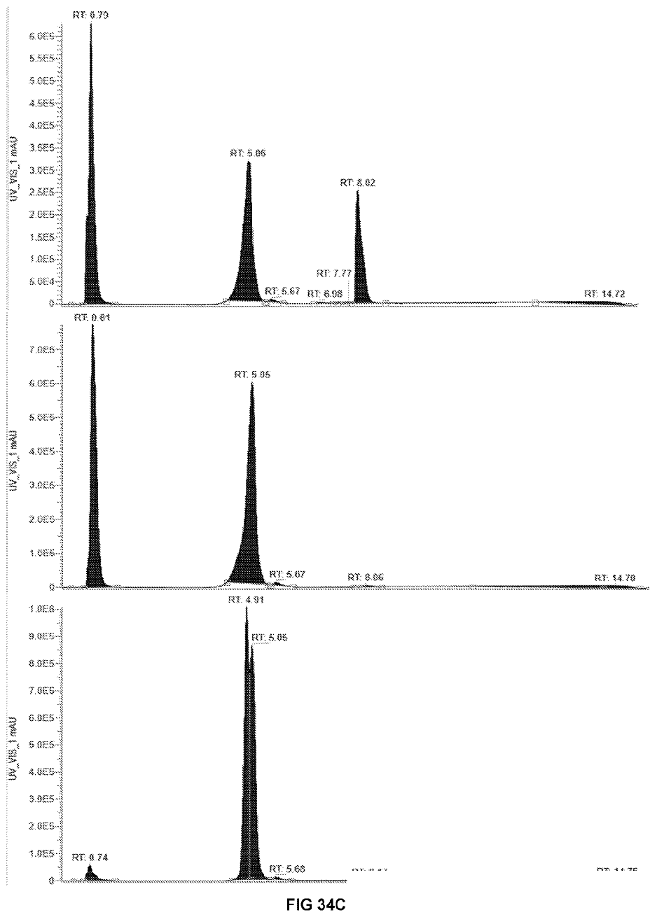

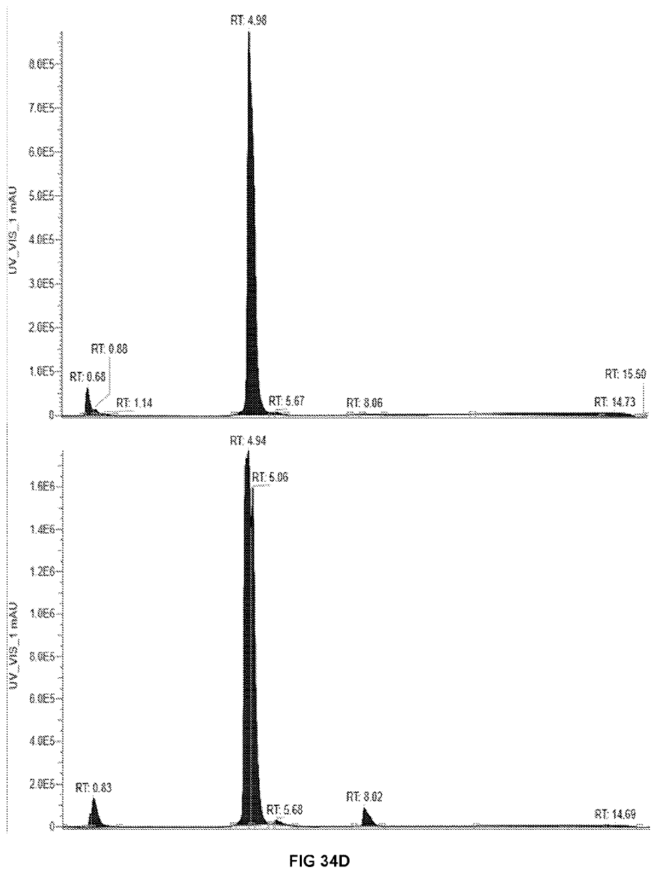

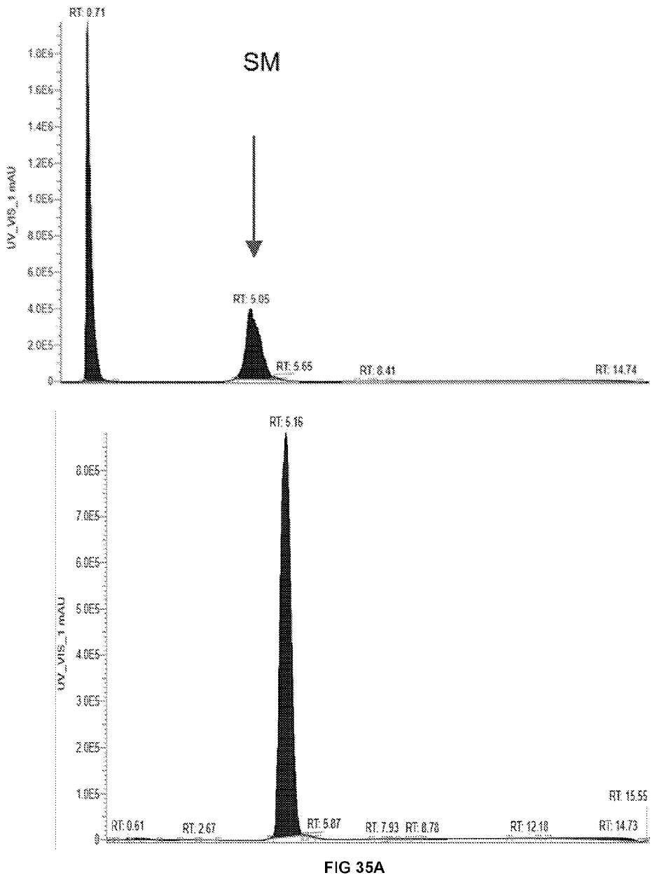

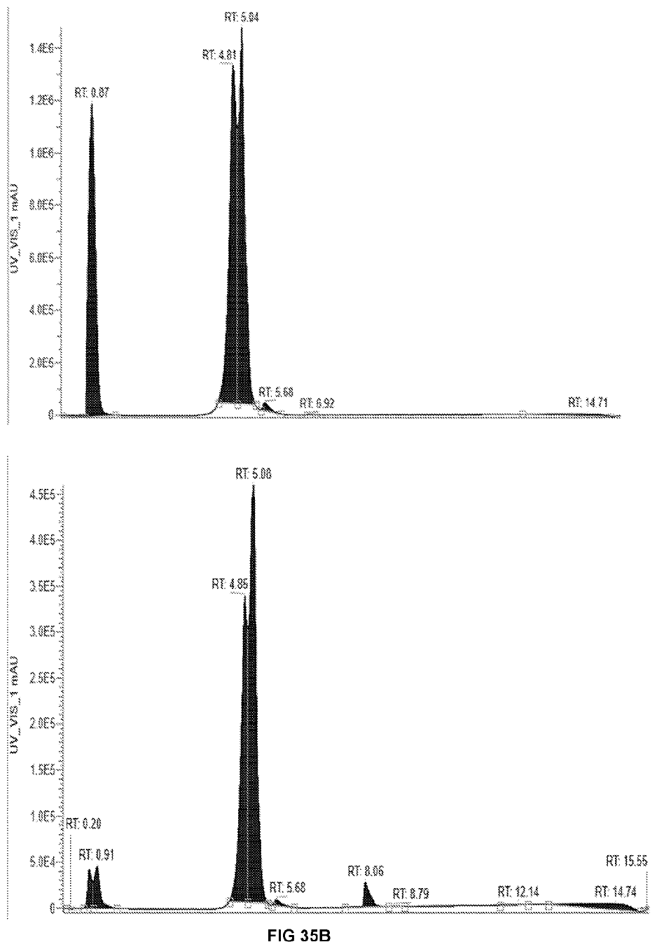

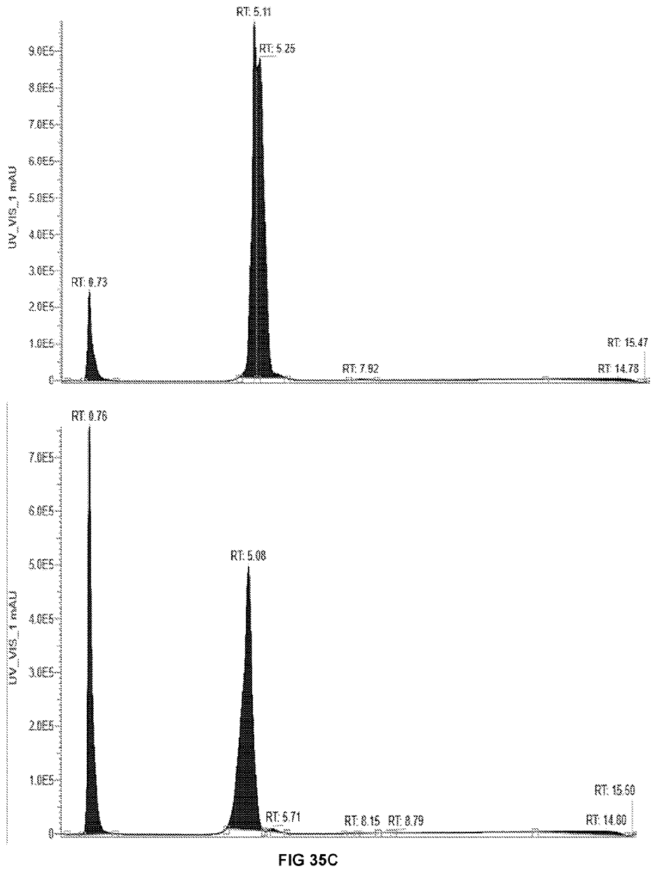

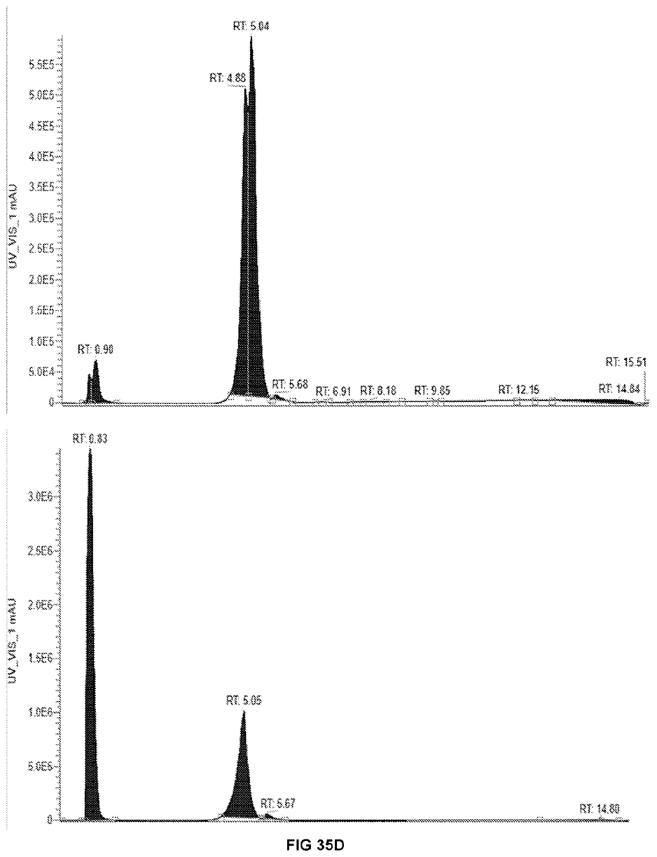

Analytical data for representative barcode-oligonucleotide barcodes is shown in FIGS. 34A-34D (barcode to heptadeconic acid) and FIGS. 35A-35D (barcode to DSPE-glutaric acid).

### Example 13—Optimised Synthesis of Barcode-Lipid Conjugates Via NHS Chemistry

The following protocol can be applied to the synthesis of barcode-lipid conjugates; the representative sequence [/5AmMC6/c*mA*amAumUcmCamUcmGu*mG*a—whereby 2′ F C/A/U is indicated with lowercase, 2′ O me A/U/G/C indicated with ‘m’, PTO indicated with *, ′5AmMC6′ represents a C6 amino modifier] was coupled to palmitic acid as a proof of concept.

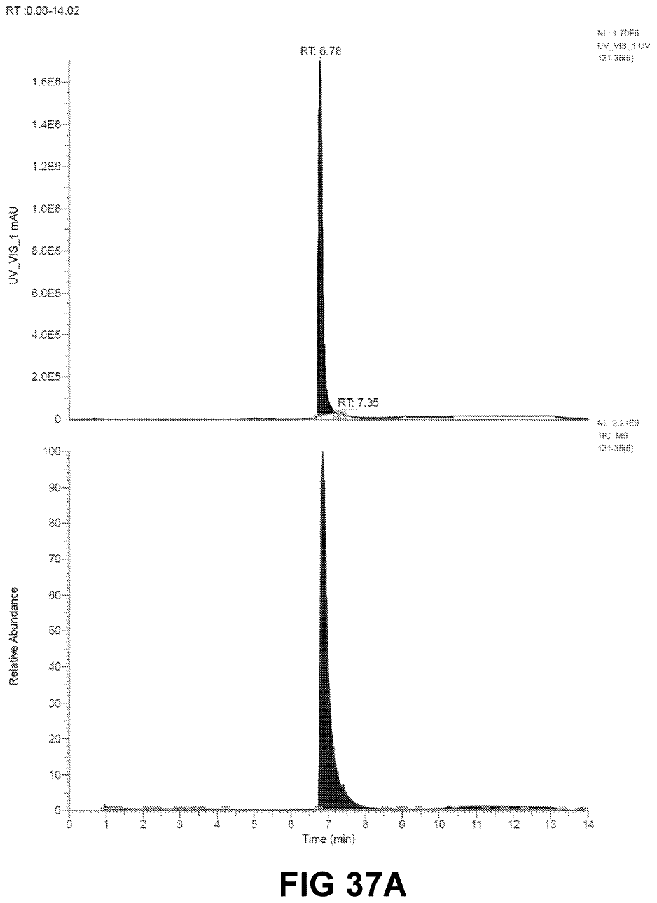

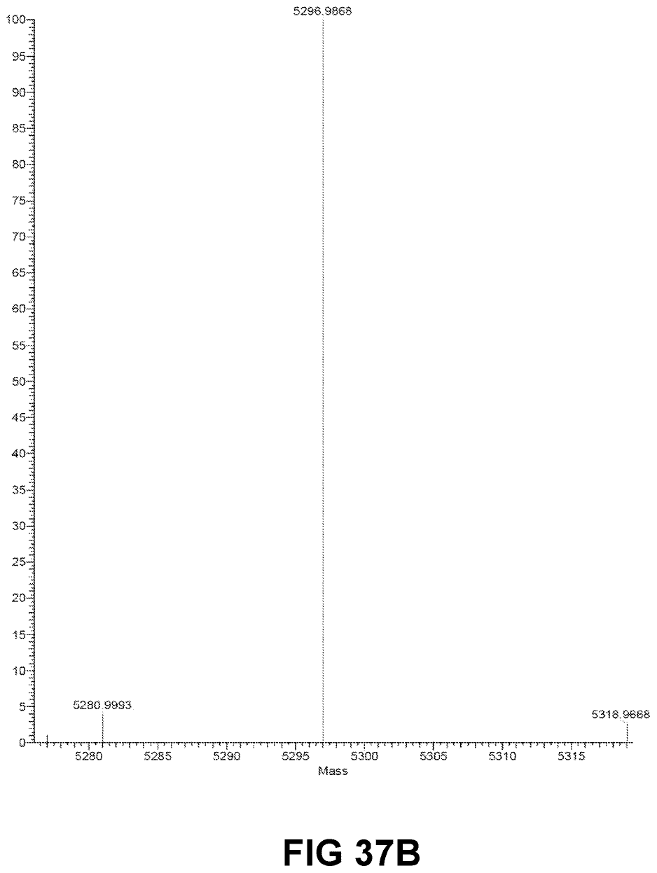

An amine modified oligonucleotide/barcode (4 μmol) was dissolved in 600 μL of water, followed by the addition of DMF (3.3 mL), 500 mM sodium bicarbonate buffer (250 μL) and DIPEA (50 μL). NHS-labelled lipid (20 equiv.) in 800 μL DMF was then added to the reaction mixture and this was agitated at 30° C. for 2 h. Reaction progress was then monitored with RP-HPLC and after confirming full conversion, the reaction mixture can either be directly purified via preparative RP-HPLC or for higher volumes can be EtOH precipitated and resuspended in H₂O prior to HPLC purification. Alternatively, lipid conjugates can be purified directly via ultrafiltration. The associated analytical data for this example is shown in FIG. 37.

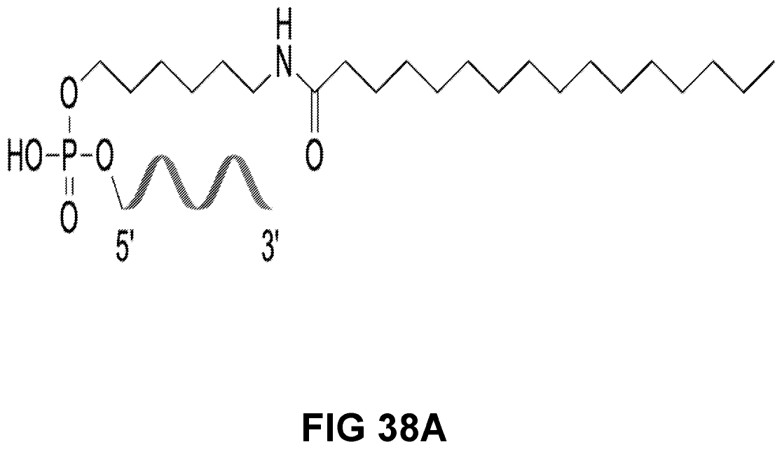

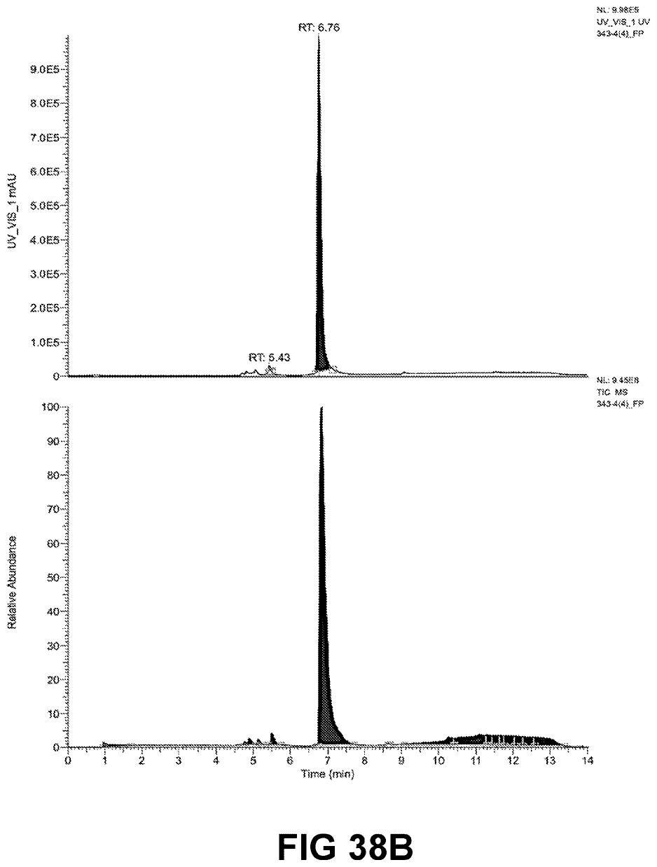

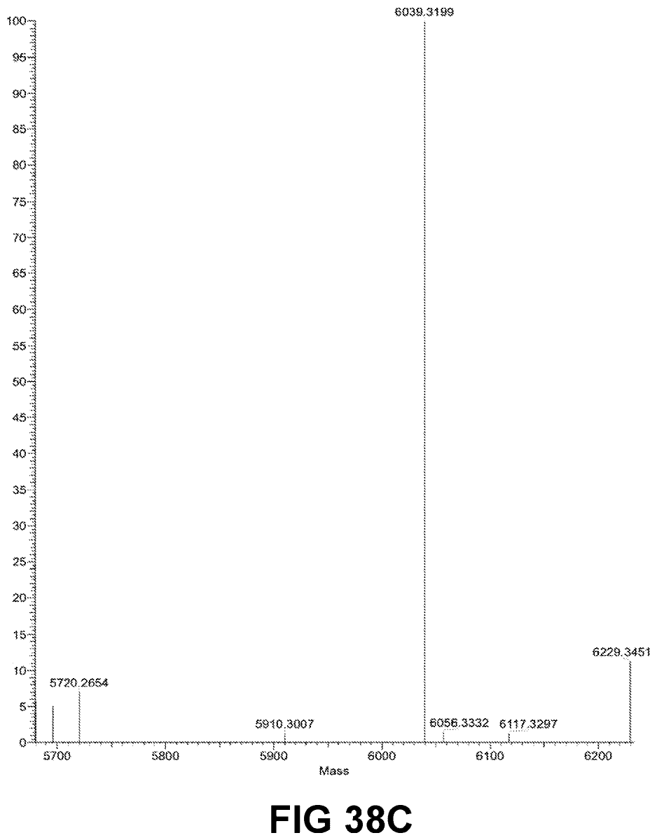

A hexadecanyl chain was added to a barcode sequence (C16//5AmMC6/mCmCmAmCmUmUmGmGmAmCmGmAmGmUmUmAmC, whereby 2′ O me A/U/G/C indicated with ‘m’ and ′5AmMC6′ represents a C6 amino modifier) with this methodology. The analytical data is shown in FIGS. 38A-38C.

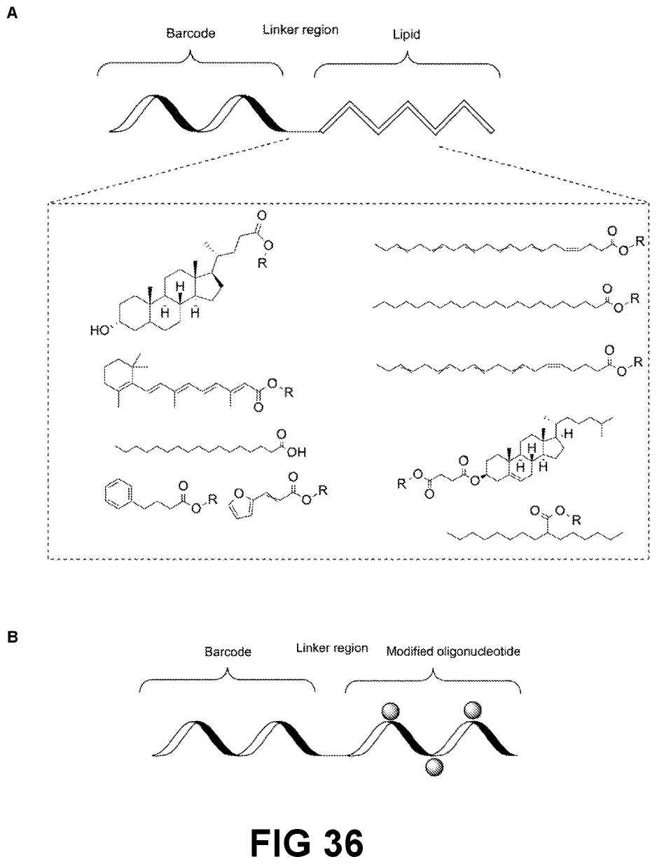

Lipid conjugates can also be prepared via phosphoramidite chemistry. This is known in the art and can be found in, for example, US 2022/0125823, which is incorporated by reference. Lipid chain lengths can be between 2-30 carbon atoms or could include, without limitation, any lipids shown in FIG. 36. A short barcode-like strand was used as a representative example of phosphoramidite chemistry applied to the synthesis of barcode-oligonucleotide constructs (c*mA*amAu(C16u)cmCamUcmGu*mG*a, whereby 2′ F C/A/U is indicated with lowercase, 2′ O me A/U/G/C indicated with ‘m’, PTO indicated with * and (C16u) is a uridine nucleotide modified at the 2′O with hexadecanyl chain. This can be attached via the 5′ with a phosphoramidite lipid modifier or internally via modification of the 2′O on the ribose.

### Example 14—Design of Barcoded Constructs

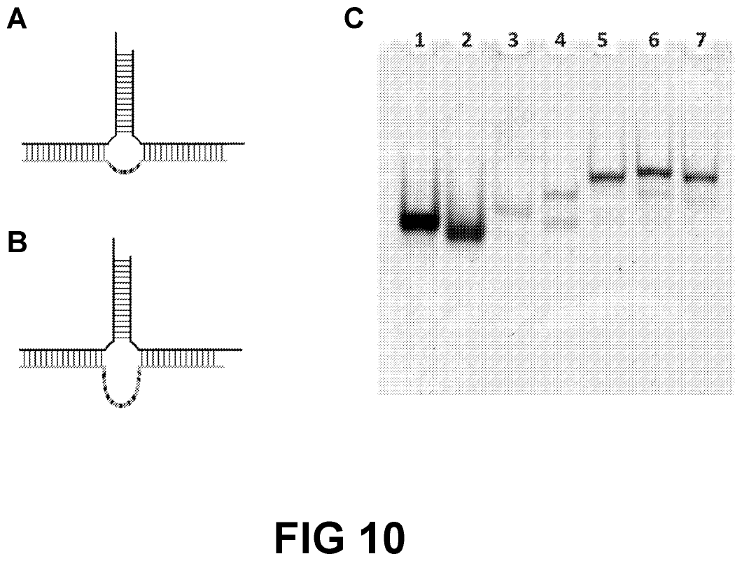

FIG. 10 shows the exemplary assembly of two DNA nanostructures comprising a 3-way junction and a nucleic acid barcode of 8 nucleotides (M-3, FIG. 10 A) or 16 nucleotides (M-4, FIG. 10 B) in length, respectively. The barcodes are included in the single-stranded loop region of one of the constituting strands. The barcoded strand of M-4 further comprises a bi-partite UMI that flanks the barcode sequence. To assemble the DNA nanostructures, stoichiometric amounts of the constituting strands were mixed in an assembly buffer (PBS+2 mM MgCl₂) at a final construct concentration of 10 μM. The mixture was then heated at 95° C. for 5 min followed by a gradual cooldown to 4° C. (2.5° C./min) using a PCR thermocycler. One pmol of the mixture were loaded on a native PAGE gel (6%), electrophoresed in 1× TBMg (890 mM Tris Borate+20 mM Mg(OAc)2, pH=8.3) at a constant voltage of 100 V and gel bands were visualized with GelRed. Two μL of glycerin (70% in H₂O) were added to the samples before loading.

The resulting gel image (FIG. 10 (C)) suggests that both nanostructures assemble well with minor upper and lower bands present.

### Example 15—Alkyl Phosphonamidites

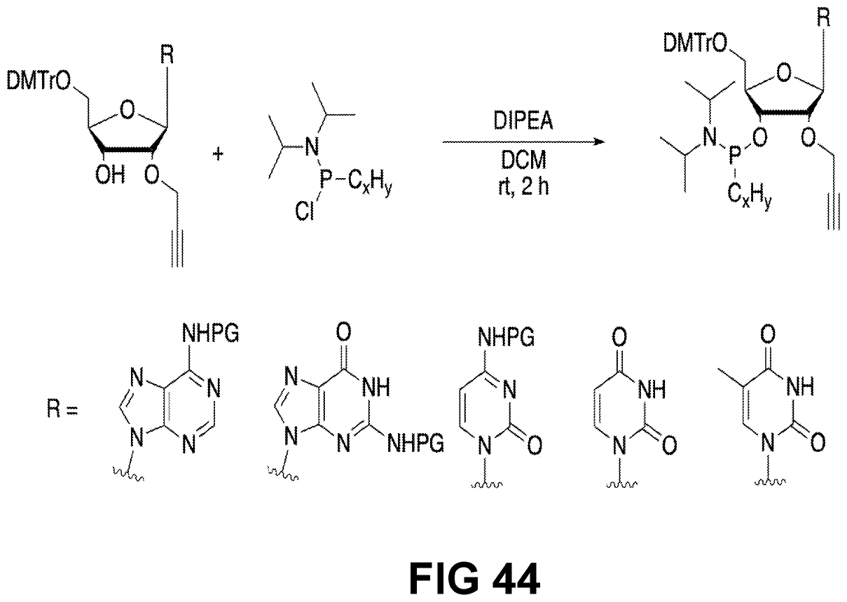

The synthesis of reagents to convert alcohols to alkyl phosphonamidites is outlined in FIG. 43. An alkyl bromide is first treated with Mg in Et₂O, followed by subsequent treatment with CdCl₂ and PCl₃ to form the dichloro(alkyl)phosphane. This is then further treated with 1 equiv. diisopropylamine to form the phosphorylating reagent. This reagent can then be used under conventional phosphitylating conditions, as outlined in FIG. 44.

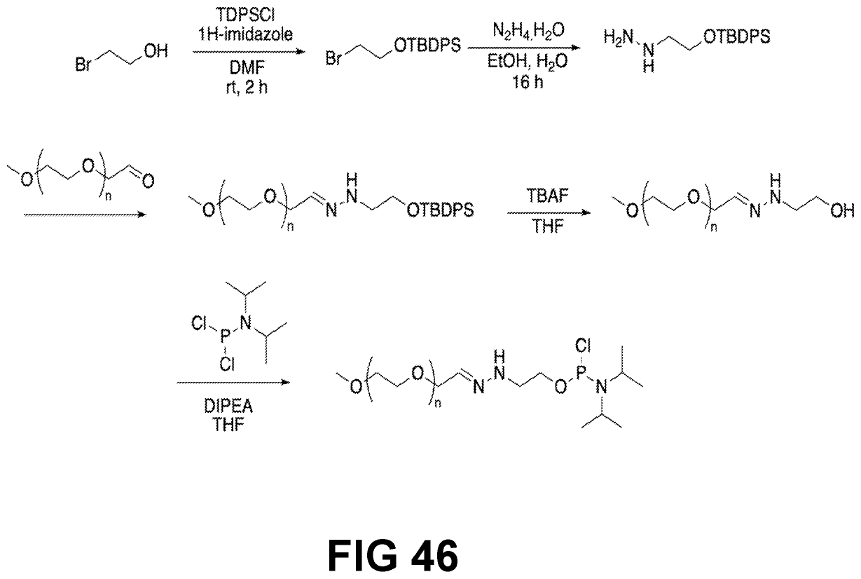

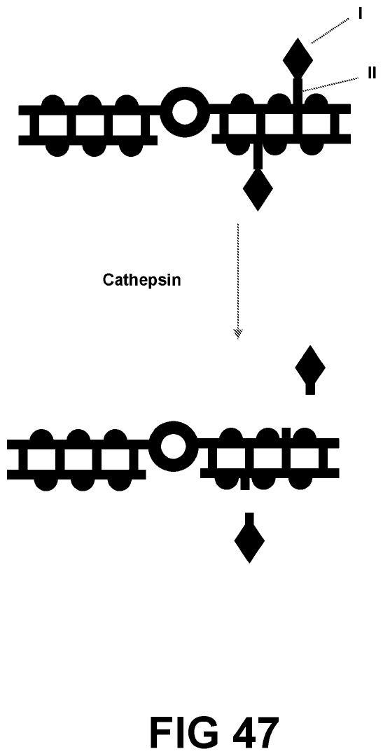

Modified phosphitylating reagents can be made simply by reacting the alcohol with PCl₃ to form the intermediate dichlorophosphane, followed by subsequent treatment with 1 equiv. diisopropylamine to form the phosphitylating reagent (FIG. 45). The synthesis of an example hydrazone-based reagent is given in FIG. 46. TBDPS-protected 2-bromoethan-1-ol is converted to a hydrazine, which is then reacted with a PEG-aldehyde, followed by the subsequent removal of the TBDPS with TBAF. This is then treated with 1,1-dichloro-N,N-diisopropylphosphanamine to afford the modified phosphitylating reagent.

The phosphitylating reagents can then be used to generate nucleoside phosphonamidites via conventional coupling methodologies. Such methodologies are known in the art and are given, for example, in US 2006/287260. These alkyl phosphonamidites can then be incorporated into oligonucleotides via conventional P(III) chemistry.

### Example 16—General Small Molecule Synthetic Procedures

### General Experimental

¹H NMR spectra were recorded at 400 MHz. ¹³C NMR spectra were recorded at 100 MHz. Chemical shifts (6) are quoted in units of parts per million (ppm) downfield from tetramethylsilane and are referenced to a residual solvent peak. (CDCl₃ (δ_{H}: 7.26, δc: 77.0)). Coupling constants (J) are quoted in units of Hertz (Hz). The following abbreviations are used within ¹H NMR analysis: s=singlet, d=doublet, t=triplet, q=quartet, pent=pentet, m=multiplet, dd=doublet of doublets, dt=doublet of triplets. Spectra recorded at 400 (¹H NMR) and 100 (¹³C NMR) were carried out by the Imperial College London Department of Chemistry NMR Service.

Low- and high-resolution mass spectrometry (EI, CI, FAB) were recorded at Imperial College London. Measurements carried out by the Imperial College Department of Chemistry Mass Spectrometry Service used a Micromass Platform II and Micromass AutoSpec-Q spectrometer.

Flash column chromatography was carried out on BDH silica gel 60, particle size 0.040-0.063 mm. Thin layer chromatography (TLC) was performed on pre-coated aluminum backed or glass backed plates (Merck Kieselgel 60 F254), and visualized with ultraviolet light (254 nm) or potassium permanganate (KMnO₄), vanillin or phosphomolybdic acid (PMA) stains.

### Example 17—Electrostatic Modifier Small Molecule Synthesis

4-(Hexyloxy))anilinium chloride

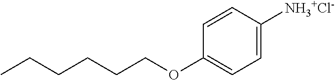

Following a previously reported method (G. Chakraborty, K. Balinin, G. Portale, M. Loznik, E. Polushkin, T. Weil, A. Herrmann, Electrostatically PEGylated DNA enables salt-free hybridization in water, Chem. Sci. 10 (2019) 10097-10105. https://doi.org/10.1039/c9sc02598g.), 4-(hexyloxy)aniline (5 g, 30 mmol) was dissolved in Et₂O (100 mL) with vigorous stirring. Freshly prepared HCl gas, which was obtained by mixing sulfuric acid and brine, was then bubbled into the reaction mixture. The solution became turbid and a precipitate of the title compound was formed. HCl gas was bubbled through the solution for a further 30 min to ensure complete conversion. The precipitate was then collected by filtration and washed with Et₂O (2×100 mL), followed by drying under vacuum to afford the title compound as a light purple solid (5.2 g, 90%). ¹H NMR (400 MHz, DMSO) δ 10.33 (s, 3H), 7.37-7.28 (m, 2H), 7.05-6.98 (m, 2H), 3.96 (t, J=6.5 Hz, 2H), 1.76-1.64 (m, 2H), 1.41 (td, J=8.4, 7.6, 5.1 Hz, 2H), 1.30 (dq, J=7.2, 3.7 Hz, 4H), 0.91-0.83 (m, 3H); ¹³C NMR (101 MHz, DMSO) δ 158.6, 124.9, 124.5, 115.7, 68.3, 31.5, 29.0, 25.6, 22.5, 14.4. NMR consistent with literature.

### Example 18—CuAAC of Modifications to Mergos and Barcodes

CuAAC reactions were performed with an optimized bpba ligand (S. I. Presolski, V. Hong, S.-H. Cho, M. G. Finn, Tailored Ligand Acceleration of the Cu-Catalyzed Azide-Alkyne Cycloaddition Reaction: Practical and Mechanistic Implications, J. Am. Chem. Soc. 132 (2010) 14570-14576. https://doi.org/10.1021/ja105743g.). Reactions were performed in 1:1 mixtures TEAA 2 M:DMSO. Propargyl modified oligonucleotide stocks were prepared in H₂O at concentrations around 200-300 pM. Sodium ascorbate stocks solutions were freshly prepared in TEAA before each experiment. Azide stocks solutions were prepared in DMSO. Cu₂SO₄:bpba 1:1 stock solution was prepared in TEAA:DMSO 1:1 mixture.

The reactions were performed with 20-40 M concentration of oligonucleotide, 2.5-5 eq of azide, 3.75-7.5 eq of sodium ascorbate, and 3.75-7.5 eq of Cu₂SO₄:bpba, added in this order. After Cu:bpba addition, the solutions were purged with N2 and agitated for 4 h. The conversion was confirmed by LC-MS. For the analysis of the samples, 10 μL of crude reaction were added to a low volume HPLC vial together with 5 L of EDTA.

Oligonucleotides were purified via ultrafiltration with VivaSpin centrifugal concentrators (cut-off membrane 50% higher than the mass of the oligonucleotide, Hydrostat membrane) after EtOH precipitation and treatment with 0.5 M EDTA (0.1-0.5 volumes). The membrane was first pre-wet with H₂O and the sample was loaded above. The filter was then centrifuged at 2500 g for 10-30 min, followed by subsequent washes with water (×3).

### Example 19—Cathepsin-Sensitive Linkers

The synthesis of a convergent dipeptide intermediate is outlined in FIG. 48 and FIG. 49. The Fmoc-protected dipeptide pairs can be made via simple amidation chemistry or are commercially available. To the Fmoc-protected dipeptides, bis(4-nitrophenyl) carbonate is added in the presence of DIPEA in DMF. 5-Norbornene-2-methylamine is then added in the presence of DIPEA in DMSO to form the norbornene-PABC unit. Removal of the Fmoc is then achieved with piperidine. This compound can then be treated with a PEG of desired length via an amidation reaction. To form that NHS linker, the PEG hydroxyl is reacted with a succinate to form a terminal carboxylic acid, followed by TSTU treatment to form the NHS. To form the phosphoramidite, the PEG hydroxyl is treated directly with 2-cyanoethyl N,N-diisopropylchlorophosphoramidite in the presence of DIPEA.

Incorporation into Oligonucleotides and Coupling

### i) NHS Chemistry

NHS reactions were performed in a 0.3:0.1:0.5:0.1 mixture Bicarbonate 0.5 M: NaCl 1 M: DMSO/DMF: MB water. Amine modified oligonucleotide stocks were prepared in H₂O at concentrations around 400-1000 M. Cleavable linker NHS with norbornene functionality stock solutions were prepared in DMSO/DMF. The reactions were performed with 200-500 pM concentration of oligonucleotide and 5-10 equiv. of NHS-linker. The conversion was confirmed by LC-MS. For the analysis of the samples, samples were precipitated with EtOH and NaOAc 3M (10% volume of reaction mixture).

Oligonucleotides were purified via ultrafiltration with VivaSpin centrifugal concentrators (cut-off membrane 50% higher than the mass of the oligonucleotide, Hydrostat membrane) after EtOH precipitation followed by treatment with 0.5 M EDTA (0.1-0.5 volumes). The membrane was first pre-wet with H₂O and the sample was loaded above. The filter was then centrifuged at 2500 g for 10 min, followed by subsequent washes with water (×3).

### ii) Phosphoramidite Chemistry

Additions of phosphoramidite were performed in standard SPS conditions (C. Jin, A. H. EI-Sagheer, S. Li, K. A. Vallis, W. Tan, T. Brown, Engineering Enzyme-Cleavable Oligonucleotides by Automated Solid-Phase Incorporation of Cathepsin B Sensitive Dipeptide Linkers, Angew. Chemie Int. Ed. 61 (2022) 1-7. https://doi.org/10.1002/anie.202114016.) and deprotected by saturated ammonium hydroxide solution (55° C., 4-17 h) or AMA (55° C. 4 h) followed by evaporation and RP HPLC purification.

IEDDA conjugation via the linker norbornene can be carried out in using the method outlined in these examples.

### Example 20—Formation of Electrostatic Formulations

The electrostatic formulation of Mergo was carried out according to procedures previously reported for DNA (G. Chakraborty, K. Balinin, G. Portale, M. Loznik, E. Polushkin, T. Weil, A. Herrmann, Electrostatically PEGylated DNA enables salt-free hybridization in water, Chem. Sci. 10 (2019) 10097-10105. https://doi.org/10.1039/c9sc02598g.).

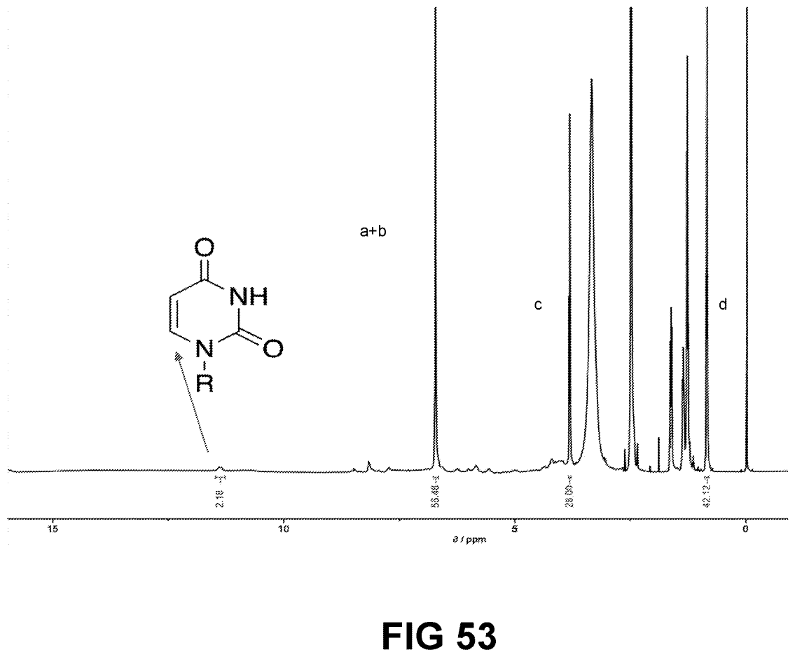

The first step involves the formation of an oligonucleotide-ANI complex. 4-(Hexyloxy))anilinium chloride (100 mM) is added to the oligonucleotide (200 μM) with an excess of three ANI molecules per negatively charged phosphate. For example, a 34 nt oligonucleotide (18.2 nmol) will have 33 charges. therefore 3×33×18.2 nmol=1.8 μmol of ANI is added. Upon addition of the ANI, a precipitate is immediately formed and the solution is mixed thoroughly at 800 rpm for 1 h at rt. It is then centrifuged at 13,000 rpm for 10 minutes. The pellet is then washed three times via the addition of water (1 mL) and further centrifugation. The oligonucleotide-ANI complex is then freeze-dried (¹H NMR is shown in FIG. 53).

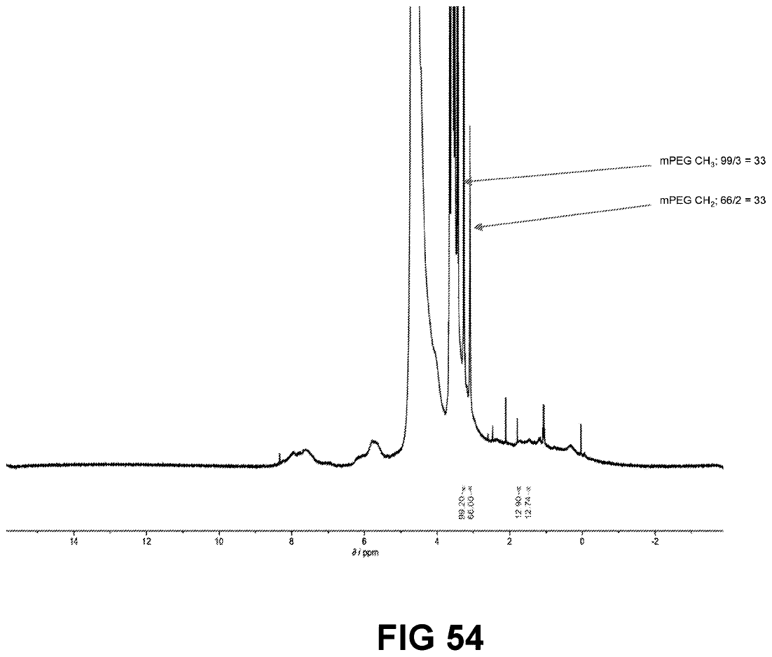

The second step involves the formation of the oligonucleotide-PEG complex. The freeze-dried oligonucleotide-ANI complex is resuspended in MeOH (1 mL), followed by the addition of a threefold excess of the PEG (100 mM) per negatively charged phosphate. For example, a 34 nt oligonucleotide (18.2 nmol) will have 33 charges, therefore 3×33×18.2 nmol=1.8 μmol of PEG is added. The mixture is allowed to shake for 2 h at 800 rpm. It is then diluted to a 1:1 mixture of MeOH/H₂O and concentrated with a vivaspin centrifugation column. This concentrate is then washed with a 1:1 mixture of MeOH/H₂O (×5) and the residual MeOH is removed in a speed-vac. The remaining aqueous solution is then freeze-dried to obtain the resultant oligonucleotide-PEG complex (1H NMR shown in FIG. 54).

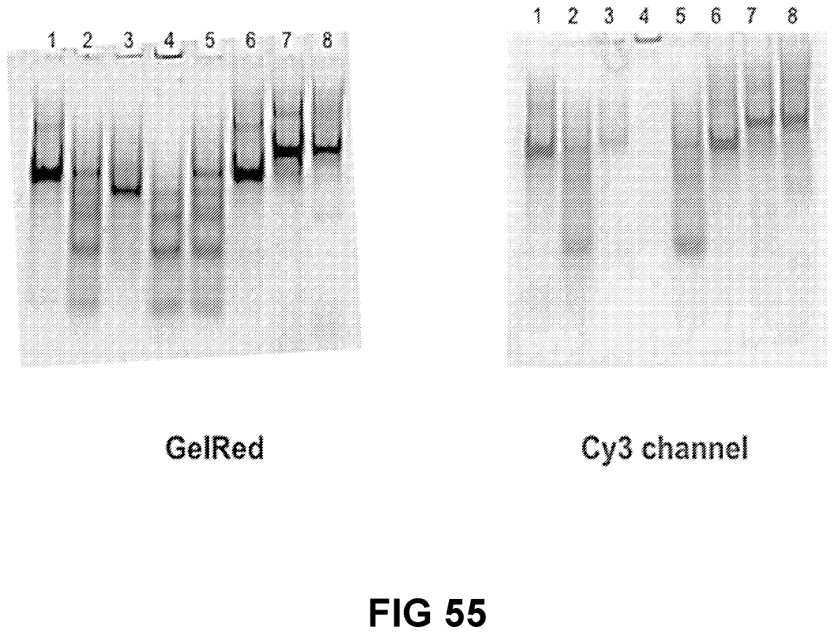

This methodology has been previously used with DNA. The present disclosure discloses the use of this method for oligonucleotides with extremely hydrophobic modifications, including a 58 nt strand consisting of 2′F-modified U and C, 5′ Cy3 and 19 chloroquines that were attached via CuAAC. This oligonucleotide was insoluble in water in the absence of the electrostatic PEG modification and can be incorporated into Mergo constructs as shown in FIG. 55 (6% native PAGE showing Mergo assemblies with an electrostatic PEG layer).

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### EQUIVALENTS

Various modifications of the disclosure and many further embodiments thereof, in addition to those shown and described herein, will become apparent to those skilled in the art from the full contents of this document, including references to the scientific and patent literature cited herein. The subject matter herein contains important information, exemplification, and guidance that can be adapted to the practice of this disclosure in its various embodiments and equivalents thereof.

What is claimed is:

1. A composition comprising:
a nucleic acid analyte further comprising a nucleic acid barcode, wherein said nucleic acid barcode allows for the detection and unambiguous identification of the nucleic acid analyte in a biological sample, and wherein said nucleic acid barcode is not encapsulated in a lipid nanoparticle formulation.

2. The composition of claim 1, wherein said nucleic acid analyte and/or said nucleic acid barcode comprise DNA, RNA, XNA, or a combination thereof.

3. The composition of claim 2, wherein said nucleic acid barcode is between 5-500 nucleotides in length.

4. The composition of claim 3, wherein said nucleic acid barcode is between 5-30 nucleotides in length.

5. The composition of claim 4, wherein said nucleic acid barcode is between 6-12 nucleotides in length.

6. The composition of claim 3, wherein said nucleic acid barcode comprises a single-stranded portion, a double-stranded portion, a triple-stranded portion, a quadruple-stranded portion, a quintuple-stranded portion or combinations thereof.

7. The composition of claim 6, wherein said nucleic acid barcode is single-stranded.

8. The composition of claim 6, wherein said nucleic acid barcode is double-stranded.

9. The composition of claim 6, wherein said nucleic acid barcode comprises one or more modified nucleotides, and wherein each of said one or more modified nucleotides is independently modified at the ribose group, the phosphate group, the nucleobase group, or a combination thereof.

10. The composition of claim 9, wherein each nucleotide of said nucleic acid barcode is a modified nucleotide.

11. The composition of claim 9, wherein the modification at the ribose group is independently for each occurrence selected from 2′-O-methyl, 2′-fluoro, 2′-F-arabino, 2′-methoxyethyl, 2′-amino, 2′-deoxy, 2′-O-allyl, locked nucleic acid, unlocked nucleic acid, 2′,4′-constrained 2′-O-ethyl-bridged nucleic acid, arabinose, hexose, cyclohexenyl nucleic acid, hexitol nucleic acid, glycol nucleic acid, 4′-thioribonucleoside, and 4′-C-aminomethyl-2′-O-methyl.

12. The composition of claim 9, wherein the modification at the phosphate group is independently for each occurrence selected from phosphorothioate, phosphorodithioate, alkylated phosphorothioate, boranophosphate, methylphosphonate, phosphoramidate, or amide backbone.

13. The composition of claim 9, wherein the modification at the nucleobase group is independently for each occurence selected from 5-methylcytosine, 5-hydroxymethylcytosine, 5-methyluracil, 5-ribosyluracil (pseudouracil), 7-methylguanine, inosine, xanthine, hypoxanthine, 3-methylcytidine, dihydrouridine, N6-methyldeoxyadenosine, N4-methylcytosine, 5-hydroxymethylcytosine, 5-formylcytosine, and 5-carboxylcytosine.

14. The composition of claim 9, wherein said nucleic acid barcode is a suboptimal substrate for nuclease-mediated degradation from one or more nucleases having enzyme commission numbers ranging from EC 3.1.11 to EC 3.1.31, such that the nucleic acid barcode is substantially protected from nuclease-mediated.

15. The composition of claim 14, wherein said nucleic acid barcode is a viable substrate of one or more nucleic acid binding enzymes having enzyme commission numbers other than EC 3.1.11 to EC 3.1.31.

16. The composition of claim 15, wherein said one or more nucleic acid binding enzymes are ligases that form phosphoric-ester bonds (E.C. 3.5.1).

17. The composition of claim 15, wherein said one or more nucleic acid binding enzymes are nucleotidyltransferases (E.C. 2.7.7).

18. The composition of claim 17, wherein said nucleotidyltransferases are selected from DNA-directed RNA polymerase (EC 2.7.7.6), DNA-directed DNA polymerase (EC 2.7.7.7), polynucleotide adenylyltransferase (EC 2.7.7.19), DNA nucleotidylexotransferase (terminal deoxyribonucleotidyl transferase) (EC 2.7.7.31), RNA-directed RNA polymerase (EC 2.7.7.48), RNA-directed DNA polymerase (reverse transcriptase) (EC 2.7.7.49), mRNA guanylyltransferase (mRNA capping enzyme) (EC 2.7.7.50), and combinations and recombinant and engineered versions thereof.

19. The composition of claim 15, wherein said one or more nucleic acid binding enzymes are one or more polynucleotide 5′-hydroxyl-kinase (EC 2.7.1.78).

20. The composition of claim 15, wherein said nucleic acid barcode comprises alternating 2′-O-methyl and 2′-fluoro nucleotides.

21. The composition of claim 15, wherein said nucleic acid barcode comprises at least 2, 3, 4, 5, 6, 7, 8, 9, or 10 contiguous 2′-O-methyl nucleotides.

22. The composition of claim 15, wherein said nucleic acid barcode comprises at least 2, 3, 4, 5, 6, 7, 8, 9, or 10 consecutive 2′-methoxyethyl nucleotides.

23. The composition of claim 15, wherein said nucleic acid barcode comprises at least one locked nucleic acid nucleotide.

24. The composition of claim 15, wherein each internucleotide linkage of said nucleic acid barcode is a phosphodiester internucleotide linkage or a phosphorothioate internucleotide linkage.

25. The composition of claim 24, wherein at least one internucleotide linkage of said nucleic acid barcode is a phosphorothioate internucleotide linkage.

26. The composition of claim 25, wherein the nucleotides at positions 1-2, 1-3, 1-4, 1-5, 1-6, 1-7 or 1-8 from the 3′ end of said nucleic acid barcode are connected to adjacent nucleotides via phosphorothioate linkages.

27. The composition of claim 25, wherein the nucleotides at positions 1-2, 1-3, 1-4, 1-5, 1-6, 1-7 or 1-8 from the 5′ end of said nucleic acid barcode are connected to adjacent nucleotides via phosphorothioate linkages.

28. The composition of claim 25, wherein the terminal 2, 3, 4, 5, 6, 7, 8, 9 or 10 nucleotides at both the 5′ and 3′ terminus of said nucleic acid barcode, independently, are linked with phosphorothioate internucleotide linkages.

29. The composition of any one of claims 1 to 28, wherein the nucleic acid analyte comprises a nucleic acid therapy (NAT).

30. The composition of any one of claims 1 to 29, wherein the nucleic acid analyte comprises a functional sequence which is recognized by a complementary hybridization or capture probe.

31. The composition of any one of claims 1 to 29, wherein the nucleic acid analyte is covalently conjugated to the nucleic acid barcode.

32. The composition of any one of claims 1 to 29, wherein the nucleic acid analyte is hybridized to the nucleic acid barcode.

33. The composition of any one of claims 1 to 29, wherein the nucleic acid barcode sequence is integrated within the sequence of the nucleic acid analyte.

34. The composition of any one of claims 1 to 33, wherein the biological sample is a sample obtained from a person that received formulation comprising the nucleic acid analyte.

35. The composition of claim 34, wherein the nucleic acid barcode of the nucleic acid analyte in the formulation is not encapsulated in a lipid nanoparticle formulation.

36. The composition of claim 35, wherein the nucleic acid analyte in the formulation is not encapsulated in a lipid nanoparticle formulation.

37. The composition of any one of claims 1 to 36, wherein the biological sample comprises cells.

38. The composition of claim 37, wherein cells in the biological sample internalize the nucleic acid analyte.

39. A composition comprising:
(i) a nucleic acid nanostructure self-assembled from two or more oligonucleotide strands that are not nucleic acid therapies (NATs), said nanostructure not encapsulated in the lipid nanoparticle formulation;
(ii) a cargo molecule that is linked to said nucleic acid nanostructure; and
(iii) optionally a nucleic acid barcode that is part of or linked to said nucleic acid nanostructure.

40. The composition of claim 39, wherein each of the two or more oligonucleotide strands of said nucleic acid nanostructure, independently, comprises DNA, RNA, XNA or a combination thereof.

41. The composition of claim 40, wherein at least one of the two or more oligonucleotide strands of said nucleic acid nanostructure comprises one or more modified nucleotides, and wherein each of said one or more modified nucleotides is independently modified at the ribose group, the phosphate group, the nucleobase group, or a combination thereof.

42. The composition of claim 41, wherein each of the at least one modified nucleotides is independently selected from a 2′-O-methyl modified nucleotide, a 2′-fluoro modified nucleotide, a 2′-deoxy nucleotide, a 2′-hydroxyl nucleotide, a 2′-F-arabino nucleotide, a 2′-methoxyethyl modified nucleotide, a locked nucleotide, an unlocked nucleotide, a constrained ethyl nucleotide [2′,4′-constrained 2′-O-ethyl-bridged nucleic acid], a 2′-amino-modified nucleotide, a 2′-O-allyl modified nucleotide, a morpholino nucleotide, a phosphoramidate nucleotide, an arabinose modified nucleotide, a cyclohexenyl modified nucleotide, a hexose modified nucleotide, a hexitol modified nucleotide, an altritol modified nucleotide, a glycol modified nucleotide, a 4′ thiol modified nucleotide, a 4′-C-aminomethyl-2′-O-methyl modified nucleotide, a nucleotide comprising a phosphorothioate group, a nucleotide comprising a phosphorodithioate group, a nucleotide comprising an alkylated phosphorothioate group, a nucleotide comprising a methylphosphonate group, a nucleotide comprising a boranophosphate group, a nucleotide comprising a phosphoramidate group, a nucleotide comprising an amide group, a nucleotide comprising a 5′-phosphate, a 5-methylcytosine nucleotide, a 5-hydroxymethylcytosine nucleotide, a 5-methyluracil nucleotide, a 5-ribosyluracil (pseudouracil) nucleotide, a 7-methylguanine nucleotide, an inosine nucleotide, a xanthine nucleotide, a hypoxanthine nucleotide, a 3-methylcytidine nucleotide, a dihydrouridine nucleotide, a N6-methyldeoxyadenosine nucleotide, a N4-methylcytosine nucleotide, a 5-hydroxymethylcytosine nucleotide, a 5-formylcytosine nucleotdie, a 5-carboxylcytosine nucleotide, a 3′-terminal deoxy-thymine (dT) nucleotide, an abasic nucleotide, and combinations thereof.

43. The composition of claim 41, wherein:
each nucleotide of the nucleic acid nanostructure is modified; or
each nucleotide of the nucleic acid nanostructure and/or each nucleotide of the barcode is modified.

44. The composition of claim 41, wherein said nucleic acid nanostructure comprises one or more 2′O-modified nucleotides, one or more phosphorothioate internucleotide linkages or a combination thereof.

45. The composition of claim 44, wherein the one or more 2′O modifications, the one or more phosphorothioate internucleotide linkages or the combination of both are used to modulate the physicochemical properties of the nucleic acid nanostructure.

46. The composition of claim 40, wherein between 1 and 500 cargo molecules are linked to said nucleic acid nanostructure.

47. The composition of claim 46, wherein 1, 2, 3, 4, 5, 6, 7, 8, 9 or 10 cargo molecules are linked to said nucleic acid nanostructure.

48. The composition of claim 46, wherein each of the at least two or more oligonucleotide strands of the nucleic acid nanostructure is independently linked to one cargo molecule.

49. The composition of claim 40, wherein the cargo molecule is selected from the group comprising one or more NAT, nucleic acid barcode, peptide, protein, antibody, therapeutic small molecule, lipid, cholesterol, synthetic polymer, amino acid, amino acid analogue, PEG and hydrocarbon chain.

50. The composition of claim 49, wherein at least one of the two or more oligonucleotides of said nucleic acid nanostructure is linked to one or more NAT.

51. The composition of claim 45, wherein the one or more NAT is an ASO, an siRNA, a gRNA/CRISPR, an saRNA, or an mRNA.

52. The composition of claim 49, wherein the cargo molecule comprises a NAT and a nucleic acid barcode.

53. The composition of claim 40, wherein at least one of the two or more oligonucleotide strands of the nucleic acid nanostructure is conjugated to the cargo molecule via a click reaction selected from the group comprising CuAAC, SPAAC, RuAAC, IEDDA, SuFEx, SPANC, hydrazone/oxime ether formation, thiol-ene radical reaction, thiol-yne radical reaction, thiol-Michael addition reaction, thiol-isocyanate reaction, thiol-epoxide click reaction, nucleophilic ring opening reaction (spring-loaded reactions), and traceless Staudinger ligation.

54. The composition of claim 53, wherein the nucleic acid nanostructure is conjugated to the cargo molecule via CuAAC.

55. The composition of claim 53, wherein the nucleic acid nanostructure is conjugated to the cargo molecule via IEDDA.

56. The composition of claim 40, wherein the nucleic acid nanostructure comprises at least one structural motif selected from duplex, triplex, quadruplex, hairpin loop, internal loop/bulge/helix-loop-helix (such as kink-turn, S-turn T-loop), multi-branched loop/multi-junction, pseudoknot, kissing loop, A-minor, A-A platform, tetraloop, ribose zipper and combinations thereof.

57. The composition of claim 56, wherein at least one of the two or more oligonucleotide strands of said nucleic acid nanostructure has either partial or complete sequence complementarity to at least one other oligonucleotide of the nucleic acid nanostructure.

58. The composition of claim 57, wherein the nucleic acid nanostructure is self-assembled from two oligonucleotide strands and comprises a single duplex motif.

59. The composition of claim 57, wherein the nucleic acid nanostructure is self-assembled from three or more oligonucleotide strands and comprises a single duplex motif.

60. The composition of claim 57, wherein the nucleic acid nanostructure is self-assembled from two or more oligonucleotide strands and comprises at least one duplex motif and at least one hairpin loop motif.

61. The composition of claim 57, wherein the nucleic acid nanostructure is self-assembled from n (or more) oligonucleotide strands and comprises one n-way junction motif, where n can be any integer between 3 and 20.

62. The composition of claim 57, wherein the nucleic acid nanostructure is self-assembled from 10 or fewer oligonucleotide strands and comprises two or more n-way junction motifs, where n can be any integer between 3 and 20.

63. The composition of claim 57, wherein the nucleic acid nanostructure comprises one or more circularized oligonucleotide strands.

64. The composition of claim 39, wherein the cargo molecule is the nucleic acid barcode.

65. The composition of claim 39, wherein the nucleic acid barcode is incorporated within at least one of the two or more oligonucleotide strands of the nucleic acid nanostructure.

66. The composition of claim 65, wherein the nucleic acid barcode is incorporated at the 5′ or the 3′ terminus within at least one of the two or more oligonucleotide strands of the nucleic acid nanostructure.

67. The composition of claim 65, wherein the nucleic acid barcode is incorporated in an internal loop or bulge motif of the nucleic acid nanostructure.

68. The composition of claim 65, wherein the nucleic acid barcode and its reverse complement are incorporated in a duplex motif of the nucleic acid nanostructure.

69. The composition of any one of claims 39 to 68, wherein each of the two or more oligonucleotide strands of said nucleic acid nanostructure, independently, are between 5 to 500 nucleotides in length.

70. The composition of claim 69, wherein each of the two or more oligonucleotide strands of said nucleic acid nanostructure, independently, are between 5 to 80 nucleotides in length.

71. The composition of claim 69, wherein each of the two or more oligonucleotide strands of said nucleic acid nanostructure, independently, are no more than 35 nucleotides in length.

72. A composition comprising an oligonucleotide of formula (I), or a salt thereof: 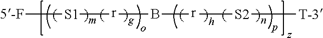
wherein:
F is an optional 5′ terminal cargo that is either absent or present;
T is an optional 3′ terminal cargo that is either absent or present;
z is 1 or any positive integer greater than 1;
and p are each independently, and independently for each occurrence, 0 or any positive integer greater than 0;
m, g, h, and n are each independently, and independently for each occurrence, 0 or 1;
for any o>l, g≤m for each but the first occurrence;
for any p>l, h≤n for each but the first occurrence;
S1 and S2 are nucleotide sequences, each independently comprising at least 1 and not more than 100,000 nucleotides in length, where for any o>1 or p>1 or z>1 or combinations thereof:
(i) each occurrence of S1, independently, represents a nucleic acid sequence identical to or different to the nucleic acid sequence of the first occurrence of S1;
(ii) each occurrence of S2, independently, represents a nucleic acid sequence identical to or different to the nucleic acid sequence of the first occurrence of S2;
r is a covalent or non-covalent linkage, where for any o>1 or p>1 or z>1 or combinations thereof each occurrence of r represents a linkage identical to or different to the linkage of the first occurrence of r; and
B is a nucleic acid barcode comprising a nucleic acid sequence, where for z>1 each occurrence of B, independently, comprises a nucleic acid sequence identical to or different to the nucleic acid sequence of the first occurrence of B; and
wherein said oligonucleotide of formula (I) is not encapsulated in a lipid nanoparticle formulation.

73. The composition of claim 72, wherein the oligonucleotide of formula (I) comprises a NAT.

74. The composition of claim 72, wherein the oligonucleotide of formula (I) comprises one or more modified nucleotides, and wherein each of said one or more modified nucleotides is independently modified at the ribose group, the phosphate group, the nucleobase group, or a combination thereof.

75. The composition of claim 72, wherein r is a bioconjugation linkage formed as product of a conjugation reaction, and is independently for each occurrence selected from an acid anhydride, an alkane, an alkene, an alkyne, an amide, an amine, a disulfide, a dithiourethane, an ether, ethylene glycol, an ester, a glucosyl, a hydrazone, an imine, an iminophosphorane, an isoxazoline, a ketone, an oxime, a phosphodiester, a phosphoramidate, a polyamide, a pyridazine, a pyrrolidine, a sulfonate ester, a sulfonamide, a thioether, a thiourethane, and a triazole linkage.

76. The composition of claim 75, wherein r comprises a cleavable linkage.

77. The composition of claim 72, wherein r is a hybridization linkage.

78. The composition of claim 72, wherein S1 and/or S2 independently comprise a nucleic acid sequence selected from a NAT, a nucleic acid nanostructure, a random nucleotide sequence, a synthetic non-therapeutic nucleic acid, a probe hybridization sequence, a primer binding sequence, a cellular RNA sequence and combinations thereof.

79. The composition of claim 78, wherein the nucleic acid sequence of at least one of S1 and S2 independently comprises an mRNA, gRNA/CRISPR, saRNA, siRNA, ASO, miRNA, ribozyme, or aptamer sequence.

80. The composition of claim 78, wherein the nucleic acid sequence of at least one of S1 and S2 independently comprises an oligonucleotide sequence of a nucleic acid nanostructure self-assembled from two or more oligonucleotide strands that are not NATs.

81. The composition of claim 78, wherein
(i) the nucleic acid sequence of at least one of S1 and S2 independently comprises a unique molecular identifier sequence of random nucleotides; and
(ii) each random nucleotide of the unique molecular identifier is optionally modified at the ribose group, the phosphate group, the nucleobase group or combinations thereof.

82. The composition of claim 78, wherein the nucleic acid sequence of at least one of S1 and S2 independently comprises a sequence complementary to a hybridisation probe selected from a biotinylated capture probe, a molecular beacon probe, a ligation probe, a Scorpion probe, a TaqMan probe, an LNA probe, a cycling probe, and an RNAscope probe.

83. The composition of claim 78, wherein the nucleic acid sequence of at least one of S1 and S2, independently, is designed to have no homology with endogenous nucleic acid sequences of the biological subject of interest.

84. The composition of claim 78, wherein the nucleic acid sequence at least one of S1 and S2, independently, comprises one or more modified nucleotides.

85. The composition of claim 72, wherein the optional cargo molecules F and T are, independently, selected from the group comprising NAT, peptide, protein, antibody, small molecule, lipid, cholesterol, synthetic polymer, amino acid, amino acid analogue, fluorophore, multivalent moiety, PEG and hydrocarbon chain.

86. The composition of claim 85, wherein at least one of said cargo molecules F and T, independently, comprises one or more GalNAc derivatives attached through a monovalent, bivalent or trivalent linker.

87. The composition of claim 85, wherein at least one of said cargo molecules F and T, independently, comprises one or more lipid derivatives attached through a cleavable or non-cleavable linker.

88. The composition of claim 85, wherein at least one of said cargo molecules F and T, independently, comprises a cell-penetrating peptide through a cleavable or non-cleavable linker.

89. The composition of claim 72, wherein F comprises a nucleotide modified with a 5′ terminal modification selected from 5′ App, 5′ spacer, 5′ fluorophore, 5′ quencher, 5′ phosphate, 5′ biotin, 5′ digoxigenin and chemical analogs thereof.

90. The composition of claim 72, wherein T comprises a nucleotide modified with a 3′ terminal modification selected from 3′ inverted dT, 3′ spacer, 3′ fluorophore, 3′ quencher, 3′ phosphate, 3′ biotin and chemical analogs thereof.

91. The composition of claim 72, wherein z is 1.

92. The compound of claim 72 or 91, wherein o is 0 and p is 1.

93. The composition of claim 92, wherein h is 0 and n is 1.

94. The composition of claim 92, wherein both h and n are 1.

95. The compound of claim 72 or 91, wherein o is 1 and p is 0.

96. The composition of claim 95, wherein m is 1 and g is 0.

97. The composition of claim 95, wherein both m and g are 1.

98. The compound of claim 72 or 91, wherein both o and p are 1.

99. The composition of claim 98, wherein both m and n are 1, and both g and h are 0.

100. The composition of claim 98, wherein both m and n are 1, and g is 1, and h is 0.

101. The composition of claim 98, wherein both m and n are 1, and g is 0, and h is 1.

102. The composition of claim 72, wherein
(i) the nucleic acid sequence of at least one of S1 and S2 comprises one or more modified nucleotides arranged in a modification pattern;
(ii) B is of the same length as either S1 or S2; and
(ii) B comprises the same modification pattern as either S1, S2 or both S1 and S2.

103. The composition of any one of claims 67 to 97, wherein the oligonucleotide of formula (I) comprises part of a library of barcoded nucleic acid analytes for use in a multiplex screening assay.

104. The composition of claim 103, wherein each of the barcoded nucleic acid analytes of said library comprises a NAT for use in a multiplex assay that achieves screening for the delivery of NATs.

105. The composition of claim 103, wherein each of the barcoded nucleic acid analytes of said library comprises a nucleic acid barcode sequence B that differs in at least one nucleotide from all other sequences of B.

106. The composition of claim 105, wherein B comprises the sequence of a NAT selected from ASO, siRNA, saRNA and gRNA/CRISPR, such that a plurality of different NAT sequences can be screened in a multiplex assay.

107. The composition of claim 106, wherein z is 1 and both o and p are 0.

108. The composition of claim 105, wherein B comprises an identifier sequence that corresponds to a particular modification pattern of the nucleic acid sequence of S1, S2 or both S1 and S2 for allowing screening of a plurality of differently modified oligonucleotides in a multiplex assay.

109. The composition of claim 105, wherein B comprises an identifier sequence that corresponds to a particular cargo molecule F or T or both for allowing screening of a plurality of oligonucleotides linked to different cargo molecules in a multiplex assay.

110. A composition comprising:
an alkyl phosphonamidite of formula (II): 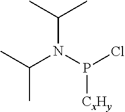
wherein CₓH_{Y} denotes an alkyl chain with the general formula CₙH₂ₙ₋₁, where x can be anywhere between 1 and 20 and x can be anywhere between 3 and 39.

111. The composition of claim 110, wherein the methyl phosphonamidite is incorporated into an oligonucleotide.

112. The composition of claim 111, wherein the oligonucleotide is incorporated into a nucleic acid nanostructure.

113. A composition comprising:
an alkyl phosphonamidite of formula (III): 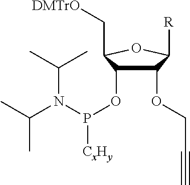
wherein R is a nucleotide base and CₓH_{Y} denotes an alkyl chain with the general formula CₙH₂ₙ₋₁, where x can be anywhere between 1 and 20 and x can be anywhere between 3 and 39.

114. The composition of claim 113, wherein the methyl phosphonamidite is incorporated into an oligonucleotide.

115. The composition of claim 114, wherein the oligonucleotide is incorporated into a nucleic acid nanostructure.

116. A composition comprising:
a phosphitylating reagent of formula (IV): 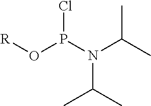
wherein R is a moiety that can modulate the physicochemical properties of an oligonucleotide. This includes moieties selected from the group consisting of O-alkylamino, O-alkylalkoxy, protected O-alkylamino, O-alkylaminoalkyl, O-alkyl imidazole, O-alkyl guanidine, polyamines of the formula (O-alkyl)ₘ, where m is about 1 to 20 and polyethers of the formula (O-alkyl)ₘ, where m is about 1 to 20. Preferred ethers are polyethylene glycols (PEGs).

117. The composition of claim 116, wherein the phosphitylating reagent is used to convert an alcohol-bearing moiety to a phosphoramidite of formula (V): 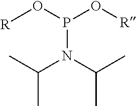
wherein R is a moiety that can modulate the physicochemical properties of an oligonucleotide. This includes moieties selected from the group consisting of O-alkylamino, O-alkylalkoxy, protected O-alkylamino, O-alkylaminoalkyl, O-alkyl imidazole, O-alkyl guanidine, polyamines of the formula (O-alkyl)ₘ, where m is about 1 to 20 and polyethers of the formula (O-alkyl)ₘ, where m is about 1 to 20. Preferred ethers are polyethylene glycols (PEGs). R″ is the alcohol-bearing molecule. R″ could be any molecule selected from the group consisting of, but not limited to, nucleotides, nucleosides, alkyl chains, PEGs, lipids, cholesterols, polymers, peptides, amino acids.

118. The composition of claim 117, wherein the phosphoramidite is incorporated into an oligonucleotide.

119. The composition of claim 118, wherein the oligonucleotide is incorporated into a nucleic acid nanostructure.

120. The composition of claim 116, wherein there is a labile linker between the modification (R), and the P(III) center, as given by general formula (VI): 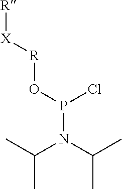
wherein R is a moiety that can modulate the physicochemical properties of an oligonucleotide. This includes moieties selected from the group consisting of O-alkylamino, O-alkylalkoxy, protected O-alkylamino, O-alkylaminoalkyl, O-alkyl imidazole, O-alkyl guanidine, polyamines of the formula (O-alkyl)ₘ, where m is about 1 to 20. X is a labile linker. Linkers include those, but are not limited to, moieties selected from the group consisting of thiol cleavable linkers such as dithiobismaleimidoethane, 1,4-bis[3-(2-pyridyldithio)propionamido]butane and 3-(2-pyridyldithio)propionyl hydrazide or base-cleavable linkers such as bis[2-(N-succinimidyl-oxycarbonyloxy)ethyl]sulfone or hydroxylamine-cleavable linkers such as (ethylene glycol bis(succinimidyl succinate)), oximes, hydrazones or cathepsin-responsive dipeptides. R″ is a moiety that can mask the properties of R. This includes moieties from the group consisting of, but is not limited to, polyethers of the formula (O-alkyl)ₘ, where m is about 1 to 20, carbohydrates and hydrophilic peptides.

121. A composition comprising:
a nucleic acid nanostructure not formulated in a lipid nanoparticle comprising or linked to a nucleic acid barcode, and
optionally, a cargo molecule that is covalently linked to the nucleic acid nanostructure.

122. The composition of claim 121, wherein the cargo molecule is selected from the group consisting of a NAT, nucleic acid barcode, peptide, protein, antibody, therapeutic small molecule, lipid, cholesterol, synthetic polymer, amino acid, amino acid analogue, PEGS and hydrocarbon chain.

123. The composition of claim 121, wherein the cargo molecule is covalently linked to the nucleic acid nanostructure by a cleavable linker.

124. The composition of claim 123, wherein the cleavable linker is a dipeptide selected from the group consisting of Tyr-Arg, Phe-Arg, Val-Gln, Val-Cit, Tyr-Met, Leu-Gln, Val-Arg, Met-Thr, Phe-Gln, Thr-Thr, Val-Thr, Ala-Ala, Val-Met, Leu-Met, Ala-Asn, Val-Ala, Phe-Met.

125. The composition of claim 124, wherein the cleavable linker is cleaved enzymatically by an enzyme selected from the group consisting of cathepsin A, cathepsin B, cathepsin, C, cathepsin D, cathepsin E, cathepsin F, cathepsin G, cathepsin H, cathepsin K, cathepsin L1, cathepsin L2, cathepsin O, cathepsin S, cathepsin W and cathepsin Z.

126. The composition of claim 124, wherein the dipeptide is further attached to a moiety that will allow for traceless release of the cargo.

127. The composition of claim 126, wherein the moiety that allows for traceless release of the cargo is para-aminocarbamate.

128. The composition of claim 126, wherein the moiety that allows for traceless release is selected from the group consisting of benzyl carbamate, hydroxylbenzylamine, hydrazone, disulfide and pyrophosphate diester.

129. The composition of claim 124, wherein the dipeptide unit has a phosphoramidite incorporated within the structure to allow for attachment to the oligonucleotide by solid-phase oligonucleotide synthesis.

130. The composition of claim 124, wherein the dipeptide unit has a reactive click handle incorporated within the structure to allow for attachment to the oligonucleotide via click chemistry.

131. The composition of claim 129, wherein the dipeptide unit has a further reactive click handle incorporated within the structure to allow for attachment to the cargo via click chemistry.

132. The composition of claim 131, wherein the reactive is groups are orthogonal to each other and are reactive in a reaction selected from the group consisting of CuAAC, SPAAC, RuAAC, IEDDA, SuFEx, SPANC, hydrazone/oxime ether formation, thiol-ene radical reaction, thiol-yne radical reaction, thiol-Michael addition reaction, thiol-isocyanate reaction, thiol-epoxide click reaction, nucleophilic ring opening reaction (spring-loaded reactions), and traceless Staudinger ligation.

133. The composition of claim 130, wherein the dipeptide unit has both a phosphoramidite moiety and a click moiety. The click moiety is reactive in a reaction selected from the group consisting of CuAAC, SPAAC, RuAAC, IEDDA, SuFEx, SPANC, hydrazone/oxime ether formation, thiol-ene radical reaction, thiol-yne radical reaction, thiol-Michael addition reaction, thiol-isocyanate reaction, thiol-epoxide click reaction, nucleophilic ring opening reaction (spring-loaded reactions), and traceless Staudinger ligation.

134. A composition comprising:
a nucleic acid nanostructure, and
a layer of hydrophilic molecules that are electrostatically bound to the nucleic acid phosphate backbone.

135. The composition of claim 134, wherein the hydrophilic layer consists of multiple molecules with the same identity. This number is given by n(P)-X, wherein n(P) is the number of phosphate moieties in the molecule and X can be any number between 1 and n(P).

136. The composition of claim 134, wherein the hydrophilic layer consists of multiple molecules with different identities. There could be 1, 2, 3, 4, 5, 6, 7, 8, 9, 10 or more different types of molecules in varied molar ratios.

137. The composition of claim 134, wherein the nucleic acid nanostructure comprises DNA, RNA, or xeno-nucleic acid (XNA).

138. The composition of claim 134, wherein the electrostatic molecule contains a primary amine that can displace 4-(hexyloxy)anilinium on the nucleic acid phosphate backbone.

139. The composition of claim 138, wherein the electrostatic molecule is a PEG molecule with the general formula (VII): 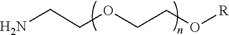
wherein R is H or Me.

140. The composition of claim 138, wherein the electrostatic molecule is a PEG molecule with the general formula (VIII):

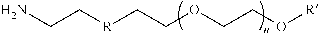

141. The composition of claim 140, wherein R is a functional group that will allow for cleavage of the hydrocarbon chain in response to a range of stimuli, selected from the group consisting of disulfide, hydrazone, hydrazine, acetal, benzoic imine or thioketal.

142. The composition of claim 140, wherein R′ is a hydrogen or methyl group.

143. The composition of claim 138, wherein the electrostatic molecule is a PEG molecule with the general formula (IX):

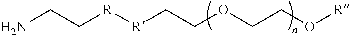

144. The composition of claim 143, wherein R is defined as the “physicochemical modulator”, which is subsequently masked by the PEG moiety. This moiety can include, but is not limited to, spermine, ethylenediamine, methylethylenediamine, ethylethylenediamine, imidazole, spermine-imidazole-4-imine, N-ethyl-N′-(3-dimethylaminopropyl)-guanidinyl ethylene imine, dimethylaminoethyl acrylate, amino vinyl ether, 4-imidazoleacetic acid, diethylaminopropylamide, sulfonamides (e.g. sulfadimethoxine sulfamethoxazole, sulfadiazine, sulfamethazine), amino ketals, N-2-hydroxylpropyltimehyl ammonium chloride, imidazole-4-imines, methyl-imidazoles, 2-(aminomethyl)imidazole, 4-(aminomethyl)imidazole, 4(5)-(hydroxymethyl)imidazole, N-(2-aminoethyl)-3-((2-aminoethyl)(methyl)amino)propanamide, 2-(2-ethoxyethoxy)ethan-1-amine, bis(3-aminopropyl)amine, [N,N-dimethylamino)ethoxy]ethyl, N-(2-aminoethyl)-3-((2-aminoethyl)(ethyl)amino)propanamide, (N-(aminoethyl)carbamoyl)methyl, N-(2-((2-aminoethyl)amino)ethyl)acetamide 3,3′-((2-aminoethyl)azanediyl)bis(N-(2-aminoethyl)propanamide), guanidyl benzylamide, [3-(guanidinium)propyl], dimethylethanolamine, 1-(2,2-dimethyl-1,3-dioxolan-4-yl)-N,N-dimethylmethanamine, 2-(2,2-dimethyl-1,3-dioxolan-4-yl)-N,N-dimethylethan-1-amine, N-(2-((2-(2-aminoethoxy)propan-2-yl)oxy)ethyl)acetamide, aminobutyl, aminoethyl, 1-(2-aminoethyl)-3-(3-(dimethylamino)propyl)-2-ethylguanidine, 1-(3-amino-3-oxopropyl)-2,4,6-trimethylpyridin-1-ium, 1-(1,3-bis(carboxyoxy)propan-2-yl)-2,4,6-trimethylpyridin-1-ium, guanidinylethyl amine, ether hydroxyl triazole, or a β-aminoester or an alkyl chain between 1 and 30 carbons in length.

145. The composition of claim 133, wherein R′ is defined as a functional group that will allow for cleavage of the hydrocarbon chain in response to a range of stimuli, selected from the group consisting of disulfide, hydrazone, hydrazine, acetal, benzoic imine or thioketal.

146. The composition of claim 138, wherein the electrostatic molecule is a branched PEG molecule with the general formula:

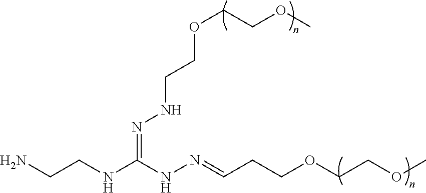

147. The composition of claim 146, wherein the PEG chain length (n) could be anywhere between 1, 2, 3, 4, 5, 6, 7, 8, 9, 10, 15, 20, 30 units long or more.

148. The compositions of claims 139, 140, 143 and 146, wherein the electrostatic molecules are instead covalently bound to the nucleic acid nanostructure.

149. The composition of claim 134, wherein the electrostatic molecule is a sugar molecule that has been functionalized with a primary amine.

150. Composition of claim 149, wherein the sugar molecule is selected from the group consisting of D-Ribulose 5-phosphate, D-Mannuronic acid, L-Fuconic acid, 1-Deoxy-D-xylulose-5-phosphate, D-Fructose-1,2-cyclic-6-bisphosphate, D-Erythritol 4-phosphate, D-galactose 6-phosphate, 2-Deoxy-D-ribonic acid, 6-Phosphogluconic acid, β-L-Fucose 1-phosphate bis(cyclohexylammonium), D-Fructose 1,6-bisphosphate, D-Mannose 6-phosphate, D-Xylonic acid, L-Glyceric acid, L-Threonic acid, 6-phospho-D-galactonate, N-Acetyl-D-glucosamine, D-Glyceric acid, D-Mannose, D-Ribose, Agaric acid, D-Mannitol, Dulcitol, L-Sorbose, D-Glucose diethylmercaptal, Dhurrin, Levoglucosenone, α-Homonojirimycin, Arbutin, Fusicoccin, L-(−)-Dithiothreitol, L-(+)-Ribose, D-(−)-Lactic acid, D-(−)-Tagatose, D-Mannoheptose, D-Psicose, D-(+)-Fucose, D-(+)-Sorbose, DL-Arabinose, L-(−)-Galactose, L-(+)-Erythrulose, L-(−)-Fucose, 3-Deoxyglucosone, D-(+)-Arabitol, D-Mannoheptulose, DL-Xylose, epi-Inositol, L-(−)-Glucose, L-(−)-Mannose, L-(+)-Gulose, Lactulose, Methyl α-D-galactopyranoside, 1,5-Anhydro-D-sorbitol, Isopropyl β-D-thioglucopyranoside, L(+)-Erythrose, Methyl-β-D-galactopyranoside, Methyl-β-D-thiogalactoside, N-Acetylmuramic acid, β-D-Allose, D-(+)-Talose, D-Glucosaminic acid, Deoxyfuconojirimycin, 2-Deoxy-L-ribose, L-Galactono-1,4-lactone, L-Idaro-1,4-lactone, N-Acetyl-D-glucosamine, (−)-Sinigrin hydrate, 5-Thio-D-glucose, D-Threono-1,4-lactone, D-Xylono-1,4-lactone, D-(−)-Erythrose, L-Threono-1,4-lactone, Methyl α-D-mannopyranoside, D-Glucosone, N-Glycolylneuraminic acid, 2-Deoxy-D-ribose, 3-O-Methyl-D-glucopyranose, D-Glucosamine 3-sulfate, D-Altrose, D-(−)-Fructose, D-(+)-Xylose, Glycerol, D-Galactose, L-(−)-Sorbose, meso-Erythritol, Methyl α-D-glucopyranoside, Dextrose, L-Rhamnose, N-Acetyl-D-galactosamine, L-(+)-Arabinose.

151. The composition of claim 134, wherein the electrostatic molecule is an oligosaccharide, consisting of 0, 1, 2, 3, 4, 5, 6, 7, 8, 9, 10, 15, 20 repeating units or more.

152. The composition of claim 151, wherein the monomeric sugar molecule is selected from the group consisting of D-Ribulose 5-phosphate, D-Mannuronic acid, L-Fuconic acid, 1-Deoxy-D-xylulose-5-phosphate, D-Fructose-1,2-cyclic-6-bisphosphate, D-Erythritol 4-phosphate, D-galactose 6-phosphate, 2-Deoxy-D-ribonic acid, 6-Phosphogluconic acid, β-L-Fucose 1-phosphate bis(cyclohexylammonium), D-Fructose 1,6-bisphosphate, D-Mannose 6-phosphate, D-Xylonic acid, L-Glyceric acid, L-Threonic acid, 6-phospho-D-galactonate, N-Acetyl-D-glucosamine, D-Glyceric acid, D-Mannose, D-Ribose, Agaric acid, D-Mannitol, Dulcitol, L-Sorbose, D-Glucose diethylmercaptal, Dhurrin, Levoglucosenone, α-Homonojirimycin, Arbutin, Fusicoccin, L-(−)-Dithiothreitol, L-(+)-Ribose, D-(−)-Lactic acid, D-(−)-Tagatose, D-Mannoheptose, D-Psicose, D-(+)-Fucose, D-(+)-Sorbose, DL-Arabinose, L-(−)-Galactose, L-(+)-Erythrulose, L-(−)-Fucose, 3-Deoxyglucosone, D-(+)-Arabitol, D-Mannoheptulose, DL-Xylose, epi-Inositol, L-(−)-Glucose, L-(−)-Mannose, L-(+)-Gulose, Lactulose, Methyl α-D-galactopyranoside, 1,5-Anhydro-D-sorbitol, Isopropyl β-D-thioglucopyranoside, L(+)-Erythrose, Methyl-β-D-galactopyranoside, Methyl-β-D-thiogalactoside, N-Acetylmuramic acid, β-D-Allose, D-(+)-Talose, D-Glucosaminic acid, Deoxyfuconojirimycin, 2-Deoxy-L-ribose, L-Galactono-1,4-lactone, L-Idaro-1,4-lactone, N-Acetyl-D-glucosamine, (−)-Sinigrin hydrate, 5-Thio-D-glucose, D-Threono-1,4-lactone, D-Xylono-1,4-lactone, D-(−)-Erythrose, L-Threono-1,4-lactone, Methyl α-D-mannopyranoside, D-Glucosone, N-Glycolylneuraminic acid, 2-Deoxy-D-ribose, 3-O-Methyl-D-glucopyranose, D-Glucosamine 3-sulfate, D-Altrose, D-(−)-Fructose, D-(+)-Xylose, Glycerol, D-Galactose, L-(−)-Sorbose, meso-Erythritol, Methyl α-D-glucopyranoside, Dextrose, L-Rhamnose, N-Acetyl-D-galactosamine, L-(+)-Arabinose.

153. The composition of claim 134, wherein the electrostatic molecule is a hydrophilic peptide.

154. The composition of claim 153, wherein the N-terminus is conjugated to a linker or PEG spacer with a primary amine terminus.

155. The composition of claim 134, wherein the electrostatic molecule is a hydrophilic polymer with the general formula:

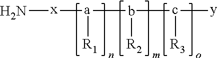

156. The composition of claim 155, wherein x is a linker that may or may not be attached to a chain transfer agent, y is the polymer terminus, a, b and c are polymer blocks consisting of repeating units R₁, R₂ and R₃. These may be block copolymers or statistical or random copolymers, n, m and o are integers between 1 and 500.

157. The composition of claim 156, wherein R₁=R₂=R₃.

158. The composition of claim 156, wherein R₁≠R₂≠R₃.

159. The composition of claim 156, wherein R₁=R₂≠R₃.

160. The composition of claim 156, wherein R₁≠R₂=R₃.

161. A composition comprising
a nucleic acid nanostructure, and
a layer of molecules that can mediate endosomal escape, which are electrostatically bound to the nucleic acid phosphate backbone.

162. The composition of claim 161, wherein the electrostatic molecules are compounds, or derivatives, of the group selected from chloroquine, 1-[1-(6-Chloroquinolin-4-yl)piperidin-4-yl]piperidin-3-ol, 1-(7-chloroquinolin-4-yl)piperidin-4-ol, 2-[4-(7-Chloroquinolin-4-yl) morpholin-2-yl]ethanamine, [1-(7-Chloroquinolin-4-yl)piperidin-3-yl]methanol, 1R,2R)-2-N-(7-Chloroquinolin-4-yl)cyclohexane-1,2-diamine, (1S,2S)-2-N-(7-chloroquinolin-4-yl)cyclohexane-1,2-diamine, N′-(7-chloroquinolin-4-yl)-N-cyclohexylethane-1,2-diamine, N-(4-aminobutyl)-7-chloroquinolin-4-amine, (1R,2S)-2-N-(7-chloroquinolin-4-yl)cyclohexane-1,2-diamine, N′-(7-Chloroquinolin-4-yl)-N-ethylpropane-1,3-diamine, N-(7-Chloroquinolin-4-yl)-N′,N′-dimethylbutane-1,4-diamine, N4-(7-chloroquinolin-4-yl)-n1-methylpentane-1,4-diamine, Didesethyl Chloroquine, 3-N-(7-chloroquinolin-4-yl)-1-N,1-N-dimethylbutane-1,3-diamine, N′-(7-Chloroquinolin-4-yl)-N-(2-methylpropyl)propane-1,3-diamine, Desethyl Chloroquine, N′-(7-chloroquinolin-4-yl)-N-cyclohexylpropane-1,3-diamine, 1-(7-Chloroquinolin-4-yl)-N,N-dimethylpiperidin-3-amine, N-(7-chloroquinolin-4-yl)-N′,N′-diethylpropane-1,3-diamine, (3R)-1-(7-chloroquinolin-4-yl)azepan-3-amine, N-(7-chloroquinolin-4-yl)-N′-propan-2-ylpropane-1,3-diamine, 1-(7-chloroquinolin-4-yl)azepan-3-amine, N-(7-Chloroquinolin-4-yl)-N′,N′-di(propan-2-yl)ethane-1,2-diamine, N-(7-chloroquinolin-4-yl)-N′,N′-dimethylpropane-1,3-diamine, N′-(7-Chloroquinolin-4-yl)-N,N-diethylethane-1,2-diamine, 1-N-(7-Chloroquinolin-4-yl)-2-N,2-N-dimethylpropane-1,2-diamine, (1-Quinolin-4-ylpiperidin-4-yl) methanamine, N-(6-Chloroquinolin-4-yl)-N′,N′-diethylpropane-1,3-diamine, N-(7-Chloroquinolin-4-yl)-N′-methylpropane-1,3-diamine, (E)-N-(7-Chloroquinolin-4-yl)-N′,N′-diethylbut-2-ene-1,4-diamine, 7-Chloro-N-(5-pyrrolidin-1-ylpentan-2-yl)quinolin-4-amine. ethyl (8-((2-(dimethylamino)ethyl)amino)-2,3-diphenylpyrido[2,3-b]pyrazin-6-yl)carbamate or ethyl (8-((2-(dimethylamino)ethyl)amino)-2,3-diphenylpyrido[2,3-b]pyrazin-6-yl)carbamate.

163. The composition of claim 162, wherein the molecules that mediate endosomal escape are formulated with any of the hydrophilic molecules given in claims 139, 140, 143, 146, 149, 153 or 155. These could be formulated with 1, 2, 3, 4, 5, 6, 7, 8, 9, 10 or more different types of molecules in varied molar ratios.

164. A composition comprising
a nucleic acid nanostructure, and
a layer of primary amine-containing small molecules that have a therapeutic effect.

165. The composition of claim 164, wherein the small molecule is selected from the group consisting of aciclovir, adefovir dipivoxil, alfuzosin, amiloride, aminosalicylic acid, amisulpride, amlexanox, amprenavir, amrinone, anileridine, azacitidine, benzocaine, bleomycin, bromfenac, cefdinir, cefditoren, cefepime, cefixime, cefmenoxime, cefotaxime, cefpodoxime, ceftazidime, ceftriaxone, chloriprocaine, cidofovir, cladribine, clenbuterol, clofarabine, cytarabine, dactinomycin, dapsone, darunavir, decitabine, doxazosin, emtricitabine, entecavir, famciclovir, flucytosine, fludarabine, fosamprenavir, ganciclovir, gemcitobine, imiquimod, lamivudine, lamotrigine, lenalidomide, mesalazine, methotrexate, metoclopramide, minoxidil, mitomycin, nelerabine, oxybuprocaine, pemetrexed, penciclovir, phenazopyridine, pramipexole, prazosin, procainamide, procaine, proflavine, proparacaine, pyrimethamine, riluzole, S-adenosylmethionine, sparfloxacin, sulfacetamide, sulfanilamide, tacrine, tamibarotene, tenofovir, terazosin, tetrahydrobiopterin, tetrahydrofolic acid, thiamine, thioguanine, triamterene, trimethoprim, tremexate, valaciclovir, valganciclovir, vidarabine, zalcitabine.

166. The composition of claim 164, wherein the therapeutic molecules are formulated with any of the hydrophilic molecules given in claims 125, 129, 132, 135, 138, 142 or 144. These could be formulated with 1, 2, 3, 4, 5, 6, 7, 8, 9, 10 or more different types of molecules in varied molar ratios.

167. The composition of claim 134, wherein the hydrophilic layer is formulated with electrostatic binders from any of the hydrophilic molecules given in claims 128, 129, 132. 135, 138, 142 or 144, plus a lipid selected from the group consisting of amine derivatives of cationic lipids including, but not limited to DOTMA, DOTAP, DOSPA or ePC; Amine derivatives of ionizable lipids including, but not limited to, DLin-MC3-DMA, ALC-0315, Lipid-H (SM-102), A2-Iso5-2DC18, BAME-O16B, 9A1P9, C12-200, cKK-E12, OF-Deg-Lin, 306Oᵢ₁₀, TT3, or FTT5; Or amine derivatives of other types of lipids, including but not limited to, DSPC, PEG₂₀₀₀-DMG, ALC-0159, cholesterol, DC-cholesterol, β-sitosterol and BHEM-cholesterol.
